(19) United States
(12) Patent Application Publication
(10) Pub. No.: US 2026/0263646 A1
(43) Pub. Date: Sep. 10, 2026

(54) CROSSLINKING ANTIBODIES

(71) Applicant: Enlaza Therapeutics, Inc., La Jolla, CA (US)

(72) Inventors: Sergio DURON, La Jolla, CA (US); Jason ROLAND, La Jolla, CA (US); Jerod PTACIN, La Jolla, CA (US); Analeah HEIDT, La Jolla, CA (US); Jun LIU, La Jolla, CA (US)

(21) Appl. No.: 18/723,031

(22) PCT Filed: Dec. 22, 2022
(86) PCT No.: PCT/US2022/082277

§ 371 Date: Jun. 21, 2024

Related U.S. Application Data
(60) Provisional application No. 63/293,025, filed on Dec. 22, 2021.
(60) Provisional application No. 63/346,799, filed on May 27, 2022.
(60) Provisional application No. 63/388,072, filed on Jul. 11, 2022.

(51) Int. Cl. A61K 51/10; A61K 47/54; A61K 47/68; A61K 49/00; A61K 51/04; A61P 35/00

(52) U.S. Cl. CPC A61K 51/1093; A61K 47/542; A61K 47/6849; A61K 47/6851; A61K 47/6853; A61K 47/6855; A61K 47/6865; A61K 47/6869; A61K 49/0002; A61K 51/0497; A61P 35/00

(57) ABSTRACT

Provided herein are compositions and methods for cross-linking single domain antibodies to targets with conjugates. Further provided herein are conjugates comprising targeting domains, wherein the targeting domain comprises an unnatural amino acid. Further provided herein are methods of treating disease with cross-linking single domain and multispecific antibodies.

### CROSS-REFERENCE

This application claims the benefit of U.S. Provisional Patent Application No. 63/293,025 filed on Dec. 22, 2021; U.S. Provisional Patent Application No. 63/346,799 filed on May 27, 2022; and U.S. Provisional Patent Application No. 63/388,072 filed on Jul. 11, 2022, each of which is incorporated by reference in its entirety.

### BACKGROUND

Proteins primarily use non-covalent interactions within or between proteins since amino acid side chains of proteins usually cannot form covalent bonds with each other, except for cysteine which generates relatively weak disulfide bonds that are reversible. Thus, a latent bioreactive unnatural amino acid (UAA) that is able to specifically react with multiple natural amino acid residues on a target protein would expand upon the diversity of proteins amenable to covalent bonding in vivo, which can make possible to enhance existing protein properties or evolve new functions through harnessing the novel covalent linkages. Furthermore, covalent linkages between proteins would allow irreversible capture of protein-protein interactions in vivo, which can be useful for protein identification, drug discovery, irreversible antagonists and payload delivery.

### BRIEF SUMMARY

Provided herein are conjugates comprising a targeting domain and a payload, wherein the targeting domain comprises at least one unnatural amino acid (UAA) residue, wherein the targeting domain is configured to bind to a target, and wherein the UAA residue is in sufficient proximity to form a covalent bond with the target when the targeting domain is bound. Further provided herein are conjugates wherein the payload is attached to an amino acid at position n+x from the UAA residue, wherein n is the position of the amino acid and x is at least 1. Further provided herein are conjugates wherein the payload is attached to an amino acid at position n−x from the UAA residue, wherein n is the position of the amino acid and x is at least 1. Further provided herein are conjugates wherein when the targeting domain is bound to the target, the UAA residue is within 5-20 angstroms of the target. Further provided herein are conjugates wherein the targeting domain binds to a cell surface molecule. Further provided herein are conjugates wherein the UAA residue is configured to form a covalent bond with a histidine, lysine, or tyrosine residue of the target. Further provided herein are conjugates wherein the UAA residue comprises a fluoro sulfate moiety. Further provided herein are conjugates wherein the UAA residue comprises an aryl-fluoro sulfate moiety. Further provided herein are conjugates wherein the UAA residue comprises Formula I:

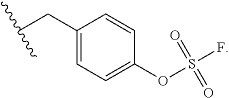 [SMILES: *Cc1ccc(OS(=O)(=O)F)cc1]

Further provided herein are conjugates wherein the UAA of the UAA residue has the structure:

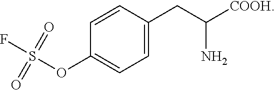 [SMILES: NC(Cc1ccc(OS(=O)(=O)F)cc1)C(=O)O]

Further provided herein are conjugates wherein the UAA of the UAA residue has the structure:

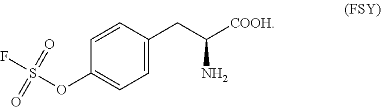 [SMILES: N[C@@H](Cc1ccc(OS(=O)(=O)F)cc1)C(=O)O]

Further provided herein are conjugates wherein the UAA residue comprises Formula II:

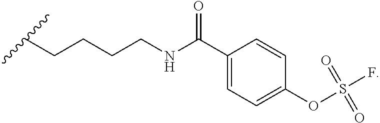 [SMILES: *CCCCNC(=O)c1ccc(OS(=O)(=O)F)cc1]

Further provided herein are conjugates wherein the UAA of the UAA residue has the structure:

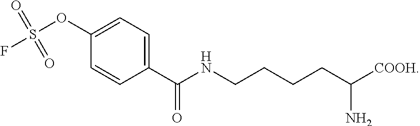 [SMILES: NC(CCCCNC(=O)c1ccc(OS(=O)(=O)F)cc1)C(=O)O]

Further provided herein are conjugates wherein the UAA of the UAA residue has the structure:

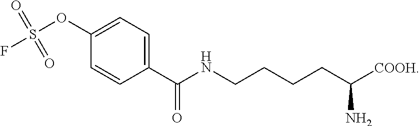 [SMILES: N[C@@H](CCCCNC(=O)c1ccc(OS(=O)(=O)F)cc1)C(=O)O]

Further provided herein are conjugates wherein the UAA residue comprises Formula III:

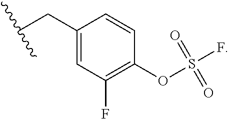 [SMILES: *Cc1ccc(OS(=O)(=O)F)c(F)c1]

Further provided herein are conjugates wherein the UAA of the UAA residue has the structure:

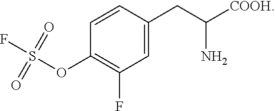 [SMILES: NC(Cc1ccc(OS(=O)(=O)F)c(F)c1)C(=O)O]

Further provided herein are conjugates wherein the UAA of the UAA residue has the structure:

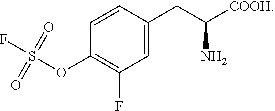 [SMILES: N[C@@H](Cc1ccc(OS(=O)(=O)F)c(F)c1)C(=O)O]

Further provided herein are conjugates wherein the UAA of the UAA residue has a structure of Formula (IA):

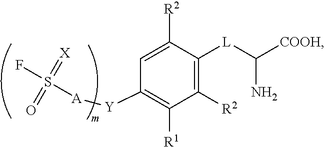

wherein,
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   R¹ is hydrogen, fluoro, or iodo; R² is hydrogen or methyl;
   L is —(CH₂)ₚ— or —C(O)NH—(CH₂)ₚ—; p is an integer of 1 to 6; and
   wherein when Y is —O— or —NR—, m is 1; when Y is —N═, m is 2.

Further provided herein are conjugates wherein the UAA of Formula (IA) has a structure of Formula (IA-a):

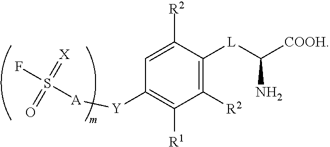

Further provided herein are conjugates wherein the UAA of Formula (IA) has a structure of Formula (IA-b):

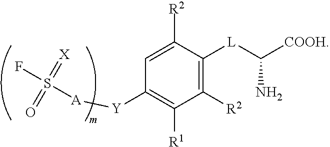

Further provided herein are conjugates wherein the UAA of Formula (IA) has a structure of Formula (IB):

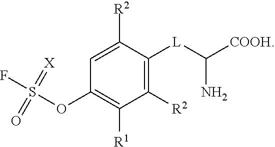

Further provided herein are conjugates wherein the UAA of Formula (IA) has a structure of Formula (IC):

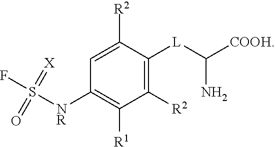

Further provided herein are conjugates wherein the UAA of Formula (IA) has a structure of Formula (ID):

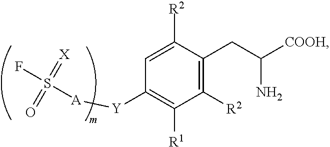

wherein:
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   R¹ is hydrogen, fluoro, or iodo; R² is hydrogen or methyl; and
   wherein when Y is a bond, —O— or —NR—, m is 1; when Y is —N═, m is 2.

Further provided herein are conjugates wherein the UAA of Formula (IA) has a structure of Formula (IE):

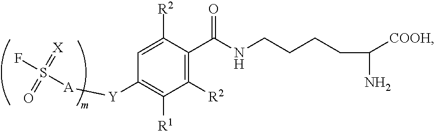

wherein:
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   R¹ is hydrogen, fluoro, or iodo; R² is hydrogen or methyl; and
   wherein when Y is a bond, —O— or —NR—, m is 1; when Y is —N═, m is 2.

Further provided herein are conjugates wherein the UAA of Formula (IA) has a structure of Formula (IIA):

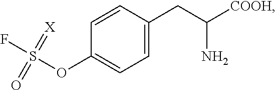

wherein:
   X is independently O or NR′; and
   R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Further provided herein are conjugates wherein the UAA of Formula (IA) has a structure of Formula (IIB):

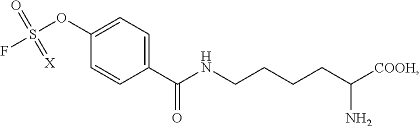

wherein:
   X is independently O or NR′; and
   R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Further provided herein are conjugates wherein the UAA of the UAA residue has a structure of

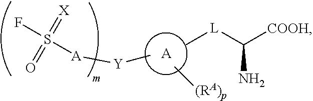

wherein,
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   Ring A is a 5- to 6-membered aryl or heteroaryl;
   each R^{A} is independently —OH, —OR^{X}, halogen, NHR^{X}, N(R^{X})₂, or optionally substituted alkyl; each R^{X} is optionally substituted alkyl;
   L is —(CH₂)ₚ— or —C(O)NH—(CH₂)ₚ—; p is an integer of 1 to 6; and
   wherein when Y is a bond, —O— or —NR—, m is 1; when Y is —N═, m is 2.

Further provided herein are conjugates wherein the payload comprises an imaging agent, a radioligand agent or a cytotoxic agent. Further provided herein are conjugates wherein the cytotoxic moiety comprises a small molecule drug, or a chemotherapeutic drug. Further provided herein are conjugates wherein the radioligand agent is selected from the group consisting of ¹⁵³Sm, ¹⁷⁷Lu, ⁹⁰Y, ¹³¹I, ¹⁴⁹Tb, ²¹¹At, ²¹²Pb/²¹²Bi, ²¹³Bi, ²²³Ra, ²²⁵Ac, and ²²⁷Th. Further provided herein are conjugates wherein the radioligand agent further comprises a chelator. Further provided herein are conjugates wherein the radioligand agent is selected from the group consisting of ^{99m}Tc, ¹³¹I, ²⁰¹Tl, ¹¹¹In, and ⁶⁷Ga. Further provided herein are conjugates wherein the payload is attached to the conjugate with a linker. Further provided herein are conjugates wherein the linker comprises a polymer. Further provided herein are conjugates wherein the linker is a cleavable or non-cleavable linker. Further provided herein are conjugates wherein the linker is 0.01 kDa to 2.5 kDa. Further provided herein are conjugates wherein the linker is 0.01 kDa to 2.5 kDa. Further provided herein are conjugates wherein the linker is linear, branched, multimeric, or dendrimeric. Further provided herein are conjugates wherein the linker is a bifunctional or multifunctional linker or a bifunctional or multifunctional polymer. Further provided herein are conjugates wherein the linker comprises a water soluble polymer. Further provided herein are conjugates wherein the water soluble polymer is polyethylene glycol (PEG). Further provided herein are conjugates wherein the PEG has a molecular weight between 0.1 kDa and 2.5 kDa. Further provided herein are conjugates wherein the PEG comprises 1-8 monomers. Further provided herein are conjugates wherein the targeting domain comprises an antibody, antibody fragment, or an antigen binding domain. Further provided herein are conjugates wherein the targeting domain comprises an antigen binding domain, the conjugate comprises a CDR region, and the at least one UAA residue is within or in the proximity of the CDR region. Further provided herein are conjugates wherein the UAA residue is comprised within the CDR region. Further provided herein are conjugates wherein the targeting domain comprises a single domain antibody (sdAb). Further provided herein are conjugates wherein the cell-surface molecule is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM.

Also provided herein are conjugates comprising (i) a payload and (ii) an engineered single domain antibody (sdAb), comprising a sdAb having a CDR region and at least one unnatural amino acid (UAA) residue within or in the proximity of the CDR region, wherein the sdAb comprises any one of SEQ ID NOS: 1-4 or 16-64. Further provided herein are conjugates wherein the UAA residue comprises a fluorosulfate moiety. Further provided herein are conjugates wherein the UAA residue comprises an aryl-fluoro sulfate moiety. Further provided herein are conjugates wherein the UAA residue comprises Formula I:

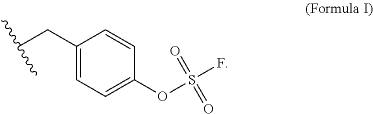 [SMILES: *Cc1ccc(OS(=O)(=O)F)cc1]

Further provided herein are conjugates wherein the UAA of the UAA residue has the structure:

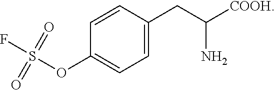 [SMILES: NC(Cc1ccc(OS(=O)(=O)F)cc1)C(=O)O]

Further provided herein are conjugates wherein the UAA of the UAA residue has the structure:

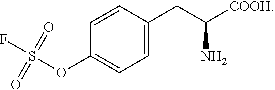 [SMILES: N[C@@H](Cc1ccc(OS(=O)(=O)F)cc1)C(=O)O]

Further provided herein are conjugates wherein the UAA residue comprises Formula II.

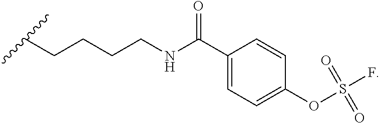 [SMILES: *CCCCNC(=O)c1ccc(OS(=O)(=O)F)cc1]

Further provided herein are conjugates wherein the UAA of the UAA residue has the structure:

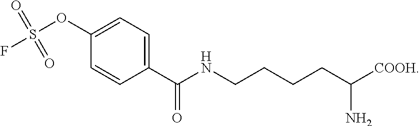 [SMILES: NC(CCCCNC(=O)c1ccc(OS(=O)(=O)F)cc1)C(=O)O]

Further provided herein are conjugates wherein the UAA has the structure:

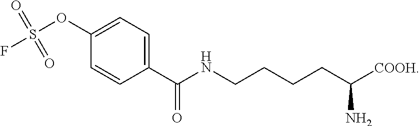 [SMILES: N[C@@H](CCCCNC(=O)c1ccc(OS(=O)(=O)F)cc1)C(=O)O]

Further provided herein are conjugates wherein the UAA residue comprises Formula III:

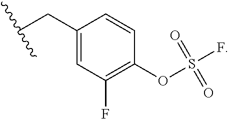 [SMILES: *Cc1ccc(OS(=O)(=O)F)c(F)c1]

Further provided herein are conjugates wherein the UAA of the UAA residue has the structure:

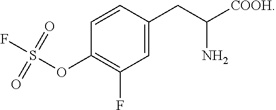 [SMILES: NC(Cc1ccc(OS(=O)(=O)F)c(F)c1)C(=O)O]

Further provided herein are conjugates wherein the UAA of the UAA residue has the structure:

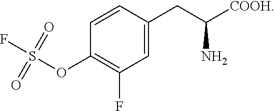 [SMILES: N[C@@H](Cc1ccc(OS(=O)(=O)F)c(F)c1)C(=O)O]

Further provided herein are conjugates wherein the UAA of the UAA residue has a structure of Formula (IA):

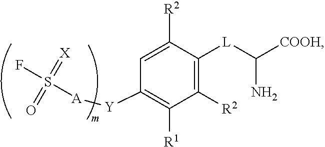

wherein,
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   R¹ is hydrogen, fluoro, or iodo; R² is hydrogen or methyl;
   L is —(CH₂)ₚ— or —C(O)NH—(CH₂)ₚ—; p is an integer of 1 to 6; and
   wherein when Y is —O— or —NR—, m is 1; when Y is —N═, m is 2.

Further provided herein are conjugates wherein the UAA of Formula (IA) has a structure of Formula (IA-a):

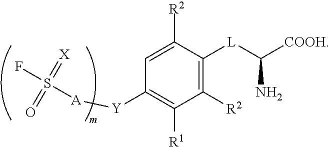

Further provided herein are conjugates wherein the UAA of Formula (IA) has a structure of Formula (IA-b):

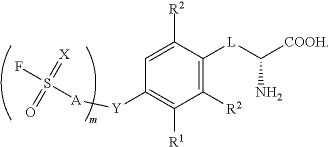

Further provided herein conjugates wherein the UAA of Formula (IA) has a structure of Formula (IB):

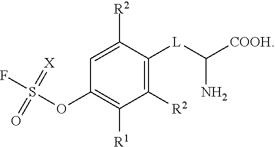

Further provided herein are conjugates wherein the UAA of Formula (IA) has a structure of Formula (IC):

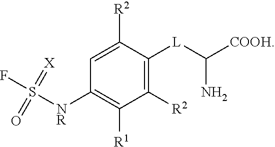

Further provided herein are conjugates wherein the UAA of Formula (IA) has a structure of Formula (ID):

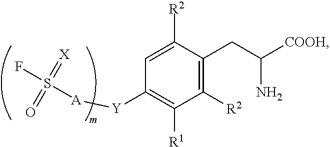

wherein:
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   R¹ is hydrogen, fluoro, or iodo; R² is hydrogen or methyl; and
   wherein when Y is a bond, —O— or —NR—, m is 1; when Y is —N═, m is 2.

Further provided herein are conjugates wherein the UAA of Formula (IA) has a structure of Formula (IE):

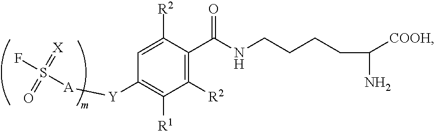

wherein:
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   R¹ is hydrogen, fluoro, or iodo; R² is hydrogen or methyl; and
   wherein when Y is a bond, —O— or —NR—, m is 1; when Y is —N═, m is 2.

Further provided herein are engineered sdAbs wherein the UAA of Formula (IA) has a structure of Formula (IIA):

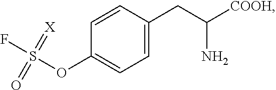

wherein:
   X is independently O or NR′; and
   R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Further provided herein are engineered sdAbs wherein the UAA of Formula (IA) has a structure of Formula (IIB):

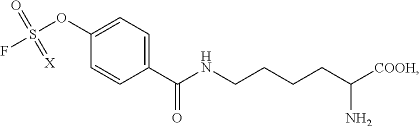

wherein:
   X is independently O or NR′; and
   R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Further provided herein are conjugates wherein the UAA of the UAA residue has a structure of

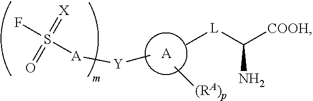

wherein,
   each X is independently O or NR′;
   Y is a bond, —O—, —NR—, or —N═;
   A is a bond or —(CH₂)ₙ—; m is 1 or 2; n is an integer of 1 to 4;
   each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
   Ring A is a 5- to 6-membered aryl or heteroaryl;
   each R^{A} is independently —OH, —OR^{X}, halogen, NHR^{X}, N(R^{X})₂, or optionally substituted alkyl; each R^{X} is optionally substituted alkyl;
   L is —(CH₂)ₚ— or —C(O)NH—(CH₂)ₚ—; p is an integer of 1 to 6; and
   wherein when Y is a bond, —O— or —NR—, m is 1; when Y is —N═, m is 2.

Further provided herein are conjugates wherein the engineered sdAb comprises SEQ ID NO:1, and wherein the UAA residue is comprised in any one of SEQ ID NOS: 5, 6, and 7. Further provided herein are conjugates wherein the UAA residue is present at an amino acid position selected from the group consisting of 26, 28, 29, 30, 99, 102, 103, 105, 108, 110, 111, 112, 113, 114, and 115 relative to SEQ ID NO:1. Further provided herein are conjugates wherein the engineered sdAb comprises SEQ ID NO:2, and wherein the UAA residue is comprised in any one of SEQ ID NOS:8, 9, and 10. Further provided herein are conjugates wherein the UAA residue is present at an amino acid position selected from the group consisting of 50, 52, 53, 54, 56, 58, and 100 relative to SEQ ID NO:2. Further provided herein are conjugates wherein the engineered sdAb comprises SEQ ID NO:3, and wherein the UAA residue is comprised in any one of SEQ ID NOS: 11, 12, and 13. Further provided herein are conjugates wherein the UAA residue is present at an amino acid position selected from the group consisting of 58, 62, 101, 103, and 107 relative to SEQ ID NO:3. Further provided herein are conjugates wherein the engineered sdAb comprises SEQ ID NO: 16, and wherein the UAA is present at an amino acid position of 109 relative to SEQ ID NO: 16. Further provided herein are conjugates wherein the engineered sdAb comprises SEQ ID NO:23 and wherein the UAA is present at an amino acid position selected from the group consisting of 52, 53, 54, 55, 56, 58, 60, 62, and 64 relative to SEQ ID NO: 23. Further provided herein are conjugates wherein the engineered sdAb comprises SEQ ID NO:24 and wherein the UAA is present at an amino acid position selected from the group consisting of 53, 55, 56, 57, 58, 60, 64, and 67 relative to SEQ ID NO: 24.

Further provided herein are conjugates wherein the payload is not linked via the UAA residue sidechain. Further provided herein are conjugates wherein the payload comprises an imaging agent, a radioligand agent or a cytotoxic agent. Further provided herein are conjugates wherein the cytotoxic moiety comprises a small molecule drug or a chemotherapeutic drug. Further provided herein are conjugates wherein the radioligand agent is selected from the group consisting of ¹⁵³Sm, ¹⁷Lu, ⁹⁰Y, ¹³¹I, ¹⁴⁹Tb, ²¹¹At, ²¹²Pb/²¹²Bi, ²¹³Bi, ²²³Ra, ²²⁵Ac, and ²²⁷Th. Further provided herein are conjugates wherein the radioligand agent further comprises a chelator. Further provided herein are conjugates wherein the imaging agent comprises a fluorophore, or a radioligand agent selected from the group consisting of ^{99m}Tc, ¹³¹I, ²⁰¹Tl, ¹¹¹In, and ⁶⁷Ga.

Provided herein are methods comprising administering a conjugate described herein, wherein the conjugate covalently binds a target on the surface of a cell. Further provided herein are methods wherein the cell comprises a tumor cell. Further provided herein are methods wherein the conjugate kills or inhibits the growth of the tumor cell. Further provided herein are methods wherein the target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Muc1, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. Provided herein are methods of treating a disease or condition comprising administering a conjugate described herein. Further provided herein are methods wherein the disease comprises one or more of PCa (prostate cancer), CRPCa (castration resistant prostate cancer), solid tumors (neovasculature), NSCLC (non-small cell lung cancer), HNSCC (head and neck squamous cell carcinoma), ESCC (esophageal cancer) GC (gastric cancer), CRC (colorectal cancer), SCLC (small cell lung cancer), MPM (mesothelioma), PDAC (Pancreatic ductal adenocarcinoma), ALL (Acute Lymphoblastic Leukemia), AML (Acute Myeloid Leukemia), MDS (Myelodysplastic syndromes), MSI-high tumors, melanoma, DLBCL (diffuse large B cell lymphoma), endometrial cancer, cervical cancer, bladder cancer, BrCa (breast cancer), TNBC (triple negative breast cancer), NE-PCa (Neuroendocrine prostate cancer), GBM (glioblastoma), and RCC (Renal cell carcinoma).

Provided herein are methods of manufacturing a conjugate described herein, comprising: generating the targeting domain comprising at least one unnatural amino acid; and conjugating the targeting domain to a payload, optionally via a linker. Further provided herein are methods wherein the targeting domain comprising at least one unnatural amino acid is synthesized in-vivo. Further provided herein are methods wherein generating comprises use of an orthogonal tRNA synthetase/suppressor tRNA pair. Further provided herein are methods wherein generating comprises the orthogonal tRNA synthetase/suppressor tRNA pair is derived from pyrrolysine tRNA synthetase/tRNA^{Pyl}. Further provided herein are methods wherein the orthogonal tRNA synthetase comprises SEQ ID NOs: 84, 87, 92, or variant thereof.

Provided herein are methods of delivering a cytotoxic payload to a cell comprising administering a conjugate described herein, wherein the conjugate covalently binds a target on the surface of the cell, thereby delivering the cytotoxic payload. Further provided herein are methods wherein the cell is a tumor cell. Further provided herein are methods wherein the cell is comprised within a tumor microenvironment. Further provided herein are methods wherein the cell is comprised within a mammalian subject. Further provided herein are methods wherein the cell is comprised within a human subject. Further provided herein are methods wherein the conjugate kills or inhibits the growth of the tumor cell. Further provided herein are methods wherein the target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Muc1, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. Further provided herein are methods wherein the human subject suffers from or is diagnosed with a disease or condition selected from the group consisting of PCa (prostate cancer), CRPCa (castration resistant prostate cancer), solid tumors (neovasculature), NSCLC (non-small cell lung cancer), HNSCC (head and neck squamous cell carcinoma), ESCC (esophageal cancer) GC (gastric cancer), CRC (colorectal cancer), SCLC (small cell lung cancer), MPM (mesothelioma), PDAC (Pancreatic ductal adenocarcinoma), ALL (Acute Lymphoblastic Leukemia), AML (Acute Myeloid Leukemia), MDS (Myelodysplastic syndromes), MSI-high tumors, melanoma, DLBCL (diffuse large B cell lymphoma), endometrial cancer, cervical cancer, bladder cancer, BrCa (breast cancer), TNBC (triple negative breast cancer), NE-PCa (Neuroendocrine prostate cancer), GBM (glioblastoma), and RCC (Renal cell carcinoma).

Provided herein are conjugates comprising (i) a first targeting domain, (ii) a second targeting domain, and (iii) a payload, wherein the first targeting domain comprises at least one first unnatural amino acid (UAA) whereby the first targeting domain is capable of covalently binding to a first target at the site of the UAA and the second targeting domain is configured to bind a second target. In some embodiments, the first target and the second target are on the same cell. In some embodiments, the first targeting domain comprises an antibody, antibody fragment, or an antigen binding domain. In some embodiments, the first targeting domain comprises a single domain antibody (sdAb). In some embodiments, the first UAA is comprised within or within proximity of a region of the first targeting domain that interfaces with the first target. In some embodiments, the second targeting domain comprises an antibody, antibody fragment, or an antigen binding domain. In some embodiments, the second targeting domain comprises a single domain antibody (sdAb). In some embodiments, the first targeting domain and the second targeting domain are linked to form a fusion protein. In some embodiments, the first targeting domain and the second targeting domain are linked by chemical conjugation. In some embodiments, the first targeting domain and the second domain are linked by a linker. In some embodiments, the first targeting domain and the second targeting domain bind the same target. In some embodiments, the first targeting domain and the second targeting domain bind to different epitopes of the same target. In some embodiments, the first targeting domain and the second targeting domain bind to the same epitope of the same target. In some embodiments, the same target is a monomer. In some embodiments, the same target is a multimeric molecule. In some embodiments, the first targeting domain and the second targeting domain bind different targets. In some embodiments, the first target is a first cell surface molecule. In some embodiments, the second target is a second cell surface molecule. In some embodiments, the at least one first UAA comprises a fluoro sulfate moiety. In some embodiments, the at least one UAA comprises an aryl-fluoro sulfate moiety. In some embodiments, the at least one first UAA comprises Formula I:

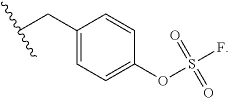 [SMILES: *Cc1ccc(OS(=O)(=O)F)cc1]

In some embodiments, the at least one first UAA has the structure:

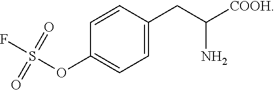 [SMILES: NC(Cc1ccc(OS(=O)(=O)F)cc1)C(=O)O]

In some embodiments, the at least one first UAA has the structure:

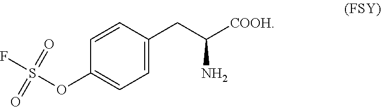 [SMILES: N[C@@H](Cc1ccc(OS(=O)(=O)F)cc1)C(=O)O]

In some embodiments, the at least one first UAA comprises Formula II:

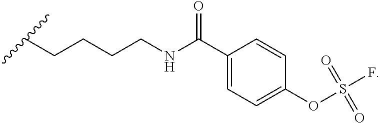 [SMILES: *CCCCNC(=O)c1ccc(OS(=O)(=O)F)cc1]

In some embodiments, the at least one first UAA has the structure:

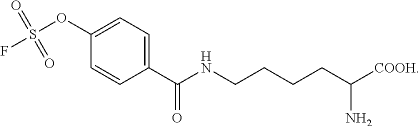 [SMILES: NC(CCCCNC(=O)c1ccc(OS(=O)(=O)F)cc1)C(=O)O]

In some embodiments, the at least one first UAA has the structure:

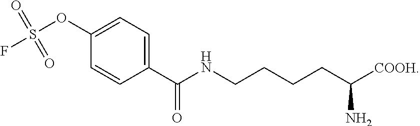 [SMILES: N[C@@H](CCCCNC(=O)c1ccc(OS(=O)(=O)F)cc1)C(=O)O]

In some embodiments, the at least one first UAA comprises Formula III:

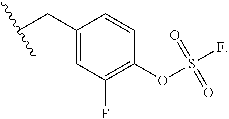 [SMILES: *Cc1ccc(OS(=O)(=O)F)c(F)c1]

In some embodiments, the at least one first UAA has the structure:

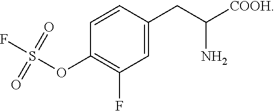 [SMILES: NC(Cc1ccc(OS(=O)(=O)F)c(F)c1)C(=O)O]

In some embodiments, the at least one first UAA has the structure:

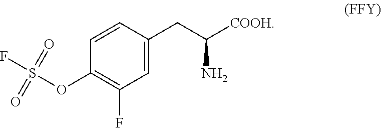 [SMILES: N[C@@H](Cc1ccc(OS(=O)(=O)F)c(F)c1)C(=O)O]

In some embodiments, the at least one first UAA has a structure of Formula (IA):

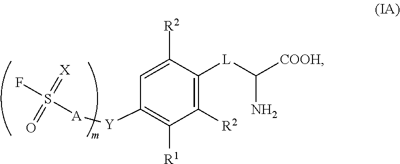

wherein, each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; L is —(CH₂)ₚ— or —C(O)NH—(CH₂)ₚ—; p is an integer of 1 to 6; and wherein when Y is —O— or —NR—, m is 1; when Y is —N═, m is 2. In some embodiments, the at least one first UAA has a structure of Formula (IA-a):

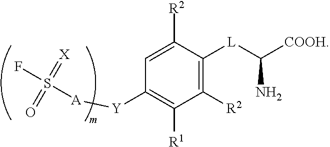

In some embodiments, the at least one first UAA has a structure of Formula (IA-b):

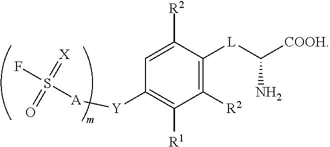

In some embodiments, the at least one first UAA has a structure of Formula (IB):

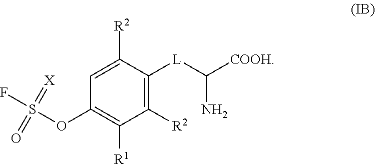

In some embodiments, the at least one first UAA has a structure of Formula (IC):

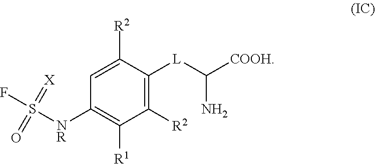

In some embodiments, the at least one first UAA has a structure of Formula (ID):

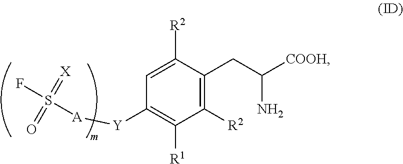

wherein: each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; and wherein when Y is a bond, —O— or —NR—, m is 1; when Y is —N═, m is 2. In some embodiments, the at least one first UAA has a structure of Formula (IE):

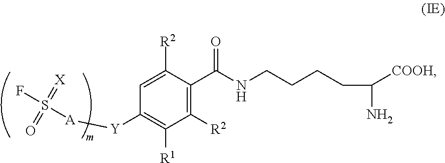

wherein: each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; and wherein when Y is a bond, —O— or —NR—, m is 1; when Y is —N═, m is 2. In some embodiments, the at least one first UAA has a structure of Formula (IIA):

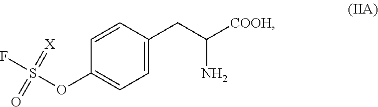

wherein: X is independently O or NR′; and R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, the at least one first UAA has a structure of Formula (IIB):

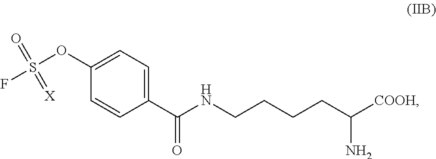

wherein: X is independently O or NR′; and R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, the at least one first UAA has a structure of

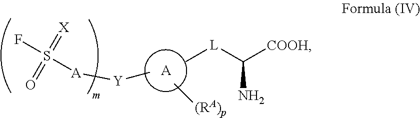

wherein, each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; Ring A is a 5- to 6-membered aryl or heteroaryl; each R^{A} is independently —OH, —OR^{X}, halogen, NHR^{X}, N(R^{X})₂, or optionally substituted alkyl; p is 0, 1, 2, 3 or 4; each R^{X} is optionally substituted alkyl; L is —(CH₂)ₚ— or —C(O)NH—(CH₂)ₚ—; p is an integer of 1 to 6; and wherein when Y is a bond, —O— or —NR—, m is 1; when Y is —N═, m is 2. In some embodiments, the first cell surface molecule is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Muc1, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some embodiments, the second cell surface molecule is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Muc1, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some embodiments, the second domain comprises a second UAA, whereby the second domain is capable of covalently binding to the second target at the site of the second UAA. In some embodiments, the second UAA is different from the at least one UAA comprised in the first targeting domain. In some embodiments, the second UAA is the same as the at least one UAA comprised in the first targeting domain. In some embodiments, the second UAA comprises a fluoro sulfate moiety. In some embodiments, the second UAA comprises an aryl-fluoro sulfate moiety. In some embodiments, the second UAA comprises Formula I:

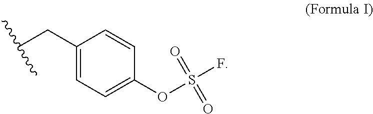 [SMILES: *Cc1ccc(OS(=O)(=O)F)cc1]

In some embodiments, the second UAA has the structure:

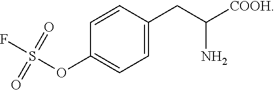 [SMILES: NC(Cc1ccc(OS(=O)(=O)F)cc1)C(=O)O]

In some embodiments, the second UAA has the structure:

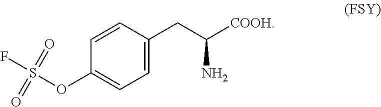 [SMILES: N[C@@H](Cc1ccc(OS(=O)(=O)F)cc1)C(=O)O]

In some embodiments, the second UAA comprises Formula II:

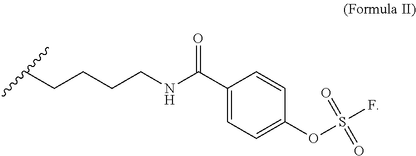 [SMILES: *CCCCNC(=O)c1ccc(OS(=O)(=O)F)cc1]

In some embodiments, the second UAA has the structure:

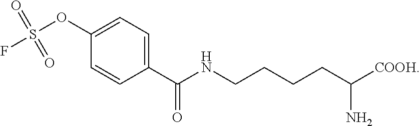 [SMILES: NC(CCCCNC(=O)c1ccc(OS(=O)(=O)F)cc1)C(=O)O]

In some embodiments, the second UAA has the structure:

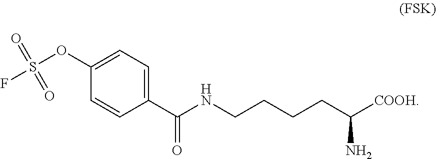 [SMILES: N[C@@H](CCCCNC(=O)c1ccc(OS(=O)(=O)F)cc1)C(=O)O]

In some embodiments, the second UAA comprises Formula III:

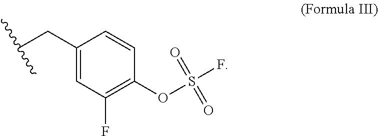 [SMILES: *Cc1ccc(OS(=O)(=O)F)c(F)c1]

In some embodiments, the second UAA has the structure:

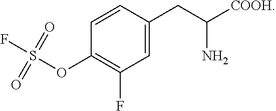 [SMILES: NC(Cc1ccc(OS(=O)(=O)F)c(F)c1)C(=O)O]

In some embodiments, the second UAA has the structure:

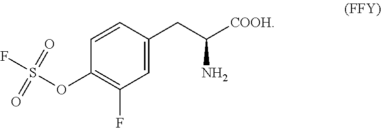 [SMILES: N[C@@H](Cc1ccc(OS(=O)(=O)F)c(F)c1)C(=O)O]

In some embodiments, the second UAA has a structure of Formula (IA):

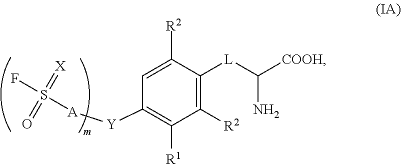

wherein, each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; L is —(CH₂)ₚ— or —C(O)NH—(CH₂)ₚ—; p is an integer of 1 to 6; and wherein when Y is —O— or —NR—, m is 1; when Y is —N═, m is 2. In some embodiments, the second UAA has a structure of Formula (IA-a):

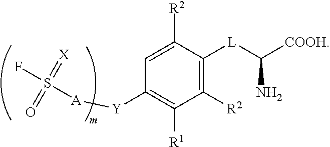

In some embodiments, the second UAA has a structure of Formula (IA-b):

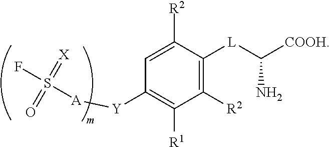

In some embodiments, the second UAA has a structure of Formula (IB):

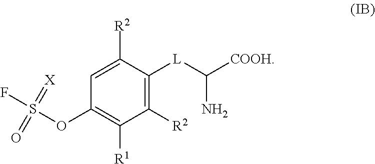

In some embodiments, the second UAA has a structure of Formula (IC):

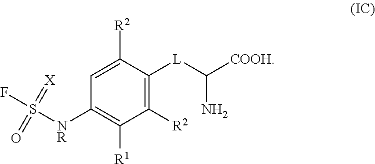

In some embodiments, the second UAA has a structure of Formula (ID):

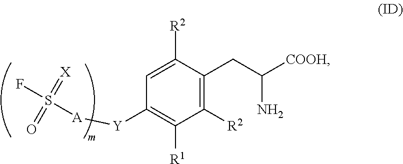

wherein: each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; and wherein when Y is a bond, —O— or —NR—, m is 1; when Y is —N═, m is 2. In some embodiments, the second UAA has a structure of Formula (IE):

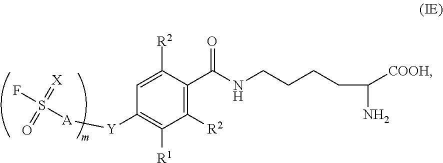

wherein: each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; and wherein when Y is a bond, —O— or —NR—, m is 1; when Y is —N═, m is 2. In some embodiments, the second UAA has a structure of Formula (IIA):

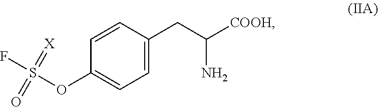

wherein: X is independently O or NR′; and R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, the second UAA has a structure of Formula (IIB):

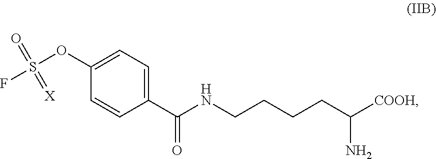

wherein: X is independently O or NR′; and R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, the second UAA has a structure of

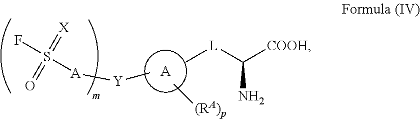

wherein, each X is independently O or NR′; Y is a bond, —O—, —NR—, or —N═; A is a bond or —(CH₂)ₙ—; m is 1 or 2; n is an integer of 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; Ring A is a 5- to 6-membered aryl or heteroaryl; each R^{A} is independently —OH, —OR^{X}, halogen, NHR^{X}, N(R^{X})₂, or optionally substituted alkyl; p is 0, 1, 2, 3 or 4; each R^{X} is optionally substituted alkyl; L is —(CH₂)ₚ— or —C(O)NH—(CH₂)ₚ—; p is an integer of 1 to 6; and wherein when Y is a bond, —O— or —NR—, m is 1; when Y is —N═, m is 2. In some embodiments, the first targeting domain comprises any one of SEQ ID NOS: 1-4, or 16-64. In some embodiments, the first targeting domain comprises a sequence having at least 70% sequence identity to any one of SEQ ID NOS: 1-4, or 16-64. In some embodiments, the second targeting domain comprises any one of SEQ ID NOS: SEQ ID NOS: 1-4, or 16-64. In some embodiments, the second targeting domain comprises a sequence having at least 70% identity to any one of SEQ ID NOs: 1-4, or 16-64. In some embodiments, the conjugate comprises SEQ ID NO: 66-72. In some embodiments, the conjugate comprises a sequence having at least 70% sequence identity to SEQ ID NO: 66-72. In some embodiments, the conjugate comprises any one of SEQ ID NOS: 1-4, or 16-64. In some embodiments, the conjugate comprises a sequence having at least 70% sequence identity to any one of SEQ ID NOs: 1-4, or 16-64. In some embodiments, the conjugate comprises a sequence having at least 70% sequence identity to SEQ ID NOs: 66-72. In some embodiments, at least one of the first targeting domain and the second targeting domain comprise SEQ ID NO: 1. In some embodiments, the UAA is present at an amino acid position selected from the group consisting of 26, 28, 29, 30, 99, 102, 103, 105, 108, 110, 111, 112, 113, 114, and 115 relative to SEQ ID NO:1. In some embodiments, at least one of the first targeting domain and the second targeting domain comprise SEQ ID NO: 2. In some embodiments, the UAA is present at an amino acid position selected from the group consisting of 50, 52, 53, 54, 56, 58, and 100 relative to SEQ ID NO:2. In some embodiments, at least one of the first targeting domain and the second targeting domain comprise SEQ ID NO: 3. In some embodiments, the UAA is present at an amino acid position selected from the group consisting of 58, 62, 101, 103, and 107 relative to SEQ ID NO:3. In some embodiments, at least one of the first targeting domain and the second targeting domain comprise SEQ ID NO: 16. In some embodiments, the at least one of the first targeting domain and the second targeting domain comprising SEQ ID NO: 16 further comprises an unnatural amino acid at position 109 relative to SEQ ID NO: 16. In some embodiments, the at least one of the first targeting domain and the second targeting domain comprise SEQ ID NO: 18. In some embodiments, the payload comprises an imaging agent, a radioligand agent or a cytotoxic agent. In some embodiments, the cytotoxic moiety comprises a small molecule drug, or a chemotherapeutic drug. In some embodiments, the radioligand agent is selected from the group consisting of ¹⁵³Sm, ¹⁷Lu, ⁹⁰Y, ¹³¹I, ¹⁴⁹Tb, ²¹¹At, ²¹²Pb/²¹²Bi, ²¹³Bi, ²²³Ra, ²²⁵Ac, and ²²⁷Th. In some embodiments, the radioligand agent further comprises a chelator. In some embodiments, the radioligand agent is selected from the group consisting of ^{99m}Tc, ¹³¹I, ²⁰¹Tl, ¹¹¹In, and ⁶⁷Ga. In some embodiments, the payload is attached to the conjugate with a linker. In another aspect, provided herein are methods comprising administering the conjugates provided herein, wherein the conjugates covalently binds the first target on the surface of a first cell. In some embodiments, the conjugate binds the second target on the surface of the first cell. In some embodiments, the first targeting domain and the second targeting domain bind to the same target. In some embodiments, the first targeting domain and the second targeting domain bind to the same epitope of the same target. In some embodiments, the first targeting domain and the second targeting domain bind to different epitopes of the same target. In some embodiments, the first targeting domain and the second targeting domain bind to different targets on the surface of the first cell. In some embodiments, the second domain comprises a second UAA, and wherein the second UAA covalently binds to the second target. In some embodiments, the first cell is a tumor cell. In some embodiments, the conjugate kills or inhibits the growth of the tumor cell when bound to the first target. In some embodiments, the conjugate kills or inhibits the growth of the tumor cell when bound to the second target. In some embodiments, the conjugate kills or inhibits the growth of the tumor cell when bound to the first target and the second target. In some embodiments, the first target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some embodiments, the second target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some embodiments, the payload is a radiolabel agent or a cytotoxic agent. In some embodiments, the payload is an imaging agent. In some embodiments, the method images or identifies the first cell when the conjugate is bound to the first target and the second target on the first cell. In yet another aspect, provided herein are methods of manufacturing the conjugates provided herein comprising: (a) synthesizing the first targeting domain comprising at least one unnatural amino acid in vivo; and (b) conjugating the payload to the first targeting domain or the second targeting domain, optionally via a linker. In some embodiments, the method further comprising synthesizing the second targeting domain in vivo as a fusion protein to the first targeting domain. In some embodiments, synthesizing comprises use of an orthogonal tRNA synthetase/suppressor tRNA pair. In some embodiments, synthesizing comprises the orthogonal tRNA synthetase/suppressor tRNA pair is derived from pyrrolysine tRNA synthetase/tRNA^{Pyl}.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1E show various SDS-PAGE analysis of crosslinking between FSY-modified sdAbs with FSY at different positions and PSMA with a molar ratio of approximately 7:1 sdAb to PSMA (PSMA final concentration was 0.125 mg/mL, 1.25 uM) at 37° C. FIG. 1A depicts a gel showing C1 constructs comprising FSY in the presence or absence of target PSMA. PSMA-C1-FSY crosslinking, PSMA, and C1-FSY are labeled. FIG. 1B depicts a gel showing C1 constructs comprising FSY in the presence or absence of target PSMA. PSMA-C1-FSY crosslinking, PSMA, and C1-FSY are labeled. FIG. 1C depicts a gel showing C1 constructs comprising FSY in the presence or absence of target PSMA. PSMA-C1-FSYcrosslinking, PSMA, and C1-FSY are labeled. FIG. 1D depicts a gel showing C1 constructs comprising FSY in the presence or absence of target PSMA. PSMA-C1-FSYcrosslinking, PSMA, and C1-FSY are labeled. FIG. 1E depicts a gel showing C1 constructs comprising FSY in the presence or absence of target PSMA. PSMA-C1-FSYcrosslinking, PSMA, and C1-FSY are labeled.

FIGS. 2A-2C show various SDS-PAGE analysis of crosslinking between C2 (a PSMA-specific sdAb) and PSMA with a molar ratio of 8:1 sdAb:PSMA at 37° C. FIG. 2A depicts a gel showing C2 constructs comprising FSY at positions 50-61 in the presence of target PSMA, as well as PSMA and wild type C2-CDR2 pool controls. Thick arrows indicate the presence of cross-linked products. PSMA and C2 are labeled with thin arrows. FIG. 2B depicts a gel showing C2 constructs comprising FSY in the presence of target PSMA. FIG. 2C depicts a gel showing C2 constructs comprising FSY in the presence of target PSMA, as well as wild type C2-CDR3 pool control. Thick arrows indicate the presence of cross-linked products. PSMA and C2-FSY are labeled with thin arrows.

FIG. 3A shows the SDS-PAGE analysis of the kinetics of crosslinking between FSY-modified sdAbs (FSY at positions 28 or 102 of C1) and PSMA over 180 minutes at 37° C., with a molar ratio of 5:1 sdAbs to PSMA (PSMA final concentration was 0.125 mg/mL, 1.25 uM).

FIG. 3B shows a kinetic plot of PSMA crosslinking percentage vs. time for the crosslinking experiment between FSY-modified sdAbs and PSMA. The y-axis is labeled % of PSMA crosslinked (0 to 60 at 10 unit intervals), and the x-axis is labeled Time (min, 0 to 180 at 30 mins intervals). Constructs tested were C1-28FSY (circles); C1-102FSY (squares); C1-112FSY (triangle); C1-113FSY (inverted triangle).

FIG. 4 shows a diagram of monomeric C1-related sdAb constructs. C1 (wild type sequence comprising two cysteine disulfide linkages); C39 (C1-C101A/C104A, one cysteine disulfide linkage is replaced with two alanines, removing the disulfide linkage); C1-102FSY (construct C1 with histidine 102 replaced with unnatural amino acid FSY); and C39-102FSY (C1-C101A/C104A/H102(FSY), replacement of both C101 and C104 cysteines with alanine, and histidine 102 with FSY).

FIG. 5A shows the SDS-PAGE analysis of the kinetics of crosslinking between either a biparatopic construct C40-102FSY (created by linking the C39-102FSY sequence with a copy of C39 without FSY), or a monoparatopic construct of C39-102FSY with PSMA over 180 minutes at 37° C. Bands corresponding to the C39-102FSY, C40-102FSY, receptor, and crosslink species are labeled. The coupling rate is increased for the biparatopic construct as compared to the monoparatopic construct.

FIG. 5B shows a kinetic plot of PSMA crosslinking vs. time for the crosslinking between either a biparatopic construct C40-102FSY or a monoparatopic construct C39-102FSY with PSMA.

FIGS. 6A and 6B show SDS-PAGE analyses of crosslinking between various FSY-modified C3 constructs. FIG. 6A shows results of C3 constructs with FSY at positions 26-35, and 50-60; the crosslinked product for construct C3-58FSY is indicated with an asterisk. FIG. 6B shows results of C3 constructs with FSY at positions 61-66, and 99-113); crosslinked products were observed at least for construct C3-62FSY, C3-101FSY, C3-103FSY, C3-107FSY.

FIG. 7A shows SDS-PAGE analyses of crosslinking between various pooled FSK-modified C8 constructs and FAP receptor. FIG. 7B shows SDS-PAGE analyses of crosslinking between various individual FSK-modified C8 constructs and FAP receptor.

FIG. 7C shows the SDS-PAGE analysis of the kinetics of crosslinking between various FSK modified C8 constructs and FAP receptor.

FIG. 8A-8D shows SDS-PAGE analyses of crosslinking between various FSY-modified C9 constructs and the Her3 receptor. FIG. 8A shows results of pools of C9 constructs with FSY at various positions and the Her3 receptor. FIG. 8B shows results of C9 constructs with FSY at positions 52-68 and the Her3 receptor. FIG. 8C shows results of C9 constructs with FSY at position 53, 55, 56, 57, 58, 60, 64 or 67 and the Her3 receptor. FIG. 8D shows the SDS-PAGE analysis of the kinetics of crosslinking between FSY modified C9 construct (C9-55FSY) and Her3 receptor.

FIG. 9 shows results of binding assays of C2-54FSY and C2-54TYR sdAbs to human prostate tumor cell lines LNCaP (PSMA+) and PC3 (PSMA−) using flow cytometry.

FIGS. 10A and 10B show SDS-PAGE analyses of FSY modified sdAbs crosslinking to cells. FIG. 10A shows a western blot using LNCaP cells and C2-54 FSY incubated at different concentrations and time points. Blots are analyzed using anti-PSMA antibody (top panel), anti-sdAb antibody (middle panel), and anti-GAPDH (bottom panel) as a loading control. FIG. 10B shows a western blot using LNCaP cells and C2-54TYR and showed no crosslinking with PSMA. The rightmost lane is a control showing a sample of C2-54FSY incubated at 1 uM with LNCaP cells for 9 hours. FIG. 10C shows a graph of the kinetics of crosslinking at various concentrations of C2-54FSY at different time points.

FIG. 11A shows western blot analysis of crosslinking kinetics of C2-54FSY and C3-101FSY at 1 uM and 24 nM with PSMA in LNCaP cells for different times. The top panel shows the blot with anti-PSMA antibody, while the bottom panel shows the anti-GAPDH control. FIG. 11B shows a graph of the crosslinking kinetics of C2-54FSY and C3-101FSY to PSMA on LNCaP cells.

FIGS. 12A and 12B show the study design and results of in vivo crosslinking assays. FIG. 12A shows the study design and western blots of LNCaP and PC3tumor tissue samples from mice administered with C2-54 TYR and C2-54 FSY. The top panel shows the PSMA region of the gel blotted with anti-VHH antibody, the middle panel shows the free VHH region of the gel blotted with anti-VHH antibody, and the bottom panel shows the anti-GAPDH blot. The left panel shows samples from LNCaP tumor bearing animals, while the right panel shows the PC3 samples. Crosslinking is only observed in LNCaP (PSMA+) tumors and in the presence of C2-54FSY. FIG. 12B shows a graph of plasma concentration of C2-54 TYR and C2-54 FSY after dosing.

FIG. 13 shows an SDS-PAGE analysis of crosslinking kinetics for C8-54FSY, C8-55FSY, and C8-56FSY (left to right). Band positions representing crosslinking and C8-FSY are labeled. For each construct the lanes represent (left to right) 0, 15, 30, 60, 120, 180 minutes, and ladder.

FIG. 14 shows an SDS-PAGE analysis of crosslinking for various C15-FSY containing constructs. Left gel: lanes (left to right) represent constructs with FSY substitution at positions: 2, 4, 5, 6, 7, 8, 9, 10, 11, 12, 33, 34, 35, ladder, and Her2. Right gel: lanes (left to right) represent the constructs with FSY substitution at positions: 36, 37, 66, 67, 68, 69, wt ladder, and Her2. Band positions representing crosslinking and the FSY comprising constructs are labeled.

FIG. 15 shows an SDS-PAGE analysis of cross linking for biparatopic construct C38-FSY with Her2-Fc. Lanes (left to right) are times in hours 0, 0.5, 1.5, 2.5, 3.5, 4.5, 5.5, Her2, and ladder. Band positions representing crosslink, Her2-Fc, and C38-FSY are labeled.

FIG. 16 depicts a plot of fluorescence vs. concentration derived from binding studies via flow cytometry for constructs in A431 and Colo320DM cells: A431-C23-TYR, filled squares; A431-C23-FSY, filled triangles; Colo320DM-C23-TYR, “x”-symbols; and Colo320DM-C23-FSY, open circles. The y-axis is labeled AF680-GeoMean from 0 to 200,000 at 40,000 unit intervals. The x-axis is labeled Concentration (M) from 10⁻¹¹ to 10⁻⁵ on a log base 10 scale.

FIG. 17 shows intratumoral free- and EGFR-crosslinked AF680-labeled compounds C23-TYR and C23-FSY in a time-dependent manner in A431 (EGFR+) and COLO320DM (EGFR−) tumors. Free AF-680 labeled C23-TYR and C23-FSY was detected in the ~15 kD region (bottom panel) and the EGFR crosslinked sdAb band in the ~175 kD region (top panel). Time-dependent crosslinking of EGFR was observed with C23-FSY but not C23-TYR in EGFR+A431 tumors. Neither free-sdAb retention nor crosslinking was observed in the EGFR-COLO320DM tumors.

FIG. 18A depicts a plot of photons/s/g tissue across triplicate biological replicates from A431 tumors derived from animals administered C23-TYR or C23-FSY test articles. Values from individual animals are shown, the center bar shows the mean intensity and error bars indicate SEM (* indicates p=0.024, ** indicates p=0.002 by paired t test). The C23-FSY was present at a significantly higher level in the A431 tumors as compared with the non-FSY containing C23-TYR protein at both the 8 and 24 hour time points. The y-axis is labeled Photos/s/g tissue from 0 to 1.5×10¹⁰ at 0.5×10¹⁰ intervals. The x-axis is labeled Time after dose (hr) at 8 and 24 hr times. Stars indicated statistical significance via Paired t test (two tailed) (*=p≤0.05, **=p≤0.005).

FIG. 18B depicts a plot of quantitative ex vivo fluorescence intensities of A431 and COLO320DM tumors from animals administered C23-FSY at 8 and 24 hours post dose. Plot shows photons/s/g tissue across triplicate biological replicates. Values from individual animals are shown, center bar shows the mean intensity, error bars indicate SEM. The C23-FSY was present at a significantly higher level in the A431 tumors as compared with the amounts present in the EGFR-COLO320DM tumors at both the 8 and 24 hour time points, demonstrating the specificity of the tumor locking of the C23-FSY protein. The y-axis is labeled Photons/s/g tissue from 0 to 1×10¹⁰ at 0.5×10¹⁰ intervals. The x-axis is labeled Time after dose (hr) at 8 and 24 hr times for each set of bars. The left set are A431 model tumors and the right set are Colo320DM model tumors.

FIG. 19 shows a fluorescence image of SDS PAGE gels showing tumor associated free sdAb and PSMA-crosslinked sdAb in a time-dependent manner. Mice bearing LNCaP tumors were administered C30-TYR or C30-FSY in triplicate. At designated time points post-treatment, tumors were harvested and processed for gel electrophoresis to detect fluorophore-conjugated sdAb test articles. Free (non-crosslinked) sdAb-AF680 was detected in the ~20 kD region (bottom panel) and the PSMA-crosslinked sdAb-AF680 species for C30-FSY migrated in the 100 kD region (top panel). The lane indicated with * shows vehicle sample.

FIG. 20 shows the quantitative analysis of tumor associated free and PSMA-crosslinked test articles C30-TYR and C30-FSY. Fluorescence band intensity of the free and PSMA-crosslinked species bands from the gel above were quantitated via densitometry and comparison to a standard curve. The total intratumoral test article concentration (free and PSMA-crosslinked, pg/mg tumor tissue) is plotted vs time (h). Data points represented with * indicate samples that were below the limit of detection and quantitation. The tumor exposure of the C30-FSY was increased approximately 3× relative to the non-covalent C30-TYR.

FIG. 21A depicts a plot of a comparison of the cytotoxicity of the C26-54TYR and C26-54FSY test articles in the PC3PIP (PSMA positive) and PC3flu (PSMA negative) cell lines showing concentration versus cell viability curves for sdAbs conjugated to MMAE. The y-axis is labeled % viability from 0 to 120 at 20 unit intervals. The x-axis is labeled Test article concentration (M) from 10⁻¹¹ to 10⁻⁶ at log base 10 intervals.

FIG. 21B depicts a plot of a comparison of the cytotoxicity of the C28-101TYR and C28-101FSY test articles in the PC3PIP (PSMA positive) and PC3flu (PSMA negative) cell lines showing concentration versus cell viability curves for sdAbs conjugated to MMAE. The y-axis is labeled % viability from 0 to 120 at 20 unit intervals. The x-axis is labeled Test article concentration (M) from 10⁻¹¹ to 10⁻⁶ at log base 10 intervals.

FIG. 22 depicts an SDS-PAGE analysis of FSY crosslinking kinetics, where 52FSY and 54FSY variants of C17 were incubated with Her2 receptor and examined for crosslinking efficiency. The lanes 1-5 represent C17-52FSY at 0, 30, 60, 120, and 180 min; lanes 6-10 represent C17-54FSY at 0, 30, 60, 120, and 180 min; lanes 11 and 12 are FcHer2 and ladder, respectively. Band positions corresponding to C17-FSY, receptor, and crosslinking products are labeled.

FIG. 23A depicts a plot of a comparison of the cytotoxicity using a 5 hour washout of the C33-52TYR, C33-52FSY, C33-54TYR and C33-54FSY test articles showing concentration versus cell viability curves for sdAbs conjugated to MMAE in BT474 cells. The y-axis is labeled % viability from −20 to 120 at 20 unit intervals. The x-axis is labeled Test Article Concentration (nM) from 10⁻³ to 10³ at log base 10 intervals.

FIG. 23B depicts a plot of a comparison of the cytotoxicity using a continuous 6 day exposure of the C33 test articles showing concentration versus cell viability curves for sdAbs conjugated to MMAE in BT474 cells. The y-axis is labeled % viability from −20 to 120 at 20 unit intervals. The x-axis is labeled Test Article Concentration (nM) from 10⁻² to 10² at log base 10 intervals.

FIG. 24A depicts plots of PSMA crosslinking for monoparatopic and biparatopic constructs (C3-101FSY, square; C34-FSY, triangle; C36-FSY, circle) The y-axis is labeled Crosslinked PSMA (Total %) from 0 to 100 at 20 unit intervals. The x-axis is labeled Test Article Concentration (nM) from 0.01 to 1000 at base 10 intervals. The plot corresponds to cross-linking at 1 hour.

FIG. 24B depicts plots of PSMA crosslinking for monoparatopic and biparatopic constructs (C3-101FSY, square; C34-FSY, triangle; C36-FSY, circle). The y-axis is labeled Crosslinked PSMA (Total %) from 0 to 100 at 20 unit intervals. The x-axis is labeled Test Article Concentration (nM) from 0.01 to 1000 at base 10 intervals. The plot corresponds to cross-linking at 6 hours.

FIG. 25A depicts a western blot analysis of FSY crosslinking kinetics at varying concentrations of C3-101FSY and C34-FSY constructs at 1 hour and 6 hour time points. Lanes 1-6 represent C3-101FSY at 1 hour; lanes 7-12 represent C34-FSY at 1 hour; lanes 13-18 represent C3-101FSY at 6 hours, and lanes 19-24 represent C34-FSY at 6 hours. Each grouping of six lanes depicts increasing construct concentration (left to right): UTC, 0.1, 1, 10, 100, 1000 nM. The top set of blots depicts a-PSMA and the bottom set of blots depicts a-GAPDH. Band positions for GAPDH, PSMA, crosslink-monomer, and crosslink dimer are labeled.

FIG. 25B depicts a western blot analysis of FSY crosslinking kinetics at varying concentrations of C3-101FSY and C36-FSY constructs at 1 hour and 6 hour time points. Lanes 1-6 represent C3-101FSY at 1 hour; lanes 7-12 represent C36-FSY at 1 hour; lanes 13-18 represent C3-101FSY at 6 hours, and lanes 19-24 represent C36-FSY at 6 hours. Each grouping of six lanes depicts increasing construct concentration (left to right): UTC, 0.1, 1, 10, 100, 1000 nM. The top set of blots depicts a-PSMA and the bottom set of blots depicts a-GAPDH. Band positions for GAPDH, PSMA, crosslink-monomer, and crosslink dimer are labeled.

FIG. 26A depicts an SDS-PAGE analysis of crosslinking kinetics between EGFR and monoparatopic construct C4-109FSY. The t half-max was approximately 120 minutes. Bands corresponding to the cross-linked product, EGFR, and sdAb monomer are labeled. A cartoon of a receptor engaged with a monomeric FSY-containing sdAb is shown to right.

FIG. 26B depicts an SDS-PAGE analysis of crosslinking kinetics between EGFR and biparatopic construct C37-FSY. Bands corresponding to the cross-linked product, EGFR, and sdAb dimer are labeled. A cartoon depicting a receptor engaged with a biparatopic FSY-containing sdAb construct is shown to right.

FIG. 27 depicts a plot of EGFR crosslinking kinetics for monoparatopic construct C4-109FSY (squares) or biparatopic construct C37-FSY (circles). The y-axis is labeled EGFR Crosslinking %, from 0 to 100%. The x-axis is labeled Time (min) from 0 to 400 at 100 minute intervals.

FIG. 28A-C depicts exemplary conjugates described herein comprising a first targeting domain (e.g., constructs C1-C4) and a second targeting domain (e.g., constructs C1-C4) wherein the conjugate comprise an unnatural amino acid (e.g., FSY). One exemplary conjugate also comprises a payload. Exemplary conjugates can include an unnatural amino acid on the first targeting domain, second targeting domain, or both targeting domains

### DETAILED DESCRIPTION OF THE INVENTION

Antibodies, antibody fragments and antibody-related constructs, such as antibody-drug conjugates (ADCs), can be useful tools for research and clinical applications. However, in some instances use of these molecules is limited by on/off rates for a target as well as stability. Provided herein are compositions and methods for specific and covalent binding of conjugates to targets. In some instances, a conjugate comprises at least one targeting domain with at least one unnatural amino acid (UAA) residue in the proximity of the interface between the target and the targeting domain, such that when the targeting domain and target are bound, a covalent bond is formed between the target and the UAA residue. In some instances, a conjugate comprises at least one targeting domain and at least one payload. In some instances, the targeting domain comprises at least one unnatural amino acid (UAA) residue in the proximity of the interface between the target and the targeting domain, such that when the targeting domain and target are bound, a covalent bond is formed between target and the UAA residue. In some instances, once the conjugate reaches its targeted cell/tumor, the payload is now covalently bound to the target via the targeting domain. In some instances, covalent interactions eliminate or reduce off rates of conjugates binding to targets, increasing contact with attached payloads.

In some instances, a conjugate comprises (i) a first targeting domain, and (ii) second targeting domain, wherein the first or second targeting domain comprise at least one unnatural amino acid (UAA). In some instances, the first or the second targeting domain comprises at least one UAA that is present in the proximity of the interface between a target and the first or the second targeting domain, and when the first or the second targeting domain and the target are bound, a covalent bond is formed between the target and the at least one UAA. In some instances, the conjugate further comprises a payload. In some instances, when the conjugate reaches its targeted cell, such as a cell within a tumor or tumor microenvironment, the payload is now covalently bound to the target via one of the targeting domains (e.g., the first or the second target domain).

In some instances, covalent interactions eliminate or reduce off rates of conjugates binding to targets, or otherwise stabilize contact to the target. In further instances, the first and the second targeting domains in the conjugates provided herein target the same or different targets. In some cases, only the second targeting domain, but not the first targeting domain, comprises a UAA. In some cases, only the first targeting domain, but not the second targeting domain, comprises a UAA. In some cases, both the first targeting domain and the second targeting domain comprises a UAA. In some instances, the payload may be attached to the first targeting domain or the second targeting domain. In some cases, the payload is attached to the first targeting domain. In some cases, the payload is attached to the second targeting domain.

### Conjugates

Conjugates may comprise a targeting domain and a payload. In some embodiments, the payload is attached to the conjugate with a linker. In some instances, the conjugate comprises at least one unnatural amino acid. In some instances, the targeting domain is configured to bind to a target and one of the unnatural amino acids within the targeting domain forms a covalent bond with the target.

Conjugates provided herein may comprise (i) a first targeting domain, and (ii) a second targeting domain. Conjugates provided herein may comprise (i) a first targeting domain, (ii) a second targeting domain, and (iii) a payload. Conjugates herein may include a first targeting domain and a second targeting domain, wherein the first targeting domain and the second targeting domain are configured to bind to a same cell. In some instances, the conjugate comprises at least one unnatural amino acid (UAA) comprised in the first targeting domain, in the second targeting domain, or each of the first targeting domain and the second targeting domain comprises at least one UAA. In some instances, a targeting domain is configured to bind to a target and one of the UAAs within the targeting domain (e.g., the first and/or the second targeting domain) forms a covalent bond with the target. In some instances, the first targeting domain comprises a UAA and is configured to form a covalent bond with a first target, such that at least one UAA is present in the first targeting domain in the proximity of the interface between the first target and the first targeting domain. In some instances, the first target and the first targeting domain are bound, and a covalent bond is formed between the first target and the UAA in the first targeting domain of the conjugate. In some instances, the second targeting domain comprises a UAA and is configured to form a covalent bond with a second target, such that at least one UAA is present in the second targeting domain in the proximity of the interface between the second target and the second targeting domain. In some instances, the second target and the second targeting domain are bound, and a covalent bond is formed between the second target and the UAA in the second targeting domain of the conjugate. In some instances, the first target is the same as the second target. In some instances, the first target and the second target are on the surface of the same cell.

In some embodiments, the first targeting domain and the second targeting domain bind the same target, such as on the surface of a cell, and the engagement of one of the targeting domains brings the other targeting domain in proximity to its corresponding target. In some cases, the first targeting domain is bound to the first target and the second targeting domain is bound to the second target, and at least one of the first and second targeting domains comprises a UAA and the UAA forms a covalent bond with the corresponding target. In some instances, one targeting domain forms a covalent bond with its corresponding target and the other targeting domain is bound noncovalently to its corresponding target. In some instances, both the first targeting domain and the second targeting domain are each bound covalently to their corresponding targets.

The conjugates provided herein may be configured to bind to more than one target. In some instances, the second targeting domain may bind a different target than the first targeting domain. In some instances, the conjugate comprises at least 2, 3, 4, 5, 6, or more than 7 targeting domains. In some instances, the targeting domains (e.g., the first targeting domain and the second targeting domain) are attached to each other via a linker (e.g., a chemical linker, fusion protein, or other linker provided herein). Multiple targeting domains in the conjugates provided herein (e.g., the first targeting domain and the second targeting domain) in some instances can provide for monoparatopic or biparatopic constructs. In some instances, the first targeting domain and the second targeting domain bind the same target. In some instances, the first targeting domain and the second targeting domain bind different epitopes of the same target. In some instances, the first targeting domain and the second targeting domain bind different targets. In some instances, the first targeting domain and the second targeting domain are linked to form a fusion protein.

Targeting domains (e.g., the first targeting domain or the second targeting domain) may guide payloads attached to the conjugate to a target. In some instances, the targeting domain comprises an antibody or fragment thereof. In some instances, a targeting domain comprises a monospecific Fab2, bispecific Fab2, trispecific Fab3, monovalent IgG, scFv, bispecific diabody, trispecific triabody, scFv-Fc, nanobody (i.e., single domain antibody, sdAb), minibody, IgNAR, V-NAR, hcIgG, VhH, or peptibody, Darpin, monobody/FN3, VNAR, Repebody, Darpin. In some instances, the targeting domain comprises a nanobody. In some embodiments, the targeting domain comprises a single domain antibody (sdAb). In some instances, a targeting domain comprises one or more CDR regions. In some embodiments, the targeting domain binds to a cell surface molecule. In some instances, a conjugate is biparatopic. In some embodiments, the first targeting domain and the second targeting domain each comprise an antibody or antigen binding fragment, and the structure of such antibody or antigen binding fragment may be the same or different. In some instances, the first targeting domain can be a single chain (e.g., Fv) antibody fragment and the second targeting domain can be a sdAb, or in other instances, the first targeting domain can be a sdAb and the second targeting domain can be a sdAb.

In some instances, the targeting domain (e.g., the first targeting domain and/or the second targeting domain) is an antibody mimic such as an affibody, DARPin or a mini binder.

In some instances, a target comprises a cell surface protein. In some instances, a target comprises PSMA. In some instances, a targeting domain (e.g., the first targeting domain and/or the second targeting domain) comprises any one of SEQ IDS: 1-4, or 16-64. In some instances, a targeting domain comprises a sequence having at least 99%, 98%, 97%, 95%, 90%, 85%, 80%, 70%, or at least 65% identity with any one of SEQ IDS: 1-4, or 16-64. In some instances, a targeting domain comprises a sequence having at least 99%, 98%, 97%, 95%, 90%, 85%, 80%, 70%, or at least 65% identity with any one of SEQ IDS: 1-4, or 16-64 and at least one unnatural amino acid.

Unnatural amino acids may be located at any position in the conjugate. In some instances, one or both of the first or the second targeting domain (e.g., the first targeting domain or the second targeting domain) comprises an unnatural amino acid. In some instances, the targeting domain is an antibody or antigen binding fragment comprising one or more complementary determining regions (CDRs). In some instances, one or more unnatural amino acids is comprised within or within proximity of a CDR in only one or in both of the targeting domains. In some instances, the targeting domain comprises an unnatural amino acid. In some instances, the targeting domain comprises C1, C2, or C3. In some instances, the conjugate comprises C1 with an unnatural amino acid in CDR1, CDR2, or CDR3. In some instances, the conjugate comprises C1 with an unnatural amino acid at any one of positions 26, 28, 29, 30, 99, 102, 103, 105, 108, 110, 111, 112, 113, 114, and 115. In some instances, the conjugate comprises C1 with an unnatural amino acid at any one of positions 28, 102, 112, and 113. In some instances, the conjugate comprises C2 with an unnatural amino acid in CDR1, CDR2, or CDR3. In some instances, the conjugate comprises C2 with an unnatural amino acid at any one of positions 50, 52, 53, 54, 56, 58, or 100. In some instances, the conjugate comprises C4 with an unnatural amino acid in CDR1, CDR2, or CDR3. In some instances, the conjugate comprises C4 with an unnatural amino acid at position 109. In some instances, the conjugate comprises C3 with an unnatural amino acid in CDR1, CDR2, or CDR3. In some instances, the conjugate comprises C3 with an unnatural amino acid at any one of positions 58, 62, 101, 103, or 107. In some instances, the conjugate comprises C8 with an unnatural amino acid at any one of positions 52, 53, 54, 55, 56, 58, 60, 62, 64. In some instances, the conjugate comprises C9 with an unnatural amino acid at any one of positions 53, 55, 56, 57, 58, 60, 64, 67. In some instances, a conjugate comprises a single domain antibody (sdAb), comprising sdAb having a CDR region and at least one unnatural amino acid (UAA) residue within or in the proximity of the CDR region, wherein the sdAb comprises any one of SEQ ID Nos. 1-4, or 16-64. In some instances, a conjugate comprises a single domain antibody (sdAb), comprising sdAb having a CDR region and at least one unnatural amino acid (UAA) residue within or in the proximity of the CDR region, wherein the sdAb comprises a sequence having 99%, 98%, 97%, 95%, 90%, 85%, 80%, 70%, or at least 65% identity with any one of SEQ ID Nos. 1-4, or 16-64. In some embodiments, the CDR region comprises one or more SEQ ID Nos. 5-13. In some embodiments, the CDR region comprises a sequence having at least 99%, 98%, 97%, 95%, 90%, 85%, 80%, 70%, or at least 65% identity with one or more SEQ ID Nos. 5-13.

In some instances, a conjugate comprises a tag, such as for purification (e.g., a His6 tag). In some instances, a conjugate does not comprise a tag, or the tag is removed prior to administration. In some instances, the conjugate comprises a leader sequence such as for expression or secretion. In some instances, a conjugate does not comprise a leader sequence, or the leader sequence is removed from the first targeting domain or second targeting domain prior to formation of the conjugate, prior to attachment of the payload, or prior to administration of the conjugate.

In some instances, the conjugate comprises a signal sequence. The signal sequence may allow for expression, folding, or oxidation of the conjugate in a bacterial cell. In some instances, the signal sequence may allow for the expressed conjugate in a bacterial cell to be transported to another location or environment to promote the folding or function of the conjugate. In some instances, the signal sequence may be a PelB sequence. In some instances, the signal sequence may allow the transport of the conjugate to the periplasm of a bacterial cell. In some instances, the environment may be oxidizing or reducing such to allow for the formation of disulfide or the reduction of disulfides.

### Targets

Conjugates provided herein may be configured to bind to one or more targets. In some instances, the conjugate is configured with a single targeting domain comprising a UAA and the conjugate binds to a target. In some instances, the conjugate is configured with two (or at least two) targeting domains and the first and second targeting domains bind to the one or more targets. In some instances, first targeting domain binds to a first target and the second targeting domain binds to a second target. In some instances, the UAA comprised in a targeting domain (e.g., a single targeting domain or the first targeting domain or the second targeting domain) forms a covalent bond between the targeting domain (e.g., a single targeting domain, or the first targeting domain or the second targeting domain) and the target (the corresponding target for the targeting domain(s), e.g., the first or second target). In some instances, the conjugate comprises a first targeting domain comprising a first UAA and second targeting domain comprising a second UAA. Such UAAs are in some instances the same or in other instances are different. In some instances, a target is a cell surface molecule (i.e., present in whole or in part on the outer surface of a cell). In some instances, a first targeting domain and a second targeting domain bind to the same target, such as the same cell surface molecule. In some instances, a first targeting domain and a second targeting domain each bind to different targets, such as binding to different cell surface molecules. The different cell surface molecules may be on a same cell.

In some instances, a target is a cell surface molecule present on a tumor cell. A target in some instances is a monomer. In some instances, the target is comprised in a multimeric structure of homogenous or heterogenous units. In some instances, the target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM.

In some instances, the conjugate comprises the first targeting domain and the second targeting domain and both targeting domains bind to the same target, wherein the target is a cell surface molecule on a tumor cell. For example, both targeting domains bind to a target selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some instances, the conjugate comprises the first targeting domain and the second targeting domain bind to different targets, where one or more of the different targets are cell surface molecules on a tumor cell. For example, the first and second targeting domains bind to different targets each of which is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Muc1, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some instances, the conjugate carries a payload to the cell surface when the first or second target (or both) are bound. In some instances, the conjugate does not have a payload and the conjugate acts as a blocker or antagonist when covalently bound to the first target, second target, or to both the first and second target.

In some instances, the conjugate comprises a single targeting domain and the targeting domain binds to a target, wherein the target is a cell surface molecule on a tumor cell. For example, the targeting domain binds to a target selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some instances, the single targeting domain carries a payload the target. In some instances, the single targeting domain does not have a payload and the conjugate acts as a blocker or antagonist when covalently bound to the target.

Targets that are engaged by targeting domains may comprise a variety of structures. In some instances, a target comprises one or more epitopes that can be engaged by a targeting domain. In some instances, a target comprises multiple epitopes and can be engaged by multiple targeting domains, e.g., such as by the first targeting domain binding to a first epitope and the second targeting domain binding to a second epitope. In some instances, a target comprises a monomer. In some instances, a target comprises a single chain peptide. In some instances, a target comprises a multimeric molecule. In some instances, a multimeric molecule comprises two or more sub-units. In some instances, sub-units have the same structure. In some instances, sub-units are different structures, or a combination of the same and different structures. In some instances, a multimeric molecule comprises two or more molecules in a complex. In some instances, a multimeric molecule comprises two proteins that complex or interact with each other.

### Exemplary Targeting Domains and Exemplary Conjugates

Conjugates provided herein comprise targeting domains that can be assembled from molecules that engage with a target. In some instances, the targeting domain (a single targeting domain or the first targeting domain and/or the second targeting domain) comprises an antigen-binding region, wherein the antigen binding region engages with a specific target. Such antigen binding regions can comprise CDRs, such as the 3 CDRs generally found in a heavy chain or light chain of an antibody. In some instances, the targeting domain comprises an antigen binding fragment comprising CDRs, such as a VHH (also called a nanobody or single domain antibody). In some instances, the single domain antibody comprises one or more UAAs. In some instances, a single targeting domain or for a conjugate with multiple targeting domains, one of the targeting domains (e.g., the first targeting domain or the second targeting domain) is selected from a single domain antibody of Table 1, such as C1, C2, C3, C4, or C5. In some instances, if conjugates with more than one targeting domain, each of the targeting domains (e.g., the first targeting domain and the second targeting domain) is independently selected from a single domain antibody of Table 1, such as any of C1, C2, C3, C4, or C5, and one or more UAAs is present in a CDR or in the proximity of a CDR of the single domain antibody. In some instances, the first targeting domain is selected from a single domain antibody of Table 1, such as any of C1, C2, C3, C4, or C5. In some instances, the second targeting domain is selected from a single domain antibody of Table 1, such as any of C1, C2, C3, C4, or C5.

In some instances, the conjugate comprises a single targeting domain, a first targeting domain, a second targeting domain or both targeting domains comprising construct C1. In some instances, the conjugate comprises construct C1 with an unnatural amino acid in CDR1, CDR2, or CDR3. In some instances, the conjugate comprises construct C1 with an unnatural amino acid at any one of positions 26, 28, 29, 30, 99, 102, 103, 105, 108, 110, 111, 112, 113, 114, and 115 in SEQ ID NOs: 1, 4, 19, or 73. In some instances, the conjugate comprises construct C1 with an unnatural amino acid at any one of positions 28, 102, 112, and 113 in SEQ ID NOs: 1, 4, 19, or 73. In some instances, a conjugate comprises a single domain antibody (sdAb), and at least one unnatural amino acid (UAA) within or in the proximity of a CDR region within the sdAb. In some instances, such sdAb comprises SEQ ID NO: 1. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 1. In some instances, a conjugate comprises a single targeting domain, a first targeting domain or a second targeting domain or both having at least 99%, 98%, 97%, 95%, 90%, 85%, 80%, 70%, or at least 65% identity to SEQ ID NO: 1.

In some instances, the conjugate comprises a single targeting domain, a first targeting domain, a second targeting domain or both targeting domains comprising construct C2. In some instances, the conjugate comprises construct C2 with an unnatural amino acid in CDR1, CDR2, or CDR3. In some instances, the conjugate comprises construct C2 with an unnatural amino acid at any one of positions 50, 52, 53, 54, 56, 58, or 100 of SEQ ID NO: 2 or 22. In some instances, a conjugate comprises a single domain antibody (sdAb), comprising sdAb and at least one unnatural amino acid (UAA) within or in the proximity of a CDR region within the sdAb, wherein the sdAb comprises SEQ ID NO: 2. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 2. In some instances, a conjugate comprises a single targeting domain, a first targeting domain or second targeting domain or both having at least 99%, 98%, 97%, 95%, 90%, 85%, 80%, 70%, or at least 65% sequence identity to SEQ ID NO: 2.

In some instances, the conjugate comprises a single targeting domain, a first targeting domain, a second targeting domain or both targeting domains comprising construct C3. In some instances, the conjugate comprises construct C3 with an unnatural amino acid in CDR1, CDR2, or CDR3. In some instances, the conjugate comprises construct C3 with an unnatural amino acid at any one of positions 58, 62, 101, 103, or 107 of SEQ ID NO: 3. In some instances, the conjugate comprises an unnatural amino acid in CDR1, CDR2, or CDR3 of C3. In some instances, the conjugate comprises an unnatural amino acid at position 109. In some instances, a conjugate comprises a single domain antibody (sdAb) and at least one unnatural amino acid (UAA) within or in the proximity of a CDR region within the sdAb, wherein the sdAb comprises SEQ ID NO: 3. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 3. In some instances, a conjugate comprises a single targeting domain, a first targeting domain or second targeting domain or both having at least 99%, 98%, 97%, 95%, 90%, 85%, 80%, 70%, or at least 65% sequence identity to SEQ ID NO: 3.

In some instances, the conjugate comprises a single targeting domain, a first targeting domain, a second targeting domain or both targeting domains comprising construct C4. In some instances, the conjugate comprises construct C4 with an unnatural amino acid in CDR1, CDR2, or CDR3. In some instances, a conjugate comprises a single domain antibody (sdAb), and at least one unnatural amino acid (UAA) within or in the proximity of a CDR region within the sdAb, wherein the sdAb comprises SEQ ID NO: 16. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 16. In some instances, a conjugate comprises a single targeting domain, a first targeting domain or second targeting domain or both having at least 99%, 98%, 97%, 95%, 90%, 85%, 80%, 70%, or at least 65% sequence identity to SEQ ID NO: 16.

In some instances, the conjugate comprises a single targeting domain, a first targeting domain, a second targeting domain or both targeting domains comprising construct C5. In some instances, the conjugate comprises construct C5 with an unnatural amino acid in CDR1, CDR2, or CDR3. In some instances, a conjugate comprises a single domain antibody (sdAb), and at least one unnatural amino acid (UAA) within or in the proximity of the CDR region within the sdAb, wherein the sdAb comprises SEQ ID NO: 18. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 18. In some instances, a conjugate comprises a single targeting domain, a first targeting domain or second targeting domain or both having at least 99%, 98%, 97%, 95%, 90%, 85%, 80%, 70%, or at least 65% sequence identity to SEQ ID NO: 18.

Conjugates described herein may comprise two or more targeting domains. In some instances, the two or more targeting domains (e.g., the first targeting domain or the second targeting domain) are attached to each other via a linker. In some instances, the conjugate is a fusion protein. In some instances, the linker comprises linker L1 (SEQ ID NO: 14). In some instances, the conjugate comprises any one of SEQ ID NOS: 65-72. In some instances, the conjugate has at least 99%, 98%, 97%, 95%, 90%, 85%, 80%, 70%, or at least 65% sequence identity to any one of SEQ ID NO: 65-72.

In some instances, a first targeting domain comprises any one of SEQ ID NOS: 1-4, or 16-64. In some instances, the first targeting domain comprises a sequence having at least 70% sequence identity to any one of SEQ ID NOS: 1-4, or 16-64. In some instances, the second targeting domain comprises any one of SEQ ID NOS: 1-4, or 16-64. In some instances, the second targeting domain comprises a sequence having at least 700 sequence identity to any one of SEQ TD NOs: 1-4, or 16-64. In some instances, the conjugate is configured with a single targeting domain and the single targeting domain comprises any one of SEQ ID NOS: 1-4, or 16-64. In some instances, the single targeting domain comprises a sequence having at least 700% sequence identity to any one of SEQ TD NOs: 1-4, or 16-64.

In some instances, the conjugate comprises SEQ TD NO: 65-72. In some instances, the conjugate comprises a sequence having at least 700% sequence identity to any one of SEQ TD NOS: 65-72. In some instances, the conjugate comprises any one of SEQ ID NOs: 1-4, or 16-64. In some instances, the conjugate comprises a sequence having at least 700% sequence identity to any one of SEQ ID NOs: 1-4 or 16-64. In some instances, the conjugate comprises a sequence having at least 700% sequence identity to SEQ TD NOs: 65-72.

In some instances, amino acid sequences associated with conjugates described herein are in Table 1A.

TABLE 1A
Amino Acid Sequences

| | SEQ | |
|---|---|---|
| Construct | ID | |
| Name | NO | Sequence |
| C1 | 1 | QVQLQESGGGSVQAGGSLRLSCTAPGYTDSN |
| | | YYMSWFRQAPGKEREWVAGVNTGRGSTSY |
| | | ADSVKGRFTISQDNAKNTMFLQMNSLKPEDT |
| | | AIYYCAVAACHFCDSLPKTQDEYILWGQGTQ |
| | | VTVSSAAAYPYDVPDYGS |
| C2 | 2 | EVQLVESGGGLVQPGGSLTLSCAASRFMISEY |
| | | SMHWVRQAPGKGLEWVSTINPAGTTDYAES |
| | | VKGRFTISRDNAKNTLYLQMNSLKPEDTAVY |
| | | YCDGYGYRGQGTQVTVSS |
| C3 | 3 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRDYSLSSSSDDFDYWGQGTQVTVSSA |
| | | AAYPYDVPDYGS |
| C1- | 4 | QVQLQESGGGSVQAGGSLRLSCTAPGYTDSN |
| C101A/C1 | | YYMSWFRQAPGKEREWVAGVNTGRGSTSY |
| 04A/H102 | | ADSVKGRFTISQDNAKNTMFLQMNSLKPEDT |
| (FSY) | | AIYYCAVAAAXFADSLPKTQDEYILWGQGTQ |
| | | VTVSSAAAYPYDVPDYGSC |
| C1- | 5 | GYTDSNYYMS |
| CDR1_26 | | |
| -35 | | |
| C1- | 6 | VNTGRGSTSYADSVKG |
| CDR2_50 | | |
| -66 | | |
| C1- | 7 | AACHFCDSLPKTQDEYIL |
| CDR3_99 | | |
| -116 | | |
| C2- | 8 | RFMISEYSMH |
| CDR1_26 | | |
| -35 | | |
| C2- | 9 | TINPAGTTDYAESVKG |
| CDR2_50 | | |
| -65 | | |
| C2- | 10 | DGYGY |
| CDR3_96 | | |
| -100 | | |
| C3_CDR1 | 11 | GWPYSTYSMN |
| _26-35 | | |
| C3_CDR2 | 12 | GISSTMSGIIFAESKAG |
| _50-65 | | |
| C3_CDR3 | 13 | RRDYSLSSSSDDFDY |
| _99-113 | | |
| L1 | 14 | GGGGSGGGGS |
| PelB | 15 | MKYLLPTAAAGLLLLAAQPAMAMG |
| C4 | 16 | QVKLEESGGGSVQTGGSLRLTCAASGRTSRS |
| | | YGMGWFRQAPGKEREFVSGISWRGDSTGYA |
| | | DSVKGRFTISRDNAKNTVDLQMNSLKPEDTA |
| | | IYYCAAAAGSAWYGTLYEYDYWGQGTQVT |
| | | VSS |
| C4- | 17 | QVKLEESGGGSVQTGGSLRLTCAASGRTSRS |
| 109FSY | | YGMGWFRQAPGKEREFVSGISWRGDSTGYA |
| | | DSVKGRFTISRDNAKNTVDLQMNSLKPEDTA |
| | | IYYCAAAAGSAWYGTLXEYDYWGQGTQVT |
| | | VSS |
| C5 | 18 | QVQLQESGGGLVQPGGSLRLSCAASGRTFSS |
| | | YAMGWFRQAPGKQREFVAAIRWSGGYTYYT |
| | | DSVKGRFTISRDNAKTTVYLQMNSLKPEDTA |
| | | VYYCAATYLSSDYSRYALPQRPLDYDYWGQ |
| | | GTQVTVSSLE |
| C1- | 19 | QVQLQESGGGSVQAGGSLRLSCTAPGYTDSN |
| C101A/C1 | | YYMSWFRQAPGKEREWVAGVNTGRGSTSY |
| 04A | | ADSVKGRFTISQDNAKNTMFLQMNSLKPEDT |
| | | AIYYCAVAAAHFADSLPKTQDEYILWGQGTQ |
| | | VTVSSAAAYPYDVPDYGSC |
| C2- | 20 | EVQLVESGGGLVQPGGSLTLSCAASREMISEY |
| A54(FSY) | | SMHWVRQAPGKGLEWVSTINPXGTTDYAES |
| | | VKGRFTISRDNAKNTLYLQMNSLKPEDTAVY |
| | | YCDGYGYRGQGTQVTVSS |
| C6 | 21 | EVQLVESGGGLVQPGGSLKLSCAASGFTFNK |
| | | YAMNWVRQAPGKGLEWVARIRSKYNNYAT |
| | | YYADSVKDRFTISRDDSKNTAYLQMNNLKTE |
| | | DTAVYYCVRHGNFGNSYISYWAYWGQGTLV |
| | | TVSS |
| C7 | 22 | EVQLVESGGGLVQPGESLTLSCVVAGSIFSFA |
| | | MSWYRQAPGKERELVARIGSDDRVTYADSV |
| | | KGRFTISRDNIKRTAGLQMNSLKPEDTAVYY |
| | | CNAQTDLRDWTVREYWGQGTQVTVSSSPST |
| | | PPTPSPSTPP |
| C8 | 23 | QVQLQESGGGLVQTGGSLRLSCAASGSIFVG |
| | | NAMGWYRQALGNQRELVAGITSDGITYYPD |
| | | SVKGRFTISRDNDKNTIYLQMNSLKPEDTAV |
| | | YYCNLWPPRIGFASWGQGTQVTVSSAAAYP |
| | | YDVPDYGS |
| C9 | 24 | QVQLVQSGGGLVQAGGSLRLSCAFSGRTFSM |
| | | YTMGWFRQAPGKEREFVAANRGRGLSPDIA |
| | | DSVNGRFTISRDNAKNTLYLQMDSLKPEDTA |
| | | VYYCAADLQYGSSWPQRSSAEYDYWGQGTT |
| | | VTVSS |
| C10 | 25 | QVQLVQSGGGLVQAGGSLRLSCAPSGSIFGIR |
| | | TMDWYRQAPGKERELVARITMDGRVFHADS |
| | | VKGRFSGSRDGASNAVYLQMNSLKPDDTAV |
| | | YYCRYSGLTSREDYWGPGTQVTVSS |
| C11 | 26 | QVQLVQSGGGLVHPGGSLRLSCAASGIDLSL |
| | | YRMRWYRQAPGKERDLVALITDDGTSYYED |
| | | SVKGRFTITRDNPSNKVFLQMNSLKPEDTAV |
| | | YYCNAETPLSPVNYWGQGTQVTVS |
| C12 | 27 | EVQLLESGGGEVQPGGSLRLSCAASETFGVV |
| | | FTLGWYRQAPGKGREFVARVTGTDTVDYAE |
| | | SVKGRFTISSDFARNTVYLQMNSLRAEDTAV |
| | | YYCNTGAYWGQGTLVTVKPSPSTPPTPSPSTP |
| | | P |
| C14 | 28 | QVQLVESGGGSVQAGGSLRLSCAASGDTYGS |
| | | YWMGWFRQAPGKEREGVAAINRGGGYTVY |
| | | ADSVKGRFTISRDTAKNTVYLQMNSLRPDDT |
| | | ADYYCAASGVLGGLHEDWFNYWGQGTQVT |
| | | VSS |
| C15 | 29 | MDLGKKLLEAARAGQDDEVRILMANGADV |
| | | NAHDFYGITPLHLAANFGHLEIVEVLLKHGA |
| | | DVNAFDYDNTPLHLAADAGHLEIVEVLLKYG |
| | | ADVNASDRDGHTPLHLAAREGHLEIVEVLLK |
| | | NGADVNAQDKFGKTAFDISIDNGNEDLAEIL |
| | | QKLAAALE |
| C16 | 30 | RSDLGKKLLEAARAGQDDEVRILMANGADV |
| | | NAKDEYGLTPLYLATAHGHLEIVEVLLKNGA |
| | | DVNAVDAIGFTPLHLAAFIGHLEIAEVLLKHG |
| | | ADVNAQDKFGKTAFDISIGNGNEDLAEILQKL |
| | | NGS |
| C17 | 31 | QVQLQESGGGSVQAGGSLKLTCAASGYIFNS |
| | | CGMGWYRQSPGRERELVSRISGDGDTWHKE |
| | | SVKGRFTISQDNVKKTLYLQMNSLKPEDTAV |
| | | YFCAVCYNLETYWGQGTQVTVSSSPSTPPTPS |
| | | PSTPP |
| C17- | 32 | QVQLQESGGGSVQAGGSLKLTCAASGYIFNS |
| 52FSY | | CGMGWYRQSPGRERELVSRIXGDGDTWHKE |
| | | SVKGRFTISQDNVKKTLYLQMNSLKPEDTAV |
| | | YFCAVCYNLETYWGQGTQVTVSSSPSTPPTPS |
| | | PSTPP |
| C17_54 | 33 | QVQLQESGGGSVQAGGSLKLTCAASGYIFNS |
| (FSY) | | CGMGWYRQSPGRERELVSRISGXGDTWHKE |
| | | SVKGRFTISQDNVKKTLYLQMNSLKPEDTAV |
| | | YFCAVCYNLETYWGQGTQVTVSSSPSTPPTPS |
| | | PSTPP |
| C18 | 34 | EVQLVQSGGGLVQPGGSLRLSCAASGRTSSK |
| | | YAMGWFRQAPGKGTEFVATISWSDGSTYYA |
| | | DSVEGRFTISRDNAKNTVYLQMNSLKPEDTA |
| | | VYYCAAAVDVLAGTFEYEYDYWGQGTLVT |
| | | VSSSPSTPPTPSPSTPP |
| C19 | 35 | QVKLEESGGGSVQTGGSLRLTCAASGRTSRS |
| | | YGMGWFRQAPGKSREFVSGISWRGDSTGYA |
| | | DSVKGRFTISRDNAKNTVDLQMNSLKPEDTA |
| | | IYYCAAAAGSAWYGTRYEYDYWGQGTQVT |
| | | VSS |
| C19_109 | 36 | QVKLEESGGGSVQTGGSLRLTCAASGRTSRS |
| (FSY) | | YGMGWFRQAPGKSREFVSGISWRGDSTGYA |
| | | DSVKGRFTISRDNAKNTVDLQMNSLKPEDTA |
| | | IYYCAAAAGSAWYGTRXEYDYWGQGTQVT |
| | | VSS |
| C20 | 37 | QVTLRESGGGLVQAGGSLRLSCKGSGRAINT |
| | | YAMGWFRQAPGKEREFVAAISWNGGHTRYA |
| | | DSVQGRFAISRDNADNTMYLQMNSLKPEDT |
| | | AVYHCAAYSDYHRIATMEADADSWGQGTQ |
| | | VTV |
| C21 | 38 | EVQLVESGGGLVQPGGSLRLSCAASDFTFRN |
| | | YEMSWVRQAPGKGLEWVSSISGSGGSTYYA |
| | | HSLKGRFTISRDNSKNTLYLQMNSLRAEDTAI |
| | | YYCARLRDGFNNGFDYWGQGTLVTVSSLPE |
| | | TGGSPSTPPTPSPSTPP |
| C22-TYR | 39 | MGQVKLEESGGGSVQTGGSLRLTCAASGRTS |
| | | RSYGMGWFRQAPGKSREFVSGISWRGDSTG |
| | | YADSVKGRFTISRDNAKNTVDLQMNSLKPED |
| | | TAIYYCAAAAGSAWYGTRYEYDYWGQGTQ |
| | | VTVSSLPETGGSPSTPPTPSPSTPP |
| C22-FSY | 40 | MGQVKLEESGGGSVQTGGSLRLTCAASGRTS |
| | | RSYGMGWFRQAPGKSREFVSGISWRGDSTG |
| | | YADSVKGRFTISRDNAKNTVDLQMNSLKPED |
| | | TAIYYCAAAAGSAWYGTRXEYDYWGQGTQ |
| | | VTVSSLPETGGSPSTPPTPSPSTPP |
| C23-TYR | 41 | MGQVKLEESGGGSVQTGGSLRLTCAASGRTS |
| | | RSYGMGWFRQAPGKSREFVSGISWRGDSTG |
| | | YADSVKGRFTISRDNAKNTVDLQMNSLKPED |
| | | TAIYYCAAAAGSAWYGTRYEYDYWGQGTQ |
| | | VTVSSLPETGGG-(AF680) |
| C23-FSY | 42 | MGQVKLEESGGGSVQTGGSLRLTCAASGRTS |
| | | RSYGMGWFRQAPGKSREFVSGISWRGDSTG |
| | | YADSVKGRFTISRDNAKNTVDLQMNSLKPED |
| | | TAIYYCAAAAGSAWYGTRXEYDYWGQGTQ |
| | | VTVSSLPETGGG-(AF680) |
| C24 | 43 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRYFSLSSSSDDFDYWGQGTQVTVSSA |
| | | AAYPYDVPDYGS |
| C24-FSY | 44 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRXFSLSSSSDDFDYWGQGTQVTVSSA |
| | | AAYPYDVPDYGS |
| C25- | 45 | EVQLVESGGGLVQPGGSLTLSCAASREMISEY |
| 54TYR | | SMHWVRQAPGKGLEWVSTINPYGTTDYAES |
| | | VKGRFTISRDNAKNTLYLQMNSLKPEDTAVY |
| | | YCDGYGYRGQGTQVTVSSSPSTPPTPSPSTPP |
| | | C |
| C25- | 46 | EVQLVESGGGLVQPGGSLTLSCAASRFMISEY |
| 54FSY | | SMHWVRQAPGKGLEWVSTINPXGTTDYAES |
| | | VKGRFTISRDNAKNTLYLQMNSLKPEDTAVY |
| | | YCDGYGYRGQGTQVTVSSSPSTPPTPSPSTPP |
| | | C |
| C26 | 47 | EVQLVESGGGLVQPGGSLTLSCAASREMISEY |
| | | SMHWVRQAPGKGLEWVSTINPYGTTDYAES |
| | | VKGRFTISRDNAKNTLYLQMNSLKPEDTAVY |
| | | YCDGYGYRGQGTQVTVSSSPSTPPTPSPSTPP |
| | | CHHHHHH-(PEG8-VC-PABC-MMAE) |
| C26-54 | 48 | EVQLVESGGGLVQPGGSLTLSCAASREMISEY |
| FSY | | SMHWVRQAPGKGLEWVSTINPXGTTDYAES |
| | | VKGRFTISRDNAKNTLYLQMNSLKPEDTAVY |
| | | YCDGYGYRGQGTQVTVSSSPSTPPTPSPSTPP |
| | | CHHHHHH-(PEG8-VC-PABC-MMAE) |
| C27- | 49 | QVQLQESGGGSVEAGGSLRLSCARSGWPYST |
| 101TYR | | YSMNWFRQAPGKEREAVAGISSTMSGIIFAES |
| | | KAGQFTISQDNAKNTVYLQMNNLKPEDTAIY |
| | | YCAARRYFSLSSSSDDFDYWGQGTQVTVSSA |
| | | AAYPYDVPDYGSSPSTPPTPSPSTPPC |
| C27- | 50 | QVQLQESGGGSVEAGGSLRLSCARSGWPYST |
| 101FSY | | YSMNWFRQAPGKEREAVAGISSTMSGIIFAES |
| | | KAGQFTISQDNAKNTVYLQMNNLKPEDTAIY |
| | | YCAARRXFSLSSSSDDFDYWGQGTQVTVSSA |
| | | AAYPYDVPDYGSSPSTPPTPSPSTPPC |
| C28 | 51 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRYFSLSSSSDDFDYWGQGTQVTVSSA |
| | | AAYPYDVPDYGSSPSTPPTPSPSTPPCHHHHH |
| | | H-(PEG8-VC-PABC-MMAE) |
| C28-101 | 52 | QVQLQESGGGSVEAGGSLRLSCARSGWPYST |
| FSY/TYR | | YSMNWFRQAPGKEREAVAGISSTMSGIIFAES |
| | | KAGQFTISQDNAKNTVYLQMNNLKPEDTAIY |
| | | YCAARRXFSLSSSSDDFDYWGQGTQVTVSSA |
| | | AAYPYDVPDYGSSPSTPPTPSPSTPPCHHHHH |
| | | H-(PEG8-VC-PABC-MMAE) |
| C29-TYR | 53 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRYFSLSSSSDDFDYWGQGTQVTVSSA |
| | | AAYPYDVPDYGSLPETGGSPSTPPTPSPSTPP |
| C29-FSY | 54 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRXFSLSSSSDDFDYWGQGTQVTVSSA |
| | | AAYPYDVPDYGSLPETGGSPSTPPTPSPSTPP |
| C30-TYR | 55 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRYFSLSSSSDDFDYWGQGTQVTVSSA |
| | | AAYPYDVPDYGSLPETGGG-(AF680) |
| C30-FSY | 56 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRXFSLSSSSDDFDYWGQGTQVTVSSA |
| | | AAYPYDVPDYGSLPETGGG-(AF680) |
| C32- | 57 | QVQLQESGGGSVQAGGSLKLTCAASGYIFNS |
| 52TYR | | CGMGWYRQSPGRERELVSRIYGDGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGSPSTPPTPSPSTPP |
| C32- | 58 | QVQLQESGGGSVQAGGSLKLTCAASGYIFNS |
| 52FSY | | CGMGWYRQSPGRERELVSRIXGDGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGSPSTPPTPSPSTPP |
| C32- | 59 | QVQLQESGGGSVQAGGSLKLTCAASGYIFNS |
| 54TYR | | CGMGWYRQSPGRERELVSRISGYGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGSPSTPPTPSPSTPP |
| C32- | 60 | QVQLQESGGGSVQAGGSLKLTCAASGYIFNS |
| 54FSY | | CGMGWYRQSPGRERELVSRISGXGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGSPSTPPTPSPSTPP |
| C33- | 61 | QVQLQESGGGSVQAGGSLKLTCAASGYIFNS |
| 52TYR | | CGMGWYRQSPGRERELVSRIYGDGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGG-(PEG8-vc-PABC-MMAE) |
| C33- | 62 | QVQLQESGGGSVQAGGSLKLTCAASGYIFNS |
| 52FSY | | CGMGWYRQSPGRERELVSRIXGDGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGG-(PEG8-vc-PABC-MMAE) |
| C33- | 63 | QVQLQESGGGSVQAGGSLKLTCAASGYIFNS |
| 54TYR | | CGMGWYRQSPGRERELVSRISGYGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGG-(PEG8-vc-PABC-MMAE) |
| C33- | 64 | QVQLQESGGGSVQAGGSLKLTCAASGYIFNS |
| 54FSY | | CGMGWYRQSPGRERELVSRISGXGDTWHKE |
| | | SVKGRFTISQDNVKKTLYLQMNSLKPEDTAV |
| | | YFCAVCYNLETYWGQGTQVTVSSAAAYPYD |
| | | VPDYGSLPETGGG-(PEG8-vc-PABC-MMAE) |
| C34 | 65 | EVQLVESGGGLVQPGGSLTLSCAASRFMISEY |
| | | SMHWVRQAPGKGLEWVSTINPYGTTDYAES |
| | | VKGRFTISRDNAKNTLYLQMNSLKPEDTAVY |
| | | YCDGYGYRGQGTQVTVSSGGGGSGGGGSGG |
| | | GGSQVQLQESGGGSVEAGGSLRLSCARSGWP |
| | | YSTYSMNWFRQAPGKEREAVAGISSTMSGIIF |
| | | AESKAGQFTISQDNAKNTVYLQMNNLKPEDT |
| | | AIYYCAARRYFSLSSSSDDFDYWGQGTQVTV |
| | | SSAAAYPYDVPDYGSLPETGGSPSTPPTPSPST |
| | | PP |
| C34-FSY | 66 | EVQLVESGGGLVQPGGSLTLSCAASRFMISEY |
| | | SMHWVRQAPGKGLEWVSTINPYGTTDYAES |
| | | VKGRFTISRDNAKNTLYLQMNSLKPEDTAVY |
| | | YCDGYGYRGQGTQVTVSSGGGGSGGGGSGG |
| | | GGSQVQLQESGGGSVEAGGSLRLSCARSGWP |
| | | YSTYSMNWFRQAPGKEREAVAGISSTMSGIIF |
| | | AESKAGQFTISQDNAKNTVYLQMNNLKPEDT |
| | | AIYYCAARRXFSLSSSSDDFDYWGQGTQVTV |
| | | SSAAAYPYDVPDYGSLPETGGSPSTPPTPSPST |
| | | PP |
| C35 | 67 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRYFSLSSSSDDFDYWGQGTQVTVSSG |
| | | GGGSGGGGSGGGGSQVQLQESGGGSVEAGG |
| | | SLRLSCARSGWPYSTYSMNWFRQAPGKERE |
| | | AVAGISSTMSGIIFAESKAGQFTISQDNAKNT |
| | | VYLQMNNLKPEDTAIYYCAARRYFSLSSSSD |
| | | DFDYWGQGTQVTVSSAAAYPYDVPDYGSLP |
| | | ETGGSPSTPPTPSPSTPP |
| C36-FSY | 68 | QVQLQESGGGSVEAGGSLRLSCARSGWPYST |
| | | YSMNWFRQAPGKEREAVAGISSTMSGIIFAES |
| | | KAGQFTISQDNAKNTVYLQMNNLKPEDTAIY |
| | | YCAARRXFSLSSSSDDFDYWGQGTQVTVSSG |
| | | GGGSGGGGSGGGGSQVQLQESGGGSVEAGG |
| | | SLRLSCARSGWPYSTYSMNWFRQAPGKERE |
| | | AVAGISSTMSGIIFAESKAGQFTISQDNAKNT |
| | | VYLQMNNLKPEDTAIYYCAARRXFSLSSSSD |
| | | DFDYWGQGTQVTVSSAAAYPYDVPDYGSLP |
| | | ETGGSPSTPPTPSPSTPP |
| C37 | 69 | QVKLEESGGGSVQTGGSLRLTCAASGRTSRS |
| TYR or | | YGMGWFRQAPGKEREFVSGISWRGDSTGYA |
| FSY | | DSVKGRFTISRDNAKNTVDLQMNSLKPEDTA |
| | | IYYCAAAAGSAWYGTLXEYDYWGQGTQVT |
| | | VSSGGGGSGGGGSQVQLQESGGGLVQPGGSL |
| | | RLSCAASGRTFSSYAMGWFRQAPGKQREFV |
| | | AAIRWSGGYTYYTDSVKGRFTISRDNAKTTV |
| | | YLQMNSLKPEDTAVYYCAATYLSSDYSRYA |
| | | LPQRPLDYDYWGQGTQVTVSSLE |
| C38 | 70 | MDLGKKLLEAARAGQDDEVRILMANGADV |
| C15- | | NAHDFYGITPLHLAANFGHLEIVEVLLKHGA |
| C16 66 | | DVNAFXYDNTPLHLAADAGHLEIVEVLLKYG |
| wt or | | ADVNASDRDGHTPLHLAAREGHLEIVEVLLK |
| FSY) | | NGADVNAQDKFGKTAFDISIDNGNEDLAEIL |
| | | QKLGGGGSRSDLGKKLLEAARAGQDDEVRIL |
| | | MANGADVNAKDEYGLTPLYLATAHGHLEIV |
| | | EVLLKNGADVNAVDAIGFTPLHLAAFIGHLEI |
| | | AEVLLKHGADVNAQDKFGKTAFDISIGNGNE |
| | | DLAEILQKLNGS |
| C39 | 71 | QVQLQESGGGSVQAGGSLRLSCTAPGYTDSN |
| | | YYMSWFRQAPGKEREWVAGVNTGRGSTSY |
| | | ADSVKGRFTISQDNAKNTMFLQMNSLKPEDT |
| | | AIYYCAVAAAHFADSLPKTQDEYILWGQGTQ |
| | | VTVSSAAAYPYDVPDYGS |
| C40 | 72 | QVQLQESGGGSVQAGGSLRLSCTAPGYTDSN |
| | | YYMSWFRQAPGKEREWVAGVNTGRGSTSY |
| | | ADSVKGRFTISQDNAKNTMFLQMNSLKPEDT |
| | | AIYYCAVAAAHFADSLPKTQDEYILWGQGTQ |
| | | VTVSSAAAYPYDVPDYGSGGGGSGGGGSQV |
| | | QLQESGGGSVQAGGSLRLSCTAPGYTDSNYY |
| | | MSWFRQAPGKEREWVAGVNTGRGSTSYADS |
| | | VKGRFTISQDNAKNTMFLQMNSLKPEDTAIY |
| | | YCAVAAAHFADSLPKTQDEYILWGQGTQVT |
| | | VSSAAAYPYDVPDYGS |

X indicates the position of the FSY incorporation site. Sequences in the table with a His6 purification tag and/or PelB leader sequence are also embodied herein.

In some instances, DNA sequences associated with conjugates described herein are in Table 2.

TABLE 2
DNA Sequences

| | SEQ | |
|---|---|---|
| | ID | |
| Name | NO | Sequence |
| C1 | 73 | CTTTAAGAAGGAGATATACATATGAAGTAT |
| | | TTGTTGCCTACAGCGGCTGCCGGATTACTTC |
| | | TGCTTGCCGCACAGCCAGCGATGGCCCAAG |
| | | TCCAACTGCAAGAGTCGGGTGGCGGCTCTG |
| | | TTCAAGCGGGAGGCTCCTTGCGCTTGAGCT |
| | | GTACCGCTCCTGGGTACACAGACAGTAATT |
| | | ATTATATGAGCTGGTTCCGCCAAGCGCCCG |
| | | GTAAAGAACGTGAATGGGTAGCTGGTGTGA |
| | | ACACAGGACGCGGGTCGACATCCTACGCGG |
| | | ACAGTGTTAAAGGTCGCTTCACAATCTCGC |
| | | AAGATAACGCTAAGAACACAATGTTCTTGC |
| | | AAATGAACTCGCTTAAGCCAGAAGATACTG |
| | | CTATTTACTACTGCGCTGTAGCCGCCTGCCA |
| | | CTTCTGTGATAGTCTTCCAAAGACTCAAGAC |
| | | GAGTACATTTTATGGGGCCAGGGCACTCAA |
| | | GTCACAGTTTCGAGCGCCGCCGCATACCCA |
| | | TACGACGTCCCAGACTATGGCTCTCATCATC |
| | | ACCATCACCATtaaAAGCTTGGCTGTTTTGGC |
| | | GGATGAGAGAAG |
| C2 | 74 | CTTTAAGAAGGAGATATACATATGAAGTAT |
| | | TTATTACCTACAGCGGCAGCAGGGTTATTGT |
| | | TATTGGCCGCGCAGCCCGCAATGGCTGAAG |
| | | TTCAACTTGTTGAGAGCGGGGGCGGATTGG |
| | | TACAGCCGGGGGGGTCACTTACCTTATCAT |
| | | GTGCAGCATCGCGCTTCATGATCTCAGAAT |
| | | ACAGTATGCATTGGGTTCGTCAAGCGCCGG |
| | | GGAAAGGTCTTGAGTGGGTTAGTACCATCA |
| | | ACCCAGCCGGAACCACTGATTACGCGGAAA |
| | | GCGTGAAGGGCCGTTTTACCATCTCCCGCG |
| | | ACAATGCTAAAAATACTTTATACTTACAGA |
| | | TGAACAGTTTGAAACCCGAAGATACGGCGG |
| | | TGTACTACTGTGACGGGTACGGCTATCGTG |
| | | GCCAAGGAACACAAGTTACTGTATCCTCTC |
| | | ATCATCATCACCATCACtaaAAGCTTGGCTGT |
| | | TTTGGCGGATGAGAG |
| C3 | 75 | CTTTAAGAAGGAGATATACATATGAAGTAC |
| | | CTTCTGCCGACTGCCGCTGCAGGTTTGTTGT |
| | | TATTGGCCGCTCAACCAGCGATGGCCCAGG |
| | | TACAGCTGCAAGAAAGTGGTGGTGGTAGCG |
| | | TAGAGGCGGGTGGGTCTTTACGTCTGTCGT |
| | | GCGCTCGTTCGGGTTGGCCCTACTCAACGTA |
| | | TAGTATGAATTGGTTCCGTCAGGCCCCCGGT |
| | | AAAGAACGTGAGGCAGTCGCGGGGATTTCG |
| | | TCAACGATGTCTGGGATTATCTTTGCCGAGT |
| | | CCAAAGCTGGACAGTTCACTATTAGCCAAG |
| | | ATAACGCCAAGAACACAGTGTATCTTCAGA |
| | | TGAATAACCTGAAACCGGAAGACACCGCAA |
| | | TTTACTACTGCGCAGCGCGCCGTGACTACTC |
| | | CTTGTCTTCGAGCTCGGACGATTTCGATTAT |
| | | TGGGGTCAGGGGACCCAAGTCACTGTGAGT |
| | | TCCGCCGCAGCATATCCGTATGATGTACCC |
| | | GATTACGGCTCGCACCACCATCATCACCATt |
| | | aaAAGCTTGGCTGTTTTGGCGG |
| C4 | 76 | CTTTAAGAAGGAGATATACATATGAAATAC |
| | | CTGCTGCCGACCGCTGCTGCTGGTCTGCTGC |
| | | TCCTCGCTGCCCAGCCGGCGATGGCCATGG |
| | | GCCAAGTAAAACTAGAGGAATCAGGTGGA |
| | | GGGTCCGTGCAAACCGGTGGCAGTCTCCGT |
| | | TTGACCTGTGCTGCGAGCGGCCGTACCAGC |
| | | CGCAGCTACGGCATGGGTTGGTTTCGTCAG |
| | | GCACCGGGTAAGGAGCGCGAGTTCGTGTCC |
| | | GGCATCAGCTGGCGTGGTGACAGCACGGGC |
| | | TATGCGGATAGCGTTAAAGGTCGCTTCACC |
| | | ATTTCTCGTGATAATGCAAAGAACACCGTT |
| | | GATCTGCAAATGAACAGCCTGAAACCGGAA |
| | | GACACCGCTATCTACTACTGCGCCGCGGCG |
| | | GCGGGTTCTGCCTGGTATGGCACTCTGTATG |
| | | AATACGACTACTGGGGTCAGGGTACGCAGG |
| | | TTACCGTCTCGTCCCATCATCACCACCACCA |
| | | CTAAAAGCTTGGCTGTTTTGGCGGATGAGA |
| | | G |
| C4_109 | 77 | CTTTAAGAAGGAGATATACATATGAAATAC |
| | | CTGCTGCCGACCGCTGCTGCTGGTCTGCTGC |
| | | TCCTCGCTGCCCAGCCGGCGATGGCCATGG |
| | | GCCAAGTAAAACTAGAGGAATCAGGTGGA |
| | | GGGTCCGTGCAAACCGGTGGCAGTCTCCGT |
| | | TTGACCTGTGCTGCGAGCGGCCGTACCAGC |
| | | CGCAGCTACGGCATGGGTTGGTTTCGTCAG |
| | | GCACCGGGTAAGGAGCGCGAGTTCGTGTCC |
| | | GGCATCAGCTGGCGTGGTGACAGCACGGGC |
| | | TATGCGGATAGCGTTAAAGGTCGCTTCACC |
| | | ATTTCTCGTGATAATGCAAAGAACACCGTT |
| | | GATCTGCAAATGAACAGCCTGAAACCGGAA |
| | | GACACCGCTATCTACTACTGCGCCGCGGCG |
| | | GCGGGTTCTGCCTGGTATGGCACTCTGTAG |
| | | GAATACGACTACTGGGGTCAGGGTACGCAG |
| | | GTTACCGTCTCGTCCCATCATCACCACCACC |
| | | ACTAAAAGCTTGGCTGTTTTGGCGGATGAG |
| | | AG |
| C8 | 78 | CTTTAAGAAGGAGATATACATATGAAATAC |
| | | TTACTTCCAACAGCAGCCGCAGGATTATTA |
| | | CTGTTGGCCGCTCAGCCTGCAATGGCTCAA |
| | | GTGCAGTTACAAGAAAGCGGGGGCGGACTT |
| | | GTGCAAACAGGAGGGAGTCTGCGTCTTTCC |
| | | TGTGCGGCGAGTGGGTCGATCTTCGTTGGA |
| | | AATGCGATGGGCTGGTATCGCCAAGCCTTG |
| | | GGTAATCAACGTGAATTAGTCGCAGGGATC |
| | | ACGTCCGATGGGATCACATACTACCCGGAT |
| | | TCAGTTAAGGGCCGCTTTACAATCTCTCGTG |
| | | ATAACGATAAAAACACGATTTACTTACAAA |
| | | TGAATAGTTTAAAACCAGAGGATACCGCTG |
| | | TTTACTACTGCAACCTGTGGCCCCCTCGTAT |
| | | CGGGTTCGCCTCCTGGGGACAAGGAACTCA |
| | | AGTGACGGTCTCGAGTGCTGCCGCTTACCCT |
| | | TACGACGTACCGGATTACGGGTCGCATCAT |
| | | CATCATCACCACTAAAAGCTTGGCTGTTTTG |
| | | GCGGATGAGAG |
| C9 | 79 | CTTTAAGAAGGAGATATACATATGAAATAC |
| | | TTATTACCTACCGCTGCAGCGGGGCTGTTGT |
| | | TGTTGGCCGCCCAGCCAGCAATGGCTATGG |
| | | CGCAAGTCCAATTAGTTCAATCAGGCGGGG |
| | | GGTTGGTCCAGGCGGGGGGATCCTTACGTC |
| | | TGTCATGTGCTTTCAGTGGCCGCACGTTCTC |
| | | AATGTACACGATGGGGTGGTTTCGTCAAGC |
| | | TCCGGGGAAAGAACGCGAGTTTGTGGCCGC |
| | | AAATCGCGGACGTGGGTTAAGTCCCGACAT |
| | | TGCTGACAGTGTTAATGGACGCTTCACCATT |
| | | TCACGCGACAATGCTAAGAATACTCTTTAC |
| | | CTTCAGATGGACTCTCTGAAACCAGAGGAC |
| | | ACCGCCGTATATTATTGCGCCGCAGACTTAC |
| | | AGTACGGGTCATCATGGCCTCAACGTTCGT |
| | | CTGCTGAATATGATTACTGGGGGCAAGGAA |
| | | CGACAGTCACTGTATCCTCGCATCACCACC |
| | | ATCACCATtaaAAGCTTGGCTGTTTTGGCGGA |
| | | TGAGAG |
| pBAD_ | 80 | CTTGGCTGTTTTGGCGGATGAGAGAAGATT |
| fwd | | TTCAGCCTGATACAG |
| pBAD_ | 81 | GTATATCTCCTTCTTAAAGTTAAACAAAATT |
| rev | | ATTTCTAGCCCAAAAAAACGGGTATGGAG |
| pBAD- | 82 | AAGAAACCAATTGTCCATATTGCATCAGAC |
| C8 | | ATTGCCGTCACTGCGTCTTTTACTGGCTCTT |
| | | CTCGCTAACCAAACCGGTAACCCCGCTTATT |
| | | AAAAGCATTCTGTAACAAAGCGGGACCAAA |
| | | GCCATGACAAAAACGCGTAACAAAAGTGTC |
| | | TATAATCACGGCAGAAAAGTCCACATTGAT |
| | | TATTTGCACGGCGTCACACTTTGCTATGCCA |
| | | TAGCATTTTTATCCATAAGATTAGCGGATCC |
| | | TACCTGACGCTTTTTATCGCAACTCTCTACT |
| | | GTTTCTCCATACCCGTTTTTTTGGGCTAGAA |
| | | ATAATTTTGTTTAACTTTAAGAAGGAGATAT |
| | | ACATATGAAATACTTACTTCCAACAGCAGC |
| | | CGCAGGATTATTACTGTTGGCCGCTCAGCCT |
| | | GCAATGGCTCAAGTGCAGTTACAAGAAAGC |
| | | GGGGGCGGACTTGTGCAAACAGGAGGGAG |
| | | TCTGCGTCTTTCCTGTGCGGCGAGTGGGTCG |
| | | ATCTTCGTTGGAAATGCGATGGGCTGGTAT |
| | | CGCCAAGCCTTGGGTAATCAACGTGAATTA |
| | | GTCGCAGGGATCACGTCCGATGGGATCACA |
| | | TACTACCCGGATTCAGTTAAGGGCCGCTTTA |
| | | CAATCTCTCGTGATAACGATAAAAACACGA |
| | | TTTACTTACAAATGAATAGTTTAAAACCAG |
| | | AGGATACCGCTGTTTACTACTGCAACCTGTG |
| | | GCCCCCTCGTATCGGGTTCGCCTCCTGGGGA |
| | | CAAGGAACTCAAGTGACGGTCTCGAGTGCT |
| | | GCCGCTTACCCTTACGACGTACCGGATTAC |
| | | GGGTCGCATCATCATCATCACCACTAAAAG |
| | | CTTGGCTGTTTTGGCGGATGAGAGAAGATT |
| | | TTCAGCCTGATACAGATTAAATCAGAACGC |
| | | AGAAGCGGTCTGATAAAACAGAATTTGCCT |
| | | GGCGGCAGTAGCGCGGTGGTCCCACCTGAC |
| | | CCCATGCCGAACTCAGAAGTGAAACGCCGT |
| | | AGCGCCGATGGTAGTGTGGGGTCTCCCCAT |
| | | GCGAGAGTAGGGAACTGCCAGGCATCAAAT |
| | | AAAACGAAAGGCTCAGTCGAAAGACTGGG |
| | | CCTTTCGTTTTATCTGTTGTTTGTCGGTGAA |
| | | CGCTCTCCTGAGTAGGACAAATCCGCCGGG |
| | | AGCGGATTTGAACGTTGCGAAGCAACGGCC |
| | | CGGAGGGTGGCGGGCAGGACGCCCGCCATA |
| | | AACTGCCAGGCATCAAATTAAGCAGAAGGC |
| | | CATCCTGACGGATGGCCTTTTTGCGTTTCTA |
| | | CAAACTCTTTTTGTTTATTTTTCTAAATACAT |
| | | TCAAATATGTATCCGCTCATGAGACAATAA |
| | | CCCTGATAAATGCTTCAATAATATTGAAAA |
| | | AGGAAGAGTATGAGTATTCAACATTTCCGT |
| | | GTCGCCCTTATTCCCTTTTTTGCGGCATTTTG |
| | | CCTTCCTGTTTTTGCTCACCCAGAAACGCTG |
| | | GTGAAAGTAAAAGATGCTGAAGATCAGTTG |
| | | GGTGCACGAGTGGGTTACATCGAACTGGAT |
| | | CTCAACAGCGGTAAGATCCTTGAGAGTTTT |
| | | CGCCCCGAAGAACGTTTTCCAATGATGAGC |
| | | ACTTTTAAAGTTCTGCTATGTGGCGCGGTAT |
| | | TATCCCGTGTTGACGCCGGGCAAGAGCAAC |
| | | TCGGTCGCCGCATACACTATTCTCAGAATG |
| | | ACTTGGTTGAGTACTCACCAGTCACAGAAA |
| | | AGCATCTTACGGATGGCATGACAGTAAGAG |
| | | AATTATGCAGTGCTGCCATAACCATGAGTG |
| | | ATAACACTGCGGCCAACTTACTTCTGACAA |
| | | CGATCGGAGGACCGAAGGAGCTAACCGCTT |
| | | TTTTGCACAACATGGGGGATCATGTAACTC |
| | | GCCTTGATCGTTGGGAACCGGAGCTGAATG |
| | | AAGCCATACCAAACGACGAGCGTGACACCA |
| | | CGATGCCTGTAGCAATGGCAACAACGTTGC |
| | | GCAAACTATTAACTGGCGAACTACTTACTCT |
| | | AGCTTCCCGGCAACAATTAATAGACTGGAT |
| | | GGAGGCGGATAAAGTTGCAGGACCACTTCT |
| | | GCGCTCGGCCCTTCCGGCTGGCTGGTTTATT |
| | | GCTGATAAATCTGGAGCCGGTGAGCGTGGG |
| | | TCTCGCGGTATCATTGCAGCACTGGGGCCA |
| | | GATGGTAAGCCCTCCCGTATCGTAGTTATCT |
| | | ACACGACGGGGAGTCAGGCAACTATGGATG |
| | | AACGAAATAGACAGATCGCTGAGATAGGTG |
| | | CCTCACTGATTAAGCATTGGTAACTGTCAG |
| | | ACCAAGTTTACTCATATATACTTTAGATTGA |
| | | TTTAAAACTTCATTTTTAATTTAAAAGGATC |
| | | TAGGTGAAGATCCTTTTTGATAATCTCATGA |
| | | CCAAAATCCCTTAACGTGAGTTTTCGTTCCA |
| | | CTGAGCGTCAGACCCCGTAGAAAAGATCAA |
| | | AGGATCTTCTTGAGATCCTTTTTTTCTGCGC |
| | | GTAATCTGCTGCTTGCAAACAAAAAAACCA |
| | | CCGCTACCAGCGGTGGTTTGTTTGCCGGATC |
| | | AAGAGCTACCAACTCTTTTTCCGAAGGTAA |
| | | CTGGCTTCAGCAGAGCGCAGATACCAAATA |
| | | CTGTCCTTCTAGTGTAGCCGTAGTTAGGCCA |
| | | CCACTTCAAGAACTCTGTAGCACCGCCTAC |
| | | ATACCTCGCTCTGCTAATCCTGTTACCAGTG |
| | | GCTGCTGCCAGTGGCGATAAGTCGTGTCTT |
| | | ACCGGGTTGGACTCAAGACGATAGTTACCG |
| | | GATAAGGCGCAGCGGTCGGGCTGAACGGG |
| | | GGGTTCGTGCACACAGCCCAGCTTGGAGCG |
| | | AACGACCTACACCGAACTGAGATACCTACA |
| | | GCGTGAGCTATGAGAAAGCGCCACGCTTCC |
| | | CGAAGGGAGAAAGGCGGACAGGTATCCGG |
| | | TAAGCGGCAGGGTCGGAACAGGAGAGCGC |
| | | ACGAGGGAGCTTCCAGGGGGAAACGCCTGG |
| | | TATCTTTATAGTCCTGTCGGGTTTCGCCACC |
| | | TCTGACTTGAGCGTCGATTTTTGTGATGCTC |
| | | GTCAGGGGGGCGGAGCCTATGGAAAAACG |
| | | CCAGCAACGCGGCCTTTTTACGGTTCCTGGC |
| | | CTTTTGCTGGCCTTTTGCTCACATGTTCTTTC |
| | | CTGCGTTATCCCCTGATTCTGTGGATAACCG |
| | | TATTACCGCCTTTGAGTGAGCTGATACCGCT |
| | | CGCCGCAGCCGAACGACCGAGCGCAGCGA |
| | | GTCAGTGAGCGAGGAAGCGGAAGAGCGCC |
| | | TGATGCGGTATTTTCTCCTTACGCATCTGTG |
| | | CGGTATTTCACACCGCATATCGTGCACTCTC |
| | | AGTACAATCTGCTCTGATGCCGCATAGTTA |
| | | AGCCAGTATACACTCCGCTATCGCTACGTG |
| | | ACTGGGTCATGGCTGCGCCCCGACACCCGC |
| | | CAACACCCGCTGACGCGCCCTGACGGGCTT |
| | | GTCTGCTCCCGGCATCCGCTTACAGACAAG |
| | | CTGTGACCGTCTCCGGGAGCTGCATGTGTC |
| | | AGAGGTTTTCACCGTCATCACCGAAACGCG |
| | | CGAGGCAGCAGATCAATTCGCGCGCGAAGG |
| | | CGAAGCGGCATGCATAATGTGCCTGTCAAA |
| | | TGGACGAAGCAGGGATTCTGCAAACCCTAT |
| | | GCTACTCCGTCAAGCCGTCAATTGTCTGATT |
| | | CGTTACCAATTATGACAACTTGACGGCTAC |
| | | ATCATTCACTTTTTCTTCACAACCGGCACGG |
| | | AACTCGCTCGGGCTGGCCCCGGTGCATTTTT |
| | | TAAATACCCGCGAGAAATAGAGTTGATCGT |
| | | CAAAACCAACATTGCGACCGACGGTGGCGA |
| | | TAGGCATCCGGGTGGTGCTCAAAAGCAGCT |
| | | TCGCCTGGCTGATACGTTGGTCCTCGCGCCA |
| | | GCTTAAGACGCTAATCCCTAACTGCTGGCG |
| | | GAAAAGATGTGACAGACGCGACGGCGACA |
| | | AGCAAACATGCTGTGCGACGCTGGCGATAT |
| | | CAAAATTGCTGTCTGCCAGGTGATCGCTGA |
| | | TGTACTGACAAGCCTCGCGTACCCGATTATC |
| | | CATCGGTGGATGGAGCGACTCGTTAATCGC |
| | | TTCCATGCGCCGCAGTAACAATTGCTCAAG |
| | | CAGATTTATCGCCAGCAGCTCCGAATAGCG |
| | | CCCTTCCCCTTGCCCGGCGTTAATGATTTGC |
| | | CCAAACAGGTCGCTGAAATGCGGCTGGTGC |
| | | GCTTCATCCGGGCGAAAGAACCCCGTATTG |
| | | GCAAATATTGACGGCCAGTTAAGCCATTCA |
| | | TGCCAGTAGGCGCGCGGACGAAAGTAAACC |
| | | CACTGGTGATACCATTCGCGAGCCTCCGGA |
| | | TGACGACCGTAGTGATGAATCTCTCCTGGC |
| | | GGGAACAGCAAAATATCACCCGGTCGGCAA |
| | | ACAAATTCTCGTCCCTGATTTTTCACCACCC |
| | | CCTGACCGCGAATGGTGAGATTGAGAATAT |
| | | AACCTTTCATTCCCAGCGGTCGGTCGATAA |
| | | AAAAATCGAGATAACCGTTGGCCTCAATCG |
| | | GCGTTAAACCCGCCACCAGATGGGCATTAA |
| | | ACGAGTATCCCGGCAGCAGGGGATCATTTT |
| | | GCGCTTCAGCCATACTTTTCATACTCCCGCC |
| | | ATTCAGAG |
| pBAD- | 83 | AAGAAACCAATTGTCCATATTGCATCAGAC |
| C9 | | ATTGCCGTCACTGCGTCTTTTACTGGCTCTT |
| | | CTCGCTAACCAAACCGGTAACCCCGCTTATT |
| | | AAAAGCATTCTGTAACAAAGCGGGACCAAA |
| | | GCCATGACAAAAACGCGTAACAAAAGTGTC |
| | | TATAATCACGGCAGAAAAGTCCACATTGAT |
| | | TATTTGCACGGCGTCACACTTTGCTATGCCA |
| | | TAGCATTTTTATCCATAAGATTAGCGGATCC |
| | | TACCTGACGCTTTTTATCGCAACTCTCTACT |
| | | GTTTCTCCATACCCGTTTTTTTGGGCTAGAA |
| | | ATAATTTTGTTTAACTTTAAGAAGGAGATAT |
| | | ACATATGAAATACTTATTACCTACCGCTGCA |
| | | GCGGGGCTGTTGTTGTTGGCCGCCCAGCCA |
| | | GCAATGGCTATGGCGCAAGTCCAATTAGTT |
| | | CAATCAGGGGGGGGTTGGTCCAGGCGGGG |
| | | GGATCCTTACGTCTGTCATGTGCTTTCAGTG |
| | | GCCGCACGTTCTCAATGTACACGATGGGGT |
| | | GGTTTCGTCAAGCTCCGGGGAAAGAACGCG |
| | | AGTTTGTGGCCGCAAATCGCGGACGTGGGT |
| | | TAAGTCCCGACATTGCTGACAGTGTTAATG |
| | | GACGCTTCACCATTTCACGCGACAATGCTA |
| | | AGAATACTCTTTACCTTCAGATGGACTCTCT |
| | | GAAACCAGAGGACACCGCCGTATATTATTG |
| | | CGCCGCAGACTTACAGTACGGGTCATCATG |
| | | GCCTCAACGTTCGTCTGCTGAATATGATTAC |
| | | TGGGGGCAAGGAACGACAGTCACTGTATCC |
| | | TCGCATCACCACCATCACCATTAAAAGCTT |
| | | GGCTGTTTTGGCGGATGAGAGAAGATTTTC |
| | | AGCCTGATACAGATTAAATCAGAACGCAGA |
| | | AGCGGTCTGATAAAACAGAATTTGCCTGGC |
| | | GGCAGTAGCGCGGTGGTCCCACCTGACCCC |
| | | ATGCCGAACTCAGAAGTGAAACGCCGTAGC |
| | | GCCGATGGTAGTGTGGGGTCTCCCCATGCG |
| | | AGAGTAGGGAACTGCCAGGCATCAAATAAA |
| | | ACGAAAGGCTCAGTCGAAAGACTGGGCCTT |
| | | TCGTTTTATCTGTTGTTTGTCGGTGAACGCT |
| | | CTCCTGAGTAGGACAAATCCGCCGGGAGCG |
| | | GATTTGAACGTTGCGAAGCAACGGCCCGGA |
| | | GGGTGGCGGGCAGGACGCCCGCCATAAACT |
| | | GCCAGGCATCAAATTAAGCAGAAGGCCATC |
| | | CTGACGGATGGCCTTTTTGCGTTTCTACAAA |
| | | CTCTTTTGTTTATTTTTCTAAATACATTCAAA |
| | | TATGTATCCGCTCATGAGACAATAACCCTG |
| | | ATAAATGCTTCAATAATATTGAAAAAGGAA |
| | | GAGTATGAGTATTCAACATTTCCGTGTCGCC |
| | | CTTATTCCCTTTTTTGCGGCATTTTGCCTTCC |
| | | TGTTTTTGCTCACCCAGAAACGCTGGTGAA |
| | | AGTAAAAGATGCTGAAGATCAGTTGGGTGC |
| | | ACGAGTGGGTTACATCGAACTGGATCTCAA |
| | | CAGCGGTAAGATCCTTGAGAGTTTTCGCCC |
| | | CGAAGAACGTTTTCCAATGATGAGCACTTTT |
| | | AAAGTTCTGCTATGTGGCGCGGTATTATCCC |
| | | GTGTTGACGCCGGGCAAGAGCAACTCGGTC |
| | | GCCGCATACACTATTCTCAGAATGACTTGGT |
| | | TGAGTACTCACCAGTCACAGAAAAGCATCT |
| | | TACGGATGGCATGACAGTAAGAGAATTATG |
| | | CAGTGCTGCCATAACCATGAGTGATAACAC |
| | | TGCGGCCAACTTACTTCTGACAACGATCGG |
| | | AGGACCGAAGGAGCTAACCGCTTTTTTGCA |
| | | CAACATGGGGGATCATGTAACTCGCCTTGA |
| | | TCGTTGGGAACCGGAGCTGAATGAAGCCAT |
| | | ACCAAACGACGAGCGTGACACCACGATGCC |
| | | TGTAGCAATGGCAACAACGTTGCGCAAACT |
| | | ATTAACTGGCGAACTACTTACTCTAGCTTCC |
| | | CGGCAACAATTAATAGACTGGATGGAGGCG |
| | | GATAAAGTTGCAGGACCACTTCTGCGCTCG |
| | | GCCCTTCCGGCTGGCTGGTTTATTGCTGATA |
| | | AATCTGGAGCCGGTGAGCGTGGGTCTCGCG |
| | | GTATCATTGCAGCACTGGGGCCAGATGGTA |
| | | AGCCCTCCCGTATCGTAGTTATCTACACGAC |
| | | GGGGAGTCAGGCAACTATGGATGAACGAA |
| | | ATAGACAGATCGCTGAGATAGGTGCCTCAC |
| | | TGATTAAGCATTGGTAACTGTCAGACCAAG |
| | | TTTACTCATATATACTTTAGATTGATTTAAA |
| | | ACTTCATTTTTAATTTAAAAGGATCTAGGTG |
| | | AAGATCCTTTTTGATAATCTCATGACCAAAA |
| | | TCCCTTAACGTGAGTTTTCGTTCCACTGAGC |
| | | GTCAGACCCCGTAGAAAAGATCAAAGGATC |
| | | TTCTTGAGATCCTTTTTTTCTGCGCGTAATCT |
| | | GCTGCTTGCAAACAAAAAAACCACCGCTAC |
| | | CAGCGGTGGTTTGTTTGCCGGATCAAGAGC |
| | | TACCAACTCTTTTTCCGAAGGTAACTGGCTT |
| | | CAGCAGAGCGCAGATACCAAATACTGTCCT |
| | | TCTAGTGTAGCCGTAGTTAGGCCACCACTTC |
| | | AAGAACTCTGTAGCACCGCCTACATACCTC |
| | | GCTCTGCTAATCCTGTTACCAGTGGCTGCTG |
| | | CCAGTGGCGATAAGTCGTGTCTTACCGGGT |
| | | TGGACTCAAGACGATAGTTACCGGATAAGG |
| | | CGCAGCGGTCGGGCTGAACGGGGGGTTCGT |
| | | GCACACAGCCCAGCTTGGAGCGAACGACCT |
| | | ACACCGAACTGAGATACCTACAGCGTGAGC |
| | | TATGAGAAAGCGCCACGCTTCCCGAAGGGA |
| | | GAAAGGCGGACAGGTATCCGGTAAGCGGC |
| | | AGGGTCGGAACAGGAGAGCGCACGAGGGA |
| | | GCTTCCAGGGGGAAACGCCTGGTATCTTTA |
| | | TAGTCCTGTCGGGTTTCGCCACCTCTGACTT |
| | | GAGCGTCGATTTTTGTGATGCTCGTCAGGG |
| | | GGGCGGAGCCTATGGAAAAACGCCAGCAA |
| | | CGCGGCCTTTTTACGGTTCCTGGCCTTTTGC |
| | | TGGCCTTTTGCTCACATGTTCTTTCCTGCGTT |
| | | ATCCCCTGATTCTGTGGATAACCGTATTACC |
| | | GCCTTTGAGTGAGCTGATACCGCTCGCCGC |
| | | AGCCGAACGACCGAGCGCAGCGAGTCAGTG |
| | | AGCGAGGAAGCGGAAGAGCGCCTGATGCG |
| | | GTATTTTCTCCTTACGCATCTGTGCGGTATT |
| | | TCACACCGCATATGGTGCACTCTCAGTACA |
| | | ATCTGCTCTGATGCCGCATAGTTAAGCCAGT |
| | | ATACACTCCGCTATCGCTACGTGACTGGGTC |
| | | ATGGCTGCGCCCCGACACCCGCCAACACCC |
| | | GCTGACGCGCCCTGACGGGCTTGTCTGCTCC |
| | | CGGCATCCGCTTACAGACAAGCTGTGACCG |
| | | TCTCCGGGAGCTGCATGTGTCAGAGGTTTTC |
| | | ACCGTCATCACCGAAACGCGCGAGGCAGCA |
| | | GATCAATTCGCGCGCGAAGGCGAAGCGGCA |
| | | TGCATAATGTGCCTGTCAAATGGACGAAGC |
| | | AGGGATTCTGCAAACCCTATGCTACTCCGTC |
| | | AAGCCGTCAATTGTCTGATTCGTTACCAATT |
| | | ATGACAACTTGACGGCTACATCATTCACTTT |
| | | TTCTTCACAACCGGCACGGAACTCGCTCGG |
| | | GCTGGCCCCGGTGCATTTTTTAAATACCCGC |
| | | GAGAAATAGAGTTGATCGTCAAAACCAACA |
| | | TTGCGACCGACGGTGGCGATAGGCATCCGG |
| | | GTGGTGCTCAAAAGCAGCTTCGCCTGGCTG |
| | | ATACGTTGGTCCTCGCGCCAGCTTAAGACG |
| | | CTAATCCCTAACTGCTGGCGGAAAAGATGT |
| | | GACAGACGCGACGGCGACAAGCAAACATG |
| | | CTGTGCGACGCTGGCGATATCAAAATTGCT |
| | | GTCTGCCAGGTGATCGCTGATGTACTGACA |
| | | AGCCTCGCGTACCCGATTATCCATCGGTGG |
| | | ATGGAGCGACTCGTTAATCGCTTCCATGCG |
| | | CCGCAGTAACAATTGCTCAAGCAGATTTAT |
| | | CGCCAGCAGCTCCGAATAGCGCCCTTCCCC |
| | | TTGCCCGGCGTTAATGATTTGCCCAAACAG |
| | | GTCGCTGAAATGCGGCTGGTGCGCTTCATC |
| | | CGGGCGAAAGAACCCCGTATTGGCAAATAT |
| | | TGACGGCCAGTTAAGCCATTCATGCCAGTA |
| | | GGCGCGCGGACGAAAGTAAACCCACTGGTG |
| | | ATACCATTCGCGAGCCTCCGGATGACGACC |
| | | GTAGTGATGAATCTCTCCTGGCGGGAACAG |
| | | CAAAATATCACCCGGTCGGCAAACAAATTC |
| | | TCGTCCCTGATTTTTCACCACCCCCTGACCG |
| | | CGAATGGTGAGATTGAGAATATAACCTTTC |
| | | ATTCCCAGCGGTCGGTCGATAAAAAAATCG |
| | | AGATAACCGTTGGCCTCAATCGGCGTTAAA |
| | | CCCGCCACCAGATGGGCATTAAACGAGTAT |
| | | CCCGGCAGCAGGGGATCATTTTGCGCTTCA |
| | | GCCATACTTTTCATACTCCCGCCATTCAGAG |

### Payload

Conjugates may comprise a payload. In some instances, the targeting domains (e.g., the single targeting domain or the first targeting domain or the second targeting domain) comprised in the conjugate may guide a payload to a target. In some instances, the targeting domains (e.g., the single targeting domain or the first targeting domain or the second targeting domain) comprise an antibody or fragment thereof that guides a payload to a cell, such as a tumor cell. In some embodiments, the payload comprises an imaging agent, a radioligand agent or a cytotoxic agent. In some embodiments, the cytotoxic moiety comprises a small molecule drug, peptide, protein, or a chemotherapeutic drug. In some embodiments, the payload comprises a radioligand agent. In some embodiments, the radioligand agent is selected from the group consisting of ³⁵S, ³H, ¹¹¹In, ¹¹²In, ¹⁴C, ¹⁸⁶Re, ¹⁸⁸Re, ³²P, ¹⁵³Sm, ⁷⁷Lu, ⁸⁶Y, ⁸⁸Y, ⁹⁰Y, ¹³¹I, ¹²³I, ¹²⁴I, ¹²⁵I, ¹⁴⁹Tb, ²¹¹At, ²¹²Pb/²¹²Bi, ²¹³Bi, ²²³Ra, ²²⁵Ac, ⁶⁴Cu, ⁶⁷Cu, and ²²⁷Th. In some embodiments, the radioligand agent further comprises a chelator. Examples of chelators include DOTA, DOTAGA, NOTA, MACROPA, THP, and TRAP. In some embodiments, the radioligand agent is selected from the group consisting of ^{99m}Tc, ¹³¹I, ²⁰¹Tl, ¹¹¹In, and ⁶⁷Ga. In some instances, the imaging agent comprises a dye. In some instances, a conjugate comprises two or more payloads. For example, a conjugate comprises a payload dye for visualizing tissue penetration and a payload chemotherapeutic agent for killing a tumor.

The payload may be attached to the conjugate. The payload may be covalently attached to the conjugate. In some instances, the payload is not attached to the conjugate via an unnatural amino acid (UAA) residue. In some embodiments, the payload is attached to amino acids n+x (towards the C-terminus) from the UAA residue. In some embodiments, the site of attachment is defined as a position x amino acids away from the unnatural amino acid position n. In some embodiments, the payload is attached to amino acids n−x (towards the N-terminus) from the UAA residue. In some embodiments, the UAA residue is comprised within or within proximity of a region of a targeting domain (e.g., the single targeting domain or the first or second targeting domain) that interfaces with the target. In some instances, the payload is attached at least 2, 5, 10, 20, 25, 30, 40, 50, 60, 70, 80, 90, 100, or at least 125 amino acids away from the unnatural amino acid in the conjugate.

### Unnatural Amino Acids (UAA)

Unnatural amino acids (UAAs) may be incorporated into conjugates described herein. In some instances, UAA residues are present in a targeting domain of the conjugate. In some instances, UAAs are configured to covalently bind to a target. In some instances, UAAs are configured to covalently bind amino acids present in the target. In some instances, a conjugate comprises one, two, three, four, or more UAAs. In some instances, a conjugate comprises a first targeting domain comprising a first UAA and second targeting domain comprising a second UAA. In some instances, a UAA is configured within a targeting domain (e.g., the first targeting domain or the second targeting domain) to covalently bind an amino acid present in the target when the targeting domain engages the target. In some instances, such amino acids comprise nucleophilic amino acids. In some instances, UAA residues form covalent bonds with lysine, histidine, or tyrosine. In some instances, UAA residues are located in a target-binding domain. In some instances, UAAs once incorporated into conjugates are located in CDRs of a targeting domain, such as a targeting domain comprising an antibody or antigen binding fragment, for example, a single domain antibody. In some embodiments, the UAA residue comprises an aryl-fluoro sulfate moiety. In some instances, the UAA residue is genetically encoded into the conjugates described herein. In some instances, the UAA residue comprises a variant of tyrosine or lysine. In some embodiments, the unnatural amino acid residue comprises a structure of Formula I:

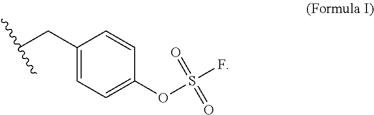 [SMILES: *Cc1ccc(OS(=O)(=O)F)cc1]

In some embodiments, the unnatural amino acid residue comprises a structure of Formula II:

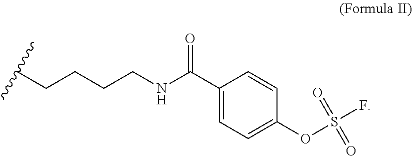 [SMILES: *CCCCNC(=O)c1ccc(OS(=O)(=O)F)cc1]

In some embodiments, the UAA residue is from the incorporation of a UAA described herein. In some embodiments, the unnatural amino acid is 2-amino-3-(4-((fluorosulfonyl)oxy)phenyl)propanoic acid:

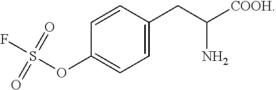 [SMILES: NC(Cc1ccc(OS(=O)(=O)F)cc1)C(=O)O]

In some embodiments, the unnatural amino acid is fluorosulfonyltyrosine (FSY):

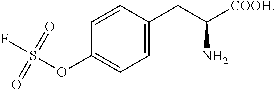 [SMILES: N[C@@H](Cc1ccc(OS(=O)(=O)F)cc1)C(=O)O]

In some embodiments, the unnatural amino acid is N6-(4-((fluorosulfonyl)oxy)benzoyl)lysine:

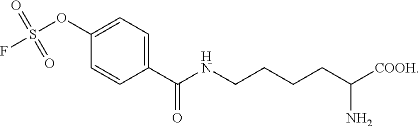 [SMILES: NC(CCCCNC(=O)c1ccc(OS(=O)(=O)F)cc1)C(=O)O]

In some embodiments, the unnatural amino acid is fluorosulfonyloxybenzoyl-L-lysine (FSK):

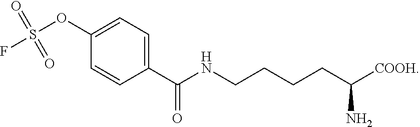 [SMILES: N[C@@H](CCCCNC(=O)c1ccc(OS(=O)(=O)F)cc1)C(=O)O]

In some embodiments, the unnatural amino acid residue comprises a structure of Formula III:

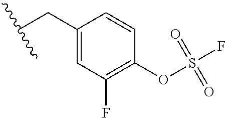 [SMILES: *Cc1ccc(OS(=O)(=O)F)c(F)c1]

In some embodiments, the unnatural amino acid is

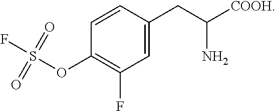 [SMILES: NC(Cc1ccc(OS(=O)(=O)F)c(F)c1)C(=O)O]

In some embodiment, the unnatural amino acid is

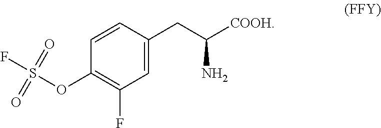 [SMILES: N[C@@H](Cc1ccc(OS(=O)(=O)F)c(F)c1)C(=O)O]

In some embodiments, the unnatural amino acid (UAA) of the UAA residue has a structure of Formula (IA):
(IA),
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, m is 1; when Y is —N═, m is 2.

In some embodiments, the UAA of Formula (IA) has a structure of Formula (IA-a):

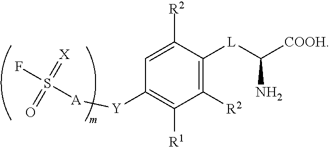

In some embodiments, the UAA of Formula (IA) has a structure of Formula (IA-b):

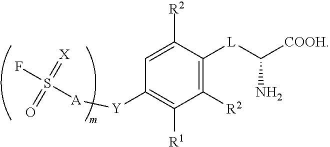

In some embodiments, the UAA of Formula (IA) has a structure of Formula (IB):

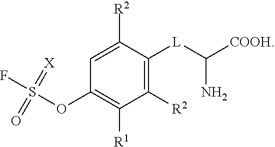

In some embodiments, the UAA of Formula (IB) has a structure of Formula (IB-a):

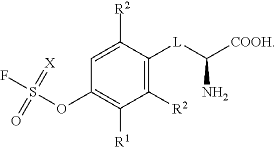

In some embodiments, the UAA of Formula (IB) has a structure of Formula (IB-b):

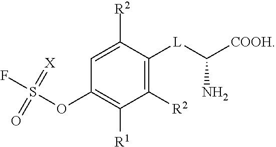

In some embodiments, the UAA of Formula (IA) has a structure of Formula (IC):

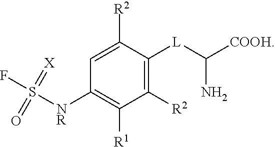

In some embodiments, the UAA of Formula (IC) has a structure of Formula (IC-a):

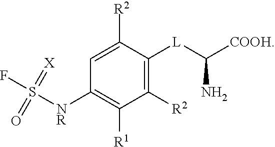

In some embodiments, the UAA of Formula (IB) has a structure of Formula (IB-b):

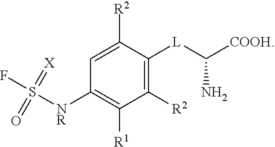

In some preferred embodiments, R is hydrogen.

In some embodiments, the UAA of Formula (IA) has a structure of Formula (ID):

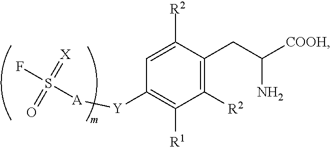

wherein each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

In some embodiments, the UAA of Formula (ID) has a structure of Formula (ID-a):

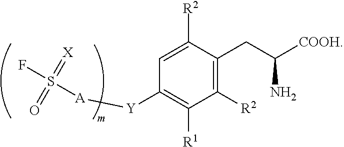

In some embodiments, the UAA of Formula (ID) has a structure of Formula (ID-b):

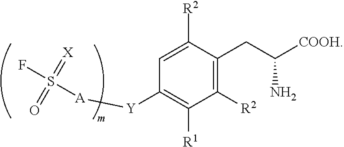

In some embodiments, the UAA of Formula (IA) has a structure of Formula (IE):

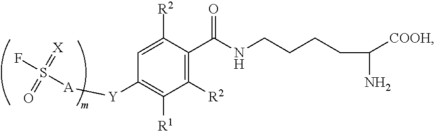

wherein each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

In some embodiments, the UAA of Formula (IE) has a structure of Formula (IE-a):

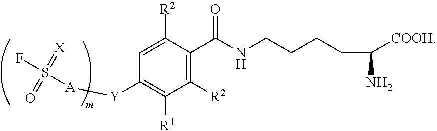

In some embodiments, the UAA of Formula (IE) has a structure of Formula (IE-b):

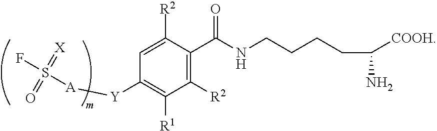

In certain embodiments, Y is a bond, —O— or —NR—, m is 1. In other embodiments, Y is —N═, m is 2. In certain preferred embodiments, Y is —O— and m is 1. In other embodiments, Y is —NR— and m is 1. In other embodiments, Y is a bond and m is 1. In other embodiments, Y is O— or —NR—, m is 1.

In some embodiments, the UAA of Formula (IA) has a structure of Formula (IIA):

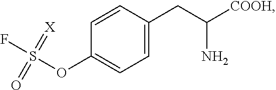

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

In some embodiments, the UAA of Formula (IIA) has a structure of Formula (IIA-a):

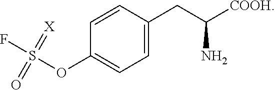

In some embodiments, the UAA of Formula (IIA) has a structure of Formula (IIA-b):

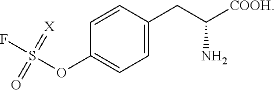

In some embodiments, the UAA of Formula (IA) has a structure of Formula (IIB):

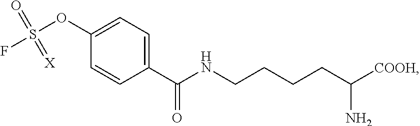

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

In some embodiments, the UAA of Formula (IIB) has a structure of Formula (IIB-a):

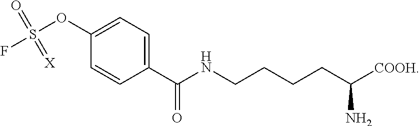

In some embodiments, the UAA of Formula (IIB) has a structure of Formula (IIB-b):

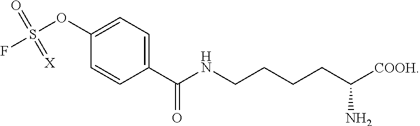

In some embodiments, the unnatural amino acid (UAA) has a structure of

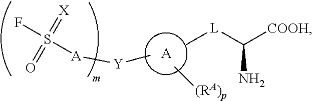

wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NHR^{X}, N(R^{X})₂, or optionally substituted alkyl; each R^{X} is optionally substituted alkyl;
L is —(CH₂)ₚ— or —C(O)NH—(CH₂)ₚ—; p is an integer of 1 to 6; and
wherein when Y is a bond, —O— or —NR—, m is 1; when Y is —N═, m is 2.

In some embodiments, A is a bond. In other embodiments, A is —(CH₂)ₙ—. In some embodiments, n is 1, 2, 3 or 4. In one embodiment, n is 1. In another embodiment, n is 2. In yet another embodiment, n is 3. In yet another embodiment, n is 4.

In some embodiments, Ring A is a 5-membered ring. In some embodiments, Ring A is a 6-membered ring. In some embodiments, Ring A is aryl. In some embodiments, Ring A is heteroaryl. In some embodiments, Ring A is 6-membered aryl. In some embodiments, Ring A is 6-membered heteroaryl. In one preferred embodiment, Ring A is phenyl.

In some embodiments, p is 0. In some embodiments, p is an integer of 1 to 4. In some embodiments, p is an integer of 1 to 3. In one embodiment, p is 1. In another embodiment, p is 2. In yet another embodiment, p is 3. In yet another embodiment, p is 4.

In some embodiments, each R^{A} is independently —OH, —OR^{X}, halogen, NHR^{X}, N(R^{X})₂, or optionally substituted alkyl; each R^{X} is optionally substituted alkyl. In some embodiments, each R^{A} is independently —OH, halo or optionally substituted alkyl. In some embodiments, each R^{A} is independently halo or optionally substituted alkyl. In some embodiments, each R^{A} is independently —OH or optionally substituted alkyl. In some embodiments, each R^{A} is independently optionally substituted alkyl. In some embodiments, each R^{A} is independently substituted alkyl. In some embodiments, each R^{A} is independently unsubstituted alkyl. In one embodiment, each R^{A} is iodo. In another embodiment, each R^{A} is methyl. In one specific embodiment, wherein p is 1, R^{A} is iodo. In another specific embodiment, wherein p is 2, each R^{A} is methyl.

In some embodiments, Y is a bond, —O—, —NR—, or —N═. In some embodiments, Y is —O—, —NR—, or —N═. In some embodiments, Y is a bond, —O—, or —NR—. In some embodiments, Y is a bond, —O—, or —N═. In some embodiments, Y is a bond, —NR—, or —N═. In some embodiments, Y is —O— or —NR—. In some embodiments, Y is —O— or —N═. In some embodiments, Y is —NR— or —N═. In one embodiment, Y is a bond. In another embodiment, Y is —O—. In yet another embodiment, Y is —NR—. In yet another embodiment, Y is —N═.

In some embodiments, L is —(CH₂)ₚ—. In other embodiments, L is —C(O)NH—(CH₂)ₚ—. In some embodiments, p is an integer of 1 to 6, 1 to 5, 1 to 4, 1 to 3, or 1 to 2. In one embodiment, p is 1 to 4. In another embodiment, p is 1 or 2. In yet another embodiment p is 1 or 4. In one preferred embodiment, L is —CH₂—. In another preferred embodiment, L is —C(O)NH—(CH₂)₄—.

In some embodiments, R is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, R is hydrogen or substituted or unsubstituted alkyl. In one preferred embodiment, R is hydrogen. In another embodiment, R is substituted or unsubstituted C₁₋₆ alkyl. In yet another embodiment, R is unsubstituted C₁₋₆ alkyl. In one embodiment R is methyl. In another preferred embodiment, R is hydrogen or methyl.

In some embodiments, R′ is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, R′ is hydrogen, substituted or unsubstituted alkyl, substituted or unsubstituted heteroalkyl, substituted or unsubstituted aryl, or substituted or unsubstituted heteroaryl. In some embodiments, R′ is hydrogen, substituted or unsubstituted alkyl, substituted or unsubstituted aryl, substituted or unsubstituted heteroaryl, or substituted or unsubstituted heterocycloalkyl. In some embodiments, R′ is hydrogen, substituted or unsubstituted alkyl, or substituted or unsubstituted aryl. In embodiment, R′ is hydrogen. In another embodiment, R′ is substituted or unsubstituted. In yet another embodiment, R′ is substituted or unsubstituted aryl.

In some embodiments, R¹ is hydrogen. In some embodiments R¹ is fluoro. In some embodiments R¹ is iodo. In some embodiments, R² is hydrogen. In other embodiments, R² is methyl. In some embodiments, R¹ is hydrogen and R² is hydrogen. In some embodiments, R¹ is fluoro and R² is hydrogen. In some embodiments, R¹ is hydrogen and R² is methyl. In some embodiments, R¹ is iodo and R² is hydrogen.

### Linkers

In some embodiments, the conjugates provided herein comprise more than one linker. In some embodiments, the targeting domains (e.g., the first and the second target domains) are connected via a first linker. The first linker and the second linker can be any linker described herein. In some embodiment, the payload is connected to one of the target domains (the first or the second target domain) via a second linker. In one preferred embodiment, the first linker is a peptide linker. In another preferred embodiment, the second linker is formed through chemical conjugation. In some embodiment, the second linker is a bifunctional linker for chemical conjugation. In another preferred embodiment, the second linker is a non-peptide linker.

In some embodiments, useful functional reactive groups for conjugating or binding a targeting domain to a payload described herein include, for example, zero or higher-order linkers. In some instances, a conjugating moiety comprises a functional reactive group that reacts with a linker (optionally pre-attached to a payload, targeting domain, or other portion of a conjugate) described herein. In some embodiments, a linker comprises a reactive group that reacts with a natural amino acid in a payload or targeting domain described herein. The targeting domains (e.g., the first target domain and the second targeting domain), or one of the targeting domains (e.g., the first target domain or the second targeting domain) and the payload may be conjugated together by reacting a nucleophilic reactive moiety on a first targeting domain with and electrophilic reactive moiety a second targeting domain, or by reacting a nucleophilic reactive moiety on a targeting domain with an electrophilic reactive moiety on the payload. In alternative embodiments, the first targeting domain and the second targeting domain, or a targeting domain (e.g., the first target domain or the second targeting domain) and the payload are conjugated together by reacting an electrophilic reactive moiety on the first targeting domain with a nucleophilic moiety on the second targeting domain, or by reacting an electrophilic reactive moiety on a targeting domain with a nucleophilic moiety on the payload. In some embodiments, an amide bond may form upon reaction of an amine on a targeting domain (e.g. an F-amine of a lysine residue) with a carboxyl group on another targeting domain, or a amine on a targeting domain and a carboxyl group on the payload. In alternative embodiments, the targeting domain and or the payload is derivatized with a derivatizing agent before conjugation

In some cases, higher-order linkers comprise bifunctional linkers, such as homobifunctional linkers or heterobifunctional linkers. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the bifunctional linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N—(γ-maleimidobutyryloxy)succinimide ester (GMBs), N—(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)-1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (ρNPDP), ρ-nitrophenyl-2-diazo-3,3,3-trifluoropropionate (PNP-DTP), sulfhydryl-reactive and photoreactive cross-linkers such as 1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some instances, the reactive functional group comprises a nucleophilic group that is reactive to an electrophilic group present on a binding moiety (e.g., on a payload moiety or on the targeting domain). Exemplary electrophilic groups include carbonyl groups-such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide. In some embodiments, an unnatural amino acid incorporated into a conjugate described herein comprises an electrophilic group.

In some embodiments, the linker is a cleavable linker. In some embodiments, the cleavable linker is a dipeptide linker. In some embodiments, the dipeptide linker is valine-citrulline (Val-Cit), phenylalanine-lysine (Phe-Lys), valine-alanine (Val-Ala) and valine-lysine (Val-Lys). In some embodiments, the dipeptide linker is valine-citrulline. The linker may comprise a cleavable sequence or a sequence that is recognized by a protease.

In various embodiments, the targeting domains (e.g., the first targeting domain and the second targeting domain) comprise a polypeptide linker sequence. The linker may be C-terminal to a targeting domain. The linker may be expressed via recombinant technology and may be encoded using a nucleic acid sequence in conjunction with the expression of the targeting domains. The linker may link the targeting domains to form a fusion protein. The presence of a linker in the conjugates may allow the targeting domains to function properly without steric interference from the other targeting domain. The linker may link a targeting domain to a tag, such as an expression tag or purification tag. The linker may be a flexible linker. The flexibility of the linker may allow the targeting domains to adopt independent conformations with minimal interference from the other targeting domain. In some embodiments, the linker is a peptide linker comprising, e.g., at least 2, 3, 4, 5, 6, 7, 8, 9, 10, 11, 12, 15, 20, 25, 30, 35, 40, 45, 50, or more amino acids. In some instances, the peptide linker comprises at most 2, 3, 4, 5, 6, 7, 8, 9, 10, 11, 12, 15, 20, 25, 30, 35, 40, 45, 50, or less amino acids. In additional cases, the peptide linker comprises about 2, 3, 4, 5, 6, 7, 8, 9, 10, 11, 12, 15, 20, 25, 30, 35, 40, 45, or 50 amino acids. In additional cases, the polypeptide linker comprises about 2, 3, 4, 5, 6, 7, 8, 9, 10, 11, 12, 15, 20, 25, 30, 35, 40, 45, or 50 amino acids. In some instances a polypeptide linker comprises (GGGGSGGGS)ₓ (SEQ ID NO: 14), wherein x is 1-10. In some embodiments, the linker is a polypeptide linker of 1, 1-2, 1-3, 1-4, 1-5, 1-6, 1-7, 1-8, 1-9, 1-10, 1-11, 1-12 amino acids in length, and longer in length. The linker can comprise SPSTPPTPSPSTPP, The polypeptide linker may be a repeat of (GGGGS)x, (GGGS)x. The linker can comprise glycine, serine, threonine, or proline. In some embodiments, the N-terminus of one targeting domain is fused to the C-terminus of the linker polypeptide and the N-terminus of the linker polypeptide is fused to the N-terminus of another targeting domain.

In various embodiments, the targeting domains (e.g., the first targeting domain and the second targeting domain) are connected or separated by a linker. The linker may be a polypeptide linker. The linker may be expressed via recombinant technology and may be encoded using a nucleic acid sequence in conjunction with the expression of the targeting domains. The linker may link the targeting domains to form a fusion protein. The presence of a linker in the conjugates may allow the targeting domains to function properly without steric interference from the other targeting domain. The linker may be a flexible linker. The flexibility of the linker may allow the targeting domains to adopt independent conformations with minimal interference from the other targeting domain. In some embodiments, the linker is a peptide linker comprising, e.g., at least 2, 3, 4, 5, 6, 7, 8, 9, 10, 11, 12, 15, 20, 25, 30, 35, 40, 45, 50, or more amino acids. In some instances, the peptide linker comprises at most 2, 3, 4, 5, 6, 7, 8, 9, 10, 11, 12, 15, 20, 25, 30, 35, 40, 45, 50, or less amino acids. In additional cases, the peptide linker comprises about 2, 3, 4, 5, 6, 7, 8, 9, 10, 11, 12, 15, 20, 25, 30, 35, 40, 45, or 50 amino acids. In additional cases, the polypeptide linker comprises about 2, 3, 4, 5, 6, 7, 8, 9, 10, 11, 12, 15, 20, 25, 30, 35, 40, 45, or 50 amino acids. In some instances a polypeptide linker comprises (GGGGSGGGS)ₓ (SEQ ID NO: 14), wherein x is 1-10. In some embodiments, the linker is a polypeptide linker of 1, 1-2, 1-3, 1-4, 1-5, 1-6, 1-7, 1-8, 1-9, 1-10, 1-11, 1-12 amino acids in length, and longer in length. The linker can comprise SPSTPPTPSPSTPP, The polypeptide linker may be a repeat of (GGGGS)x, (GGGS)x. The linker can comprise glycine, serine, threonine, or proline. In some embodiments, the N-terminus of one targeting domain is fused to the C-terminus of the linker polypeptide and the N-terminus of the linker polypeptide is fused to the N-terminus of another targeting domain.

In some embodiments, the linker comprises a self-immolative linker moiety. In some embodiments, the self-immolative linker moiety comprises p-aminobenzyl alcohol (PAB), p-aminobenzyoxycarbonyl (PABC), or derivatives or analogs thereof. In some embodiments, the linker comprises a dipeptide linker moiety and a self-immolative linker moiety. In some embodiments, the self-immolative linker moiety is such as described in U.S. Pat. No. 9,089,614 and WIPO Application No. WO2015038426.

In some embodiments, the cleavable linker is glucuronide. In some embodiments, the cleavable linker is an acid-cleavable linker. In some embodiments, the acid-cleavable linker is hydrazine. In some embodiments, the cleavable linker is a reducible linker.

In some embodiments, the linker comprises a maleimide group. In some instances, the maleimide group is also referred to as a maleimide spacer. In some instances, the maleimide group further comprises a caproic acid, forming maleimidocaproyl (mc). In some cases, the linker comprises maleimidocaproyl (mc). In some cases, linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some instances, a linker comprises at least one, at least two, at least three, at least four, at least five, at least six, at least seven, at least eight, at least nine, or ten or more of a carbonyl or dicarbonyl group, oxime group, hydroxylamine group, or protected forms thereof. The TLR-agonist linker derivative or the targeting domain can be the same or different, for example, there can be 1, 2, 3, 4, 5, 6, 7, 8, 9, 10, 11, 12, 13, 14, 15, 16, 17, 18, 19, 20, or more different sites in the derivative that comprise 1, 2, 3, 4, 5, 6, 7, 8, 9, 10, 11, 12, 13, 14, 15, 16, 17, 18, 19, 20, or more different reactive groups.

As described herein, the present disclosures provide targeting domains coupled to another molecule having the formula “targeting domain-L-Payload”, wherein L is a linking group or a chemical bond. In some embodiments, L is stable in vivo. In some embodiments, L is hydrolyzable in vivo. In some embodiments, L is metastable in vivo.

A targeting domain and payload can be linked together through L using standard linking agents and procedures known to those skilled in the art. In some aspects, targeting domain and the payload are fused directly and L is a bond. In other aspects, targeting domain and the payload are fused through a linking group L. For example, in some embodiments, targeting domain and the payload are linked together via a peptide bond, optionally through a peptide or amino acid spacer. In some embodiments, targeting domain and the payload are linked together through chemical conjugation, optionally through a linking group (L). In some embodiments, L is directly conjugated to each of targeting domain and a payload.

Chemical conjugation can occur by reacting a nucleophilic reactive group of one compound to an electrophilic reactive group of another compound. In some embodiments when L is a bond, targeting domain is conjugated to the payload either by reacting a nucleophilic reactive moiety on targeting domain with an electrophilic reactive moiety on a linker, or by reacting an electrophilic reactive moiety on targeting domain with a nucleophilic reactive moiety on a payload. In embodiments when L is a group that links targeting domain and payload together, targeting domain and/or payload can be conjugated to L either by reacting a nucleophilic reactive moiety on targeting domain and/or payload with an electrophilic reactive moiety on L, or by reacting an electrophilic reactive moiety on targeting domain and/or payload with a nucleophilic reactive moiety on L. Nonlimiting examples of nucleophilic reactive groups include amino, thiol, and hydroxyl. Nonlimiting examples of electrophilic reactive groups include carboxyl, acyl chloride, anhydride, ester, succinimide ester, alkyl halide, sulfonate ester, maleimido, haloacetyl, and isocyanate. In embodiments where targeting domain and payload are conjugated together by reacting a carboxylic acid with an amine, an activating agent can be used to form an activated ester of the carboxylic acid.

The activated ester of the carboxylic acid can be, for example, N-hydroxysuccinimide (NHS), tosylate (Tos), mesylate, triflate, a carbodiimide, or a hexafluorophosphate. In some embodiments, the carbodiimide is 1,3-dicyclohexylcarbodiimide (DCC), 1,1′-carbonyldiimidazole (CDI), 1-ethyl-3-(3-dimethylaminopropyl)carbodiimide hydrochloride (EDC), or 1,3-diisopropylcarbodiimide (DICD). In some embodiments, the hexafluorophosphate is selected from a group consisting of hexafluorophosphate benzotriazol-1-yl-oxy-tris(dimethylamino)phosphonium hexafluorophosphate (BOP), benzotriazol-1-yl-oxytripyrrolidinophosphonium hexafluorophosphate (PyBOP), 2-(1H-7-azabenzotriazol-1-yl)-1,1,3,3 -tetramethyl uronium hexafluorophosphate (HATU), and o-benzotriazole-N,N,N′,N′-tetramethyl-uronium-hexafluoro-phosphate (HBTU).

In some embodiments, a targeting domain (e.g., the first target domain and the second targeting domain) comprises a nucleophilic reactive group (e.g. the amino group, thiol group, or hydroxyl group of the side chain of lysine, cysteine, or serine) that is capable of conjugating to an electrophilic reactive group on payload or L. In some embodiments, targeting domain comprises an electrophilic reactive group (e.g. the carboxylate group of the side chain of Asp or Glu) that is capable of conjugating to a nucleophilic reactive group on payload or L. In some embodiments, targeting domain is chemically modified to comprise a reactive group that is capable of conjugating directly to payload or to L. In some embodiments, targeting domain is modified at the N-terminus or C-terminus to comprise a natural amino acid with a nucleophilic side chain. In exemplary embodiments, the N-terminus or C-terminus amino acid of targeting domain is selected from the group consisting of lysine, ornithine, serine, cysteine, and homocysteine. For example, the N-terminus or C-terminus amino acid of targeting domain can be modified to comprise a lysine residue. In some embodiments, targeting domain is modified at the N-terminus or C-terminus amino acid to comprise a natural amino acid with an electrophilic side chain such as, for example, Asp and Glu. In some embodiments, an internal amino acid of targeting domain is substituted with a natural amino acid having a nucleophilic side chain, as previously described herein. In exemplary embodiments, the internal amino acid of targeting domain that is substituted is selected from the group consisting of lysine, ornithine, serine, cysteine, and homocysteine. For example, an internal amino acid of targeting domain can be substituted with a lysine residue. In some embodiments, an internal amino acid of targeting domain is substituted with a natural amino acid with an electrophilic side chain, such as, for example, Asp and Glu.

In some embodiments, the payload comprises a reactive group that is capable of conjugating directly to targeting domain or to L. In some embodiments, the payload comprises a nucleophilic reactive group (e.g. amine, thiol, hydroxyl) that is capable of conjugating to an electrophilic reactive group on targeting domain or L. In some embodiments, the payload comprises electrophilic reactive group (e.g. carboxyl group, activated form of a carboxyl group, compound with a leaving group) that is capable of conjugating to a nucleophilic reactive group on targeting domain or L. In some embodiments, the payload is chemically modified to comprise either a nucleophilic reactive group that is capable of conjugating to an electrophilic reactive group on targeting domain or L. In some embodiments, the payload is chemically modified to comprise an electrophilic reactive group that is capable of conjugating to a nucleophilic reactive group on targeting domain or L.

In some embodiments, conjugation can be carried out through organosilanes, for example, aminosilane treated with glutaraldehyde; carbonyldiimidazole (CDI) activation of silanol groups; or utilization of dendrimers. A variety of dendrimers are known in the art and include poly (amidoamine) (PAMAM) dendrimers, which are synthesized by the divergent method starting from ammonia or ethylenediamine initiator core reagents; a sub-class of PAMAM dendrimers based on a tris-aminoethylene-imine core; radially layered poly(amidoamine-organosilicon) dendrimers (PAMAMOS), which are inverted unimolecular micelles that consist of hydrophilic, nucleophilic polyamidoamine (PAMAM) interiors and hydrophobic organosilicon (OS) exteriors; Poly (Propylene Imine) (PPI) dendrimers, which are generally poly-alkyl amines having primary amines as end groups, while the dendrimer interior consists of numerous of tertiary tris-propylene amines; Poly (Propylene Amine) (POPAM) dendrimers; Diaminobutane (DAB) dendrimers; amphiphilic dendrimers; micellar dendrimers which are unimolecular micelles of water soluble hyper branched polyphenylenes; polylysine dendrimers; and dendrimers based on poly-benzyl ether hyper branched skeleton.

In some embodiments, conjugation can be carried out through olefin metathesis. In some embodiments, the payload and the targeting domain, the payload and L, or the targeting domain and L both comprise an alkene or alkyne moiety that is capable of undergoing metathesis. In some embodiments a suitable catalyst (e.g. copper, ruthenium) is used to accelerate the metathesis reaction. Suitable methods of performing olefin metathesis reactions are described in the art. See, for example, Schafmeister et al., J. Am. Chem. Soc. 122: 5891-5892 (2000), Walensky et al., Science 305: 1466-1470 (2004), and Blackwell et al., Angew, Chem., Int. Ed. 37: 3281-3284 (1998).

In some embodiments, conjugation can be carried out using click chemistry. A “click reaction” is wide in scope and easy to perform, uses only readily available reagents, and is insensitive to oxygen and water. In some embodiments, the click reaction is a cycloaddition reaction between an alkynyl group and an azido group to form a triazolyl group. In some embodiments, the click reaction uses a copper or ruthenium catalyst. Suitable methods of performing click reactions are described in the art. See, for example, Kolb et al., Drug Discovery Today 8: 1128 (2003); Kolb et al., Angew. Chem. Int. Ed. 40:2004 (2001); Rostovtsev et al., Angew. Chem. Int. Ed. 41:2596 (2002); Tornoe et al., J. Org. Chem. 67:3057 (2002); Manetsch et al., J. Am. Chem. Soc. 126: 12809 (2004); Lewis et al., Angew. Chem. Int. Ed. 41: 1053 (2002); Speers, J. Am. Chem. Soc. 125:4686 (2003); Chan et al. Org. Lett. 6:2853 (2004); Zhang et al., J. Am. Chem. Soc. 127: 15998 (2005); and Waser et al., J. Am. Chem. Soc. 127:8294 (2005).

Indirect conjugation via high affinity specific binding partners, e.g. streptavidin/biotin or avidin/biotin or lectin/carbohydrate is also contemplated.

### Reactive Residues for Conjugation

In some embodiments, targeting domain and/or the payload are functionalized to comprise a nucleophilic reactive group or an electrophilic reactive group with an organic derivatizing agent. This derivatizing agent is capable of reacting with selected side chains or the N- or C-terminal residues of targeted amino acids on targeting domain and functional groups on the payload. Reactive groups on targeting domain and/or the payload include, e.g., aldehyde, amino, ester, thiol, a-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, targeting domain and/or the payload can be linked to each other indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Cysteinyl residues most commonly are reacted with a-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-3-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group.

Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. These methods are described in WO1987/05330, and in Aplin and Wriston, CRC Crit. Rev. Biochem., pp. 259-306 (1981).

In some embodiments, L is a bond. In these embodiments, targeting domain and the payload are conjugated together by reacting a nucleophilic reactive moiety on targeting domain with and electrophilic reactive moiety or the payload. In alternative embodiments, targeting domain and the payload are conjugated together by reacting an electrophilic reactive moiety on targeting domain with a nucleophilic moiety on the payload. In exemplary embodiments, L is an amide bond that forms upon reaction of an amine on targeting domain (e.g. an F-amine of a lysine residue) with a carboxyl group on M. In alternative embodiments, targeting domain and or the payload is derivatized with a derivatizing agent before conjugation.

In some embodiments, L is a linking group. In some embodiments, L is a bifunctional linker and comprises only two reactive groups before conjugation to targeting domain and the payload. In embodiments where both targeting domain and the payload have electrophilic reactive groups, L comprises two of the same or two different nucleophilic groups (e.g. amine, hydroxyl, thiol) before conjugation to targeting domain and the payload. In embodiments where both targeting domain and the payload have nucleophilic reactive groups, L comprises two of the same or two different electrophilic groups (e.g. carboxyl group, activated form of a carboxyl group, compound with a leaving group) before conjugation to targeting domain the payload. In embodiments where one of targeting domain or the payload has a nucleophilic reactive group and the other of targeting domain or the payload has an electrophilic reactive group, L comprises one nucleophilic reactive group and one electrophilic group before conjugation to targeting domain and the payload.

L can be any molecule with at least two reactive groups (before conjugation to targeting domain and payload) capable of reacting with each of targeting domain the payload. In some embodiments L has only two reactive groups and is bifunctional. L (before conjugation to the peptides) can be represented by Formula VI: A-L-B, wherein A and B are independently nucleophilic or electrophilic reactive groups. In some embodiments A and B are either both nucleophilic groups or both electrophilic groups. In some embodiments one of A or B is a nucleophilic group and the other of A or B is an electrophilic group.

In some embodiments, A and B may include alkene and/or alkyne functional groups that are suitable for olefin metathesis reactions. In some embodiments, A and B include moieties that are suitable for click chemistry (e.g. alkene, alkynes, nitriles, azides). Other nonlimiting examples of reactive groups (A and B) include pyridyldithiol, aryl azide, diazirine, carbodiimide, and hydrazide.

In some embodiments, L is hydrophobic. Hydrophobic linkers are known in the art. See, e.g., Bioconjugate Techniques, G. T. Hermanson (Academic Press, San Diego, CA, 1996), which is incorporated by reference in its entirety. Suitable hydrophobic linking groups known in the art include, for example, 8-hydroxy octanoic acid and 8-mercaptooctanoic acid. Before conjugation to the peptides of the composition, the hydrophobic linking group comprises at least two reactive groups (A and B), as described herein and as shown below: A-(hydrophobic linking group)-B.

In some embodiments, the hydrophobic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on targeting domain or the payload and the carboxylic acid or activated carboxylic acid can be coupled to an amine on targeting domain or the payload with or without the use of a coupling reagent. Any coupling agent known to one skilled in the art can be used to couple the carboxylic acid with the free amine such as, for example, DCC, DIC, HATU, HBTU, TBTU, and other activating agents described herein. In specific embodiments, the hydrophilic linking group comprises an aliphatic chain of 2 to 100 methylene groups wherein A and B are carboxyl groups or derivatives thereof (e.g. succinic acid). In other specific embodiments the L is iodoacetic acid.

In some instances, before conjugation to the peptides of the composition, the hydrophilic linking group comprises at least two reactive groups (A and B), as described herein and as shown below: A-(hydrophilic linking group)-B. In specific embodiments, the linking group is polyethylene glycol (PEG). The PEG in certain embodiments has a molecular weight of about 100 Daltons to about 10,000 Daltons, e.g. about 500 Daltons to about 5000 Daltons. The PEG in some embodiments has a molecular weight of about 10,000 Daltons to about 40,000 Daltons.

In some embodiments, the hydrophilic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on targeting domain or the payload and the carboxylic acid or activated carboxylic acid can be coupled to an amine on targeting domain or the payload with or without the use of a coupling reagent. Any appropriate coupling agent known to one skilled in the art can be used to couple the carboxylic acid with the amine such as, for example, DCC, DIC, HATU, HBTU, TBTU, and other activating agents described herein. In some embodiments, the linking group is maleimido-polymer (0.1-2.5 kDa)-COOH, iodoacetyl-polymer (0.1-2.5 kDa)-COOH, maleimido-polymer (0.1-2.5 kDa)-NHS, or iodoacetyl-polymer (0.1-2.5 kDa)-NHS.

In some embodiments, the linking group is comprised of an amino acid, a dipeptide, a tripeptide, or a polypeptide, wherein the amino acid, dipeptide, tripeptide, or polypeptide comprises at least two activating groups, as described herein. In some embodiments, the linking group (L) comprises a moiety selected from the group consisting of: amino, ether, thioether, maleimido, disulfide, amide, ester, thioester, alkene, cycloalkene, alkyne, triazole, carbamate, carbonate, cathepsin B-cleavable, and hydrazone.

In some embodiments, L comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers in some instances are selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

In some embodiments, L is stable in biological fluids such as blood or blood fractions. In some embodiments, L is stable in blood serum for at least 5 minutes, e.g. less than 25%, 20%, 15%, 10% or 5% of the conjugate is cleaved when incubated in serum for a period of 5 minutes. In other embodiments, L is stable in blood serum for at least 10, or 20, or 25, or 30, or 60, or 90, or 120 minutes, or 3, 4, 5, 6, 7, 8, 9, 10, 12, 15, 18 or 24 hours. In these embodiments, L does not comprise a functional group that is capable of undergoing hydrolysis in vivo. In some exemplary embodiments, L is stable in blood serum for at least about 72 hours. Nonlimiting examples of functional groups that are not capable of undergoing significant hydrolysis in vivo include amides, ethers, and thioethers.

In some embodiments, L is hydrolyzable in vivo. In these embodiments, L comprises a functional group that is capable of undergoing hydrolysis in vivo. Nonlimiting examples of functional groups that are capable of undergoing hydrolysis in vivo include esters, anhydrides, and thioesters.

In some exemplary embodiments L is labile and undergoes substantial hydrolysis within 3 hours in blood plasma at 37° C., with complete hydrolysis within 6 hours. In some exemplary embodiments, L is not labile.

In some embodiments, L is metastable in vivo. In these embodiments, L comprises a functional group that is capable of being chemically or enzymatically cleaved in vivo (e.g., an acid-labile, reduction-labile, or enzyme-labile functional group), optionally over a period of time.

In these embodiments, L can comprise, for example, a hydrazone moiety, a disulfide moiety, or a cathepsin-cleavable moiety. When L is metastable, and without intending to be bound by any particular theory, the targeting domain-L-M conjugate is stable in an extracellular environment, e.g., stable in blood serum for the time periods described above, but labile in the intracellular environment or conditions that mimic the intracellular environment, so that it cleaves upon entry into a cell. In some embodiments when L is metastable, L is stable in blood serum for at least about 24, 25, 26, 27, 28, 29, 30, 31, 32, 33, 34, 35, 36, 42, or 48 hours, for example, at least about 48, 54, 60, 66, or 72 hours, or about 24-48, 48-72, 24-60, 36-48, 36-72, or 48-72 hours.

In some instances a suitable polymer backbone has the formula X-polymer-LY, wherein polymer is poly(ethylene glycol) and X is a functional group which does not react with azide groups and Y is a suitable leaving group. Examples of suitable functional groups include, but are not limited to, hydroxyl, protected hydroxyl, acetal, alkenyl, amine, aminooxy, protected amine, protected hydrazide, protected thiol, carboxylic acid, protected carboxylic acid, maleimide, dithiopyridine, and vinylpyridine, and ketone. Examples of suitable leaving groups include, but are not limited to, chloride, bromide, iodide, mesylate, tresylate, and tosylate.

The linker may have a wide range of molecular weight or molecular length. Larger or smaller molecular weight linkers may be used to provide a desired spatial relationship or conformation between targeting domain and the linked entity or between the linked entity and its binding partner, if any. Linkers having longer or shorter molecular length may also be used to provide a desired space or flexibility between targeting domain and the linked entity, or between the linked entity and its binding partner.

In some embodiments, a linker comprises a water-soluble bifunctional linker that have a dumbbell structure that includes: a) an azide, an alkyne, a hydrazine, a hydrazide, a hydroxylamine, or a carbonyl-containing moiety on at least a first end of a polymer backbone; and b) at least a second functional group on a second end of the polymer backbone. The second functional group can be the same or different as the first functional group. The second functional group, in some embodiments, is not reactive with the first functional group. In some embodiments, water-soluble compounds comprise at least one arm of a branched molecular structure. For example, the branched molecular structure can be dendritic.

In exemplary embodiments, the polymer is linked to the targeting domain or modified targeting domain through a linker. For example, the linker can comprise one or two amino acids which at one end bind to the polymer—such as an albumin binding moiety—and at the other end bind to any available position on the polypeptide backbone. Additional exemplary linkers include a hydrophilic linker such as a chemical moiety which comprises at least 5 non-hydrogen atoms where 30-50% of these are either N or O.

Optionally, multiple targeting domain or modified targeting domain molecules may be joined by a linker polypeptide, wherein said linker polypeptide optionally is 1, 1-2, 1-3, 1-4, 1-5, 1-6, 1-7, 1-8, 1-9, 1-10, 1-11, 1-12 amino acids in length, and longer in length, wherein optionally the N-terminus of one targeting domain is fused to the C-terminus of the linker polypeptide and the N-terminus of the linker polypeptide is fused to the N-terminus of another targeting domain.

The terms “electrophilic group”, “electrophile” and the like as used herein refers to an atom or group of atoms that can accept an electron pair to form a covalent bond. The “electrophilic group” used herein includes but is not limited to halide, carbonyl and epoxide containing compounds. Common electrophiles include halides such as thiophosgene, glycerin dichlorohydrin, phthaloyl chloride, succinyl chloride, chloroacetyl chloride, chlorosuccinyl chloride, etc.; ketones such as chloroacetone, bromoacetone, etc.; aldehydes such as glyoxal, etc.; isocyanates such as hexamethylene diisocyanate, tolylene diisocyanate, meta-xylylene diisocyanate, cyclohexylmethane-4,4-diisocyanate, and derivatives of these compounds.

The terms “nucleophilic group”, “nucleophile” and the like as used herein refers to an atom or group of atoms that have an electron pair capable of forming a covalent bond. Groups of this type may be ionizable groups that react as anionic groups. The “nucleophilic group” used herein includes but is not limited to hydroxyl, primary amines, secondary amines, tertiary amines, and thiols.

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Non-limiting examples of carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other non-limiting examples of carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-limiting examples of non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, including, but not limited to, oxygen, sulfur, or nitrogen.

In some cases, a polymer used herein terminates on one end with hydroxy or methoxy, i.e., X is H or CH₃ (“methoxy PEG”). Alternatively, the polymer can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester) and aldehydes) as well as functional groups that are inert to the 20 common amino acids but that react specifically with complementary functional groups (including but not limited to, azide groups, alkyne groups). It is noted that the other end of the polymer, which is shown in the above formula by Y, will attach either directly or indirectly to a targeting domain via an amino acid. For instance, Y is an amide, carbamate, or urea linkage to an amine group (including but not limited to, the epsilon amine of lysine or the N-terminus) of the polypeptide. Alternatively, Y is a maleimide linkage to a thiol group (including but not limited to, the thiol group of cysteine). Alternatively, an alkyne group on the polymer can be reacted with an azide group present in a targeting domain to form a similar product. In some embodiments, a strong nucleophile (including but not limited to, hydrazine, hydrazide, hydroxylamine, semicarbazide) can be reacted with an aldehyde or ketone group present in a targeting domain to form a hydrazone, oxime or semicarbazone, as applicable, which in some cases can be further reduced by treatment with an appropriate reducing agent. Alternatively, the strong nucleophile can be incorporated into the targeting domain via an amino acid and used to react preferentially with a ketone or aldehyde group present in the water-soluble polymer.

Any molecular mass for a polymer can be used as practically desired, including but not limited to, from about 0.1 Daltons (Da) to 2,500 Da or more. The molecular weight of polymer may be of a wide range, including but not limited to, between about 100 Da and about 5,000 Da or more. In some instances the polymer is 50-5000 Da, 50-3000 Da, 50-2500 Da, 100-2500 Da, 250-2500 Da, 250-5000 Da, or 500-5000 Da. Branched chain polymers, including but not limited to, polymer molecules with each chain having a molecular weight ranging from 0.1-5 kDa, 0.1-4 kDa, 0.1-3 kDa, 0.1-2.5 kDa, 0.1-1.5 kDa.

Polymers may comprise azide- and acetylene-containing polymer derivatives comprising a water-soluble polymer backbone having an average molecular weight from about 800 Da to about 100,000 Da. The polymer backbone of the water-soluble polymer can be poly(ethylene glycol). However, it should be understood that a wide variety of water-soluble polymers including but not limited to poly(ethylene)glycol and other related polymers, including poly(dextran) and poly(propylene glycol), are also and that the use of the term PEG or poly(ethylene glycol) is intended to encompass and include all such molecules. The term PEG includes, but is not limited to, poly(ethylene glycol) in any of its forms, including bifunctional PEG, multiarmed PEG, derivatized PEG, forked PEG, branched PEG, pendent PEG (i.e. PEG or related polymers having one or more functional groups pendent to the polymer backbone), or PEG with degradable linkages therein.

In addition to these forms of polymer, the polymer can also be prepared with weak or degradable linkages in the backbone. For example, polymer can be prepared with ester linkages in the polymer backbone that are subject to hydrolysis. As shown below, this hydrolysis results in cleavage of the polymer into fragments of lower molecular weight: -polymer-CO₂-polymer-+H₂O àpolymer-CO₂H+HO-polymer-

Linkers may comprise a polymer, such as those comprising a water soluble backbone. In some embodiments, polymer backbones that are water-soluble comprise from 2 to about 300 termini. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da. The molecular weight of each chain of the polymer backbone may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of each chain of the polymer backbone is between about 100 Da and about 50,000 Da. In some embodiments, the molecular weight of each chain of the polymer backbone is between about 100 Da and about 40,000 Da. In some embodiments, the molecular weight of each chain of the polymer backbone is between about 1,000 Da and about 40,000 Da. In some embodiments, the molecular weight of each chain of the polymer backbone is between about 5,000 Da and about 40,000 Da. In some embodiments, the molecular weight of each chain of the polymer backbone is between about 10,000 Da and about 40,000 Da.

Appropriate physiologically cleavable linkages include but are not limited to ester, carbonate ester, carbamate, sulfate, phosphate, acyloxyalkyl ether, acetal, and ketal. Such conjugates should possess a physiologically cleavable bond that is stable upon storage and upon administration. For instance, a targeting domain or modified targeting domain linked to a polymer should maintain its integrity upon manufacturing of the final pharmaceutical composition, upon dissolution in an appropriate delivery vehicle, if employed, and upon administration irrespective of route.

The present invention also includes phosphate-based linkers with tunable stability for intracellular delivery of drug conjugates disclosed in US 2017/0182181, incorporated by reference herein. The phosphate-based linkers comprise a monophosphate, diphosphate, triphosphate, or tetraphosphate group (phosphate group) covalently linked to the distal end of a linker arm comprising from the distal to the proximal direction a tuning element, optionally a spacer element, and a reactive functional group. The phosphate group of the phosphate-based linker is capable of being conjugated to a payload and the reactive functional group is capable of being conjugated to a cell-specific targeting ligand such as an antibody. The general structure of the phosphate-based linkers is: Phosphate group-Tuning element-Optional spacer element-Functional reactive group A phosphate-based linker conjugated to a payload has the general structure: Payload-Phosphate group-Tuning element-Optional spacer element-Functional reactive group and when conjugated to a targeting ligand has the general structure Payload-Phosphate group-Tuning element-Optional spacer element-Targeting ligand. These phosphate-based linkers have a differentiated and tunable stability in blood vs. an intracellular environment (e.g. lysosomal compartment). The rate at which the phosphate group is cleaved in the intracellular environment to release the payload in its native or active form may be affected by the structure of the tuning element with further effects mediated by substitutions of the phosphate group as well as whether the phosphate group is a monophosphate, diphosphate, triphosphate, or tetraphosphate. Further, these phosphate-based linkers provide the ability to construct conjugates such as antibody-drug conjugates in which the propensity of the conjugate to form aggregates is reduced compared to conjugates in which the same payload is conjugated to the antibody or targeting ligand using a linker that is not a phosphate-based linker as disclosed herein.

In some instances a targeting domain is linked to a payload via a water soluble polymer via methods described herein. In some embodiments, the method comprises contacting an isolated targeting domain comprising a reactive amino acid side chain with a linker. In some instances a conjugate is synthesized by reacting a functional group present on the targeting domain with a reactive group present on the linker. In some instances a conjugate is synthesized by reacting a functional group present on the linker with a reactive group present on the payload. In some instances, a payload-linker moiety is conjugated to a targeting domain. In some instances, a targeting domain-linker moiety is conjugated to a payload. In some embodiments, the targeting domain is linked to a linker comprising a water soluble polymer.

In other embodiments, the targeting domain is conjugated to a payload via a linker. In some instances, the linker comprises a polymer. In some embodiments, the targeting domain is directly or indirectly conjugated to linker, polymer, or biologically active molecule. In some embodiments, the linker is a cleavable or non-cleavable linker.

In some embodiments, the linker is 0.1 kDa to 5 kDa. In other embodiments, the linker is 0.1 kDa to 2.5 kDa. In other embodiments, the linker or polymer is linear, branched, multimeric, or dendrimeric. In another embodiment, the linker or polymer is a bifunctional or multifunctional linker or a bifunctional or multifunctional polymer.

In other embodiments, the polymer is a water-soluble polymer. In other embodiments, the water-soluble polymer is polyethylene glycol (PEG). In some embodiments, the PEG has a molecular weight between 0.1 kDa and 10 kDa. In other embodiments, the PEG has a molecular weight between 0.1 kDa and 5 kDa. In other embodiments, the PEG has a molecular weight between 0.1 kDa and 4 kDa. In other embodiments, the PEG has a molecular weight between 0.1 kDa and 3 kDa. In other embodiments, the PEG has a molecular weight between 0.1 kDa and 2 kDa. In other embodiments, the PEG has a molecular weight between 0.1 kDa and 2.5 kDa. In some embodiments, the poly(ethylene glycol) molecule has a molecular weight of about 0.1 kDa to about 10 kDa. In some embodiments, the poly(ethylene glycol) molecule has a molecular weight of 0.1 kDa to 50 kDa. In some embodiments, the poly(ethylene glycol) has a molecular weight of 0.1 kDa to 2.5 kDa, or 0.2 to 2.2 kDa, or between 0.5 kDa and 2 kDa. For example, the molecular weight of the poly(ethylene glycol) polymer in some instances is about 0.5 kDa, or about 1 kDa, or about 2 kDa, or about 2.5 kDa. For example, the molecular weight of the poly(ethylene glycol) polymer in some instances is 0.1 kDa or 0.5 kDa or 1 kDa, or 2.5 kDa. In some embodiments the poly(ethylene glycol) molecule is a branched PEG. In some embodiments the poly(ethylene glycol) molecule is a branched 1K PEG. In some embodiments the poly(ethylene glycol) molecule is a branched 2.5K PEG. In some embodiments the poly(ethylene glycol) molecule is a branched 5K PEG. In some embodiments the poly(ethylene glycol) molecule is a linear PEG. In some embodiments the poly(ethylene glycol) molecule is a linear 2.5K PEG. In some embodiments the poly(ethylene glycol) molecule is a linear 10K PEG. In some embodiments the poly(ethylene glycol) molecule is a linear 2K PEG. In some embodiments the poly(ethylene glycol) molecule is a linear 0.5K PEG. In some embodiments, the molecular weight of the poly(ethylene glycol) polymer is an average molecular weight. In certain embodiments, the average molecular weight is the number average molecular weight (Mn). The average molecular weight may be determined or measured using GPC or SEC, SDS/PAGE analysis, RP-HPLC, mass spectrometry, or capillary electrophoresis.

### Methods of Treatment

Conjugates described herein may be used to treat conditions and/or diseases. In some instances, the disease comprises a proliferative disease. In some instances, the proliferative disease comprises cancer. In some instances, the cancer comprises one or more tumors. In some instances, the cancer comprises solid or liquid tumors. In some instances, conjugates are administered to kill or inhibit growth of a rapidly dividing cell, such as a tumor cell. In some instances, a method of treating a proliferative disease or condition in a subject in need thereof comprises administering to the subject a therapeutically effective amount of a conjugate described herein. In some embodiments, the proliferative disease or condition is a cancer. In some embodiments, the cancer is a solid tumor cancer. In some embodiments, the solid tumor cancer is bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, or prostate cancer. In some instances, the disease comprises PCa (prostate cancer), CRPCa (castration resistant prostate cancer), solid tumors (neovasculature), NSCLC (non-small cell lung cancer), HNSCC (head and neck squamous cell carcinoma), ESCC (esophageal cancer) GC (gastric cancer), CRC (colorectal cancer), SCLC (small cell lung cancer), MPM (mesothelioma), PDAC (Pancreatic ductal adenocarcinoma), ALL (Acute Lymphoblastic Leukemia), AML (Acute Myeloid Leukemia), MDS (Myelodysplastic syndromes), MSI-high tumors, melanoma, DLBCL (diffuse large B cell lymphoma), endometrial cancer, cervical cancer, bladder cancer, BrCa (breast cancer), TNBC (triple negative breast cancer), NE-PCa (Neuroendocrine prostate cancer), GBM (glioblastoma), and RCC (Renal cell carcinoma).

In some instances, tumor cells targeted herein overexpress one or more targets. In some instances, the target comprises surface markers or receptors. In some instances, the target is selected from one or more of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some instances, the target is selected from two or more of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Muc1, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM. In some instances, the conjugate brings a payload to the tumor cell, when the conjugate is bound to the one or more targets on the tumor cell and the payload kills, inhibits or slows the growth of the tumor cell.

In some instances, the conjugates herein are administered for imaging a particular cell or groups of cells. In some cases, the cells or group of cells are tumor cells, or present in the tumor microenvironment. In some instances, the conjugate brings a payload to the tumor cell, when the conjugate is bound to the one or more targets on the tumor cell and the payload is an imaging agent (such as a visual dye or a radiolabel), and the tumor cell is imaged as a result of the association of the conjugate with the tumor cell.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

In one embodiment, an injectable pharmaceutical composition described herein, is used in the preparation of medicaments for the treatment of diseases or conditions in a mammal that would benefit from administration of any one of the injectable pharmaceutical compositions of the conjugates disclosed. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, in which the mammal previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments, the administration of the compounds are administered chronically, that is, for an extended period of time, including throughout the duration of the patient's life in order to ameliorate or otherwise control or limit the symptoms of the patient's disease or condition.

### Methods of Manufacture

Conjugates described herein may be synthesized using in-vivo or in-vitro methods, or a combination of methods. In some cases, the method is an in vivo method. In some cases, the method is an in vitro method. In some instances, a targeting domain comprising an unnatural amino acid is synthesized in-vivo, and a payload is attached using in-vitro chemical methods.

In some cases, the method is an ex vivo method. In some cases, a conjugate described herein that comprises a natural amino acid mutation or an unnatural amino acid mutation is recombinantly produced or chemically synthesized. In some cases, the targeting domain described herein is produced recombinantly, e.g., by a host cell system or in a cell-free system.

In general, methods of making a target polypeptide that includes a non-standard amino acid are known. In some cases, the amino-acyl tRNA synthetase/tRNA pair cognate to an unnatural amino acid is orthogonal to the cellular components of the cell in which it is used. The orthogonality (and therefore the suitability) of exogenous amino-acyl tRNA synthetase/tRNA pairs is dependent on the type of host organism. Four main orthogonal aminoacyl-tRNA synthetases have been developed for genetic code expansion: the Methanococcus janaschii tyrosyl-tRNA synthetase (MjTyrRS)/tRNA_{CUA} pair, the Escherichia coli tyrosyl-tRNA synthetase (EcTyrRS)/tRNA_{CUA} pair, the E. coli leucyl-tRNA synthetase (EcLeuRS)/tRNA_{CUA} pair, and pyrrolysyl-tRNA synthetase (PylRS)/tRNA_{CUA} (tRNA^{pyl}) pairs from certain Methanosarcina. The PylRS/tRNA_{CUA} pair is orthogonal in bacteria, eukaryotic cells, and animals (see, e.g., Chin, Jason W. “Expanding and reprogramming the genetic code of cells and animals.” Annual review of biochemistry 83 (2014): 379-408).

In some case, the unnatural amino acid (UAA) provided herein is incorporated using a pyrrolysyl-tRNA synthetase (tRNA^{pyl}). The unnatural amino acid (UAA) is introduced on a transfer RNA molecule (tRNA) such that it may be used in translation. However, the attachment of unnatural amino acids to tRNA may not necessarily be accomplished by the naturally occurring aminoacyl-tRNA synthetase. Thus, engineered aminoacyl-tRNA synthetases such as engineered tRNA^{pyl} prepared and selected from a generated tRNA^{pyl} mutant library may be useful for attaching the desired UAA to tRNA so that the desired UAA can be incorporated in mutagenesis.

In some embodiments, the UAA provided herein (e.g., UAA of Formula (IA)) can be attached to a tRNA using an engineered mutant tRNA^{pyl} variant that is capable of attaching such a UAA. In some embodiments, the mutant tRNA^{pyl} variant introduces one amino acid mutation (i.e., incorporating a UAA). In other embodiments, the mutant tRNA^{pyl} variant introduces multiple amino acid mutations. In some embodiments, a library of tRNA^{pyl} variants are prepared and screened by those skilled in the art to select the appropriate tRNA^{pyl} variant for attachment of the desired UAA. In some embodiments, the tRNA^{pyl} variant used to introduce UAA(s) in the conjugates provided herein is a tRNA^{pyl} variant described in U.S. Pat. Nos. 8,735,093, 9,133,449, WO2020206341, each of which is incorporated by reference herein.

In various embodiments, a mutant pyrrolysyl-tRNA synthetase may be used. The mutant pyrrolysyl-tRNA synthetase may be derived from or be variants of pyrrolysyl-tRNA synthetase from Methanosarcina barkeri, Methanosarcina mazei, Methanosarcina alvus, or Methanosarcina jannaschii, or chimera thereof.

In some embodiments, the mutant pyrrolysyl-tRNA synthetase provided herein comprises at least 5 amino acid residues substitutions within the substrate-binding site of the mutant pyrrolysyl-tRNA synthetase. In some embodiments, the mutant pyrrolysyl-tRNA synthetase comprises at least 5 amino acid residues substitutions in the amino acid sequence of SEQ ID NO:90. In some embodiments, the substrate-binding site includes residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO: 90. In some embodiments, the at least 5 amino acid residues substitutions are selected from a substitution for alanine at position 302, a substitution for asparagine at position 346, a substitution for cysteine at position 348, a substitution for tyrosine at position 384, and a substitution for tryptophan at position 417 as set forth in the amino acid sequence of SEQ ID NO: 90. In some embodiments, the at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO: 90.

In some embodiments, the mutant pyrrolysyl-tRNA synthetase provided herein has the amino acid sequence of SEQ ID NO:84. In some embodiments, the mutant pyrrolysyl-tRNA synthetase includes an amino acid sequence of SEQ ID NO:84. In some embodiments, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:84. In some embodiments, the mutant pyrrolysyl-tRNA synthetase has an amino acid sequence that has at least 80% identity to SEQ ID NO:84. In some embodiments, the mutant pyrrolysyl-tRNA synthetase has an amino acid sequence that has at least 85% identity to SEQ ID NO:84. In some embodiments, the mutant pyrrolysyl-tRNA synthetase has an amino acid sequence that has at least 90% identity to SEQ ID NO:84. In some embodiments, the mutant pyrrolysyl-tRNA synthetase has an amino acid sequence that has at least 91% identity to SEQ ID NO:84. In some embodiments, the mutant pyrrolysyl-tRNA synthetase has an amino acid sequence that has at least 92% identity to SEQ ID NO: 84. In some embodiments, the mutant pyrrolysyl-tRNA synthetase has an amino acid sequence that has at least 93% identity to SEQ ID NO: 84. In some embodiments, the mutant pyrrolysyl-tRNA synthetase has an amino acid sequence that has at least 94% identity to SEQ ID NO: 84. In some embodiments, the mutant pyrrolysyl-tRNA synthetase has an amino acid sequence that has at least 95% identity to SEQ ID NO: 84. In some embodiments, the mutant pyrrolysyl-tRNA synthetase has an amino acid sequence that has at least 96% identity to SEQ ID NO: 84. In some embodiments, the mutant pyrrolysyl-tRNA synthetase has an amino acid sequence that has at least 97% identity to SEQ ID NO: 84. In some embodiments, the mutant pyrrolysyl-tRNA synthetase has an amino acid sequence that has at least 98% identity to SEQ ID NO: 84. In some embodiments, the mutant pyrrolysyl-tRNA synthetase has an amino acid sequence that has at least 99% identity to SEQ ID NO: 84.

In some embodiments, the mutant pyrrolysyl-tRNA synthetase provided herein has the amino acid sequence of SEQ ID NO:87. In some embodiments, the mutant pyrrolysyl-tRNA synthetase includes an amino acid sequence of SEQ ID NO:87. In some embodiments, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO: 87. In some embodiments, the mutant pyrrolysyl-tRNA synthetase has an amino acid sequence that has at least 80% identity to SEQ ID NO: 87. In some embodiments, the mutant pyrrolysyl-tRNA synthetase has an amino acid sequence that has at least 85% identity to SEQ ID NO: 87. In some embodiments, the mutant pyrrolysyl-tRNA synthetase has an amino acid sequence that has at least 90% identity to SEQ ID NO: 87. In some embodiments, the mutant pyrrolysyl-tRNA synthetase has an amino acid sequence that has at least 91% identity to SEQ ID NO: 87. In some embodiments, the mutant pyrrolysyl-tRNA synthetase has an amino acid sequence that has at least 92% identity to SEQ ID NO: 87. In some embodiments, the mutant pyrrolysyl-tRNA synthetase has an amino acid sequence that has at least 93% identity to SEQ ID NO: 87. In some embodiments, the mutant pyrrolysyl-tRNA synthetase has an amino acid sequence that has at least 94% identity to SEQ ID NO: 87. In some embodiments, the mutant pyrrolysyl-tRNA synthetase has an amino acid sequence that has at least 95% identity to SEQ ID NO: 87. In some embodiments, the mutant pyrrolysyl-tRNA synthetase has an amino acid sequence that has at least 96% identity to SEQ ID NO: 87. In some embodiments, the mutant pyrrolysyl-tRNA synthetase has an amino acid sequence that has at least 97% identity to SEQ ID NO: 87. In some embodiments, the mutant pyrrolysyl-tRNA synthetase has an amino acid sequence that has at least 98% identity to SEQ ID NO: 87. In some embodiments, the mutant pyrrolysyl-tRNA synthetase has an amino acid sequence that has at least 99% identity to SEQ ID NO: 87.

In some embodiments, the mutant pyrrolysyl-tRNA synthetase provided herein is encoded by the nucleic acid sequence of SEQ ID NO:85. In some embodiments, the mutant pyrrolysyl-tRNA synthetase is encoded by a nucleic acid sequence including the sequence of SEQ ID NO: 85. In some embodiments, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO: 85. In some embodiments, the mutant pyrrolysyl-tRNA synthetase is encoded by a nucleic acid sequence that is at least 80% identity to SEQ ID NO: 85. In some embodiments, the mutant pyrrolysyl-tRNA synthetase is encoded by a nucleic acid sequence that is at least 85% identity to SEQ ID NO: 85. In some embodiments, the mutant pyrrolysyl-tRNA synthetase is encoded by a nucleic acid sequence that is at least 90% identity to SEQ ID NO: 85. In some embodiments, the mutant pyrrolysyl-tRNA synthetase is encoded by a nucleic acid sequence that is at least 91% identity to SEQ ID NO: 85. In some embodiments, the mutant pyrrolysyl-tRNA synthetase is encoded by a nucleic acid sequence that is at least 92% identity to SEQ ID NO: 85. In some embodiments, the mutant pyrrolysyl-tRNA synthetase is encoded by a nucleic acid sequence that is at least 93% identity to SEQ ID NO: 85. In some embodiments, the mutant pyrrolysyl-tRNA synthetase is encoded by a nucleic acid sequence that is at least 94% identity to SEQ ID NO: 85. In some embodiments, the mutant pyrrolysyl-tRNA synthetase is encoded by a nucleic acid sequence that is at least 95% identity to SEQ ID NO: 85. In some embodiments, the mutant pyrrolysyl-tRNA synthetase is encoded by a nucleic acid sequence that is at least 96% identity to SEQ ID NO: 85. In some embodiments, the mutant pyrrolysyl-tRNA synthetase is encoded by a nucleic acid sequence that is at least 97% identity to SEQ ID NO: 85. In some embodiments, the mutant pyrrolysyl-tRNA synthetase is encoded by a nucleic acid sequence that is at least 98% identity to SEQ ID NO: 85. In some embodiments, the mutant pyrrolysyl-tRNA synthetase is encoded by a nucleic acid sequence that is at least 99% identity to SEQ ID NO: 85.

In some embodiments, the mutant pyrrolysyl-tRNA synthetase provided herein is encoded by the nucleic acid sequence of SEQ ID NO:88. In some embodiments, the mutant pyrrolysyl-tRNA synthetase is encoded by a nucleic acid sequence including the sequence of SEQ ID NO: 88. In some embodiments, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO: 88. In some embodiments, the mutant pyrrolysyl-tRNA synthetase is encoded by a nucleic acid sequence that is at least 80% identity to SEQ ID NO: 88. In some embodiments, the mutant pyrrolysyl-tRNA synthetase is encoded by a nucleic acid sequence that is at least 85% identity to SEQ ID NO: 88. In some embodiments, the mutant pyrrolysyl-tRNA synthetase is encoded by a nucleic acid sequence that is at least 90% identity to SEQ ID NO: 88. In some embodiments, the mutant pyrrolysyl-tRNA synthetase is encoded by a nucleic acid sequence that is at least 91% identity to SEQ ID NO: 88. In some embodiments, the mutant pyrrolysyl-tRNA synthetase is encoded by a nucleic acid sequence that is at least 92% identity to SEQ ID NO: 88. In some embodiments, the mutant pyrrolysyl-tRNA synthetase is encoded by a nucleic acid sequence that is at least 93% identity to SEQ ID NO: 88. In some embodiments, the mutant pyrrolysyl-tRNA synthetase is encoded by a nucleic acid sequence that is at least 94% identity to SEQ ID NO: 88. In some embodiments, the mutant pyrrolysyl-tRNA synthetase is encoded by a nucleic acid sequence that is at least 95% identity to SEQ ID NO: 88. In some embodiments, the mutant pyrrolysyl-tRNA synthetase is encoded by a nucleic acid sequence that is at least 96% identity to SEQ ID NO: 88. In some embodiments, the mutant pyrrolysyl-tRNA synthetase is encoded by a nucleic acid sequence that is at least 97% identity to SEQ ID NO: 88. In some embodiments, the mutant pyrrolysyl-tRNA synthetase is encoded by a nucleic acid sequence that is at least 98% identity to SEQ ID NO: 88. In some embodiments, the mutant pyrrolysyl-tRNA synthetase is encoded by a nucleic acid sequence that is at least 99% identity to SEQ ID NO: 88.

In some embodiments, the mutant pyrrolysyl-tRNA synthetase provided herein has the amino acid sequence of SEQ ID NO:92. In some embodiments, the mutant pyrrolysyl-tRNA synthetase includes an amino acid sequence of SEQ ID NO:92. In some embodiments, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO: 92. In some embodiments, the mutant pyrrolysyl-tRNA synthetase has an amino acid sequence that has at least 80% identity to SEQ ID NO: 92. In some embodiments, the mutant pyrrolysyl-tRNA synthetase has an amino acid sequence that has at least 85% identity to SEQ ID NO: 92. In some embodiments, the mutant pyrrolysyl-tRNA synthetase has an amino acid sequence that has at least 90% identity to SEQ ID NO: 92. In some embodiments, the mutant pyrrolysyl-tRNA synthetase has an amino acid sequence that has at least 91% identity to SEQ ID NO: 92. In some embodiments, the mutant pyrrolysyl-tRNA synthetase has an amino acid sequence that has at least 92% identity to SEQ ID NO: 92. In some embodiments, the mutant pyrrolysyl-tRNA synthetase has an amino acid sequence that has at least 93% identity to SEQ ID NO: 92. In some embodiments, the mutant pyrrolysyl-tRNA synthetase has an amino acid sequence that has at least 94% identity to SEQ ID NO: 92. In some embodiments, the mutant pyrrolysyl-tRNA synthetase has an amino acid sequence that has at least 95% identity to SEQ ID NO: 92. In some embodiments, the mutant pyrrolysyl-tRNA synthetase has an amino acid sequence that has at least 96% identity to SEQ ID NO: 92. In some embodiments, the mutant pyrrolysyl-tRNA synthetase has an amino acid sequence that has at least 97% identity to SEQ ID NO: 92. In some embodiments, the mutant pyrrolysyl-tRNA synthetase has an amino acid sequence that has at least 98% identity to SEQ ID NO: 92. In some embodiments, the mutant pyrrolysyl-tRNA synthetase has an amino acid sequence that has at least 99% identity to SEQ ID NO: 87.

In some embodiments, the mutant pyrrolysyl-tRNA synthetase provided herein is encoded by the nucleic acid sequence of SEQ ID NO:91. In some embodiments, the mutant pyrrolysyl-tRNA synthetase is encoded by a nucleic acid sequence including the sequence of SEQ ID NO: 91. In some embodiments, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO: 91. In some embodiments, the mutant pyrrolysyl-tRNA synthetase is encoded by a nucleic acid sequence that is at least 80% identity to SEQ ID NO: 91. In some embodiments, the mutant pyrrolysyl-tRNA synthetase is encoded by a nucleic acid sequence that is at least 85% identity to SEQ ID NO: 91. In some embodiments, the mutant pyrrolysyl-tRNA synthetase is encoded by a nucleic acid sequence that is at least 90% identity to SEQ ID NO: 91. In some embodiments, the mutant pyrrolysyl-tRNA synthetase is encoded by a nucleic acid sequence that is at least 91% identity to SEQ ID NO: 91. In some embodiments, the mutant pyrrolysyl-tRNA synthetase is encoded by a nucleic acid sequence that is at least 92% identity to SEQ ID NO: 91. In some embodiments, the mutant pyrrolysyl-tRNA synthetase is encoded by a nucleic acid sequence that is at least 93% identity to SEQ ID NO: 91. In some embodiments, the mutant pyrrolysyl-tRNA synthetase is encoded by a nucleic acid sequence that is at least 94% identity to SEQ ID NO: 91. In some embodiments, the mutant pyrrolysyl-tRNA synthetase is encoded by a nucleic acid sequence that is at least 95% identity to SEQ ID NO: 91. In some embodiments, the mutant pyrrolysyl-tRNA synthetase is encoded by a nucleic acid sequence that is at least 96% identity to SEQ ID NO: 91. In some embodiments, the mutant pyrrolysyl-tRNA synthetase is encoded by a nucleic acid sequence that is at least 97% identity to SEQ ID NO: 91. In some embodiments, the mutant pyrrolysyl-tRNA synthetase is encoded by a nucleic acid sequence that is at least 98% identity to SEQ ID NO: 91. In some embodiments, the mutant pyrrolysyl-tRNA synthetase is encoded by a nucleic acid sequence that is at least 99% identity to SEQ ID NO: 91.

In some instances, sequences associated with mutant pyrrolysyl-tRNA synthetase herein are in Table 1B.

TABLE 1B

| SEQ | | |
|---|---|---|
| ID | | |
| NO. | DESCRIPTION | SEQUENCE |
| 84 | amino acid | MDKKPLNTLISATGLWMSRTGTIHKIKHHEVSRSKIYIEMA |
| | sequence of | CGDHLVVNNSRSSRTARALRHHKYRKTCKRCRVSDEDLN |
| | FSYRS | KFLTKANEDQTSVKVKVVSAPTRTKKAMPKSVARAPKPL |
| | | ENTEAAQAQPSGSKFSPAIPVSTQESVSVPASVSTSISSISTG |
| | | ATASALVKGNTNPITSMSAPVQASAPALTKSQTDRLEVLL |
| | | NPKDEISLNSGKPFRELESELLSRRKKDLQQIYAEERENYL |
| | | GKLEREITRFFVDRGFLEIKSPILIPLEYIERMGIDNDTELSK |
| | | QIFRVDKNFCLRPMLIPNLYNYLRKLDRALPDPIKIFEIGPC |
| | | YRKESDGKEHLEEFTMLTFIQMGSGCTRENLESITDFLNH |
| | | LGIDFKIVGDSCMVLGDTLDVMHGDLELSSAVVGPIPLDR |
| | | EWGIDKPKIGA |
| | | GFGLERLLKVKHDFKNIKRAARSESYYNGISTNL* |
| 85 | nucleic acid | ATGGATAAAAAGCCTTTGAACACTCTGATTTCTGCGACC |
| | (DNA) sequence | GGTCTGTGGATGTCCCGCACCGGCACCATCCACAAAAT |
| | of FSYRS | CAAACACCATGAAGTTAGCCGTTCCAAAATCTACATTG |
| | | AAATGGCTTGCGGCGATCACCTGGTTGTCAACAACTCCC |
| | | GTTCTTCTCGTACCGCTCGCGCACTGCGCCACCACAAAT |
| | | ATCGCAAAACCTGCAAACGTTGCCGTGTTAGCGATGAG |
| | | GACCTGAACAAATTCCTGACCAAAGCTAACGAGGATCA |
| | | GACCTCCGTAAAAGTGAAGGTAGTAAGCGCTCCGACCC |
| | | GTACTAAAAAGGCTATGCCAAAAAGCGTGGCCCGTGCC |
| | | CCGAAACCTCTGGAAAACACCGAGGCGGCTCAGGCTCA |
| | | ACCATCCGGTTCTAAATTTTCTCCGGCGATCCCAGTGTC |
| | | CACCCAAGAATCTGTTTCCGTACCAGCAAGCGTGTCTAC |
| | | CAGCATTAGCAGCATTICTACCGGTGCTACCGCTTCTGC |
| | | GCTGGTAAAAGG |
| | | TAACACTAACCCGATTACTAGCATGTCTGCACCGGTACA |
| | | GGCAAGCGCCCCAGCTCTGACTAAATCCCAGACGGACC |
| | | GTCTGGAGGTGCTGCTGAACCCAAAGGATGAAATCTCT |
| | | CTGAACAGCGGCAAGCCTTTCCGTGAGCTGGAAAGCGA |
| | | GCTGCTGTCTCGTCGTAAAAAGGATCTGCAACAGATCTA |
| | | CGCTGAGGAACGCGAGAACTATCTGGGTAAGCTGGAGC |
| | | GCGAAATTACTCGCTTCTTCGTGGATCGCGGTTTCCTGG |
| | | AGATCAAATCTCCGATTCTGATTCCGCTGGAATACATTG |
| | | AACGTATGGGCATCGATAATGATACCGAACTGTCTAAA |
| | | CAGATCTTCCGTGTGGATAAAAACTTCTGTCTGCGTCCG |
| | | ATGCTGATTCCGAACTTGTACAACTATTTACGTAAACTG |
| | | GACCGTGCCCTGCCGGACCCGATCAAAATATTCGAGAT |
| | | CGGTCCTTGCTACCGTAAAGAGTCCGACGGTAAAGAGC |
| | | ACCTGGAAGAAT |
| | | TCACCATGCTGACATTCATTCAGATGGGTAGCGGTTGCA |
| | | CGCGTGAAAACCTGGAATCCATTATCACCGACTTCCTGA |
| | | ATCACCTGGGTATCGATTTCAAAATTGTTGGTGACAGCT |
| | | GTATGGTGTTAGGCGATACGCTGGATGTTATGCACGGC |
| | | GATCTGGAGCTGTCTTCCGCAGTIGTGGGCCCAATCCCG |
| | | CTGGATCGTGAGTGGGGTATCGACAAACCTAAAATCGG |
| | | TGCGGGTTTTGGTCTGGAGCGTCTGCTGAAAGTAAAAC |
| | | ACGACTTCAAGAACATCAAACGTGCTGCACGTTCCGAG |
| | | TCCTATTACAATGGTATTTCTACTAACCTGTAA |
| 86 | pEVOL-FSYRS | ACTAGTATGGATAAAAAGCCTTTGAACACTCTGATTTCT |
| | DNA sequence | GCGACCGGTCTGTGGATGTCCCGCACCGGCACCATCCA |
| | | CAAAATCAAACACCATGAAGTTAGCCGTTCCAAAATCT |
| | | ACATTGAAATGGCTTGCGGCGATCACCTGGTTGTCAACA |
| | | ACTCCCGTTCTTCTCGTACCGCTCGCGCACTGCGCCACC |
| | | ACAAATATCGCAAAACCTGCAAACGTTGCCGTGTTAGC |
| | | GATGAGGACCTGAACAAATTCCTGACCAAAGCTAACGA |
| | | GGATCAGACCTCCGTAAAAGTGAAGGTAGTAAGCGCTC |
| | | CGACCCGTACTAAAAAGGCTATGCCAAAAAGCGTGGCC |
| | | CGTGCCCCGAAACCTCTGGAAAACACCGAGGCGGCTCA |
| | | GGCTCAACCATCCGGTTCTAAATTTTCTCCGGCGATCCC |
| | | AGTGTCCACCCAAGAATCTGTTTCCGTACCAGCAAGCGT |
| | | GTCTACCAGCATTAGCAGCATTTCTACCGGTGCTACCGC |
| | | TTCTGCGCTGGTAAAAGGTAACACTAACCCGATTACTAG |
| | | CATGTCTGCACCGGTACAGGCAAGCGCCCCAGCTCTGA |
| | | CTAAATCCCAGACGGACCGTCTGGAGGTGCTGCTGAAC |
| | | CCAAAGGATGAAATCTCTCTGAACAGCGGCAAGCCTTT |
| | | CCGTGAGCTGGAAAGCGAGCTGCTGTCTCGTCGTAAAA |
| | | AGGATCTGCAACAGATCTACGCTGAGGAACGCGAGAAC |
| | | TATCTGGGTAAGCTGGAGCGCGAAATTACTCGCTTCTTC |
| | | GTGGATCGCGGTTTCCTGGAGATCAAATCTCCGATTCTG |
| | | ATTCCGCTGGAATACATTGAACGTATGGGCATCGATAAT |
| | | GATACCGAACTGTCTAAACAGATCTTCCGTGTGGATAA |
| | | AAACTTCTGTCTGCGTCCGATGCTGATTCCGAACTTGTA |
| | | CAACTATTTACGTAAACTGGACCGTGCCCTGCCGGACCC |
| | | GATCAAAATATTCGAGATCGGTCCTTGCTACCGTAAAG |
| | | AGTCCGACGGTAAAGAGCACCTGGAAGAATTCACCATG |
| | | CTGACATTCATTCAGATGGGTAGCGGTTGCACGCGTGA |
| | | AAACCTGGAATCCATTATCACCGACTTCCTGAATCACCT |
| | | GGGTATCGATTTCAAAATTGTTGGTGACAGCTGTATGGT |
| | | GTTAGGCGATACGCTGGATGTTATGCACGGCGATCTGG |
| | | AGCTGTCTTCCGCAGTTGTGGGCCCAATCCCGCTGGATC |
| | | GTGAGTGGGGTATCGACAAACCTAAAATCGGTGCGGGT |
| | | TTTGGTCTGGAGCGTCTGCTGAAAGTAAAACACGACTTC |
| | | AAGAACATCAAACGTGCTGCACGTTCCGAGTCCTATTAC |
| | | AATGGTATTTCTACTAACCTGTAAGTCGACCATCATCAT |
| | | CATCATCATTGAGTTTAAACGGTCTCCAGCTTGGCTGTT |
| | | TTGGCGGATGAGAGAAGATTTTCAGCCTGATACAGATT |
| | | AAATCAGAACGCAGAAGCGGTCTGATAAAACAGAATTT |
| | | GCCTGGCGGCAGTAGCGCGGTGGTCCCACCTGACCCCA |
| | | TGCCGAACTCAGAAGTGAAACGCCGTAGCGCCGATGGT |
| | | AGTGTGGGGTCTCCCCATGCGAGAGTAGGGAACTGCCA |
| | | GGCATCAAATAAAACGAAAGGCTCAGTCGAAAGACTGG |
| | | GCCTTGTTTGTGAGCTCCCGGTCATCAATCATCCCCATA |
| | | ATCCTTGTTAGATTATCAATTTTAAAAAACTAACAGTTG |
| | | TCAGCCTGTCCCGCTTTAATATCATACGCCGTTATACGT |
| | | TGTTTACGCTTTGAGGAATCCCATATGATACGCGGCCGC |
| | | TTTCAAACGCTAAATTGCCTGATGCGCTACGCTTATCAG |
| | | GCCTACATGATCTCTGCAATATATTGAGTTTGCGTGCTT |
| | | TTGTAGGCCGGATAAGGCGTTCACGCCGCATCCGGCAA |
| | | GAAACAGCAAACAATCCAAAACGCCGCGTTCAGCGGCG |
| | | TTTTTTCTGCTTTTCTTCGCGAATTAATTCCGCTTCGCAA |
| | | CATGTGAGCACCGGTTTATTGACTACCGGAAGCAGTGT |
| | | GACCGTGTGCTTCTCAAATGCCTGAGGCCAGTTTGCTCA |
| | | GGCTCTCCCCGTGGAGGTAATAATTGACGATATGATCA |
| | | GTGCACGGCTAACTAAGCGGCCTGCTGACTTTCTCGCCG |
| | | ATCAAAAGGCATTTTGCTATTAAGGGATTGACGAGGGC |
| | | GTATCTGCGCAGTAAGATGCGCCCCGCATTGGAAACCT |
| | | GATCATGTAGATCGAATGGACTCTAAATCCGTTCAGCCG |
| | | GGTTAGATTCCCGGGGTTTCCGCCAAATTCGAAAAGCCT |
| | | GCTCAACGAGCAGGCTTTTTTGCATGCTCGAGCAGCTCA |
| | | GGGTCGAATTTGCTTTCGAATTTCTGCCATTCATCCGCTT |
| | | ATTATCACTTATTCAGGCGTAGCAACCAGGCGTTTAAGG |
| | | GCACCAATAACTGCCTTAAAAAAATTACGCCCCGCCCT |
| | | GCCACTCATCGCAGTACTGTTGTAATTCATTAAGCATTC |
| | | TGCCGACATGGAAGCCATCACAAACGGCATGATGAACC |
| | | TGAATCGCCAGCGGCATCAGCACCTTGTCGCCTTGCGTA |
| | | TAATATTTGCCCATGGTGAAAACGGGGGCGAAGAAGTT |
| | | GTCCATATTGGCCACGTTTAAATCAAAACTGGTGAAACT |
| | | CACCCAGGGATTGGCTGAGACGAAAAACATATTCTCAA |
| | | TAAACCCTTTAGGGAAATAGGCCAGGTTTTCACCGTAAC |
| | | ACGCCACATCTTGCGAATATATGTGTAGAAACTGCCGG |
| | | AAATCGTCGTGGTATTCACTCCAGAGCGATGAAAACGT |
| | | TTCAGTTTGCTCATGGAAAACGGTGTAACAAGGGTGAA |
| | | CACTATCCCATATCACCAGCTCACCGTCTTTCATTGCCA |
| | | TACGGAATTCCGGATGAGCATTCATCAGGGGGCAAGA |
| | | ATGTGAATAAAGGCCGGATAAAACTTGTGCTTATTTTTC |
| | | TTTACGGTCTTTAAAAAGGCCGTAATATCCAGCTGAACG |
| | | GTCTGGTTATAGGTACATTGAGCAACTGACTGAAATGCC |
| | | TCAAAATGTTCTTTACGATGCCATTGGGATATATCAACG |
| | | GTGGTATATCCAGTGATTTTTTTCTCCATTTTAGCTTCCT |
| | | TAGCTCCTGAAAATCTCGATAACTCAAAAAATACGCCC |
| | | GGTAGTGATCTTATTTCATTATGGTGAAAGTTGGAACCT |
| | | CTTACGTGCCGATCAACGTCTCATTTTCGCCAAAAGTTG |
| | | GCCCAGGGCTTCCCGGTATCAACAGGGACACCAGGATT |
| | | TATTTATTCTGCGAAGTGATCTTCCGTCACAGGTATTTA |
| | | TTCGGCGCAAAGTGCGTCGGGTGATGCTGCCAACTTACT |
| | | GATTTAGTGTATGATGGTGTTTTTGAGGTGCTCCAGTGG |
| | | CTTCTGTTTCTATCAGCTGTCCCTCCTGTTCAGCTACTGA |
| | | CGGGGTGGTGCGTAACGGCAAAAGCACCGCCGGACATC |
| | | AGCGCTAGCGGAGTGTATACTGGCTTACTATGTTGGCAC |
| | | TGATGAGGGTGTCAGTGAAGTGCTTCATGTGGCAGGAG |
| | | AAAAAAGGCTGCACCGGTGCGTCAGCAGAATATGTGAT |
| | | ACAGGATATATTCCGCTTCCTCGCTCACTGACTCGCTAC |
| | | GCTCGGTCGTTCGACTGCGGCGAGCGGAAATGGCTTAC |
| | | GAACGGGGCGGAGATTTCCTGGAAGATGCCAGGAAGAT |
| | | ACTTAACAGGGAAGTGAGAGGGCCGCGGCAAAGCCGTT |
| | | TTTCCATAGGCTCCGCCCCCCTGACAAGCATCACGAAAT |
| | | CTGACGCTCAAATCAGTGGTGGCGAAACCCGACAGGAC |
| | | TATAAAGATACCAGGCGTTTCCCCCTGGCGGCTCCCTCG |
| | | TGCGCTCTCCTGTTCCTGCCTTTCGGTTTACCGGTGTCAT |
| | | TCCGCTGTTATGGCCGCGTTTGTCTCATTCCACGCCTGA |
| | | CACTCAGTTCCGGGTAGGCAGTTCGCTCCAAGCTGGACT |
| | | GTATGCACGAACCCCCCGTTCAGTCCGACCGCTGCGOCT |
| | | TATCCGGTAACTATCGTCTTGAGTCCAACCCGGAAAGAC |
| | | ATGCAAAAGCACCACTGGCAGCAGCCACTGGTAATTGA |
| | | TTTAGAGGAGTTAGTCTTGAAGTCATGCGCCGGTTAAGG |
| | | CTAAACTGAAAGGACAAGTTTTGGTGACTGCGCTCCTCC |
| | | AAGCCAGTTACCTCGGTTCAAAGAGTTGGTAGCTCAGA |
| | | GAACCTTCGAAAAACCGCCCTGCAAGGCGGTTTTTTCGT |
| | | TTTCAGAGCAAGAGATTACGCGCAGACCAAAACGATCT |
| | | CAAGAAGATCATCTTATTAATCAGATAAAATATTTCTAG |
| | | ATTTCAGTGCAATTTATCTCTTCAAATGTAGCACCTGAA |
| | | GTCAGCCCCATACGATATAAGTTGTAATTCTCATGTTTG |
| | | ACAGCTTATCATCGATAAGCTTGGTACCCAATTATGACA |
| | | ACTTGACGGCTACATCATTCACTTTTTCTTCACAACCGG |
| | | CACGGAACTCGCTCGGGCTGGCCCCGGTGCATTTTTTAA |
| | | ATACCCGCGAGAAATAGAGTTGATCGTCAAAACCAACA |
| | | TTGCGACCGACGGTGGCGATAGGCATCCGGGTGGTGCT |
| | | CAAAAGCAGCTTCGCCTGGCTGATACGTTGGTCCTCGCG |
| | | CCAGCTTAAGACGCTAATCCCTAACTGCTGGOGGAAAA |
| | | GATGTGACAGACGCGACGGCGACAAGCAAACATGCTGT |
| | | GCGACGCTGGCGATATCAAAATTGCTGTCTGCCAGGTG |
| | | ATCGCTGATGTACTGACAAGCCTCGCGTACCCGATTATC |
| | | CATCGGTGGATGGAGCGACTCGTTAATCGCTTCCATGCG |
| | | CCGCAGTAACAATTGCTCAAGCAGATTTATOGCCAGCA |
| | | GCTCCGAATAGCGCCCTTCCCCTTGCCCGGCGTTAATGA |
| | | TTTGCCCAAACAGGTCGCTGAAATGCGGCTGGTGCGCTT |
| | | CATCCGGGCGAAAGAACCCCGTATTGGCAAATATTGAC |
| | | GGCCAGTTAAGCCATTCATGCCAGTAGGCGCGCGGACG |
| | | AAAGTAAACCCACTGGTGATACCATTCGCGAGCCTCCG |
| | | GATGACGACCGTAGTGATGAATCTCTCCTGGCGGGAAC |
| | | AGCAAAATATCACTCGGTCGGCAAACAAATTCTCGTCC |
| | | CTGATTTTTCACCACCCCCTGACCGCGAATGGTGAGATT |
| | | GAGAATATAACCTTTCATTCCCAGCGGTCGGTCGATAAA |
| | | AAAATCGAGATAACCGTTGGCCTCAATCGGCGTTAAAC |
| | | CCGCCACCAGATGGGCATTAAACGAGTATCCCGGCAGC |
| | | AGGGGATCATTTTGCGCTTCAGCCATACTTTTCATACTC |
| | | CCGCCATTCAGAGAAGAAACCAATTGTCCATATTGCATC |
| | | AGACATTGCCGTCACTGCGTCTTTTACTGGCTCTTCTCG |
| | | CTAACCAAACCGGTAACCCCGCTTATTAAAAGCATTCTG |
| | | TAACAAAGCGGGACCAAAGCCATGACAAAAACGCGTA |
| | | ACAAAAGTGTCTATAATCACGGCAGAAAAGTCCACATT |
| | | GATTATTTGCACGGCGTCACACTTTGCTATGCCATAGCA |
| | | TTTTTATCCATAAGATTAGCGGATCCTACCTGACGCTTT |
| | | TTATCGCAACTCTCTACTGTTTCTCCATACCCGTTTTTTT |
| | | GGGCTAACAGGAGGAATT |
| 87 | amino acid | MTVKYTDAQIQRLREYGNGTYEQKVFEDLASRDAAFSKE |
| | sequence of | MSVASTDNEKKIKGMIANPSRHGLTQLMNDIADALVAEGF |
| | FSKRS | IEVRTPIFISKDALARMTITEDKPLFKQVFWIDEKRALRPML |
| | | APNLGSVARDLRDHTDGPVKIFEMGSCFRKESHSGMHLEE |
| | | FTMLNLFDMGPRGDATEVLKNYISVVMKAAGLPDYDLVQ |
| | | EESDVYKETIDVEINGQEVCSAAVGPTPIDAAHDVHEPWS |
| | | GAGFGLERLLTIREKYSTVKKGGASISYLNGAKIN |
| 88 | nucleic acid | ATGACCGTGAAGTACACCGACGCCCAGATCCAACGCCT |
| | (DNA) sequence | GCGCGAGTATGGTAATGGCACCTATGAACAGAAAGTTT |
| | of FSKRS | TTGAGGACCTGGCCAGCCGTGACGCCGCGTTCAGCAAG |
| | | GAAATGAGCGTGGCCAGCACCGACAACGAGAAGAAAA |
| | | TCAAGGGTATGATTGCCAATCCGAGCCGCCACGGCCTG |
| | | ACCCAGCTGATGAACGACATCGCTGATGCCCTGGTGGC |
| | | GGAGGGTTTCATTGAAGTGCGTACCCCGATCTTTATTAG |
| | | CAAGGATGCGCTGGCTCGCATGACCATCACCGAAGACA |
| | | AGCCGCTGTTCAAACAGGTGTTTTGGATTGATGAGAAA |
| | | CGTGCCCTGCGCCCGATGCTGGCGCCAAACCTGGGTAG |
| | | CGTGGCTCGTGACCTGCGCGATCACACCGACGGTCCGG |
| | | TGAAAATCTTCGAAATGGGCAGCTGCTTTCGTAAAGAG |
| | | AGCCACAGCGGTATGCACCTGGAGGAGTTCACCATGCT |
| | | GAATCTGTTTGATATGGGTCCGCGCGGCGACGCTACCG |
| | | AAGTGCTGAAGAACTACATTAGCGTGGTGATGAAAGCT |
| | | GCCGGCCTGCCGGACTACGATCTGGTGCAGGAAGAGAG |
| | | CGACGTGTACAAGGAAACCATCGACGTGGAAATTAACG |
| | | GTCAGGAAGTGTGCAGCGCGGCTGTGGGTCCAACTCCT |
| | | ATTGATGCGGCCCATGACGTGCATGAGCCGTGGAGCGG |
| | | TGCTGGTTTTGGCCTGGAACGTCTGCTGACCATCCGCGA |
| | | GAAATACTCCACCGTGAAAAAGGGCGGCGCAAGCATCT |
| | | CCTACCTGAATGGTGCCAAAATCAATTAA |
| 89 | pEVOL-FSKRS | ACTAGTATGACCGTGAAGTACACCGACGCCCAGATCCA |
| | | ACGCCTGCGCGAGTATGGTAATGGCACCTATGAACAGA |
| | | AAGTTTTTGAGGACCTGGCCAGCCGTGACGCCGCGTTCA |
| | | GCAAGGAAATGAGCGTGGCCAGCACCGACAACGAGAA |
| | | GAAAATCAAGGGTATGATTGCCAATCCGAGCCGCCACG |
| | | GCCTGACCCAGCTGATGAACGACATCGCTGATGCCCTG |
| | | GTGGCGGAGGGTTTCATTGAAGTGCGTACCCCGATCTTT |
| | | ATTAGCAAGGATGCGCTGGCTCGCATGACCATCACCGA |
| | | AGACAAGCCGCTGTTCAAACAGGTGTTTTGGATTGATG |
| | | AGAAACGTGCCCTGCGCCCGATGCTGGCGCCAAACCTG |
| | | GGTAGCGTGGCTCGTGACCTGCGCGATCACACCGACGG |
| | | TCCGGTGAAAATCTTCGAAATGGGCAGCTGCTTTCGTAA |
| | | AGAGAGCCACAGCGGTATGCACCTGGAGGAGTTCACCA |
| | | TGCTGAATCTGTTTGATATGGGTCCGCGCGGCGACGCTA |
| | | CCGAAGTGCTGAAGAACTACATTAGCGTGGTGATGAAA |
| | | GCTGCCGGCCTGCCGGACTACGATCTGGTGCAGGAAGA |
| | | GAGCGACGTGTACAAGGAAACCATCGACGTGGAAATTA |
| | | ACGGTCAGGAAGTGTGCAGCGCGGCTGTGGGTCCAACT |
| | | CCTATTGATGCGGCCCATGACGTGCATGAGCCGTGGAG |
| | | CGGTGCTGGTTTTGGCCTGGAACGTCTGCTGACCATCCG |
| | | CGAGAAATACTCCACCGTGAAAAAGGGCGGCGCAAGCA |
| | | TCTCCTACCTGAATGGTGCCAAAATCAATTAAGTCGACC |
| | | ATCATCATCATCATCATTGAGTTTAAACGGTCTCCAGCT |
| | | TGGCTGTTTTGGCGGATGAGAGAAGATTTTCAGCCTGAT |
| | | ACAGATTAAATCAGAACGCAGAAGCGGTCTGATAAAAC |
| | | AGAATTTGCCTGGCGGCAGTAGCGCGGTGGTCCCACCT |
| | | GACCCCATGCCGAACTCAGAAGTGAAACGCCGTAGCGC |
| | | CGATGGTAGTGTGGGGTCTCCCCATGCGAGAGTAGGGA |
| | | ACTGCCAGGCATCAAATAAAACGAAAGGCTCAGTCGAA |
| | | AGACTGGGCCTTGTTTGTGAGCTCCCGGTCATCAATCAT |
| | | CCCCATAATCCTTGTTAGATTATCAATTTTAAAAAACTA |
| | | ACAGTTGTCAGCCTGTCCCGCTTTAATATCATACGCCGT |
| | | TATACGTTGTTTACGCTTTGAGGAATCCCATATGATACG |
| | | CGGCCGCTTTCAAACGCTAAATTGCCTGATGCGCTACGC |
| | | TTATCAGGCCTACATGATCTCTGCAATATATTGAGTTTG |
| | | CGTGCTTTTGTAGGCCGGATAAGGCGTTCACGCCGCATC |
| | | CGGCAAGAAACAGCAAACAATCCAAAACGCCGCGTTCA |
| | | GCGGCGTTTTTTCTGCTTTTCTTCGCGAATTAATTCCGCT |
| | | TCGCAACATGTGAGCACCGGTTTATTGACTACCGGAAG |
| | | CAGTGTGACCGTGTGCTTCTCAAATGCCTGAGGCCAGTT |
| | | TGCTCAGGCTCTCCCCGTGGAGGTAATAATTGACGATAT |
| | | GATCAGTGCACGGCTAACTAAGCGGCCTGCTGACTTTCT |
| | | CGCCGATCAAAAGGCATTTTGCTATTAAGGGATTGACG |
| | | AGGGCGTATCTGCGCAGTAAGATGCGCCCCGCATTGGG |
| | | GGACGGTCCGGCGACCAGCGGGTCTCTAAAACCTAGCC |
| | | AGCGGGGTTCGACGCCCCGGTCTCTCGCCAAATTCGAA |
| | | AAGCCTGCTCAACGAGCAGGCTTTTTTGCATGCTCGAGC |
| | | AGCTCAGGGTCGAATTTGCTTTCGAATTTCTGCCATTCA |
| | | TCCGCTTATTATCACTTATTCAGGCGTAGCAACCAGGCG |
| | | TTTAAGGGCACCAATAACTGCCTTAAAAAAATTACGCC |
| | | CCGCCCTGCCACTCATCGCAGTACTGTTGTAATTCATTA |
| | | AGCATTCTGCCGACATGGAAGCCATCACAAACGGCATG |
| | | ATGAACCTGAATCGCCAGCGGCATCAGCACCTTGTCGC |
| | | CTTGCGTATAATATTTGCCCATGGTGAAAACGGGGGCG |
| | | AAGAAGTTGTCCATATTGGCCACGTTTAAATCAAAACTG |
| | | GTGAAACTCACCCAGGGATTGGCTGAGACGAAAAACAT |
| | | ATTCTCAATAAACCCTTTAGGGAAATAGGCCAGGTTTTC |
| | | ACCGTAACACGCCACATCTTGCGAATATATGTGTAGAA |
| | | ACTGCCGGAAATCGTCGTGGTATTCACTCCAGAGCGAT |
| | | GAAAACGTTTCAGTTTGCTCATGGAAAACGGTGTAACA |
| | | AGGGTGAACACTATCCCATATCACCAGCTCACCGTCTTT |
| | | CATTGCCATACGGAATTCCGGATGAGCATTCATCAGGC |
| | | GGGCAAGAATGTGAATAAAGGCCGGATAAAACTTGTGC |
| | | TTATTTTTCTTTACGGTCTTTAAAAAGGCCGTAATATCC |
| | | AGCTGAACGGTCTGGTTATAGGTACATTGAGCAACTGA |
| | | CTGAAATGCCTCAAAATGTTCTTTACGATGCCATTGGGA |
| | | TATATCAACGGTGGTATATCCAGTGATTTTTTTCTCCATT |
| | | TTAGCTTCCTTAGCTCCTGAAAATCTCGATAACTCAAAA |
| | | AATACGCCCGGTAGTGATCTTATTTCATTATGGTGAAAG |
| | | TTGGAACCTCTTACGTGCCGATCAACGTCTCATTTTCGC |
| | | CAAAAGTTGGCCCAGGGCTTCCCGGTATCAACAGGGAC |
| | | ACCAGGATTTATTTATTCTGCGAAGTGATCTTCCGTCAC |
| | | AGGTATTTATTCGGCGCAAAGTGCGTCGGGTGATGCTGC |
| | | CAACTTACTGATTTAGTGTATGATGGTGTTTTTGAGGTG |
| | | CTCCAGTGGCTTCTGTTTCTATCAGCTGTCCCTCCTGTTC |
| | | AGCTACTGACGGGGTGGTGCGTAACGGCAAAAGCACCG |
| | | CCGGACATCAGCGCTAGCGGAGTGTATACTGGCTTACT |
| | | ATGTTGGCACTGATGAGGGTGTCAGTGAAGTGCTTCATG |
| | | TGGCAGGAGAAAAAAGGCTGCACCGGTGCGTCAGCAGA |
| | | ATATGTGATACAGGATATATTCCGCTTCCTCGCTCACTG |
| | | ACTCGCTACGCTCGGTCGTTCGACTGCGGCGAGCGGAA |
| | | ATGGCTTACGAACGGGGCGGAGATTTCCTGGAAGATGC |
| | | CAGGAAGATACTTAACAGGGAAGTGAGAGGGCCGCGG |
| | | CAAAGCCGTTTTTCCATAGGCTCCGCCCCCCTGACAAGC |
| | | ATCACGAAATCTGACGCTCAAATCAGTGGTGGCGAAAC |
| | | CCGACAGGACTATAAAGATACCAGGCGTTTCCCCCTGG |
| | | CGGCTCCCTCGTGCGCTCTCCTGTTCCTGCCTTTCGGTTT |
| | | ACCGGTGTCATTCCGCTGTTATGGCCGCGTTTGTCTCAT |
| | | TCCACGCCTGACACTCAGTTCCGGGTAGGCAGTTCGCTC |
| | | CAAGCTGGACTGTATGCACGAACCCCCCGTTCAGTCCG |
| | | ACCGCTGCGCCTTATCCGGTAACTATCGTCTTGAGTCCA |
| | | ACCCGGAAAGACATGCAAAAGCACCACTGGCAGCAGCC |
| | | ACTGGTAATTGATTTAGAGGAGTTAGTCTTGAAGTCATG |
| | | CGCCGGTTAAGGCTAAACTGAAAGGACAAGTTTTGGTG |
| | | ACTGCGCTCCTCCAAGCCAGTTACCTCGGTTCAAAGAGT |
| | | TGGTAGCTCAGAGAACCTTCGAAAAACCGCCCTGCAAG |
| | | GCGGTTTTTTCGTTTTCAGAGCAAGAGATTACGCGCAGA |
| | | CCAAAACGATCTCAAGAAGATCATCTTATTAATCAGAT |
| | | AAAATATTTCTAGATTTCAGTGCAATTTATCTCTTCAAA |
| | | TGTAGCACCTGAAGTCAGCCCCATACGATATAAGTTGTA |
| | | ATTCTCATGTTTGACAGCTTATCATCGATAAGCTTGGTA |
| | | CCCAATTATGACAACTTGACGGCTACATCATTCACTTTT |
| | | TCTTCACAACCGGCACGGAACTCGCTCGGGCTGGCCCC |
| | | GGTGCATTTTTTAAATACCCGCGAGAAATAGAGTTGATC |
| | | GTCAAAACCAACATTGCGACCGACGGTGGCGATAGGCA |
| | | TCCGGGTGGTGCTCAAAAGCAGCTTCGCCTGGCTGATAC |
| | | GTTGGTCCTCGCGCCAGCTTAAGACGCTAATCCCTAACT |
| | | GCTGGCGGAAAAGATGTGACAGACGCGACGGCGACAA |
| | | GCAAACATGCTGTGCGACGCTGGCGATATCAAAATTGC |
| | | TGTCTGCCAGGTGATCGCTGATGTACTGACAAGCCTCGC |
| | | GTACCCGATTATCCATCGGTGGATGGAGCGACTCGTTAA |
| | | TCGCTTCCATGCGCCGCAGTAACAATTGCTCAAGCAGAT |
| | | TTATCGCCAGCAGCTCCGAATAGCGCCCTTCCCCTTGCC |
| | | CGGCGTTAATGATTTGCCCAAACAGGTCGCTGAAATGC |
| | | GGCTGGTGCGCTTCATCCGGGCGAAAGAACCCCGTATT |
| | | GGCAAATATTGACGGCCAGTTAAGCCATTCATGCCAGT |
| | | AGGCGCGCGGACGAAAGTAAACCCACTGGTGATACCAT |
| | | TCGCGAGCCTCCGGATGACGACCGTAGTGATGAATCTCT |
| | | CCTGGCGGGAACAGCAAAATATCACTCGGTCGGCAAAC |
| | | AAATTCTCGTCCCTGATTTTTCACCACCCCCTGACCGCG |
| | | AATGGTGAGATTGAGAATATAACCTTTCATTCCCAGCGG |
| | | TCGGTCGATAAAAAAATCGAGATAACCGTTGGCCTCAA |
| | | TCGGCGTTAAACCCGCCACCAGATGGGCATTAAACGAG |
| | | TATCCCGGCAGCAGGGGATCATTTTGCGCTTCAGCCATA |
| | | CTTTTCATACTCCCGCCATTCAGAGAAGAAACCAATTGT |
| | | CCATATTGCATCAGACATTGCCGTCACTGCGTCTTTTAC |
| | | TGGCTCTTCTCGCTAACCAAACCGGTAACCCCGCTTATT |
| | | AAAAGCATTCTGTAACAAAGCGGGACCAAAGCCATGAC |
| | | AAAAACGCGTAACAAAAGTGTCTATAATCACGGCAGAA |
| | | AAGTCCACATTGATTATTTGCACGGCGTCACACTTTGCT |
| | | ATGCCATAGCATTTTTATCCATAAGATTAGCGGATCCTA |
| | | CCTGACGCTTTTTATCGCAACTCTCTACTGTTTCTCCATA |
| | | CCCGTTTTTTTGGGCTAACAGGAGGAATT |
| 90 | Wild-type amino | MDKKPLNTLISATGLWMSRTGTIHKIKHHEVSRSKIYIEMA |
| | acid sequence of | CGDHLVVNNSRSSRTARALRHHKYRKTCKRCRVSDEDLN |
| | Methanosarcina | KFLTKANEDQTSVKVKVVSAPTRTKKAMPKSVARAPKPL |
| | mazei | ENTEAAQAQPSGSKFSPAIPVSTQESVSVPASVSTSISSISTG |
| | pyrrolysyl-tRNA | ATASALVKGNTNPITSMSAPVQASAPALTKSQTDRLEVLL |
| | synthetase | NPKDEISLNSGKPFRELESELLSRRKKDLQQIYAEERENYL |
| | | GKLEREITRFFVDRGFLEIKSPILIPLEYIERMGIDNDTELSK |
| | | QIFRVDKNFCLRPMLAPNLYNYLRKLDRALPDPIKIFEIGPC |
| | | YRKESDGKEHLEEFTMLNFCQMGSGCTRENLESIITDFLNH |
| | | LGIDFKIVGDSCMVYGDTLDVMHGDLELSSAVVGPIPLDR |
| | | EWGIDKPWIGAGFGLERLLKVKHDFKNIKRAARSESYYNG |
| | | ISTNL* |
| 91 | FSKRS-OPT | ATGACCGTGAAGTACACCGACGCCCAGATCCAACGCCT |
| | nucleotide | GCGCGAGTATGGTAATGGCACCTATGAACAGAAAGTTT |
| | sequence | TTGAGGACCTGGCCAGCCGTGACGCCGCGTTCAGCAAG |
| | | GAAATGAGCATTGCCAGCACCGACAACGAGAAGAAAAT |
| | | CAAGGGTATGATTGCCAATCCGAGCCGCCACGGCCTGA |
| | | CCCAGCTGATGAACGACATCGCTGATGCCCTGGTGGCG |
| | | GAGGGTTTCATTGAAGTGCGTACCCCGATCTTTATTAGC |
| | | AAGGATGCGCTGGCTCGCATGACCATCACCGAAGACAA |
| | | GCCGCTGTTCAAACAGGTGTTTTGGATTGATGAGAAAC |
| | | GTGCCCTGCGCCCGATGCTGGCGCCAAACCTGGGTAGC |
| | | GTGGCTCGTGACCTGCGCGATCACACCGACGGTCCGGT |
| | | GAAAATCTTCGAAATGGGCAGCTGCTTTCGTAAAGAGA |
| | | GCCACAGCGGTATGCACCTGGAGGAGTTCACCATGCTG |
| | | AATCTGTTTGATATGGGTCCGCGCGGCGACGCTACCGA |
| | | AGTGCTGGAAAACTACATTAGCGTGGTGATGAAAGTGG |
| | | CCGGCCTGCCGGACTACGATCTGGTGCAGGAAGAGAGC |
| | | GACGTGTACAAGGAAACCATCGACGTGGAAATTAACGG |
| | | TCAGGAAGTGTGCAGCGCGGCTGTGGGTCCAACTCCTA |
| | | TTGATGCGGCCCATGACGTGCATGAGCCGTGGAGCGGT |
| | | GCTGGTTTTGGCCTGGAACGTCTGCTGACCATCCGCGAG |
| | | AAATACTCCACCGTGAAAAAGGGCGGCGCAAGCATCTC |
| | | CTACCTGAATGGTGCCAAAATCAATTAA |
| 92 | FSKRS-OPT | MTVKYTDAQIORLREYGNGTYEQKVFEDLASRDAAFSKE |
| | amino acid | MSIASTDNEKKIKGMIANPSRHGLTQLMNDIADALVAEGFI |
| | sequence | EVRTPIFISKDALARMTITEDKPLFKQVFWIDEKRALRPML |
| | | APNLGSVARDLRDHTDGPVKIFEMGSCFRKESHSGMHLEE |
| | | FTMLNLFDMGPRGDATEVLENYISVVMKVAGLPDYDLVQ |
| | | EESDVYKETIDVEINGQEVCSAAVGPTPIDAAHDVHEPWS |
| | | GAGFGLERLLTIREKYSTVKKGGASISYLNGAKIN |
| 93 | PEVOL-FSKRS- | ACTAGTATGACCGTGAAGTACACCGACGCCCAGATCCA |
| | OPT | ACGCCTGCGCGAGTATGGTAATGGCACCTATGAACAGA |
| | | AAGTTTTTGAGGACCTGGCCAGCCGTGACGCCGCGTTCA |
| | | GCAAGGAAATGAGCATTGCCAGCACCGACAACGAGAA |
| | | GAAAATCAAGGGTATGATTGCCAATCCGAGCCGCCACG |
| | | GCCTGACCCAGCTGATGAACGACATCGCTGATGCCCTG |
| | | GTGGCGGAGGGTTTCATTGAAGTGCGTACCCCGATCTTT |
| | | ATTAGCAAGGATGCGCTGGCTCGCATGACCATCACCGA |
| | | AGACAAGCCGCTGTTCAAACAGGTGTTTTGGATTGATG |
| | | AGAAACGTGCCCTGCGCCCGATGCTGGCGCCAAACCTG |
| | | GGTAGCGTGGCTCGTGACCTGCGCGATCACACCGACGG |
| | | TCCGGTGAAAATCTTCGAAATGGGCAGCTGCTTTCGTAA |
| | | AGAGAGCCACAGCGGTATGCACCTGGAGGAGTTCACCA |
| | | TGCTGAATCTGTTTGATATGGGTCCGCGCGGCGACGCTA |
| | | CCGAAGTGCTGGAAAACTACATTAGCGTGGTGATGAAA |
| | | GTGGCCGGCCTGCCGGACTACGATCTGGTGCAGGAAGA |
| | | GAGCGACGTGTACAAGGAAACCATCGACGTGGAAATTA |
| | | ACGGTCAGGAAGTGTGCAGCGCGGCTGTGGGTCCAACT |
| | | CCTATTGATGCGGCCCATGACGTGCATGAGCCGTGGAG |
| | | CGGTGCTGGTTTTGGCCTGGAACGTCTGCTGACCATCCG |
| | | CGAGAAATACTCCACCGTGAAAAAGGGCGGCGCAAGCA |
| | | TCTCCTACCTGAATGGTGCCAAAATCAATTAAGTCGACC |
| | | ATCATCATCATCATCATTGAGTTTAAACGGTCTCCAGCT |
| | | TGGCTGTTTTGGCGGATGAGAGAAGATTTTCAGCCTGAT |
| | | ACAGATTAAATCAGAACGCAGAAGCGGTCTGATAAAAC |
| | | AGAATTTGCCTGGCGGCAGTAGCGCGGTGGTCCCACCT |
| | | GACCCCATGCCGAACTCAGAAGTGAAACGCCGTAGCGC |
| | | CGATGGTAGTGTGGGGTCTCCCCATGCGAGAGTAGGGA |
| | | ACTGCCAGGCATCAAATAAAACGAAAGGCTCAGTCGAA |
| | | AGACTGGGCCTTGTTTGTGAGCTCCCGGTCATCAATCAT |
| | | CCCCATAATCCTTGTTAGATTATCAATTTTAAAAAACTA |
| | | ACAGTTGTCAGCCTGTCCCGCTTTAATATCATACGCCGT |
| | | TATACGTTGTTTACGCTTTGAGGAATCCCATATGATACG |
| | | CGGCCGCTTTCAAACGCTAAATTGCCTGATGCGCTACGC |
| | | TTATCAGGCCTACATGATCTCTGCAATATATTGAGTTTG |
| | | CGTGCTTTTGTAGGCCGGATAAGGCGTTCACGCCGCATC |
| | | CGGCAAGAAACAGCAAACAATCCAAAACGCCGCGTTCA |
| | | GCGGCGTTTTTTCTGCTTTTCTTCGCGAATTAATTCCGCT |
| | | TCGCAACATGTGAGCACCGGTTTATTGACTACCGGAAG |
| | | CAGTGTGACCGTGTGCTTCTCAAATGCCTGAGGCCAGTT |
| | | TGCTCAGGCTCTCCCCGTGGAGGTAATAATTGACGATAT |
| | | GATCAGTGCACGGCTAACTAAGCGGCCTGCTGACTTTCT |
| | | CGCCGATCAAAAGGCATTTTGCTATTAAGGGATTGACG |
| | | AGGGCGTATCTGCGCAGTAAGATGCGCCCCGCATTGGG |
| | | GGACGGTCCGGCGACCAGCGGGTCTCTAAAACCTAGCC |
| | | AGCGGGGTTCGACGCCCCGGTCTCTCGCCAAATTCGAA |
| | | AAGCCTGCTCAACGAGCAGGCTTTTTTGCATGCTCGAGC |
| | | AGCTCAGGGTCGAATTTGCTTTCGAATTTCTGCCATTCA |
| | | TCCGCTTATTATCACTTATTCAGGCGTAGCAACCAGGCG |
| | | TTTAAGGGCACCAATAACTGCCTTAAAAAAATTACGCC |
| | | CCGCCCTGCCACTCATCGCAGTACTGTTGTAATTCATTA |
| | | AGCATTCTGCCGACATGGAAGCCATCACAAACGGCATG |
| | | ATGAACCTGAATCGCCAGCGGCATCAGCACCTTGTCGC |
| | | CTTGCGTATAATATTTGCCCATGGTGAAAACGGGGGCG |
| | | AAGAAGTIGTCCATATTGGCCACGTTTAAATCAAAACTG |
| | | GTGAAACTCACCCAGGGATTGGCTGAGACGAAAAACAT |
| | | ATTCTCAATAAACCCTTTAGGGAAATAGGCCAGGTTTTC |
| | | ACCGTAACACGCCACATCTTGCGAATATATGTGTAGAA |
| | | ACTGCCGGAAATCGTCGTGGTATTCACTCCAGAGCGAT |
| | | GAAAACGTTTCAGTTTGCTCATGGAAAACGGTGTAACA |
| | | AGGGTGAACACTATCCCATATCACCAGCTCACCGTCTTT |
| | | CATTGCCATACGGAATTCCGGATGAGCATTCATCAGGC |
| | | GGGCAAGAATGTGAATAAAGGCCGGATAAAACTTGTGC |
| | | TTATTTTTCTTTACGGTCTTTAAAAAGGCCGTAATATCC |
| | | AGCTGAACGGTCTGGTTATAGGTACATTGAGCAACTGA |
| | | CTGAAATGCCTCAAAATGTTCTTTACGATGCCATTGGGA |
| | | TATATCAACGGTGGTATATCCAGTGATTTTTTTCTCCATT |
| | | TTAGCTTCCTTAGCTCCTGAAAATCTCGATAACTCAAAA |
| | | AATACGCCCGGTAGTGATCTTATTTCATTATGGTGAAAG |
| | | TTGGAACCTCTTACGTGCCGATCAACGTCTCATTTTCGC |
| | | CAAAAGTTGGCCCAGGGCTTCCCGGTATCAACAGGGAC |
| | | ACCAGGATTTATTTATTCTGCGAAGTGATCTTCCGTCAC |
| | | AGGTATTTATTCGGCGCAAAGTGCGTCGGGTGATGCTGC |
| | | CAACTTACTGATTTAGTGTATGATGGTGTTTTTGAGGTG |
| | | CTCCAGTGGCTTCTGTTTCTATCAGCTGTCCCTCCTGTTC |
| | | AGCTACTGACGGGGTGGTGCGTAACGGCAAAAGCACCG |
| | | CCGGACATCAGCGCTAGCGGAGTGTATACTGGCTTACT |
| | | ATGTTGGCACTGATGAGGGTGTCAGTGAAGTGCTTCATG |
| | | TGGCAGGAGAAAAAAGGCTGCACCGGTGCGTCAGCAGA |
| | | ATATGTGATACAGGATATATTCCGCTTCCTCGCTCACTG |
| | | ACTCGCTACGCTCGGTCGTTCGACTGCGGCGAGCGGAA |
| | | ATGGCTTACGAACGGGGCGGAGATTTCCTGGAAGATGC |
| | | CAGGAAGATACTTAACAGGGAAGTGAGAGGGCCGCGG |
| | | CAAAGCCGTTTTTCCATAGGCTCCGCCCCCCTGACAAGC |
| | | ATCACGAAATCTGACGCTCAAATCAGTGGTGGCGAAAC |
| | | CCGACAGGACTATAAAGATACCAGGCGTTTCCCCCTGG |
| | | CGGCTCCCTCGTGCGCTCTCCTGTTCCTGCCTTTCGGTTT |
| | | ACCGGTGTCATTCCGCTGTTATGGCCGCGTTTGTCTCAT |
| | | TCCACGCCTGACACTCAGTTCCGGGTAGGCAGTTCGCTC |
| | | CAAGCTGGACTGTATGCACGAACCCCCCGTTCAGTCCG |
| | | ACCGCTGCGCCTTATCCGGTAACTATCGTCTTGAGTCCA |
| | | ACCCGGAAAGACATGCAAAAGCACCACTGGCAGCAGCC |
| | | ACTGGTAATTGATTTAGAGGAGTTAGTCTTGAAGTCATG |
| | | CGCCGGTTAAGGCTAAACTGAAAGGACAAGTTTTGGTG |
| | | ACTGCGCTCCTCCAAGCCAGTTACCTCGGTTCAAAGAGT |
| | | TGGTAGCTCAGAGAACCTTCGAAAAACCGCCCTGCAAG |
| | | GCGGTTTTTTCGTTTTCAGAGCAAGAGATTACGCGCAGA |
| | | CCAAAACGATCTCAAGAAGATCATCTTATTAATCAGAT |
| | | AAAATATTTCTAGATTTCAGTGCAATTTATCTCTTCAAA |
| | | TGTAGCACCTGAAGTCAGCCCCATACGATATAAGTTGTA |
| | | ATTCTCATGTTTGACAGCTTATCATCGATAAGCTTGGTA |
| | | CCCAATTATGACAACTTGACGGCTACATCATTCACTTTT |
| | | TCTTCACAACCGGCACGGAACTCGCTCGGGCTGGCCCC |
| | | GGTGCATTTTTTAAATACCCGCGAGAAATAGAGTTGATC |
| | | GTCAAAACCAACATTGCGACCGACGGTGGCGATAGGCA |
| | | TCCGGGTGGTGCTCAAAAGCAGCTTCGCCTGGCTGATAC |
| | | GTTGGTCCTCGCGCCAGCTTAAGACGCTAATCCCTAACT |
| | | GCTGGCGGAAAAGATGTGACAGACGCGACGGCGACAA |
| | | GCAAACATGCTGTGCGACGCTGGCGATATCAAAATTGC |
| | | TGTCTGCCAGGTGATCGCTGATGTACTGACAAGCCTCGC |
| | | GTACCCGATTATCCATCGGTGGATGGAGCGACTCGTTAA |
| | | TCGCTTCCATGCGCCGCAGTAACAATTGCTCAAGCAGAT |
| | | TTATCGCCAGCAGCTCCGAATAGCGCCCTTCCCCTTGCC |
| | | CGGCGTTAATGATTTGCCCAAACAGGTCGCTGAAATGC |
| | | GGCTGGTGCGCTTCATCCGGGCGAAAGAACCCCGTATT |
| | | GGCAAATATTGACGGCCAGTTAAGCCATTCATGCCAGT |
| | | AGGCGCGCGGACGAAAGTAAACCCACTGGTGATACCAT |
| | | TCGCGAGCCTCCGGATGACGACCGTAGTGATGAATCTCT |
| | | CCTGGCGGGAACAGCAAAATATCACTCGGTCGGCAAAC |
| | | AAATTCTCGTCCCTGATTTTTCACCACCCCCTGACCGCG |
| | | AATGGTGAGATTGAGAATATAACCTTTCATTCCCAGCGG |
| | | TCGGTCGATAAAAAAATCGAGATAACCGTTGGCCTCAA |
| | | TCGGCGTTAAACCCGCCACCAGATGGGCATTAAACGAG |
| | | TATCCCGGCAGCAGGGGATCATTTTGCGCTTCAGCCATA |
| | | CTTTTCATACTCCCGCCATTCAGAGAAGAAACCAATTGT |
| | | CCATATTGCATCAGACATTGCCGTCACTGCGTCTTTTAC |
| | | TGGCTCTTCTCGCTAACCAAACCGGTAACCCCGCTTATT |
| | | AAAAGCATTCTGTAACAAAGCGGGACCAAAGCCATGAC |
| | | AAAAACGCGTAACAAAAGTGTCTATAATCACGGCAGAA |
| | | AAGTCCACATTGATTATTTGCACGGCGTCACACTTTGCT |
| | | ATGCCATAGCATTTTTATCCATAAGATTAGCGGATCCTA |
| | | CCTGACGCTTTTTATCGCAACTCTCTACTGTTTCTCCATA |
| | | CCCGTTTTTTTGGGCTAACAGGAGGAATT |

In some cases, the targeting domain is recombinantly produced by a host cell system. In some cases, the host cell is a eukaryotic cell (e.g., a mammalian cell, an insect cell, a yeast cell, or a plant cell) or a prokaryotic cell (e.g., a gram-positive or gram-negative bacterium). In some cases, the eukaryotic host cell is a mammalian host cell. In some cases, the mammalian host cell is a stable cell line, or a cell line that has incorporated the genetic material of interest into its own genome and has the ability to express the product of that genetic material after multiple generations of cell division. In other cases, the mammalian host cell is a transient cell line, or a cell line that has incorporated the genetic material of interest into its own genome and does not have the ability to express the product of the genetic material after multiple generations of cell division.

Exemplary mammalian host cells include 293T cell line, 293A cell line, 293FT cell line, 293F cell, 293H cell, A549 cell, MDCK cell, CHO DG44 cell, CHO—S cell, CHO-K1 cell, Expi293F Cell™ cell, Flp-In™T-REx™293 cell line, Flp-In™-293 cell line, Flp-In™-3T3 cell line, Flp-In™BHK cell line, Flp-In™—CHO cell line, Flp-In™—CV-1 cell line, Flp-In™The Jurkat cell line, FreeStyle™293-F cells, FreeStyle™CHO—S cell, GripTite™293MSR cell line, GS-CHO cell line, Heparg™ cell, T-REx™Jurkat cell line, Per.C6 cells, T-REx™-293 cell line, T-REx™—CHO cell line and T-REx™HeLa cell line.

In some embodiments, the eukaryotic host cell is an insect host cell. Exemplary insect host cells include Drosophila S2 cell, Sf9 cell, Sf21 cell, and Cellular High Five™ cells.

In some embodiments, the eukaryotic host cell is a yeast host cell. Exemplary yeast host cells include Pichia pastoris yeast strains, such as GS115, KM71H, SMD1168H, and X-33, and Saccharomyces cerevisiae yeast strains, such as INVSCl.

In some embodiments, the eukaryotic host cell is a plant host cell. In some cases, the plant cell comprises a cell from an alga. Exemplary plant cell lines include strains from Chlamydomonas reinhardtii 137c or Synechococcus elongatus PPC 7942.

In some embodiments, the host cell is a prokaryotic host cell. Exemplary prokaryotic host cells include BL21, BL21(DE3), Mach1™, DH10B™, TOP10, DH5α, DH10BaC™, OmniMax™, MegaX™, DH12S™, INV110, TOP10F′, INVαF, TOP10/P3, ccdB Survival, PIR1, PIR2, Stb12™, Stb13™ Or Stb14™.

In some cases, suitable nucleic acid molecules or vectors for producing the targeting domains described herein include any suitable vector derived from eukaryotic or prokaryotic sources. Exemplary nucleic acid molecules or vectors include vectors from bacterial (e.g., E. coli), insect, yeast (e.g., Pichia pastoris), algal, or mammalian sources. Bacterial vectors include, for example, pACYC177, pASK75, the pBAD series of vectors, the pBADM series of vectors, the pET series of vectors, the pETM series of vectors, the pGEX series of vectors, pHAT2, pMal-C2, pMal-p2, pQE series of vectors, pRSET A, pRSET B, pRSET C, the pTrcHis2 series, pZA31-Luc, pZE21-MCS-1, pFLAG ATS, pFLAG CTS, pFLAG MAC, pFLAG Shift-12C, pTAC-MAT-1, pFLAG CTC or pTAC-MAT-2.

Insect vectors include, for example, pFastBac1, pFastBac DUAL, pFastBac ET, pFastBac HTa, pFastBac HTb, pFastBac HTc, pFastBac M30a, pFastBac M30b, pFastBac, M30c, pVL1392, pVL1393M 10, pVL1393M11, pVL1393M 12, FLAG vectors such as pPolh-FLAG1 or pPolh-MAT2, or MAT vectors such as pPolh-MAT1 or pPolh-MAT 2.

Yeast vectors include, for example, pDEST™14 a carrier, pDEST™15 a carrier, pDEST™17 a carrier, pDEST™24 carrier, a, pYES-DEST52 vector, pBAD-DEST49Target vector, pAO815 Pichia yeast vector, pFLD1 Pichia pastoris vector, pGAPZA, Pichia pastoris C vector, Pichia pastoris pPIC3.5K vector, pPIC 6A, B and Pichia pastoris C vector, pPIC9K vector, pTEF1/Zeo, pYES2 yeast vector, pYES2/CT yeast vector, pYES2/NT A, B and C yeast parent or pYES3/CT yeast vector.

Algal vectors include, for example, pChlamy-4 vectors or MCS vectors.

Mammalian vectors include, for example, transient expression vectors or stable expression vectors. Exemplary mammalian transient expression vectors include p3×FLAG-CMV 8, pFLAG-Myc-CMV19, pFLAG-Myc-CMV 23, pFLAG-CMV 2, pFLAG-CMV 6a, b, c, pFLAG-CMV 5.1, pFLAG-CMV 5a, b, c, p3×FLAG-CMV 7.1, pF-CMV 20, p3×FLAG-Myc-CMV 24, pCMV-FLAG-MAT1, pCMV-FLAG-MAT2, pBICEP-CMV 3, or pBICCMV-4. Exemplary mammalian stable expression vectors include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV14, pFLAG-Myc-CMV 22, p3×FLAG-Myc-CMV 26, pBICEP-CMV 1, or pBICEP-CMV 2. In some cases, a cell-free system is used to produce a targeting domain described herein. In some cases, cell-free systems comprise a mixture of cytoplasmic and/or nuclear components from cells and are suitable for in vitro nucleic acid synthesis. In some cases, cell-free systems utilize prokaryotic cellular components. In other cases, cell-free systems utilize eukaryotic cell components. Nucleic acid synthesis is achieved in cell-free systems based on, for example, Drosophila cells, Xenopus eggs, archaea or HeLa cells. Exemplary cell-free systems include the E. coli S30 Extract system, the E. coli T7S 30 system or XpressCF and XpressCF+.

Cell-free translation systems variously comprise components such as plasmids, mRNA, DNA, tRNA, synthetases, release factors, ribosomes, chaperones, translation initiation and elongation factors, natural and/or unnatural amino acids, and/or other components for protein expression. Such components are optionally modified to improve yield, increase synthesis rate, increase fidelity of the protein product, or incorporate unnatural amino acids. In some embodiments, the unnatural amino acid-containing targeting domains described herein are synthesized using the cell-free translation system described in U.S. Pat. No. 8,778,631, US2017/0283469, US 2018/0051065, US 2014/0315245, or U.S. Pat. No. 8,778,631. In some embodiments, the cell-free translation system comprises a modified release factor, or even one or more release factors are removed from the system. In some embodiments, the cell-free translation system comprises a reduced protease concentration. In some embodiments, the cell-free translation system comprises a modified tRNA having a reassigned codon encoding an unnatural amino acid. In some embodiments, the synthetases described herein for incorporating unnatural amino acids are used in cell-free translation systems. In some embodiments, the tRNA is preloaded with an unnatural amino acid using an enzymatic or chemical process prior to adding the tRNA to the cell-free translation system. In some embodiments, the components for the cell-free translation system are obtained from a modified organism, such as a modified bacterium, yeast, or other organism.

In some embodiments, the targeting domain is produced in a circular arrangement via an expression host system or by a cell-free system.

An orthogonal or expanded genetic code can be used to generate the targeting domains described herein, wherein one or more specific codons present in the nucleic acid sequence of a targeting domain are assigned to encode an unnatural amino acid, such that it can be genetically incorporated into a conjugate (e.g., targeting domain) through the use of an orthogonal tRNA synthetase/tRNA pair. The orthogonal tRNA synthetase/tRNA pair is capable of charging a tRNA with an unnatural amino acid, and is capable of incorporating the unnatural amino acid into a polypeptide chain in response to a codon.

In some cases, the codon is an amber, ochre, an opal codon, or a quadruple codon. In some cases, the codon corresponds to an orthogonal tRNA that will be used to carry the unnatural amino acid. In some cases, the codon is an amber codon. In other cases, the codon is an orthogonal codon.

In some cases, the codon is a quadruple codon, which can be decoded by the orthogonal ribosomal ribo-Q1. In some cases, the quadruple codons are as described in Neumann et al, “Encoding multiple unnatural amino acid analysis of a quadruplet-decoding ribosome,” Nature, 464(7287):441-444 (2010).

In some cases, a codon used in the present disclosure is a recoded codon, e.g., a synonymous codon or a rare codon that is replaced with a replacement codon. In some cases, the recoded codons are as described in Napolitano et al, “Emergent rules for codon choice isolated by editing of a ray array code in Escherichia coli,” PNAS, 113(38): E5588-5597 (2016). In some cases, the recoded codons are as described in Ostrov et al., “Design, synthesis, and testing translated a 57-code gene,” Science 353(6301): 819. sub.822 (2016).

### Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, g-carboxyglutamate, and O-phosphoserine. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “non-naturally occurring amino acid” and “unnatural amino acid” refer to amino acid analogs, synthetic amino acids, and amino acid mimetics which are not found in nature, e.g. amino acid residues containing arylamide, vinyl sulfonamide, sulfonyl fluoride, aryl sulfonyl fluoride, and 4-sulfotetrafluorophenyl (STP) esters.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, g-carboxyglutamate, and O-phosphoserine. In aspects, the amino acid side chain may be a non-natural amino acid side chain. In aspects, the amino acid side chain is H,

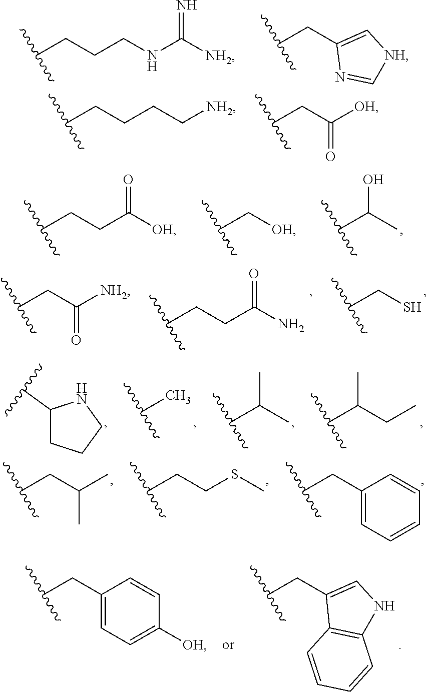

In some embodiments, the unnatural amino acid side chain is

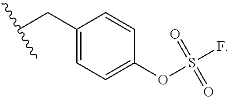 [SMILES: *Cc1ccc(OS(=O)(=O)F)cc1]

In some embodiments, the unnatural amino acid side chain is

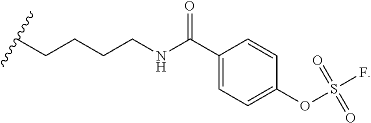 [SMILES: *CCCCNC(=O)c1ccc(OS(=O)(=O)F)cc1]

The term “non-natural amino acid side chain” or “unnatural amino acid side chain” or “Uaa” refers to the functional substituent of compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptanecarboxylic acid hydrochloride, cis-6-Amino-3-cyclohexene-1-carboxylic acid hydrochloride, cis-2-amino-2-methylcyclohexanecarboxylic acid hydrochloride, cis-2-amino-2-methylcyclopentanecarboxylic acid hydrochloride, 2-(Bocaminomethyl), benzoic acid, 2-(Boc-amino)octanedioic acid, Boc-4,5-dehydro-Leu-OH (dicyclohexylammonium), Boc-4-(Fmoc-amino)-L-phenylalanine, Boc-P-Homopyr-OH, Boc-( 2-indanyl)-Gly-OH, 4-Boc-3-morpholineacetic acid, 4-Boc-3-morpholineacetic acid, Bocpentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NH2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Bocpiperazino)-2-[4-(trifluoromethyl)phenyl]acetic acid purum, Boc-P-(2-quinolyl)-Ala-OH, NBoc-1,2,3,6-tetrahydro-2-pyridinecarboxylic acid, Boc-P-(4-thiazolyl)-Ala-OH, Bo-b-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, FmocPhe(3,5-F2)-OH, Fmoc-P-(4-thiazolyl)-Ala-OH, Fmoc-P-(2-thienyl)-Ala-OH, 4-(Hydroxymethyl)-D-phenylalanine. In some embodiments, the unnatural amino acid comprises a structure of Formula I:

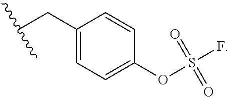 [SMILES: *Cc1ccc(OS(=O)(=O)F)cc1]

In some embodiments, the unnatural amino acid comprises a structure of Formula II:

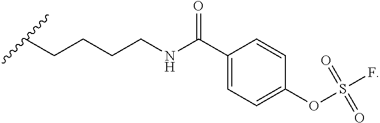 [SMILES: *CCCCNC(=O)c1ccc(OS(=O)(=O)F)cc1]

In some embodiments, the unnatural amino acid is 2-amino-3-(4-((fluorosulfonyl)oxy)phenyl)propanoic acid:

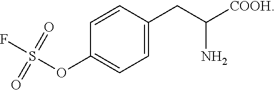 [SMILES: NC(Cc1ccc(OS(=O)(=O)F)cc1)C(=O)O]

In some embodiments, the unnatural amino acid is fluorosulfonyltyrosine (FSY):

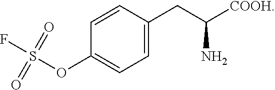 [SMILES: N[C@@H](Cc1ccc(OS(=O)(=O)F)cc1)C(=O)O]

In some embodiments, the unnatural amino acid is N6-(4-((fluorosulfonyl)oxy)benzoyl)lysine:

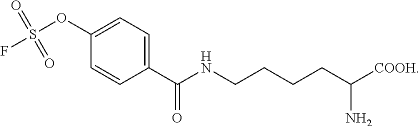 [SMILES: NC(CCCCNC(=O)c1ccc(OS(=O)(=O)F)cc1)C(=O)O]

In some embodiments, the unnatural amino acid is fluorosulfonyloxybenzoyl-L-lysine (FSK):

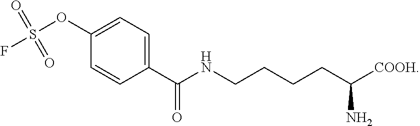 [SMILES: N[C@@H](CCCCNC(=O)c1ccc(OS(=O)(=O)F)cc1)C(=O)O]

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). (see, e.g., Creighton, Proteins (1984)).

In some embodiments, pyrrolysyl-tRNA synthetase (tRNA^{pyl}) referred to herein is an aminoacyl-tRNA synthetase that catalyzes the reaction necessary to attach an α-amino acid pyrrolysine or an analogous unnatural amino acid to the cognate tRNA, thereby allowing incorporation of pyrrolysine or analogous unnatural amino acid during proteinogenesis at amber stop codons (i.e., UAG). The wild-type tRNA^{pyl} from Methanosarcina species, which naturally incorporates pyrrolysine, is orthogonal to endogenous tRNAs and aminoacyl-tRNA synthetases in E. coli and eukaryotic cells. Using this pair, and its synthetically evolved derivatives or variants, we and others have directed the efficient incorporation of unnatural amino acids, including post-translationally modified amino acids, chemical handles, and photocaged amino acids, at specific sites in desired proteins in E. coli, yeast, and mammalian cells.

In some embodiments, tRNA^{pyl} described herein includes any recombinant or naturally-occurring form of pyrrolysyl-tRNA synthetase or variants, homologs, or isoforms thereof that maintain tRNA^{pyl} activity (e.g. within at least 30%, 40%, 50%, 60%, 70%, 80%, 90%, 95%, or 100% activity compared to wild-type tRNA^{pyl}). In some embodiments, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring pyrrolysyl-tRNA synthetase. In some embodiments, the mutant tRNA^{pyl} catalyzes the attachment of an unnatural amino acid (UAA) (e.g., a UAA of Formula (IA) such as fluorosulfate L-tyrosine (FSY)) to a tRNA^{pyl} in order that the unnatural amino acid (UAA) is incorporated.

In some embodiments, the tRNA^{pyl} provided herein is a tRNA^{pyl} derivative or variant that can be engineered by those skilled in the art. In some embodiments, the tRNA^{pyl} provided herein is a single-stranded RNA molecule containing about 70 to 90 nucleotides which fold via intrastrand base pairing to form a characteristic cloverleaf structure that carries a specific amino acid (e.g., e.g., a UAA of Formula (IA) such as FSY) and matches it to its corresponding codon on an mRNA during protein synthesis.

An “imaging ligand” or a “detectable agent” is a composition detectable by appropriate means such as spectroscopic, photochemical, biochemical, immunochemical, chemical, magnetic resonance imaging, or other physical means. For example, useful detectable agents include ³H, ¹⁴C, ¹⁸F, ³³P, ³⁵S, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹¹²In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶HO, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, ¹⁵³Sm, ¹⁷⁷Lu, ⁸⁶Y, ⁸⁸Y, ⁹⁰Y, ¹²³I, ¹²⁴I, ¹²⁵I, ¹³¹I, ¹⁴⁹Tb, ²¹²Pb/²¹²Bi, and ²²⁷Th, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to ³H, ¹⁴C, ¹⁸F, ³³P, ³⁵S, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹¹²In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶HO, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, ¹⁵³Sm ¹⁷⁷Lu, ⁸⁶Y, ⁸⁸Y, ⁹⁰Y, ¹²³I, ¹²⁴I, ¹²⁵I, ¹³¹I, ¹⁴⁹Tb, ²¹²Pb/²¹²Bi, and ²²⁷Th. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The term “linkage” or “linker” as used herein to refer to bonds or chemical moiety formed from a chemical reaction between the functional group of a linker and another molecule. Such bonds may include, but are not limited to, covalent linkages and non-covalent bonds, while such chemical moieties may include, but are not limited to, esters, carbonates, imines phosphate esters, hydrazones, acetals, orthoesters, peptide linkages, and oligonucleotide linkages. Hydrolytically stable linkages mean that the linkages are substantially stable in water and do not react with water at useful pH values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide. Linkers include but are not limited to short linear, branched, multi-armed, or dendrimeric molecules such as polymers. In some embodiments the linker may be branched. In other embodiments the linker may be a bifunctional linker. In some embodiments, the linker may be a trifunctional linker. The mechanisms for release of an agent from these linker groups include, for example, irradiation of a photolabile bond and acid-catalyzed hydrolysis. The length of the linker may be predetermined or selected depending upon a desired spatial relationship between the polypeptide and the molecule linked to it.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to Ala302 of the PylRS protein when the selected residue occupies the same essential spatial or other structural relationship as Ala302 in the PylRS protein. In embodiments, where a selected protein is aligned for maximum homology with the PylRS protein, the position in the aligned selected protein aligning with Ala302 is said to correspond to Ala302. Instead of a primary sequence alignment, a three-dimensional structural alignment can also be used, e.g., where the structure of the selected protein is aligned for maximum correspondence with the PylRS protein and the overall structures compared. In this case, an amino acid that occupies the same essential position as Ala302 in the structural model is said to correspond to the Ala302 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Antibodies” are large, complex proteins with an intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

An antibody “variant” as provided herein refers to a polypeptide capable of binding to an antigen and including one or more structural domains of an antibody or fragment thereof. Nonlimiting examples of antibody variants include single-domain antibodies (nanobodies), affibodies (polypeptides smaller than monoclonal antibodies (e.g., about 6 kDA) and capable of binding antigens with high affinity and imitating monoclonal antibodies, monospecific Fab2, bispecific Fab2, trispecific Fab3, monovalent IgGs, scFv, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO 97/49805 and WO 97/49805, which are incorporated, by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

A “single-domain antibody” or “nanobody” refers to an antibody fragment having a single monomeric variable antibody domain. Like a whole antibody, it is able to bind selectively to a specific antigen. In some embodiments, the single domain antibody is a human or humanized single domain antibody. In some embodiments, the single domain antibody is a camelid single domain antibody. A single domain antibody can be an engineered single domain antibody and can comprise an unnatural amino acid

The term “antigen” as provided herein refers to molecules capable of binding to the antibody binding domain provided herein. An “antigen binding domain” as provided herein is a region of an antibody that binds to an antigen (epitope). As described above, the antigen binding domain may include one constant and one variable domain of each of the heavy and the light chain (VL, VH, CL and CH1, respectively). In embodiments, the antigen binding domain includes a light chain variable domain and a heavy chain variable domain. In embodiments, the antigen binding domain includes light chain variable domain and does not include a heavy chain variable domain and/or a heavy chain constant domain. The paratope or antigen-binding site is formed on the N-terminus of the antigen binding domain. The two variable domains of an antigen binding domain may bind the epitope of an antigen. Antibodies exist, for example, as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

### EXAMPLES

### Example 1—Identification of Coupling Sites for PSMA sdAbs

To assess the compatibility of PSMA as a target for coupling with an FSY-containing targeting molecule using the single domain antibody (sdAb) C1 (SEQ ID NO:1), FSY sites were screened by replacement of each of the selected codons within CDR1, CDR2, and CDR3 regions with a TAG stop codon and production of the encoded protein to incorporate FSY into the appropriate position. Approximately 19 potential sites were identified, with the majority clustered in the CDR1 and CDR3 regions. These TAG sites are located in the CDR1 (site 26-35: GYTDSNYYMS, SEQ ID NO:5); CDR2 (50-66: VNTGRGSTSYADSVKG, SEQ ID NO:6), CDR3 (99-116, excluding Cys101, Cys104 AACHFCDSLPKTQDEYIL, SEQ ID NO:7). A second sdAb C2 (SEQ ID NO:2) was assessed similarly for FSY modification sites. Approximately 8 sites were found as potential placements for FSY in the C2 CDRs, with the majority clustering in CDR2 and CDR3. These TAG sites are located in the CDR1 (site 26-35 RFMISEYSMH, SEQ ID NO:8); CDR2 (50-65: TINPAGTTDYAESVKG, SEQ ID NO:9), CDR3 (96-100 DGYGY, SEQ ID NO:10).

### Example 2—Expression of FSY-Modified sdAbs

The vector pBAD sequence (Invitrogen #43001) lacking the ORF insert was PCR amplified with a forward and reverse primer (SEQ ID NO:80) and (SEQ ID NO:81), respectively.

The resulting PCR-amplified vector was gel extracted and purified using a Zymo PCR purification kit (Zymoclean Gel DNA Recovery kits Cat #D4002). The dsDNA sequences for C2 WT His and C1 WT His were ligated with the PCR-amplified pBAD-backbone and transformed into E. coli DH10b chemically-competent cells (Fisher Thermo Scientific™ DH10B Competent Cells; High Efficiency; FEREC0113). Clones were miniprepped and sequence verified using pBAD-forward primer. The expression cassettes also incorporated the PelB leader sequence and His6 tag. The DNA sequences of C1 and C2 wt are SEQ ID NO: 73 and SEQ ID NO: 74, respectively. For constructs incorporating an FSY residue, a TAG codon was engineered into the DNA sequence at the desired position for the amino acid substitution within the open reading frame of the sdAb or biparatopic construct.

Construction of the C1 TAG library. Polynucleotides (dsDNA sequences) containing a TAG codon in place of each codon within the sites identified in CDR regions of C1 (see Example 1) were synthesized by IDT. These TAG sites are located in the CDR1 (site 26-35); CDR2 (50- 66), CDR3 (99-116, excluding Cys101, Cys104). These sites are underlined in the sequence below:

(SEQ ID NO: 1)
QVQLQESGGGSVQAGGSLRLSCTAPGYTDSNYYMSWFRQAPGKEREWVAG
VNTGRGSTSYADSVKGRFTISQDNAKNTMFLQMNSLKPEDTAIYYCAVAA
CHFCDSLPKTQDEYILWGQGTQVTVSSAAAYPYDVPDYG

Polynucleotides containing each of these TAG mutants were cloned into the pBAD-backbone as described above and sequence verified.

Construction of the C2 TAG library. Polynucleotide sequences containing a single TAG codon replacing each residue in CDR regions of C2 identified in Example 1 were synthesized by IDT. These regions in the amino acid sequence of the C2 WT His protein sequence are underlined in the sequence below:

(SEQ ID NO: 2)
EVQLVESGGGLVQPGGSLTLSCAASRFMISEYSMHWVRQAPGKGLEWVST
INPAGTTDYAESVKGRFTISRDNAKNTLYLQMNSLKPEDTAVYYCDGYGY
RGQGTQVTVSS

Polynucleotides containing each of these TAG mutants were cloned into pBAD-backbone as described above and sequence verified.

Library strain generation. The pEVOL-FSYRS plasmid which expresses the engineered Mb FSYRS aminoacyl tRNA synthetase was synthesized by Genscript (Order No. U168NGE200; Cat #SC1010) (Ref: J. Am. Chem. Soc. 2018, 140, 15, 4995-4999), was transformed into chemically competent DH10b competent cells using standard methods to generate the parental strain FSYRS-DH10b. Individual plasmids encoding each C1 CDR TAG or C2 CDR TAG variant were transformed into chemically competent FSYRS-DH10b using standard methods. Single colonies were inoculated into 2×YT media (Teknova #Y0166) containing 100 ug/ml ampicillin and 34 ug/ml chloramphenicol and grown overnight shaking at 37° C. approximately 220 rpm. Overnight cultures were mixed 1:1 with sterile 50% glycerol and stored at −80° C.

Expression and purification. Strains bearing the plasmids encoding sdAbs with individual CDR residues mutated to TAG in the FSYRS-DH10b parental background were cultured in 2×YT media (Teknova #Y0166) at in the presence of 100 ug/ml ampicillin and 34 ug/ml chloramphenicol. Prior to induction, cells were grown at a temperature of 37° C. At an OD₆₀₀ 0.6, the culture was supplemented with 1 mM FSY and induced by the addition of 0.2% arabinose. At induction, cultures were moved to 25° C. while shaking at 220 RPM overnight, for a total of 16 hours.

Expressions were harvested by pelleting the cells at 2200×g for 30 minutes at 4° C. Supernatants were removed and cell pellets were weighed and stored at −80° C. Cell pellets were resuspended for lysis by adding B-PER protein extraction reagent (ThermoFisher #78243) at 4 mL/g pellet. The resuspended pellet was allowed to lyse by placing the samples on an orbital shaker at room temperature for 15 minutes at medium speed. The lysed cells were then spun down at 2200×g for 30 minutes. The soluble fraction in the supernatant was removed for further purification.

To purify the soluble fraction, HisPur Ni-NTA resin (ThermoFisher #88222) was used to capture the soluble material from lysates. Resin storage buffer was removed and equilibrated by batch washing in Wash Buffer (40 mM Sodium Phosphate, pH 7.2, 300 mM NaCl, 20 mM Imidazole). Batch washes were repeated for a total of 50× resin volume. Clarified lysate described above was added to the washed Ni-NTA resin and allowed to bind for 1 hour at room temperature, with constant rotation. After binding, protein-bound resin was batch washed at 50× the resin volume in Wash Buffer to remove unbound contamination. Protein-bound resin was then transferred to a spin column and spun briefly at 700×g to remove remaining Wash Buffer. Target protein was then eluted using a buffer with Elution Buffer (40 mM Sodium Phosphate, 7.2, 300 mM NaCl, 500 mM Imidazole). Elution Buffer was added at 2× the resin volume and allowed to incubate at room temperature for 5 minutes. The sample was spun briefly, and the eluted protein was collected in a new tube. The elution procedure was repeated twice using the same conditions. Elution fractions were pooled and quantified by A₂₈₀ on a Nanodrop 2000, blanking with Elution Buffer. The Ni-NTA purified protein was then concentrated using 0.5 mL, 3 kDa MWCO PES spin filters (ThermoFisher #88512) and buffer exchanged into 1×PBS via repeated dilution at 8-10× volumes of the sample and concentration.

### Example 3—Covalent Interaction of FSY-Modified sdAbs and PSMA

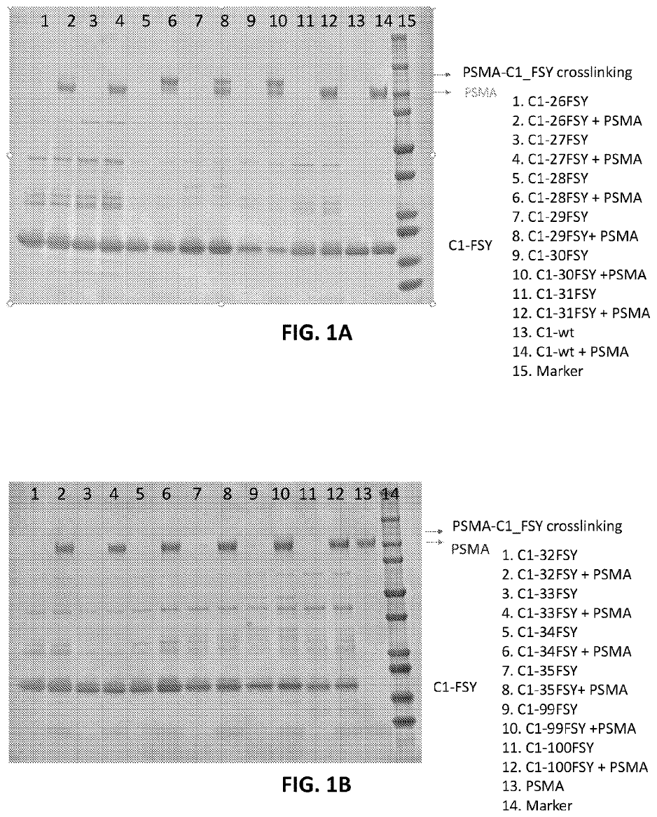

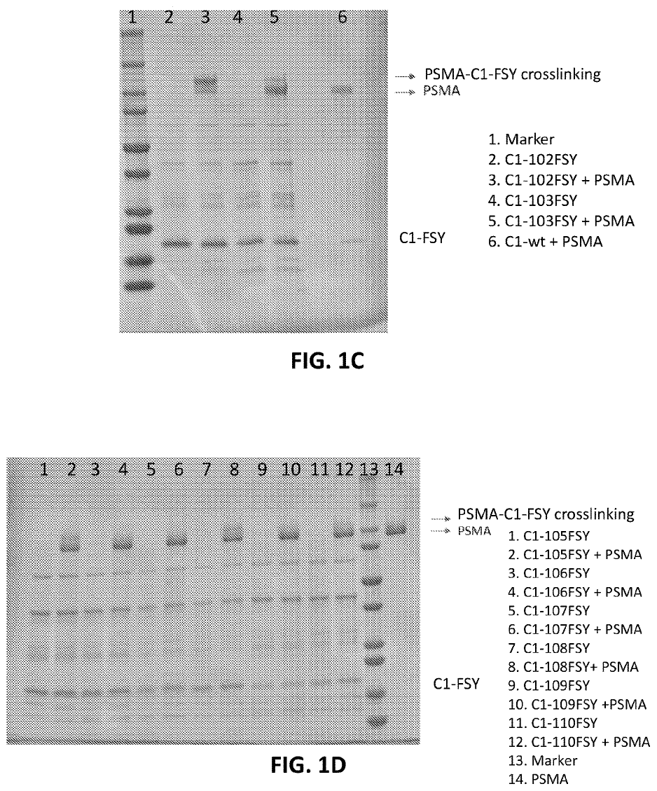

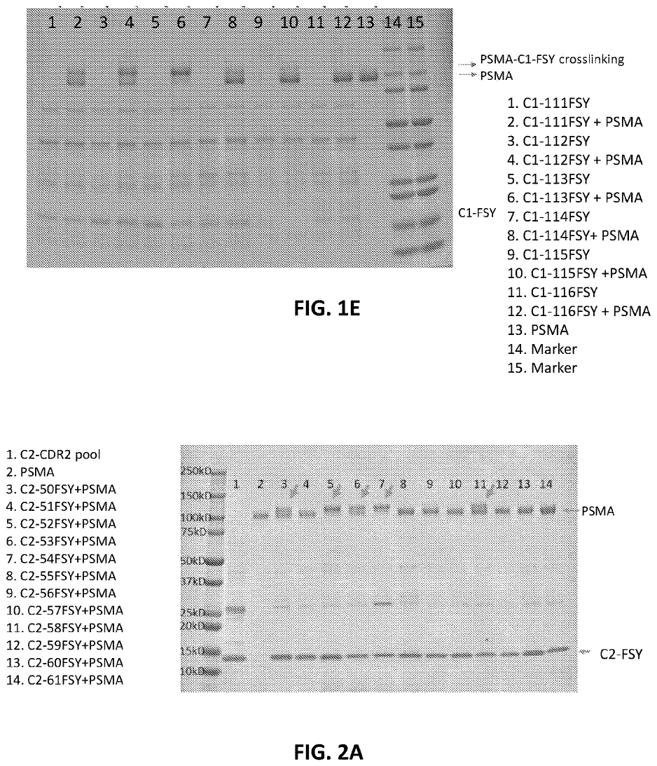

The semi-purified sdAb products from Example 2 were incubated overnight in 1×PBS with commercial PSMA (Sino Bio, cat #15877-H07H) at a molar ratio of approximately 7:1 (sdAb:PSMA, PSMA final concentration was 0.125 mg/mL, 1.25 uM) at 37° C. Crosslinking was analyzed by reducing SDS-PAGE (FIGS. 1A-1E).

Sites assessed in CDR1 and CDR3 of the C1 sdAb indicated a spectrum of cross-linking ability to PSMA as shown in Table 3 below.

TABLE 3
Crosslinking Efficiency of C1 sdAb Sites

| | Weak | Crosslinking | Crosslinking |
|---|---|---|---|
| | Crosslinking | site | site |
| CDR Region | (<50%) | (>50%) | (80-90%) |
| CDR1 | 26 | 28, 29, 30 | 28 |
| CDR3 | 99, 103, 105, 108, | 102, 112, 113 | 102, 112, 113 |
| | 111, 110, 114, 115 | | |

Site positions are listed relative to SEQ ID NO: 1.

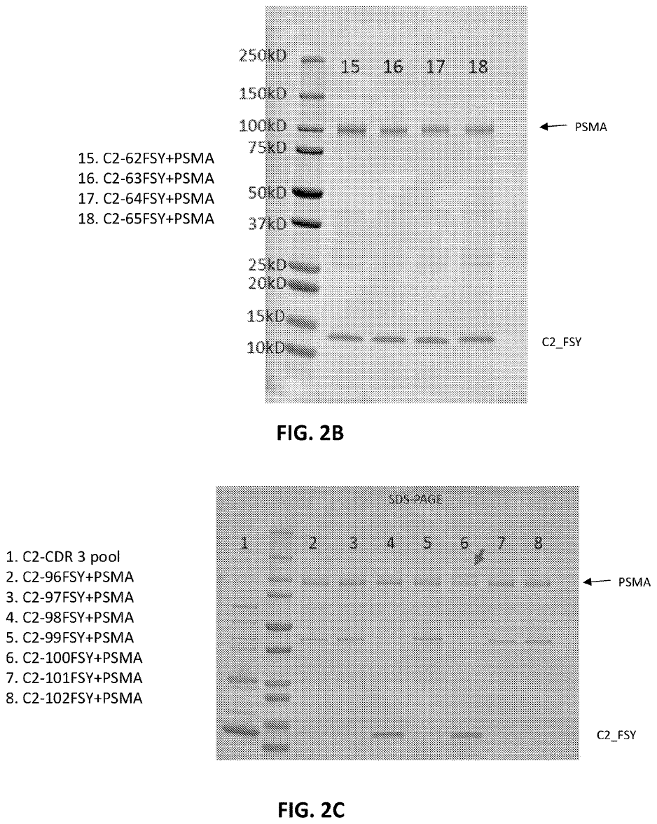

A second PSMA-specific sdAb C2, was assessed for the efficiency of crosslinking using methods similar to the methods of Example 1 with the modification that the sdAb:PSMA molar ratio was approximately 8:1. The results of the crosslinking are shown in FIGS. 2A-2C. Sites assessed in CDR2 and CDR3 of the C2 sdAb indicated a spectrum of crosslinking ability to PSMA as shown in Table 4 below.

TABLE 4
Crosslinking Efficiency of C2 sdAb Sites

| | Weak | Crosslinking | Crosslinking |
|---|---|---|---|
| | Crosslinking | site | site |
| CDR Region | (<50%) | (>50%) | (80-90%) |
| CDR2 | 56 | 50, 53, 58 | 52, 54 |
| CDR3 | 100 | 0 | 0 |

Site positions are listed relative to SEQ ID NO: 2.

### Example 4—Kinetics of Cross-Linking

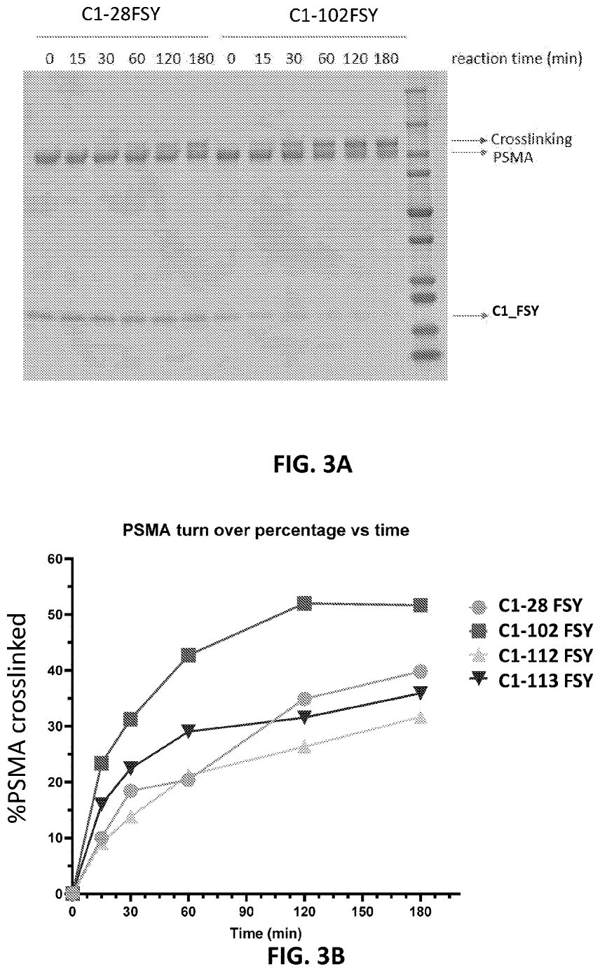

A subset of FSY-modified sdAbs were selected to assess for kinetics of PSMA crosslinking. The sdAb was incubated with PSMA at a 5:1 molar ratio (PSMA final concentration was 0.125 mg/mL, 1.25 uM). Samples were taken at time points from 0-180 minutes and the percentage of PSMA cross-linked assessed by SDS-PAGE. As shown in FIGS. 3A and 3B, the modified sdAbs exhibited a range of coupling kinetics. The crosslinking band percentage was calculated by quantifying the sdAb-PSMA crosslinking band and the PSMA band intensity was quantified using ImageJ.

### Example 5—PSMA Targeting Construct with Modified CDR3

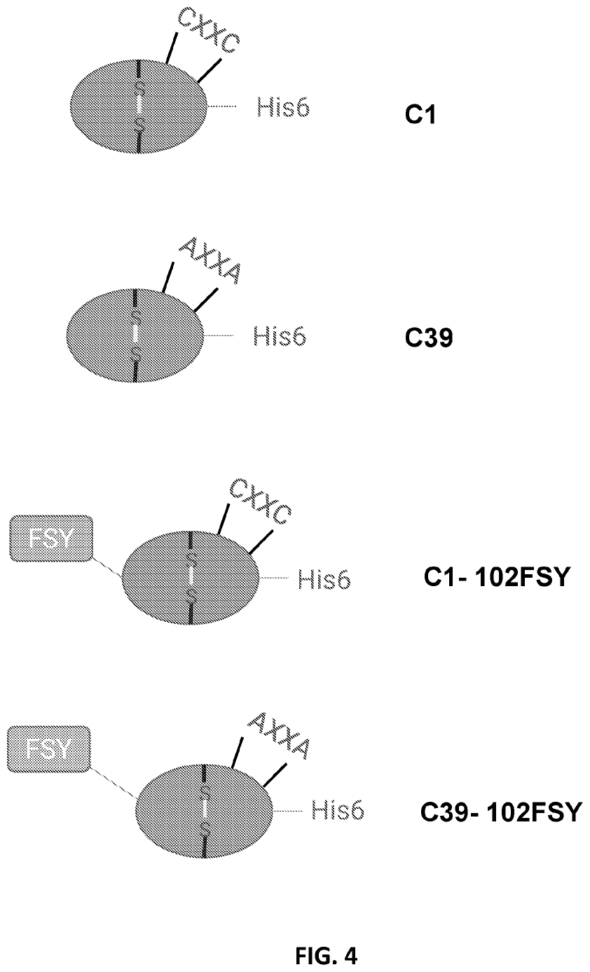

A PSMA-targeted sdAb C1 construct was generated removing two cysteine residues in CDR3 (SEQ ID NO: 7) of C1 (SEQ ID NO: 1) by modifying these residues to alanine (C101A and C104A) to create C39. This construct was then further modified to install FSY at residue 102 (C1-C101A/C104A/H102(FSY), SEQ ID NO: 4, hereafter referred to as C39-102FSY). A diagram of these sdAb constructs is shown in FIG. 4. The constructs were synthesized with a pelB leader sequence (SEQ ID NO: 15) that was cleaved off from the mature protein, and six C-terminal histidines were used for His-Tag purification. The crosslinking capability of these constructs was assessed using the method of Example 3. SDS-PAGE analysis indicated that all the constructs cross-linked at a comparable level under these conditions.

The FSY containing PSMA-targeted sdAb C39-FSY C1 was cloned and expressed as described in Example 2. A biparatopic construct (C1-C101A/C104A/H102(FSY)-L1-C1-C39, SEQ ID NO: 21, hereafter referred to as C40-FSY) was created by linking the C39-FSY C1 with an additional copy of the C1-C101A/C104A/H102H (SEQ ID NO: 71, without FSY). The construct was designed by adding a GGGGSGGGGS linker between the two sdAb amino acid sequences. The biparatopic construct was then cloned into pBAD vector and expressed as described in Example 2. The constructs were synthesized with a pelB leader sequence (SEQ ID NO: 15) that was cleaved off from the mature protein, and six C-terminal histidines were used for His-Tag purification.

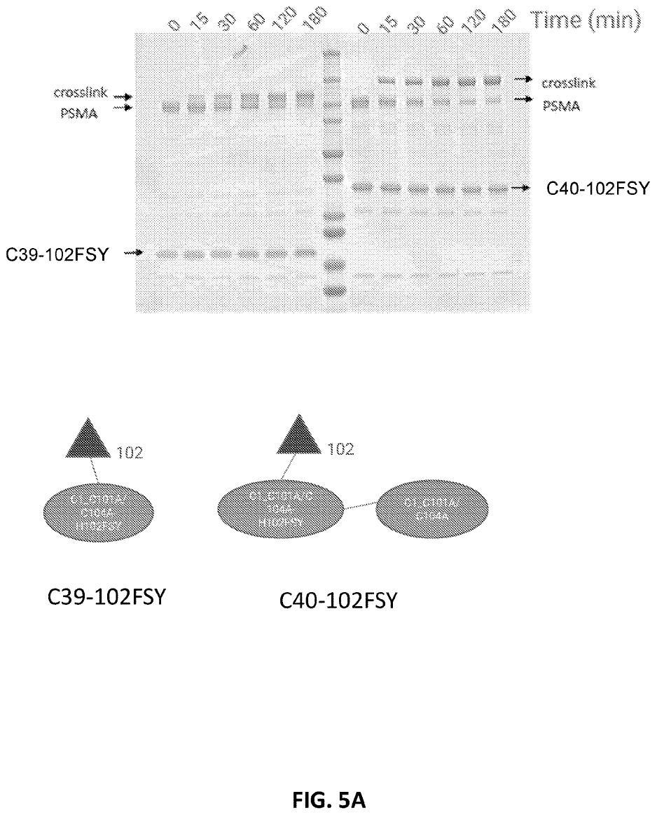

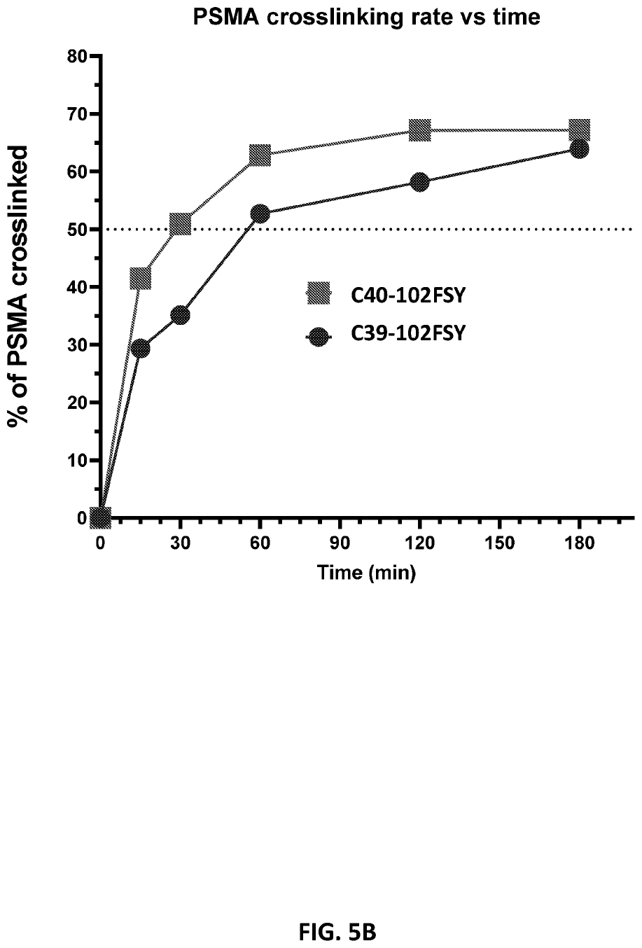

The monoparatopic sdAb and biparatopic constructs were assessed for cross-linking to PSMA by SDS-PAGE and over a time course. SDS-PAGE analysis indicated that the dimeric form C40-102FSY crosslinks two times faster than the monomeric C39-102FSY (t½ maximum crosslinking for dimer ~30 min vs for monomer ~60 min). (FIGS. 5A-5B)

C39-102FSY amino acid sequence (* is 102 FSY position, SEQ ID NO:4, alanine modifications are underlined):

QVQLQESGGGSVQAGGSLRLSCTAPGYTDSNYYMSWFRQAPGKEREWVAG
VNTGRGSTSYADSVKGRFTISQDNAKNTMFLQMNSLKPEDTAIYYCAVAA
A*FADSLPKTQDEYILWGQGTQVTVSSAAAYPYDVPDYGSCHHHHHH

### Example 6—Synthesis of Constructs

Site specific conjugation via an engineered C-terminal Cysteine residue. To enable site-specific bioconjugation to the sdAb compounds, an additional Cys residue was engineered near the C-terminus of the constructs. The sdAb-Cys compounds were prepared and formulated to 1 mg/ml in 1×PBS, pH 7.4, 5 mM EDTA. TCEP was added to 10 mM for mild reduction and incubated at room temperature for 15 minutes. The sample was then buffer exchanged using a Zeba Spin column to remove TCEP and exchange into 1×PBS, pH 7.4, 1 mM EDTA. A maleimide-reactive payload compound (such as AZDye 647 Maleimide cat #: 1122-5) was then added at 10× molar excess and incubated at 37° C. for 2 hr, or 4° C. for 16 hours. Un-reacted maleimide compound was removed from the sample using size exclusion chromatography, desalting column, dialysis or TFF. The sample was subjected to SDS-PAGE under reducing and non-reducing conditions to observe the labeling efficiency and analyzed using LC-MS to quantitate the intact mass and relative proportions of the labeled and un-labeled species.

### Example 7—Assay for Crosslinking Activity

Molecules are assayed for specific binding and crosslinking to the targeted tumor antigen. To assess cell binding potency of PSMA-targeting constructs, the specific FSY- or Tyr-containing sdAb is used to test the effect of covalent crosslinking to the specific antigen of interest. These test articles are diluted serially and associated with prostate cancer cell lines LnCAP (PSMA+) and PC3 (PSMA−) cultured in vitro. After incubation at 37° C. for different times, cells are washed and media is replaced to remove unbound material. The samples are analyzed by flow cytometry using anti-His tag antibodies or anti-VHH to measure the bound population as a function of input concentration. To measure the crosslinking efficiency and specificity, cells are collected, lysed, and western blotted using anti-his or anti-VHH antibodies to measure the covalently bound population via gel shift, and are compared with gel shift western blots using anti-PSMA antibodies to measure the total target antigen that is crosslinked to the sdAb test article.

Conjugate molecules are additionally assayed for functional activity, in this case the ability to deliver a toxic payload in vitro specifically to tumor cells that express the target antigen. Constructs containing an engineered C-terminal or proximal Cys residue are coupled to a cytotoxic payload such as MMAE or other class of highly-cytotoxic compound using the general methods described in Example 8. Dose-response curves are generated and samples are incubated with LnCAP and PC3 cells in vitro. After incubation for various periods of time, cells are washed to remove free compound, and cell viability is measured using assays such as Promega CytoxGreen, or other assays that measure cell viability via reporter compound detection, detection of viable cells by Presto Blue, CCK-8 or similar reagents, and/or measuring apoptosis via Annexin V-FITC, propidium iodide staining, or similar reagents.

### Example 8—In-Vivo Delivery

To measure the capacity of constructs to specifically deliver payload to tumor tissues in vivo, and to compare the tumor exposure of constructs versus non-covalent sdAb conjugates, a fluorophore label is used as a proxy payload to enable tracking/biodistribution measurements over time. An sdAb construct (e.g., C1, C2, or C3) with a single residue substituted to either Tyr or FSY is conjugated via engineered cysteine residues to a chemical fluorophore (Alexa647 or similar) via maleimide chemistry and purified as described in Example 6. The fluorophore-labeled sdADC conjugate molecules is administered via tail vein IV injection in male NSG mice bearing PSMA+ or − tumors, and the biodistribution of the test articles is observed over time via whole-animal imaging using an AmiX imager or similar. The tumor-specific and peripheral exposures is quantitated via image densitometry and compared between FSY and Tyr versions of the sdAb.

### Example 9: Design of Additional FSY-Modified SdAb

The C3 wt anti-PSMA sdAb was constructed by cloning C3 wt sequence (synthesized from IDT, SEQ ID NO:3) into a digested vector (synthesized by Genscript) by NdeI and HindIII restriction enzyme. The expression cassette also incorporated the PelB leader sequence and His6 tag. The C3 wt DNA sequence is shown as SEQ ID NO:75.

The amino acid sequence in the CDR region was changed to TAG codon in the plasmid at the sites selected for FSY modification. These TAG mutants in the ORF were prepared by Genscript via site-directed mutagenesis.

C3 CDR1 (26-35: GWPYSTYSMN, SEQ ID NO: 11);
CDR2 (50-65: GISSTMSGIIFAESKAG, SEQ ID NO: 12);
CDR3 (99-113: RRDYSLSSSSDDFDY, SEQ ID NO: 13).

### Example 10—Screening of FSY-Modified sdAbs

The C3 constructs from Example 9 were co-transformed with pEVOL-FSYRS into DH10b competent cells as described in Example 2. Single colonies from the transformation were picked and inoculated into 1 mL 2×YT in 24 deep well plate supplemented with 100 ug/mL Amp+34 ug/mL Cm shaking at 220 rpm, 37° C. When the OD reached 0.5, the temperature was reduced to 25° C. The expression for each well was induced by adding 0.2% arabinose+1 mM FSY. The products were expressed at 25° C. for overnight. Following the overnight, the cells were transferred into a 1.5 mL Eppendorf tube and spun by a benchtop centrifuge. The supernatant was removed. The pellet was treated with 50 uL B-per (ThermoFisher #78243) for cell lysis for 15 min. After that, the cell lysate was spun down with maximum speed by a 4° C. benchtop centrifuge.

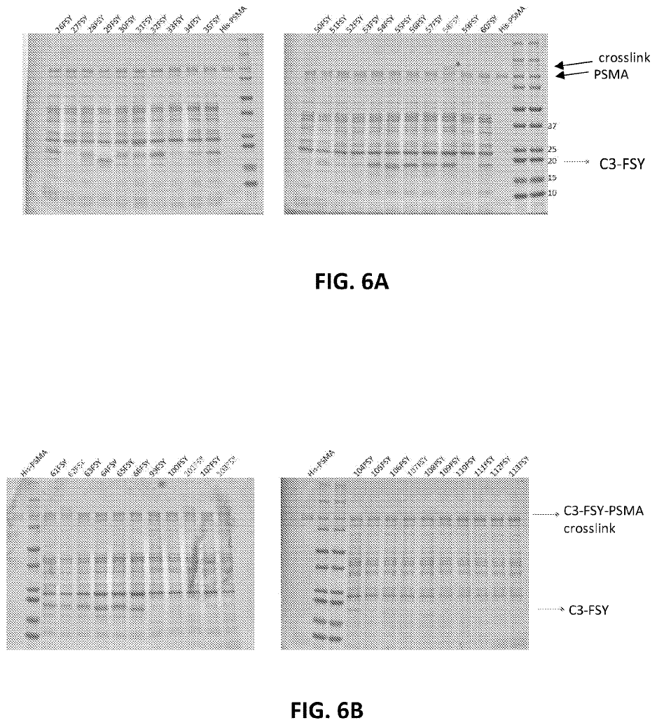

Crosslinking reaction was initiated by incubating 3 uL supernatant with 3 uL PBS or 3 uL 0.25 mg/mL PSMA. The reaction was incubated at 37° C. for a shorter time window 3 hr. After that, the incubation mixture was treated with 2XSDS-loading dye. The crosslinking was investigated by running SDS-PAGE staining with Coomassie blue. Results are shown in FIGS. 6A and 6B and summarized below in Table 5.

TABLE 5
Crosslinking Efficiency of C3 sdAb with PSMA within 3 hr

| | Weak | Crosslinking | Crosslinking |
|---|---|---|---|
| | Crosslinking | site | site |
| CDR Region | (<50%) | (>50%) | (80-90%) |
| CDR1 | 0 | 0 | 0 |
| CDR3 | 0 | 58, 62 | 0 |
| CDR3 | 101, 103, 107 | 0 | 0 |

Site positions are listed relative to SEQ ID NO: 3.

### Example 11—Identification and Kinetics of FSK-Modified FAP sdAbs

Sequence analysis (abYsis) was used to define complementarity determining region (CDR) loop positions in the Fap antibody sequence. Libraries were constructed to individually replace each CDR residue with a TAG codon and the resulting proteins were expressed under conditions to incorporate the unnatural amino acid FSK at each position.

C8 FAP library generation. To assess different FSK incorporation sites in the sdAb, a vector pBAD-C8 WT (SEQ ID NO: 82) was constructed as follows: The C8 WT sequence (SEQ ID NO: 23) was codon optimized for E. coli expression and synthesized by IDT company. The pBAD-C8 WT vector was constructed by cloning C8 WT gblock sequence (SEQ ID: NO 78) into a digested pBAD vector (Genscript) by NdeI and HindIII restriction enzyme.

Library strain generation. For expression of the corresponding synthetase, the pEVOL-FSKRS plasmid (Genscript) was synthesized, which encodes the engineered Ma FSKRS aminoacyl tRNA synthetase (SEQ ID NO: 87) pEVOL-FSKRS (SEQ ID NO: 89) was transformed into chemically competent DH10b competent cells to generate the parental strain FSKRS-DH10b. Individual plasmids encoding each C8 CDR TAG variant were transformed into chemically competent FSKRS-DH10b cells. Single colonies were inoculated into Superbroth media (Teknova #S1530) containing 100 ug/ml ampicillin and 34 ug/ml chloramphenicol and grown overnight shaking at 37° C. approximately 220 rpm.

Expression and purification. Strains bearing the plasmids encoding sdAbs with individual CDR residues mutated to TAG in the FSKRS-DH10b parental background were cultured in Superbroth media in the presence of 100 ug/ml ampicillin and 34 ug/ml chloramphenicol. A single colony from each pool was inoculated into 5 mL Superbroth media with relevant antibiotics and grown overnight at 37° C. The next day, cells were diluted at 1:10 ratio into 30 mL (3 mL to 30 mL dilution) Superbroth media, with 100 ug/ml ampicillin and 34 ug/ml chloramphenicol, 1 mM FSK, 0.2% arabinose. The cells were induced for FSK incorporation for 6 hr at 30° C. at 220 rpm. Following expression, FSK-modified sdAbs (C8-FSK) were harvested and purified following the methods described in Example 2.

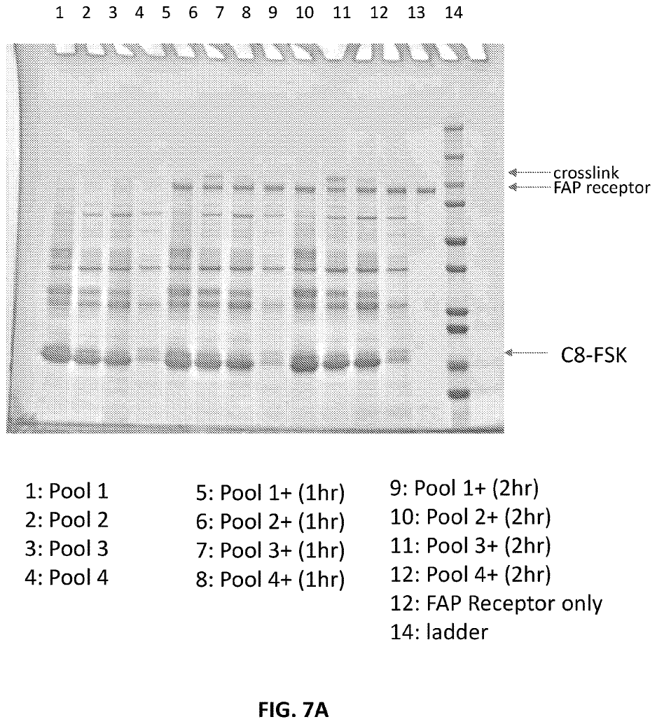

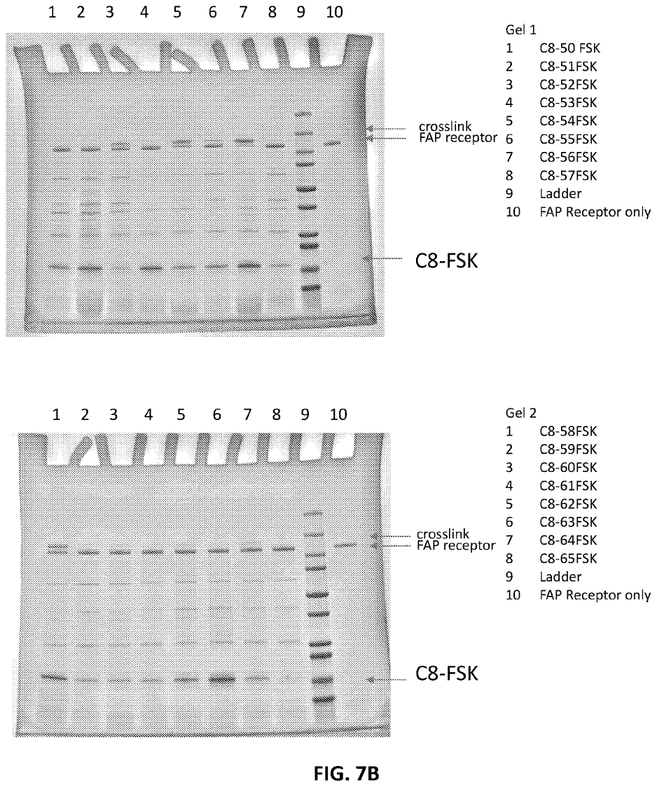

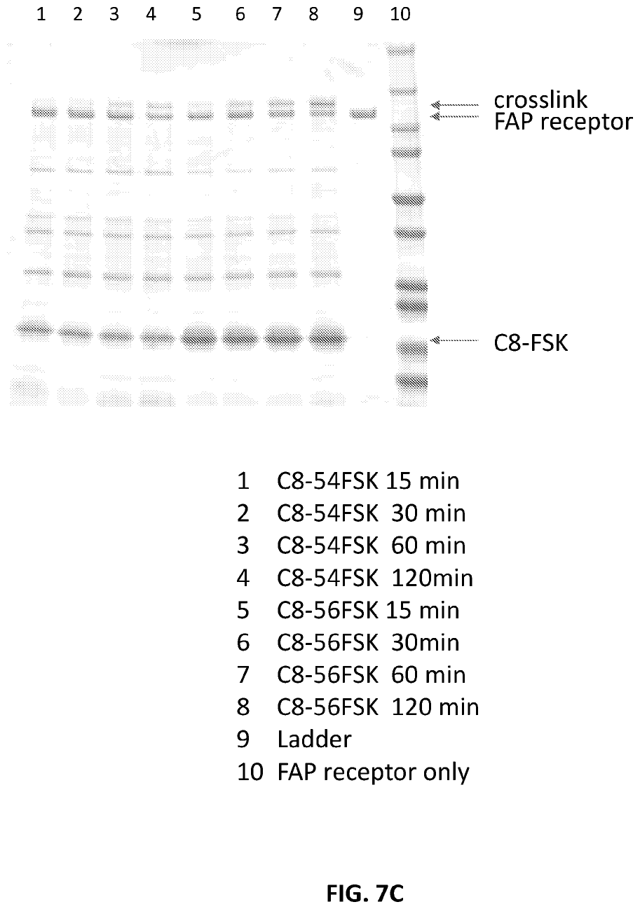

Crosslinking kinetics. FAP receptor (Acrobiosystem #AP-H5263-100 ug) was incubated with the pooled antibody mixture (Table 6) at a molar ratio ~8:1 (antibody:receptor) at 37° C., 1×PBS, pH 7.4 for 1 hr, 2 hr, or overnight. After incubation, the crosslinking reaction was stopped by adding 1× Laemmli Sample Buffer containing 100 mM DTT. The samples were heated at 95° C. for 5 mins, followed by running Tris-glcyine SDS-PAGE (4-20% Mini-PROTEAN® TGX™). The relative band intensity was subsequently quantified using ImageJ software. The results of the FSK pooled shifts demonstrated FAP receptor shifts via crosslinking activity within pools 2 and 3 (FIG. 7A).

TABLE 6
Designed CDR pool for expression C8 FSK mixture

| Pool number | CDR region |
|---|---|
| 1 | CDR1 (26-35) |
| 2 | CDR2 (50-57) |
| 3 | CDR2 (58-65) |
| 4 | CDR3 (98-106) |

Identification of the single FSK sites responsible for crosslinking with FAP. To identify the individual FSK sites responsible for crosslinking, each TAG mutant was co-transformed with pEVOL-FSKRS into DH10b cells, expressed and purified as described above. Each individual FSK mutant from the candidate pool was incubated with FAP receptor for investigation of gel shift (FIG. 7B). Nine FSK mutants in the CDR2 were found to have crosslinking with FAP (52, 53, 54, 55, 56, 58, 60, 62, 64). One site (56) was found to have the highest crosslinking yield among all the crosslinking sites within 1 hr.

Measurement of C8-FSK crosslinking kinetics to FAP receptor. To evaluate the FSK crosslinking kinetics, the C8-54FSK and C8-56FSK were incubated with FAP receptor for 15, 30, 60, and 120 minutes and examined for crosslinking yield. The 56 FSK site was found to have the highest crosslinking rate compared to the other sites, indicating that C8-56FSK has the fastest crosslinking rate (>50% crosslinking within 2 hr) (FIG. 7C).

### Example 12—Identification and Kinetics of FSY-Modified Her3 SdAbs

C9 library generation. Sequence analysis was used (abYsis) to define complementarity determining region (CDR) loop positions in the Her-3 antibody sequence. Libraries were constructed to individually replace each CDR residue with the TAG codon and expressed under conditions to incorporate the unnatural amino acid FSY at each position.

Vector pBAD-C9 WT was constructed. The C9 WT sequence (SEQ ID NO:24) was codon optimized for E. coli expression. The pBAD-C9 WT vector (SEQ ID NO:83) was constructed by cloning C9 gblock sequence (SEQ ID NO:79) into a digested pBAD vector (Genescript cat #SC1010) by NdeI and HindIII restriction enzyme. The library of C9-FSY variants was created as described in Example 2, except pEVOL-FSYRS plasmid and individual mutant pBAD-C9 mutants were transformed and expressed in BL21 cells rather than DH10b cells. Expression of FSY-Modified sdAbs (C9-FSY) followed the methods described in Example 2 with the exception that FSY-modified C9 sdAbs were expressed in and purified from parental strain FSYRS-BL21.

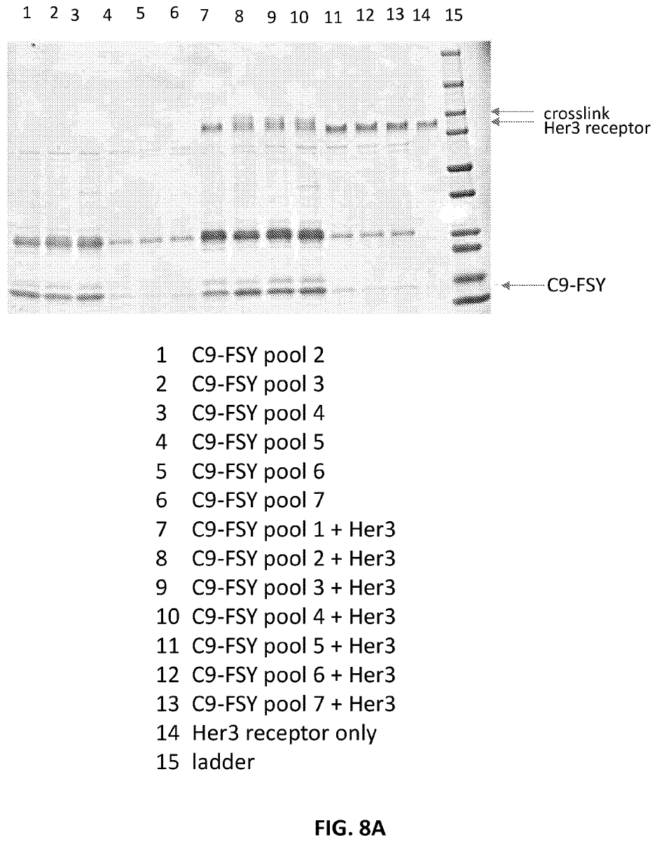

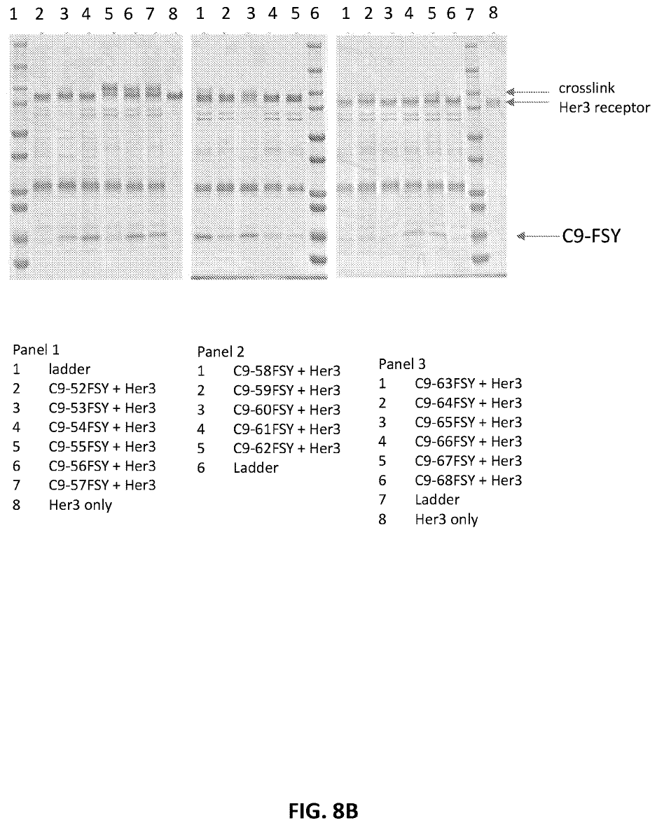

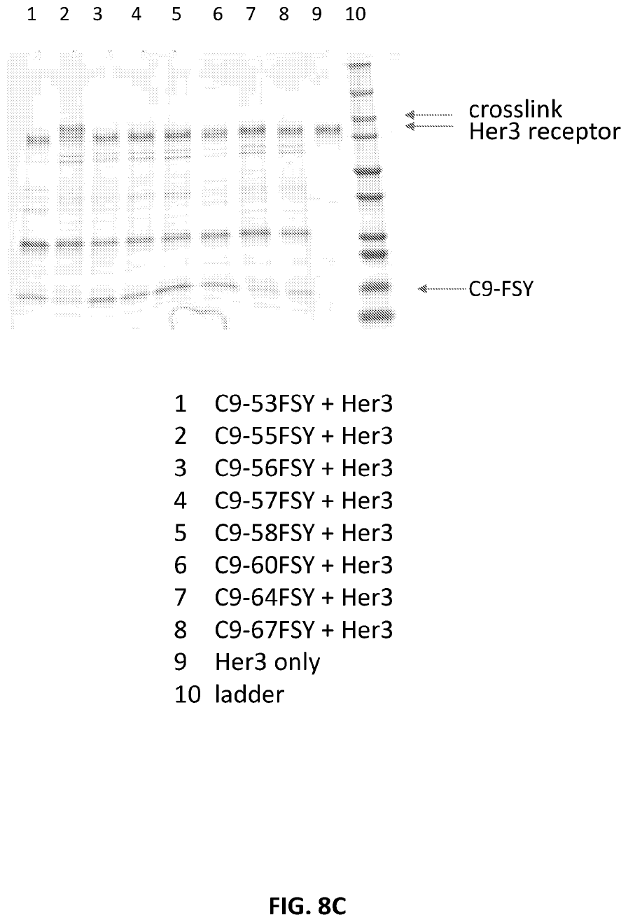

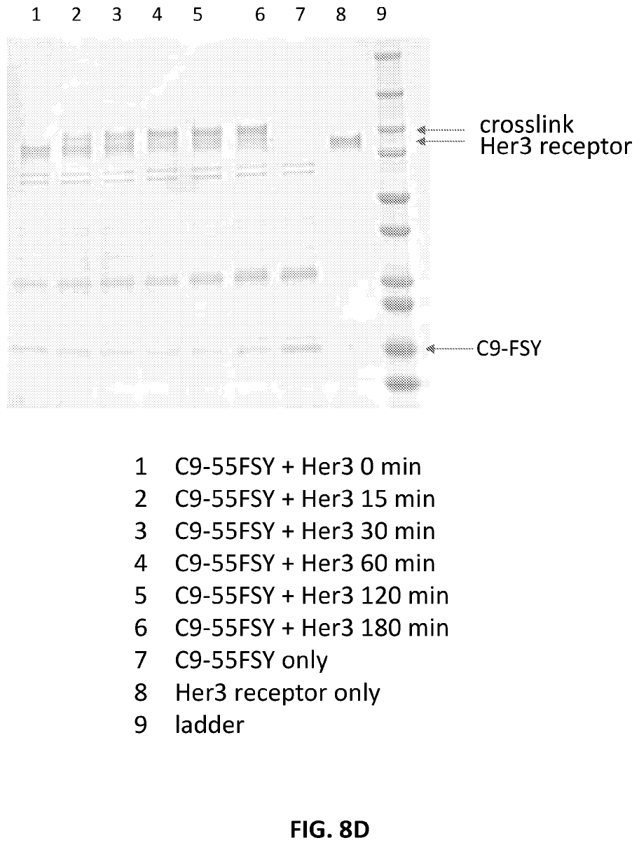

Identification of the pool responsible for crosslinking. To initially identify regions of CDRs that contain crosslinking compatible FSY sites, strains containing plasmids with TAG sites in multiple CDR regions were initially pooled (Table 7). Pooled strains were expressed and purified via Ni-NTA affinity, normalized for concentration, and assessed for crosslinking via SDS-PAGE gel shifts as described in Examples 2. C9-FSY pool mixture was incubated with Her3 (Acrobiosystem #ER3-H5223-100 ug) at a 3:1 ratio (3 ug C9 FSY to 1 ug Her3 receptor) at 37° C., 1×PBS, pH 7.4 overnight. The reactions were run on 4-20% Mini-PROTEAN® TGX™ and then stained with Coomassie blue. The results of the pooled shifts demonstrated Her3 receptor crosslinking activity within pools 2, 3, and 4 (FIG. 8A).

TABLE 7
Designed CDR pool for expression C9 FSY mixture

| Pool number | CDR region |
|---|---|
| 1 | CDR1 (28-37) |
| 2 | CDR2 (52-58) |
| 3 | CDR2 (59-63) |
| 4 | CDR2 (64-68) |
| 5 | CDR3 (101-106) |
| 6 | CDR3 (107-112) |
| 7 | CDR3 (113-118) |

Identification of the single FSY sites responsible for crosslinking with Her3. To identify each individual FSY site responsible for crosslinking, each TAG mutant was co-transformed with pEVOL-FSYRS into BL21 cells, expressed and purified as described above. Each individual FSY mutant from the candidate pool was incubated with Her3 receptor at 37° C. overnight. The crosslinking was assessed by running the reactions on 4-20% Mini-PROTEAN® TGX™ with Coomassie blue staining. (FIG. 8B). Eight C9-FSY mutants in the CDR2 were found to support crosslinking with Her3 (53, 55, 56, 57, 58, 60, 64, 67). Two sites (55, 57) were found to have the highest crosslinking yield among all the crosslinking sites.

Identification of C9-FSY crosslinking site with fastest rate. To identify the FSY crosslinking site with the fastest rate, C9 with FSY sites identified above were incubated with the Her3 receptor (Acrobiosystem) and crosslinking yield was determined. C9-FSY mutants were incubated with Her3 receptor at a molar ratio of 8:1, in 1×PBS, pH 7.4. The incubation was stopped by adding to a final 1×SDS-loading dye containing 100 mM DTT after 1 hr and the reactions analysed by 4-20% Mini-PROTEAN® TGX™ with Coomassie blue staining.

The unique 55 FSY site was found to have the highest crosslinking yield (>50% within 1 hr) compared to the other sites, indicating that 55 FSY has the fastest crosslinking rate (FIG. 8C). The C9-55FSY was further analysed by incubating it with Her3 receptor and stopping the reaction over a time course of 0, 15, 30, 60, 120, and 180 minutes. As shown in FIG. 8D, the crosslinking proceeded rapidly and was detected within 15 minutes, with more than 50% crosslinking within 1 hr.

### Example 13—Binding Affinity of FSY-Modified and Unmodified C2 sdAb

The C2 sdAb with FSY at position 54 (C2-54FSY) was selected to compare binding affinity with the C2 sdAb with tyrosine at the same position (C2-54TYR). Proteins were prepared as in Example 2, and then further purified via FPLC size exclusion chromatography (HiLoad 16/600 Superdex 200 pg size exclusion column Cytiva #28989335). 1×DPBS was used as the running buffer and the protein was collected by isocratic elution. For each C2 sdAb, the monomer peak fractions were analysed by reducing SDS-PAGE and were pooled and dialyzed into anion exchange running buffer (20 mM Tris, 7.5 and 20 mM NaCl), overnight at 4° C. To remove endotoxin, the dialyzed sample pool was run over a HiTrap Q XL, 1 ml column (Cytiva #17515801) in flow through mode with the endotoxin binding to the column. The flowthrough was collected and checked for endotoxin levels. Finally, the fully purified sample was dialyzed into 1×DPBS as the final formulation buffer. Samples were aliquoted and stored at −80 C.

Binding kinetics were measured using biolayer interferometry with AHC sensor (Sartorius Item #18-5060). PSMA (50 nM) with a Fe tag (Acrobiosystem PSA-H5264-100 ug) with either C2-54FSY or C2-54TYR sdAb (protein concentrations 400 nM, 200 nM, 100 nM, 50 nM). The steps for OCTET measured were carried out as baseline: 60 s; loading receptor: 300 s, washout non-binding receptor: 300 s; loading sdAb for association: 100 s; dissociation: 600 s. C2-54TYR had a KD of 9.1 nM and C2-54FSY had a KD of 10.9 nM, indicating that incorporation of FSY in place of tyrosine at position 54 did not change the binding affinity.

### Example 14—Cell Binding Assay

The C2-54FSY and C2-54TYR sdAbs were assessed for binding to human prostate tumor cell lines LNCaP (PSMA+) and PC3 (PSMA−) using flow cytometry. Proteins from Example 13 were formulated with FACS buffer (1×PBS+2% HI-FBS) to 3 μM (1000 μL) and then serially diluted 5× (200 μL sdAb to 800 μL FACS buffer) to generate 8 concentration points for the assay, with the lowest concentration at 0.0000384 μM. Control sdAb (Human PSMA Alexa Fluor Alexa Fluor® 647-conjugated antibody) was formulated with FACS buffer (1×PBS+2% HI-FBS) to 1 μM (450 μL) and then serially diluted 3× (150 μL test article to 300 μL FACS buffer) with the lowest concentration was 0.00045 μM.

Binding assay. LNCaP and PC3 cell lines (ATCC) were maintained in RPMI-1640 and F12K media supplemented with 10% heat inactivated fetal bovine serum (Thermo Fisher) in a humidified environment with 5% C02 at 37° C. The cells were harvested at exponential growth phase, counted and aliquoted to v-bottom 96-well plate with 100 μL cells/well. Cells were pelleted by centrifugation. C2 sdAbs and control sdAb samples were added to cells and incubated on ice for one hour. For cells treated with C2 sdAbs, after incubation, cells were washed with FACS buffer twice, and incubated with 100 μL of ice-cold FACS buffer containing secondary antibody at 5 μg/mL (Alexa Fluor 647 AffiniPure Goat Anti-Alpaca IgG, VHH domain) on ice for 30 minutes. Cells were then washed with FACS buffer twice and fixed with 4° C. fixation buffer (Thermofisher, Cat. No. 0082249) for 30 minutes. For cells treated with control sdAb, after incubation, cells were washed with FACS buffer twice, and fixed with 4° C. fixation buffer (Thermofisher, Cat. No. 0082249) for 30 minutes. After fixation, cells were washed twice and analyzed with Attune flow cytometry (ThermoFisher).

Raw data (FCS files) were analyzed (Flowjo 10.7.2). FSC-A and SSC-A were used to separate total cells and intact cells were gated with FSC-A and SSC-A. Then singlets were gated out with FSC-A against FSC-H. Singlets were separated with Alexa Fluror-647. Geometric mean intensity of Alexa Fluror-647 signal intensity of samples were used for further analysis and plotting. Mean value and STDEV value of Geometric Mean Intensity of Alexa Fluror-647 of samples were calculated (Microsoft® Excel® for Microsoft 365 MSO Version 2202 Build 16.0.14931.20128 64-bit). Curve fit and plotting were performed with ‘log(agonist) vs. response—variable slope (four parameters)’ (GraphPad Prism 9.2.0).

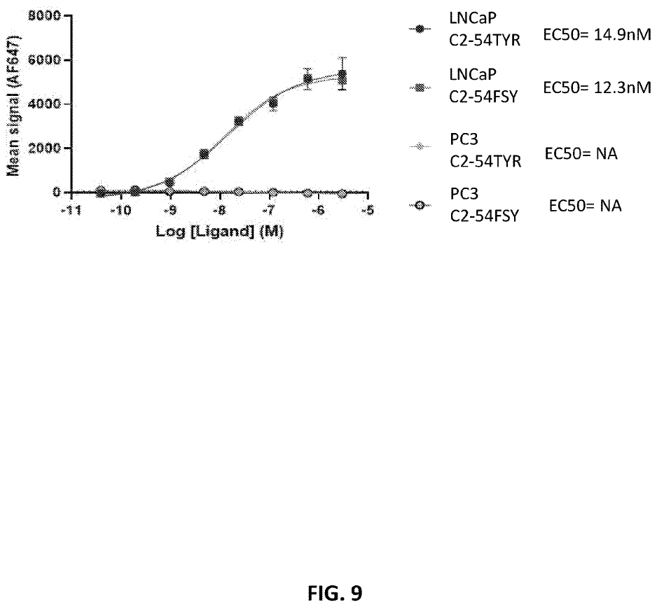

C2-54TYR and C2-54FSY bound to LNCaP cells in a dose dependent manner but did not exhibit binding to the PSMA negative PC3 cells (FIG. 9). Binding affinities for C2-54TYR and C2-54FSY were measured from EC50 values after flow staining of LNCaP cells (Table 8).

TABLE 8
Binding affinity of C2 sdAbs in LNCaP cells

| | C2-54TYR | C2-54FSY |
|---|---|---|
| EC50 | 14.88 nM | 12.25 nM |
| EC50 Range | 8.83 nM to 27.05 nM | 8.98 nM to 16.88 nM |
| R squared | 0.978 | 0.99 |

### Example 15—Crosslinking of FSY-Modified sdAbs to Target Cells

Crosslinking of C2-54TYR, C2-54FSY and C3-101FSY were compared for cell binding. LNCaP and PC3 cells were cultured as described in Example 14. The cells were plated in 6-well plates in their growth media at the density of 500,000 cells/well. At 48 hours after seeding, the culture medium was removed and 0.8 mL culture medium with the sdAbs was added at indicated concentrations and time points (see Tables 9 and 10). Cells were rinsed twice (1 mL/well 1×PBS) and 0.2 mL/well 0.25% EDTA-Trypsin (Gibco, Cat. No. 25200-056) was added. After five minutes incubation in the incubator, 1 ml complete medium were added to neutralize the trypsin. Cells were rinsed and pelleted.

Cells were lysed in RIPA buffer supplemented with protease inhibitor cocktail (Santa Cruz Biotechnology, Cat. No. SC-24948A). The denatured samples were analyzed by electrophoresis in 4-20% Criterion TGX Gel (Bio-Rad, Cat. No. 5671095) followed by western blot with kit (Bio-Rad, Cat. No. 1704271) and Pierce Fast Western Blot Kits (Thermo Scientific, Cat. No. 35060, 35061) and using the primary antibody anti-human PSMA (Invitrogen, Cat. No. 37-3900) or anti-alpaca VHH (Jackson ImmunoResearch, Cat. No. 128-035-232), the internal standard anti-GAPDH (Cell Signaling Technology, Cat. No. CST-2118).

Images were acquired by Azure Biosystem C600. GIMP 2.10.28 was used to process images. Density of bands in western blot was determined with ImageJ 1.51j8 following the instructions (imagej.nih.gov/ij/docs/guide/146-30.html #infobox:Densitometry, Section 30.13). Percentages of PSMA crosslinked versus total PSMA were calculated with following formula: Density of crosslinked PSMA/(density of crosslinked PSMA+density of uncrosslinked PSMA)×100%.

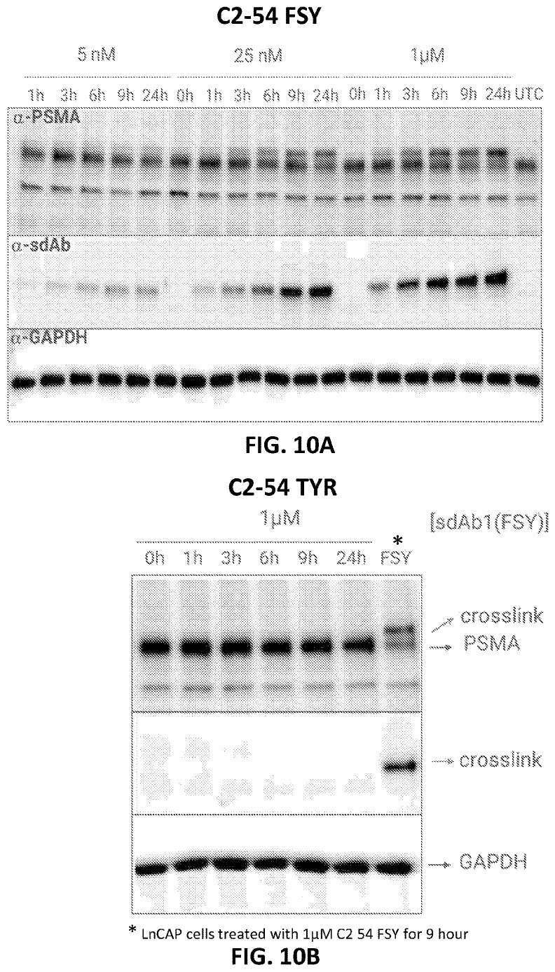

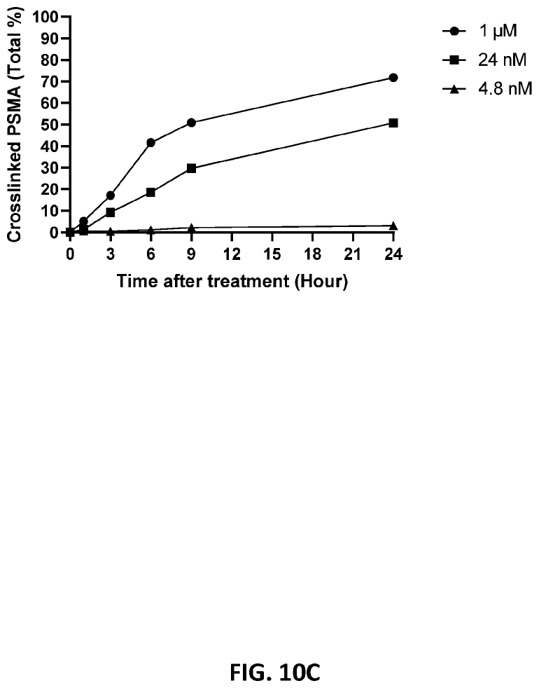

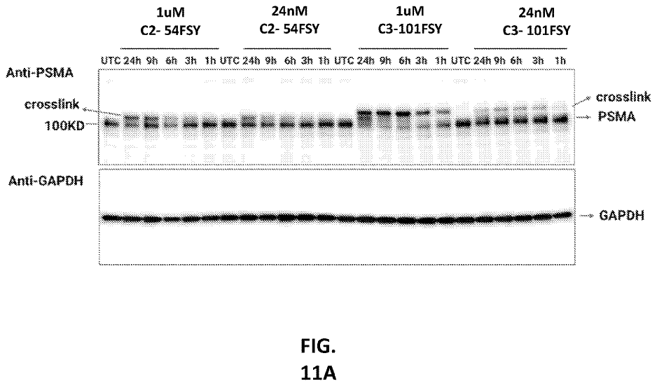

A predominant band detected with anti-PSMA antibody (~100 kD) was observed in the western blot from LNCaP cells (FIG. 10A), but no band with similar molecular weight was detected in PC3 cells (FIG. 10B), which demonstrated the specificity of the anti-PSMA antibody and confirmed the specificity of PSMA expression in LNCaP cells. In LNCaP cells treated with C2-54FSY, an additional band above PSMA was detected. The intensity of the band increased in a time and concentration dependent manner, indicating specific crosslinking. However, in LNCaP cells treated with various concentrations of C2-54TYR for various times, similar intensity of non-crosslinked PSMA band but not the crosslinked band was observed, showing that the sdAb without FSY did not crosslink to PSMA in the LNCaP cells (FIG. 10B). Quantitation of the crosslinking indicated that C2-54TYR crosslinked to PSMA in a time and dose dependent manner in LNCaP cells. (Table 9 and FIG. 10C)

TABLE 9
Crosslinking kinetics of C2-54FSY with PSMA in LNCaP cells

Concentration

| | | | |
|---|---|---|---|
| Time after Treatment | 1 μM | 24 nM | 4.8 nM |

| | |
|---|---|
| (hour) | Crosslinked PSMA (Total PSMA %) |

| | | | |
|---|---|---|---|
| 0 | 0.0 | 0.0 | 0.0 |
| 1 | 5.1 | 1.4 | 0.5 |
| 3 | 17.1 | 9.2 | 0.5 |
| 6 | 41.7 | 18.5 | 1.2 |
| 9 | 50.9 | 29.7 | 2.2 |
| 24 | 71.8 | 50.8 | 3.1 |

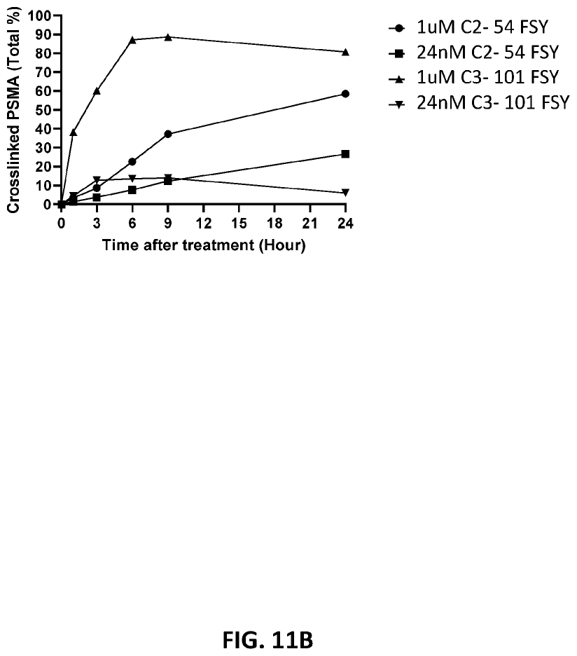

In LNCaP cells treated with C3-101FSY, a similar pattern was observed, the intensity of the crosslinked band (PSMA-sdAb) increased in a time and concentration dependent manner. Because the molecular weight of C3-101FSY is higher than C2-54FSY, the crosslinked band migrated with a higher apparent molecular weight than the crosslinking band of C2-54FSY (FIG. 11A). A comparison of crosslinking kinetics of C2-54FSY and C3-101FSY is shown in Table 10 and FIG. 11B.

TABLE 10
Crosslinking kinetics of C2-54FSY and
C3-101FSY with PSMA in LNCaP cells

| | Treatment | | | |
|---|---|---|---|---|
| | 1 μM | 24 nM | 1 μM | 24 nM |
| Time after | C2-54FSY | C2-54FSY | C3-101FSY | C3-101FSY |
| Treatment (hour) | Crosslinked PSMA (Total PSMA %) | | | |
| 0 | 0.0 | 0.0 | 0.0 | 0.0 |
| 1 | 3.5 | 1.4 | 38.2 | 4.4 |
| 3 | 8.6 | 3.8 | 60.2 | 12.8 |
| 6 | 22.6 | 7.7 | 87.2 | 13.5 |
| 9 | 37.2 | 12.4 | 88.6 | 14.0 |
| 24 | 58.5 | 26.6 | 80.8 | 6.0 |

### Example 16—In Vivo Crosslinking Assay

This study evaluated in vivo crosslinking, systemic, and intratumoral exposure of C2-54TYR and C2-54FSY with PSMA in two mouse xenograft tumor models, LNCaP and PC3. LNCaP and PC3 cell lines (ATCC) were maintained in RPMI-1640 and F12K media respectively supplemented with 10% heat inactivated fetal bovine serum (Thermo Fisher) in a humidified environment with 5% C02 at 37° C. The cells were harvested at exponential growth phase and centrifuged at 335×g in a refrigerated centrifuge and the medium aspirated. For cell inoculation, cell pellets were re-suspended in 100 ul of serum-free F-12K medium for PC3 or RPMI for LNCaP plus 100 μL of Matrigel. 200 μL containing 5 million PC3 cells or 3 million LNCaP cells were implanted into the flanks of male NSG mice (Jackson Labs). When tumor sizes reached ~200 mm³, C2-54TYR and C2-54FSY were injected through the tail vein. Peripheral blood samples were collected via cheek bleeding. After 6 hours, mice were sacrificed, and both peripheral blood samples and tumors were harvested, weighed and frozen at −80° C.

Tumor sample (~30 to 40 mg) was added with 1 ml RIPA buffer with protease inhibitor cocktail (Santa Cruz Biotechnology, Cat. No. SC-24948A) and then homogenized with Qiagen Tissuelyser II Sample Disruptor. After homogenization, samples were centrifuged at 12,000 rpm for 10 mins at 4° C. Supernatant was kept, and pellet was discarded. The centrifugation process was repeated once (twice in total).

Protein concentration of tumor lysate was quantified using the BCA protein assay kit (Thermo Scientific, Cat. No. 23225). The samples were formulated with RIPA buffer to the same concentration and were then heated at 100° C. for 10 min after adding 6× reduced loading buffer (Alfa Aesar, Cat. No. J61337). The denatured samples were analyzed by electrophoresis in 4-20% Criterion TGX Gel (Bio-Rad, Cat. No. 5671095) followed by western blot (Bio-Rad, Cat. No. 1704271 and Thermo Scientific, Cat. No. 35060, 35061) using anti-PSMA and anti-VHH to detect crosslinking and anti-GAPDH antibody as a loading control. Images were acquired by Azure Biosystem C600. GIMP 2.10.28 was used to process images. Plasma was subjected to ELISA and data were processed and plotted (GraphPad Prism 9.2.0). Mean value and STDEV were calculated and significance of plasma concentration was assessed using unpaired two-tail student t test.

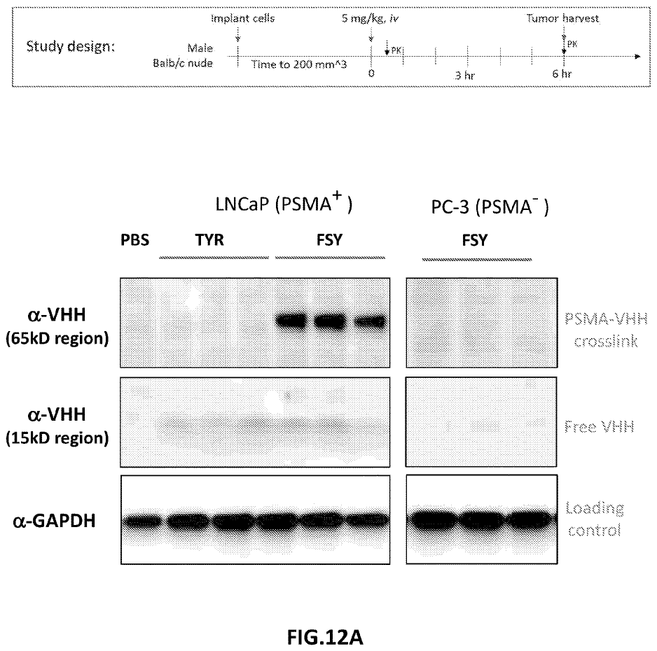

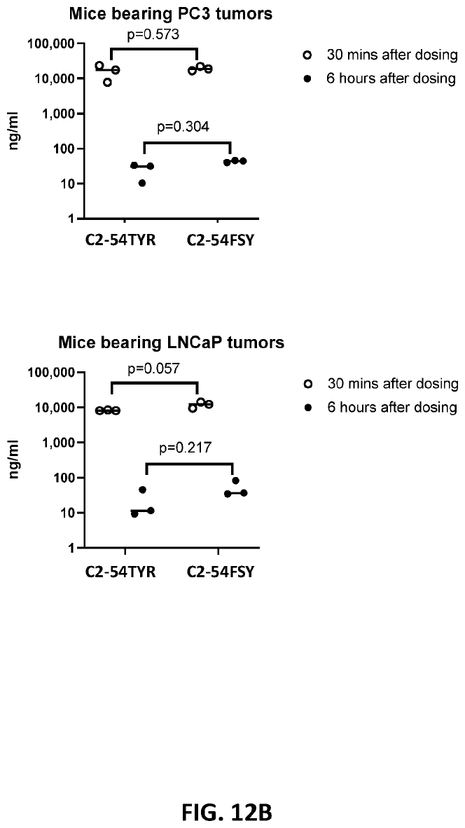

Samples from LNCaP and PC3 tumors derived from animals administered C2-FSY or C2-TYR were prepared as described above and western blotted with anti-VHH antibodies to detect the non-crosslinked C2 compounds (15 kD region of the blot) and the crosslinked C2 compounds (~100 kD region above PSMA). In LNCaP tumor samples from animals administered C2-54FSY, anti-VHH western blot detected a band above the PSMA region (~100 kD) in all samples, while no crosslink band was observed in the C2-54TYR or vehicle samples (FIG. 12A), indicating specific and reproducible crosslinking by C2-54FSY. No anti-VHH signal was observed in PC3 tumor samples, while differential patterns of bands were observed in LNCaP samples treated with C2-54TYR and C2-54FSY. In the ~15 kD region of the blot, which shows the un-crosslinked/free sdAb, both C2-54TYR and C2-54FSY treated LNCaP samples showed bands of similar intensity, demonstrating the presence of similar levels of non-crosslinked sdAb. An additional band was observed that migrated at ~100 kD specifically in the C2-54FSY treated LNCaP tumor tissues, while no band was observed in this region in PC3 samples or LNCaP samples from C2-54TYR treated animals, indicating crosslinking of C2-54FSY to PSMA.

To estimate and compare systemic exposure of the sdAbs, plasma concentrations of C2-54TYR and C2-54FSY were measured at 30 minutes and 6 hours after dosing using ELISA (Table 11 and FIG. 12B).

TABLE 11
Plasma concentrations (ng/ml) of C2-54TYR and C2-54FSY

| | | 30 mins after dosing | | | | 6 hours after dosing | | | |
|---|---|---|---|---|---|---|---|---|---|
| | | Groups | | | Mean ± STDEV | Groups | | | Mean ± STDEV |
| Mice | C2- | 7746.7 | 23430.0 | 17463.3 | 16213.3 ± 7916.0 | 33.2 | 31.2 | 10.3 | 24.9 ± 12.7 |
| bearing | 54TYR | | | | | | | | |
| PC3 | C2- | 18713.3 | 16696.7 | 22113.3 | 19174.4 ± 2737.6 | 43.9 | 39.9 | 45.7 | 43.2 ± 3.0 |
| tumor | 54FSY | | | | | | | | |
| Mice | C2- | 8530.0 | 8146.7 | 8163.3 | 8280 ± 216.7 | 9.3 | 11.5 | 45.7 | 22.2 ± 20.4 |
| bearing | 54TYR | | | | | | | | |
| LNCaP | C2- | 12246.7 | 14313.3 | 9463.3 | 12007.8 ± 2433.8 | 34.5 | 82.9 | 36.8 | 51.4 ± 27.3 |
| tumor | 54FSY | | | | | | | | |

The results show that, after a single IV dose, C2-54TYR and C2-54FSY access the tumor and accumulate in a PSMA-dependent manner. However, only C2-54FSY specifically crosslinks to PSMA in the tumor.

### Example 17 Construction of FSK-Modified and FSY-Modified sdAbs for Target-Specific Crosslinking

Libraries and library strains were constructed, expressed, and purified following the methods generally of Examples 2 and 11. To initially identify regions of CDRs that contain sites compatible with FSY or FSK insertion, strains containing plasmids with TAG codons within the coding region of CDR regions were constructed. Table 12 shows the sdAbs and CDR sequences used for the screening.

TABLE 12
sdAbs with FSY and FSK insertions

| | | | | FSY | FSK |
|---|---|---|---|---|---|
| sdAb | Target | CDR | CDR amino acid sequence | insertion | insertion |
| C14 | CEA5 | CDR1 (26-35) | GDTYGSYWMG | x | x |
| (SEQ | | CDR2 (50-66) | AINRGGGYTVYADSVKG | x | x |
| ID NO: | | CDR3 (99-112) | SGVLGGLHEDWFNY | x | x |
| 28) | | | | | |
| C8 | FAP | CDR1 (26-35) | GSIFVGNAMG | x | |
| (SEQ | | CDR2 (50-65) | GITSDGITYYPDSVKG | x | |
| ID NO: | | CDR3 (98-106) | WPPRIGFAS | x | |
| 23) | | | | | |
| C12 | Folate | CDR1 (26-35) | ETFGVVFTLG | x | |
| (SEQ | Receptor | CDR2 (50-65) | RVTGTDTVDYAESVKG | x | |
| ID NO: | alpha | CDR3 (98-100) | GAY | x | |
| 27) | | | | | |
| C11 | MSLN | CDR1 (26-35) | GIDLSLYRMR | x | x |
| (SEQ | | CDR2 (50-65) | LITDDGTSYYEDSVKG | x | x |
| ID NO: | | CDR3 (98-106) | ETPLSPVNY | x | x |
| 26) | | | | | |
| C10 | MSLN | CDR1 (26-35) | GSIFGIRTMD | x | |
| (SEQ | | CDR2 (50-65) | RITMDGRVFHADSVKG | x | |
| ID NO: | | CDR3 (98-106) | SGLTSREDY | x | |
| 25) | | | | | |
| C18 | HER3 | CDR1 (26-35) | GRTSSKYAMG | x | x |
| (SEQ | | CDR2 (50-66) | TISWSDGSTYYADSVEG | x | |
| ID NO: | | CDR3 (99-113) | AVDVLAGTFEYEYDY | x | |
| 34) | | | | | |
| C20 | CD123 | CDR1 (26-35) | GRAINTYAMG | x | |
| (SEQ | | CDR2 (50-66) | AISWNGGHTRYADSVQ | x | |
| ID NO: | | | G | | |
| 37) | | CDR3 (99-114) | YSDYHRIATMEADADS | x | |
| C21 | 5T4 | CDR1 (26-35) | DFTFRNYEMS or | x | x |
| (SEQ | | | DFTFRSYEMS | | |
| ID NO: | | CDR2 (50-66) | SISGSGGSTYYAHSLKG | x | x |
| 38) | | CDR3 (99-109) | LRDGFNNGFDY | x | x |

Screening. Strains containing plasmids with TAG codons within the coding region of CDR regions were screened in pools and crosslinking was assayed following the methods generally of Examples 11 and 12. Pools showing crosslinking activity to the target were further analysed by assessing the individual FSY or FSK mutants for crosslinking. Individual mutants showing crosslinking are shown in Table 13.

TABLE 13
Identification of FSY and FSK insertions for crosslinking sdAbs

| | Target for | | |
|---|---|---|---|
| | crosslinking | | |
| sdAb | assay (human) | FSY positions | FSK positions |
| C14 | CEACAM5 | 55 | 52 |
| C8 | FAP | 54, 55, 56 | 52, 53, 54, 55, 56, |
| | | | 58, 60, 62, 64 (example |
| | | | 11 above) |
| C12 | Folate Receptor | 58 | not tested |
| | alpha (FOLRa) | | |
| C11 | MSLN | 53, 54, 101, 102, | 53 |
| | | 103, 104, 105 | |
| C10 | MSLN | 29, 30, 31, 32, | not tested |
| | | 33, 52, 53, 54, | |
| | | 100, 101 | |
| C18 | HER3 | 33, 101, 103 | 30, 31, 32, 35 |
| C20 | CD123 | 56, 57, 58 | not tested |
| C21 | 5T4 | 59 | 27 |

Reagents used for targets of crosslinking assays. CEACAM5 was purchased from Acrobiosystem (#CE5-HF255-25 μg), and Sinobiological (#11077-H02H-100 μg). FAP receptor was purchased from Acrobiosystem (#AP-H5263-100 ug). Human FolRa (Folate receptor alpha) receptor was purchased from Acrobiosystem (#FO1-H5253-100 ug). MSLN (mesothelin) was purchased from Sinobiological (Cat: 13128-HNCH, Cat: 13128-H01H-B) and Acrobiosystem (#MSN-H526x-100 ug). The human MSLN extracellular domain was purchased from Acrobiosystem (#MSN-H5253-100 ug, #MSN-HF223-25 ug). Her3 extracellular domain was purchased from Acrobiosystem (#ER3-H5223-100 ug). Human CD123 protein extracellular domain was purchased from SinoBiological (#10518-H02H-50 ug). The human 5T4 extracellular domain was purchased from Acrobiosystem (#TPG-H5253-100 ug).

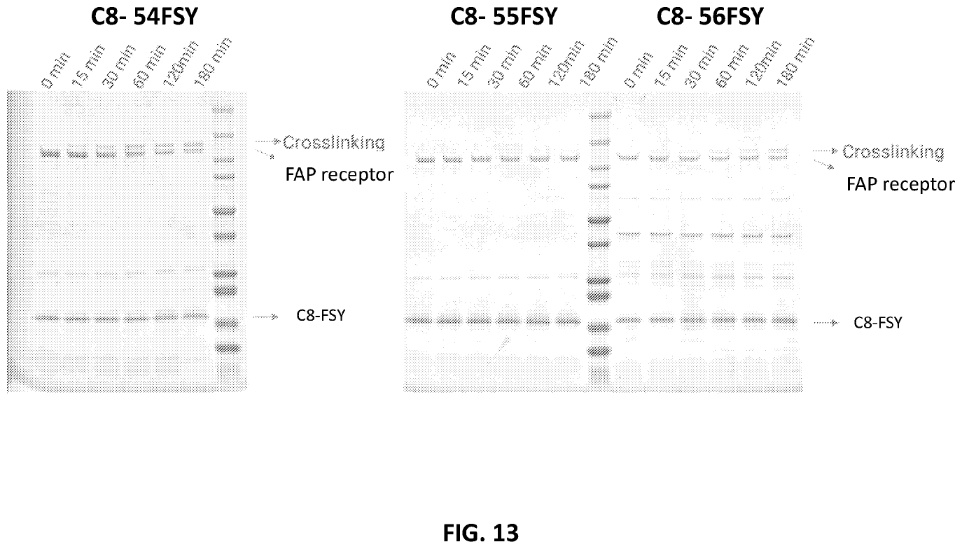

For the C8 FSY sdAbs, kinetics of crosslinking was analysed following the methods of Example 11. FIG. 13 shows the cross-linking from 0-180 minutes. The C8-54 FSY site was found to have the highest crosslinking rate compared to the other sites (~50% crosslinking within 2 hr). Similarly, the crosslinking rate of C18-FSY and C18-FSK sdAbs to HER3 receptor was compared. As between positions 33, 101, and 103, position 101 FSY was observed to achieve the highest crosslinking efficiency and rate. For the C18 sdAb FSK variants at positions 30, 32, 35, position 35 FSK was observed to achieve the highest crosslinking efficiency and rate to Her3. An assessment of 3 sites in C20 FSY sdAbs indicated that position 57 achieved the highest crosslinking efficiency and rate to CD123.

### Example 18 Identification and Kinetics of FSY-Modified DARPins

A Her2-binding DARPin (C15) was screened for the ability to crosslink through insertion of an FSY amino acid. Libraries and library strains were constructed, expressed, and purified following the methods generally of Example 2. The C15 WT sequence (SEQ ID NO 29) is shown in Table 14 with the regions screened by FSY insertion shown in bold and amino acid positions of the FSY substitutions shown in the second row of the table.

TABLE 14
The amino acid position for substitution of FSY
into the HER2 DARPin

| | |
|---|---|
| DARPin | MDLGKKLLEAARAGQDDEVRILMANGADVNAHDFYG |
| C15 wt | ITPLHLAANFGHLEIVEVLLKHGADVNAFDYDNTPL |
| amino | HLAADAGHLEIVEVLLKYGADVNASDRDGHTPLHLA |
| acid | AREGHLEIVEVLLKNGADVNAQDKFGKTAFDISIDN |
| sequence | GNEDLAEILQKLAAALEHHHHHH |
| FSY | 2, 4, 5, 6, 7, 8, 9, 10, 11, 12, 33, |
| sites | 34, 35, 36, 37, 66, 67, 68, 69 |
| in the | |
| library | |

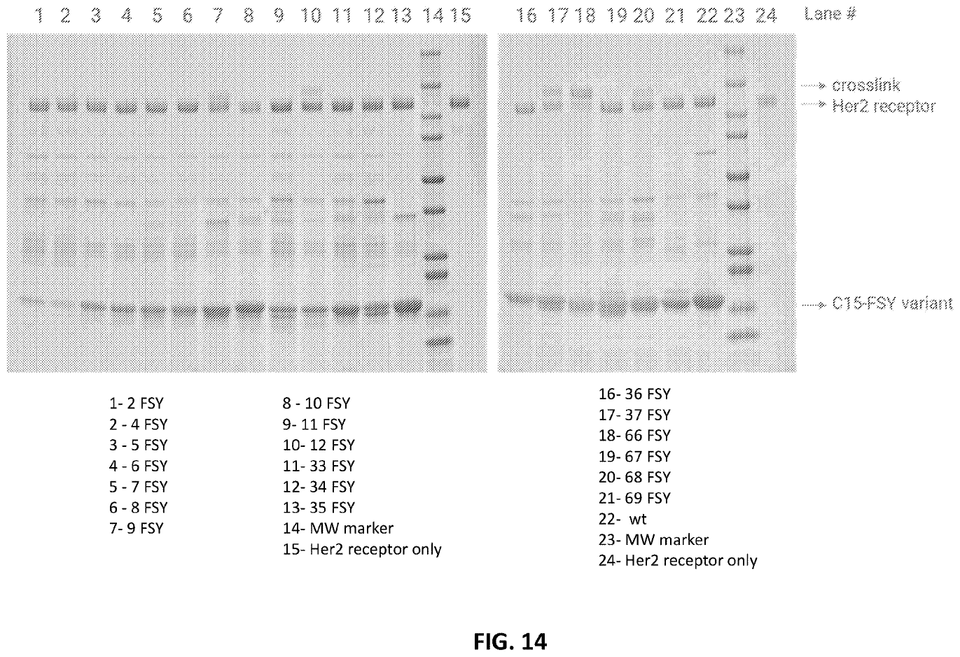

Each individual C15-FSY mutant was incubated with Her2 receptor for crosslinking efficiency following the methods generally of Example 11. Sites 9, 12, 37, 66, 68 in C15 demonstrated crosslinking with Her2 receptor as shown in FIG. 14. To identify the FSY crosslinking site with the fastest rate, DARPins with FSY sites identified above were incubated with Fc-tagged Her2 (Acrobiosystem: Acrobiosystem: #HER2-H5253-100 ug) and examined for crosslinking at 15 and 30 minutes. The C15-66 FSY site was found to have higher crosslinking yield compared to the other sites.

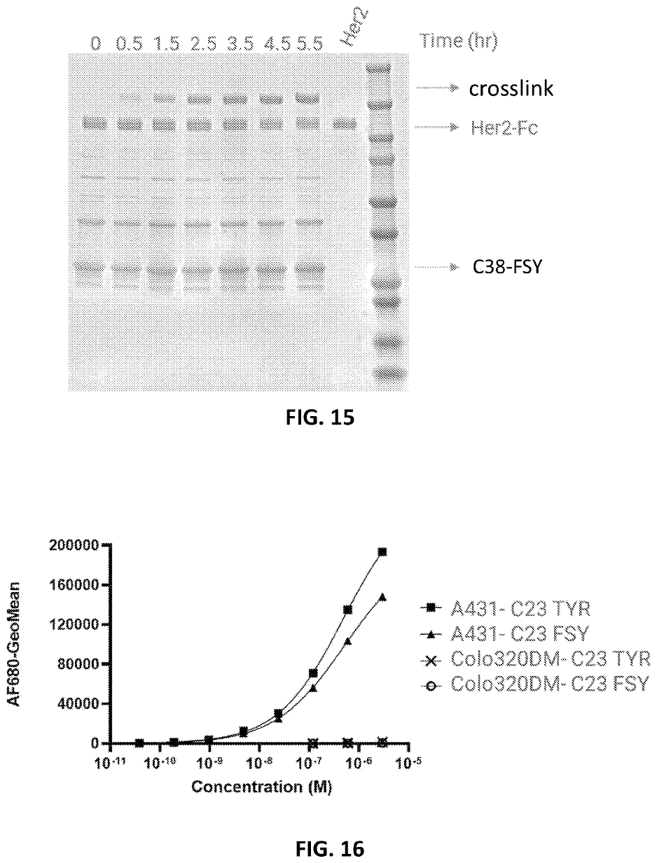

A biparatopic DARPin (C15-66FSY-C16, hereafter referred to as C38-FSY) was constructed by fusing one copy of the DARPin without FSY substitution C16 (SEQ ID NO 30) to the C-terminus of C15-66FSY with a 5 amino acid GGGGS linker to make a fusion protein. The C38-FSY protein was incubated with the Fc-tagged Her2 and examined for crosslinking following the methods generally of Example 11. The biparatopic C38-FSY crosslinked Her2 in a time dependent manner, with 50% crosslinking within 2.5 hr, as shown in FIG. 15.

### Example 19 Cell Binding with a Targeted Imaging Payload

C22-TYR and C22-FSY (SEQ ID Nos. 39 and 40, respectively) were expressed and purified following the methods generally of Example 2, with the exception that following elution from the Ni-NTA resin, the purified protein was then buffer exchanged into anion exchange running buffer (20 mM Tris, 7.5 and 20 mM NaCl), overnight at 4 C using a dilution factor of 1:100. The dialyzed sample pool was passed over a HiTrap Q XL, 5 ml column (Cytiva #17515801) in flow through mode with the endotoxin binding to the column. The sdAb monomer flowthrough fractions were analyzed by reducing SDS-PAGE and pooled. The purified C22-TYR and C22-FSY were then conjugated to AZ Dye 680 (Click Chemistry Tools, Catalog #1578-25).

Sortase mediated ligation was used to conjugate AZ Dye680 (Click Chemistry Tools, Catalog #1578-25) to C22-FSY and C22-TYR to create C23-TYR and C23-FSY. Sortase gblock sequence was codon optimized and ordered by IDT, cloned into pBAD vector and prepared as described (PubMed: 21697512). To conjugate sdAb constructs to AZ Dye 680, 20 mg of proteins purified above in PBS pH 7.4 were incubated with 1 mM CaCl₂, 0.5 mM AZ Dye 680, 2 mg sortase at 30° C. in the shaker (final concentration of sdAb was ~0.7 mg/mL). After 2 hours, 2 mM MTSET was added to quench the reaction. The quenched reaction was batch bound to 2 mL Ni resin (PBS equilibrated) to remove sortase and unconjugated material, nutating at RT, 30 min. The beads were spun to facilitate separation of resin from unbound fraction (700 g×10 min, 20° C.) and the flow through was collected by loading to a glass column and washing beads with PBS to collect remaining protein. The flowthrough and wash were combined and concentrated (5K MWCO, 4 k rcf, 1° C., 1.5 h) and loaded on onto the HiLoad 26/600 Superdex 75 pg (Cytiva, #28-9893-34) to remove unconjugated dye. Fractions were collected and analyzed by SDS-PAGE and pooled and stored at −80° C.

The resulting conjugates C23-TYR and C23-FSY were assessed for binding to human epidermoid squamous carcinoma lines A431 (EGFR+) and the human colorectal cancer cell line COLO320DM (EGFR−) by flow cytometry. Proteins were formulated with FACS buffer (1×PBS+2% HI-FBS+5 mM EDTA) to 30 μM (10× final) and then serially diluted 5× (20 μL sdAb to 80 μL FACS buffer) to generate 8 concentration points for the assay, with the lowest concentration at 0.000384 μM (10× final). A431 and COLO320DM cell lines (ATCC) were both maintained in RPMI-1640 supplemented with 10% heat inactivated fetal bovine serum (Thermo Fisher) in a humidified environment with 5% CO₂ at 37° C. The cells were harvested at exponential growth phase, counted, resuspended in ice-cold FACS buffer at 1.1×10⁶/mL and aliquoted into a v-bottom 96-well plate (Corning 3897) at 90 μL/well. The C23-TYR and C23-FSY proteins were added to A431 cells, 10 uL/well bringing final concentration to 1×. To control for specificity of binding to EGFR+ cells, test articles of the same dilution range were added to the COLO320DM cells. After incubation on ice for 2 hours, cells were pelleted at 2000 rpm for two minutes, then were washed with 200 μL FACS buffer twice, then resuspended in 100 μL of ice-cold FACS buffer and analyzed with NovoCyte flow cytometer (ACEA/Aglient).

Raw data (FCS files) were analyzed with FlowJo 10.7.2. FSC-A and SSC-A were used to identify total intact cells, then singlets were gated out with FSC-A against FSC-H. Geometric mean fluorescence intensity of AZ680 of samples were used for further analysis and plotting. Geometric mean fluorescence intensity (GeoMean or GeoMFI) for the AZ680 channel was used to calculate EC50 in GraphPad Prism (V9.3.1) using ‘log(agonist) vs. response—variable slope (four parameters).

C23-FSY and C23-TYR bound to A431 cells in a dose-dependent manner but did not exhibit binding to the EGFR negative COLO320DM cells as shown in FIG. 16. Binding affinities for C23-FSY and C23-TYR were measured from EC50 values after staining and flow cytometry of A431 cells as shown in Table 15.

TABLE 15
Binding affinity of C23-FSY and C23-
TYR AF680 conjugates on A431cells

| | C23-TYR | C23-FSY |
|---|---|---|
| EC50 (nM) | 495 | 518 |
| EC50 Range | 415.2-613.1 | 444.7-618.2 |
| R squared | 0.99 | 1.00 |

### Example 20 In Vivo Tumor Locking with a Targeted Imaging Payload

This study evaluated in vivo crosslinking of fluorescently labelled sdAbs. C22-FSY and C22-TYR were modified to create the AZ680 conjugates C23-FSY and C23-TYR (SEQ ID Nos. 42, 41, respectively) following the methods of Example 19. In vivo crosslinking of C23-FSY and C23-TYR to EGFR was evaluated in the A431 epidermoid squamous carcinoma xenograft tumor model. The human colorectal cancer xenograft model COLO320DM served as an EGFR-control. Both cell lines were cultured according to the supplier's protocol (ATCC). On the day of injection, cells were harvested, washed in serum-free media, counted, and resuspended in cold serum-free media. A431 cell pellets were re-suspended in 100 mL of serum-free DMEM, and COLO320DM 100 μL RPMI1640, then cells were mixed with 100 mL of Matrigel resulting in a final concentration of 5×10⁶ viable cells/100 μL. A volume of 200 mL containing 10 million A431 or COLO 320DM cells were implanted into the right upper flank of female Balb/c nude mice, age 6-8 weeks (Charles River). When tumor sizes reached ~200 mm³, 5 mg/kg of C23-FSY, C23-TYR or vehicle control (PBS) were injected through the tail vein in a volume of 10 mL/kg. Peripheral blood samples were collected via cheek bleeding at 0.5 hours after dosing for confirmation of dosing accuracy.

Biodistribution of the test articles was studied at 2 different timepoints as indicated in the Table 16. Animals were euthanized, tumors were harvested, weighed and placed on ice to be IVIS imaged ex vivo, and thereafter snap frozen for western blot analysis.

TABLE 16
Timepoints for biodistribution analysis

| | Number of | | |
|---|---|---|---|
| | mice per | Timepoints | Non-terminal serum |
| Group | timepoint | (hours) | sampling for PK |
| Vehicle | 1 | 8, 24 | 0.5 h for all mice |
| C23-TYR | 3 | 8, 24 | 0.5 h for all mice |
| C23-FSY | 3 | 8, 24 | 0.5 h for all mice |

Tissue processing. A piece of tumor (~30 to 50 mg) was weighed and chopped into small pieces with razor blade. Chopped tumor tissue was put in CK28-R tubes (Bertin Instruments, Cat.P000916LYSK0-A) and 0.5 ml T-PER buffer (Thermo Scientific Cat. 78510) with Halt Protease & Phosphatase Inhibitor Cocktail (Thermo Scientific Cat. 78446) was added. Tissue was then homogenized with the Precellys 24 Tissue Homogenizer. After homogenization, samples were centrifuged at 12,000×rpm for 10 mins at 4° C. Then supernatant was retained, and pellet was discarded. The centrifugation process was repeated once more (twice in total).

Gel imaging. Protein concentration of tumor lysates was quantified using Pierce Rapid Gold BCA Protein Assay Kit (Thermo Scientific Cat. A53225) following the manufacturer's instructions. A 7-point 2-fold dilution of BSA starting at 1 mg/mL BSA from the kit was used as standard. The samples were formulated to the same concentration (3.6 mg/mL) with T-PER buffer and then heated at 95° C. for 10 min in 1× reduced loading buffer, yielding a final concentration of 3 mg/mL. Next, 30 ug total protein of each sample was loaded for electrophoresis on a 4-20% Criterion TGX Gel (Bio-Rad, Cat.5671085). Images were acquired by Azure Biosystem C600 with NIR-700 channel. GIMP 2.10.28 was used to process images.

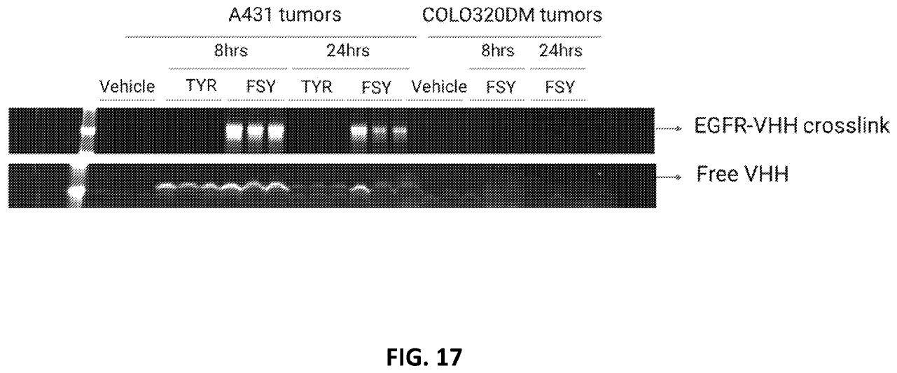

FIG. 17 shows intratumoral free- and EGFR-crosslinked sdAb in a time-dependent manner in A431 (EGFR+) and COLO320DM (EGFR−) tumors. Free sdAb was detected in the ~15 kD region (bottom panel, labeled free VHH) and the EGFR crosslinked sdAb band in the ~175 kD region (top panel, labeled VHH-EGFR crosslink). Time-dependent crosslinking of EGFR was observed with C23-FSY but not C23-TYR in EGFR+A431 tumors. Neither free-sdAb retention nor crosslinking was observed in the EGFR-COLO320DM tumors.

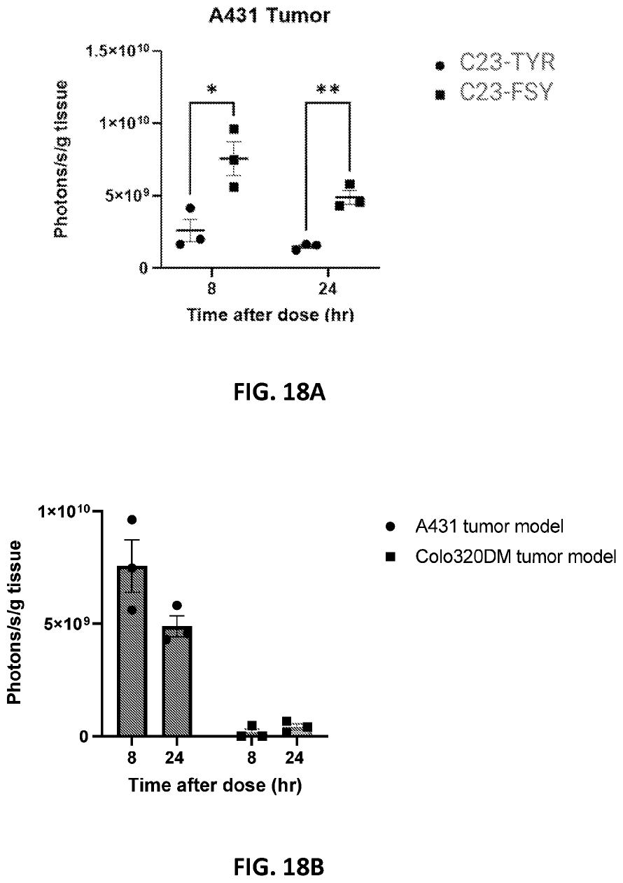

Ex Vivo IVIS Imaging data analysis: Tumors were dissected and imaged with AMI HTX; imaging data were analyzed with Aura software (Spectral Instruments Imaging, Version 4.0.7). Average radiant efficiency values were obtained from the creation of ROIs around individual tumors (Ellipse ROI tool in Aura). Total Emission (Photons/s) was measured and recorded. Total Emission values were normalized to tissue weights (Photons/s/g). Statistical significance was determined in Prism (v 9.1.0 (221)) using paired T-test (*=p≤0.05, **=p≤0.005). FIG. 18 shows ex vivo imaging of A431 and COLO320DM tumor tissues at 8 and 24 hours post-administration of C23-FSY and C23-TYR.

FIG. 18A shows photons/s/g tumor tissue across triplicate biological replicates. Values from individual animals are shown, the center bar shows the mean intensity and error bars indicate SEM (* indicates p=0.024, ** indicates p=0.002 by paired t test). The C23-FSY was present at a significantly higher level in the A431 tumors as compared with C23-TYR protein at both the 8 and 24 hour time points. After 24 hours the C23-TYR was observed at very low levels in the A431 tumor tissue, while C23-FSY, particularly the EGFR-VHH crosslinked species, was prominent (FIG. 17, 18A). As the test articles have similar affinities (FIG. 16), these results together show that the tumor AUC of the FSY-containing sdAb can be increased by covalency.

FIG. 18B shows quantitative ex vivo fluorescence intensities of A431 and COLO320DM tumors from animals administered C23-FSY at 8 and 24 hours post dose. Plot shows photons/s/g tumor tissue across triplicate biological replicates. Values from individual animals are shown, center bar shows the mean intensity, error bars indicate SEM. The C23-FSY was present at a significantly higher level in the A431 tumors as compared with the amounts present in the EGFR-COLO320DM tumors at both the 8 and 24 hour time points, demonstrating the specificity of the tumor locking of the C23-FSY protein.

These results show that a targeting domain, such as a sdAb, with a crosslinking-compatible unnatural amino acid can carry a payload (here an imaging dye) to a tumor with targeting specificity and also improve the retention of the payload at the target site as compared to a targeting domain without the crosslinking-compatible unnatural amino acid.

### Example 21 In Vivo PSMA Tumor Locking with a Targeted Imaging Payload

This study evaluated in vivo PSMA crosslinking, systemic, and intratumoral exposure of fluorescently labelled sdAbs in the LNCaP mouse prostate cancer xenograft tumor model.

PSMA-targeted sdAbs conjugated to AZ Dye 680 were prepared using C29-101TYR and C29-101FSY (SEQ ID NO: 53 or 54), with either TYR or FSY at position 101 following the methods of Example 19 to create C30-TYR and C30-FSY (SEQ ID NOs: 55, 56, respectively). LNCaP cells were maintained in RPMI-1640 media supplemented with 10% heat inactivated fetal bovine serum in a humidified environment with 5% CO2 at 37° C. On the day of implantation, cells were harvested, washed in serum-free media, counted, and resuspended in cold serum-free media. Cell pellets were re-suspended in 50 μL of serum-free RPMI and mixed with 50 μL of Matrigel resulting in a final concentration of 5×10⁶ viable cells/100 μL. A volume of 100 μL containing 5 million LNCaP cells was implanted into the right upper flanks of male NU/J mice (Jackson Labs). When tumor sizes reached ~200 mm3, 5 mg/kg of either C30-TYR or C30-FSY were injected through the tail vein in a volume of 10 mL/kg. Peripheral blood samples were collected via cheek bleeding at 0.5 hours after dosing for confirmation of dosing accuracy. Biodistribution of the test articles were studied at 8 different timepoints (pre-dosing, 15 mins, 1 h, 6 h, 24 h, 48 h, 72 h and 96 h after dosing) as shown in Table 17. After euthanization, tumor tissue was harvested, weighed, IVIS imaged ex vivo, and thereafter snap frozen for western blot analysis.

TABLE 17
Timepoints for PK sampling

| | Number of | | |
|---|---|---|---|
| | mice per | Timepoints for | Non-terminal serum |
| Group | timepoint | sacrifices (hours) | sampling for PK |
| Vehicle | 1 | 0.25, 1, 6, 24, | 0.5 h for all mice except |
| | | 48, 72, 96 | 0.25 h terminal |
| C30-TYR | 3 | 0.25, 1, 6, 24, 48 | 0.5 h for all mice except |
| | | | 0.25 h terminal |
| C30-FSY | 3 | 0.25, 1, 6, 24, 48, | 0.5 h for all mice except |
| | | 72, 96 | 0.25 h terminal |

Tissue processing, protein quantification and normalization were performed using the methods of Example 20. For quantification of free and PSMA-crosslinked sdAb test article, 2-fold 16-point standard curves of C30-FSY were prepared in T-PER buffer starting at 500 ng/mL. Standards were heated at 95° C. for 10 min in 1× reduced loading buffer. Based on estimation of the test article concentration in the samples, different range of standards was used for different gels. A standard curve was generated using the band area of standards against their concentrations using the linear fit function in Excel. Density of bands in images was determined with ImageJ 1.51j8 following the instructions, (https://imagej.nih.gov/ij/docs/guide/146-30.html #infobox:Densitometry, Section 30.13). After obtaining the area of the band of the sample with ImageJ, amount of TA in the lane was calculated with the formula deducted from the linear fit of standard curve. As 30 μg of total protein was loaded, the amount of TA in the lane is also amount of TA in 30 μg of total protein. Tumor sdAb concentration (pg sdAb/mg tumor tissue)=sdAb amount in lane (pg)*(protein concentration (mg/ml)*(0.5 ml*1000/30 μg)/tumor tissue weight (mg). The resulting standard curve contained at least 5 points with R2>0.99. Area under curve (AUC) was calculated with GraphPad Prism using the trapezoid rule wherein the area between two adjacent points is calculated as ΔX*([(Y1+Y2)/2]-Baseline](https://www.graphpad.com/guides/prism/latest/statistics/stat_area_under_the_curve.htm). The AUC for C30-TYR was 1676 and the AUC for C30-FSY was 4902.

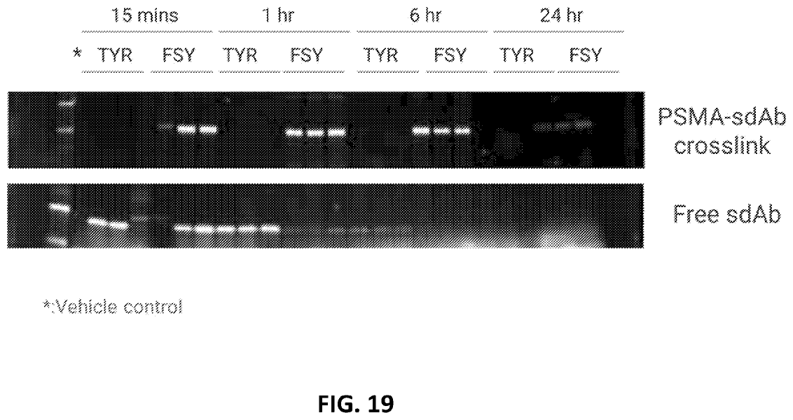

FIG. 19 shows a fluorescence image of SDS PAGE gels showing tumor associated free sdAb and PSMA-crosslinked sdAb in a time-dependent manner post administration of C30-TYR and C30-FSY. At designated time points post-treatment, tumors were harvested and processed for gel electrophoresis to detect fluorophore-conjugated sdAb test articles. Free (non-crosslinked) sdAb-AF680 was detected in the ~20 kD region (bottom panel) and the PSMA-crosslinked sdAb-AF680 species for C30-FSY migrated in the 100 kD region (top panel). The lane indicated with * shows vehicle sample.

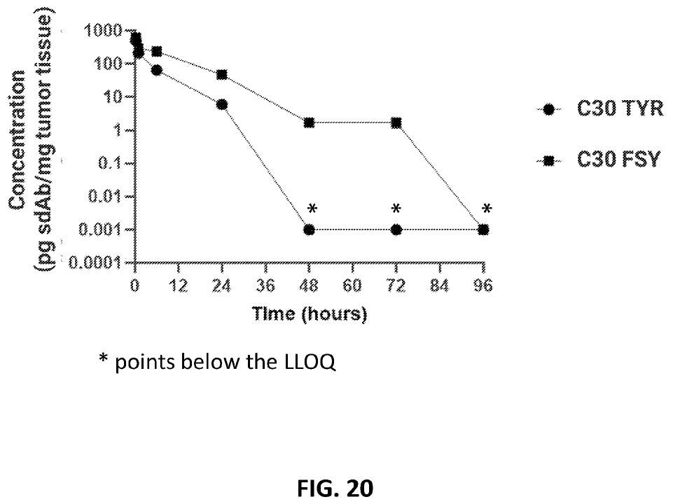

FIG. 20 shows the quantitative analysis of tumor associated free and PSMA-crosslinked test articles. Fluorescence band intensity of the free and PSMA-crosslinked species bands from the gel above were quantitated via densitometry and comparison to a standard curve. The total intratumoral test article concentration (free and PSMA-crosslinked, pg/mg tumor tissue) is plotted vs time (h). Data points represented with * indicate samples that were below the limit of detection and quantitation. The tumor exposure of the C30-FSY was increased approximately 3× relative to the non-covalent C30-TYR.

### Example 22 Delivery of a Cytotoxic Payload

C25 and C27 sdAbs were prepared with and without FSY substitutions to generate C25-54TYR, C25-54FSY, C27-101TYR and C27-101FSY (SEQ ID Nos: 45, 46, 49, 50 respectively). The proteins were expressed and purified following the methods generally of Example 2, with the exception that following elution from the Ni-NTA resin, the purified protein was then buffer exchanged into anion exchange running buffer (20 mM Tris, 7.5 and 20 mM NaCl), overnight at 4 C using a dilution factor of 1:100. The dialyzed sample pool was passed over a HiTrap Q XL, 5 ml column (Cytiva #17515801) in flow through mode with the endotoxin binding to the column. The sdAb monomer flowthrough fractions were analyzed by reducing SDS-PAGE and pooled.

The sdAb constructs were conjugated to MC-PEG8-VC-PABC-MMAE (structure shown below) to create C26-54 TYR, C26-54 FSY, C28-101TYR and C28-101FSY. The samples were first reduced with 1 mM EDTA and 1.5 equivalents of TCEP (10 mM in deoxygenated water) and incubated at room temperature for 1 hour. After reduction, any residual TCEP was removed using a Zeba Spin desalting column (Thermo P/N 89891, 7K MWCO), using 1 mM EDTA in 1×PBS as the equilibration buffer. Propylene glycol (PG) and dimethylacetamide (DMA) was added to the reaction mixture (Cf=10% v/v) to improve linker payload solubility and 1.5 equivalents of linker-payload was added. The reaction was vortexed gently and incubated at 4° C. overnight. The conjugate mixture was added to a pre-hydrated dialysis cassette (~1 ml Sample:3500 mL buffer volume) and dialyzed for a minimum of 2 hours. The dialysis buffer was replaced and sample was dialyzed overnight at 4° C. prior to removing sample for analysis by A280 and analytical SEC. Unconjugated linker payload was removed using Cytiva PD-10 columns (4.3 mL of Sephadex G-25 sorbent per cartridge) following standard PD-10 spin protocol (equilibration buffer: 1×PBS, pH 7.4). After collection of eluate, the column was rinsed with an additional 1 mL of 1×PBS, pH 7.4. After purification on PD-10, samples were subjected to a final dialysis step using Thermo Scientific Slide-A-Lyzer mini dialysis devices (3.5 k MWCO) using 50 mL of 1×PBS. All samples were passed through a 0.2 μm sterile PES filter and stored at −20 C.

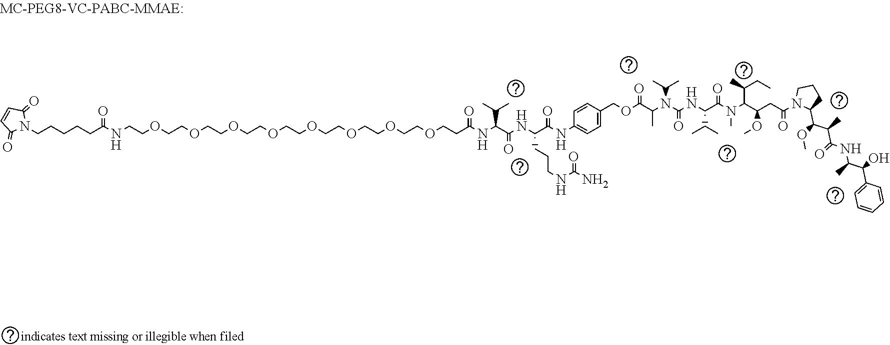

To measure the capacity of the FSY-containing sdAb conjugates to specifically deliver a cytotoxic payload to immortalized PSMA-expressing tumor cell lines in vitro versus non-covalent sdAb conjugates, the cell permeable payload monomethyl auristatin was conjugated to sdAbs with a drug to antibody ratio (DAR) of 1. The PC3pip cell line engineered to express PSMA, and PC3flu PSMA negative cell lines (provided by professors Xinning Wang and Warren D. Heston, Case Western Reserve University, Cleveland, OH) were maintained in growth media consisting of RPMI-1640 (Thermo Scientific, 11875-903) supplemented with 10% heat inactivated fetal bovine serum (Thermo Fisher, FB-12) in a humidified environment with 5% CO₂ at 37° C. Cells were harvested at exponential growth phase using TrypLE Express (Thermo Scientific, 14175-095), and seeded at 800 cells/well in 100 μL growth media in a black 96-well flat clear bottom plate (Costar, 3603). Cells were then incubated overnight in a humidified environment with 5% CO₂ at 37° C. to allow cells to adhere to the plate. The sdAb-MMAE conjugates listed in the table below were diluted in growth media to 50× final concentration and diluted in 3-fold increments to create a 10-point dilution series. A volume of 2 μL of each of the 50× test article dilution series was added to wells in triplicate to achieve a final concentration of 1× (top concentrations for each series indicated in Table 18).

TABLE 18
Test article conjugates

| Name | Top concentration in dose response (1x) |
|---|---|
| C26-54TYR | 158 nM |
| C26-54FSY | 137 nM |
| C28-101TYR | 208 nM |
| C28-101FSY | 127 nM |

Plates were returned to the incubator for 4 days, after which cell viability was assessed. CellTiter-Glo (Promega, G5737) was added to all plates at 50 μL/well, then plates were shaken for one minute at 1000 rpm at room temp. Luminescence was then read on the Victor X5 plate reader (PerkinElmer) and reported as relative luminescence units (RLU). Viability was calculated in Excel (Microsoft® Excel® for Microsoft 365 MSO, Version 2202 Build 16.0.14931.20128, 64-bit) as % viability=100*(treated RLU/untreated RLU). Data were plotted as % viability versus concentration of test article in GraphPad Prism (V9.3.1) and IC50 was calculated using log [inhibitor] vs. response—variable slope (four parameters).

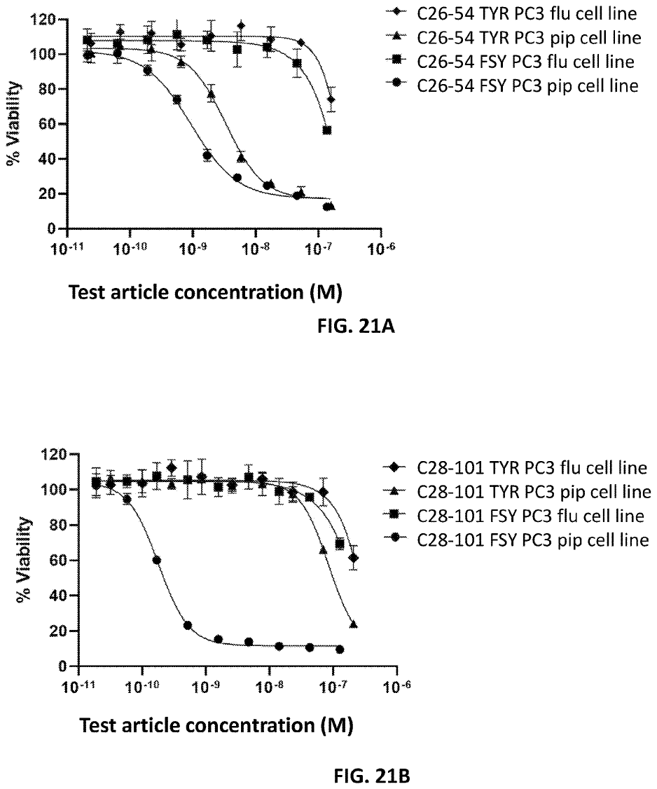

Extremely low-level cytotoxic activity was observed for the sdAb-PEG8-VC-PABC-MMAE conjugates toward the PC3flu cell line, consistent with the lack of PSMA target expression on this cell line. C26-54TYR, with an estimated affinity of 14.9 nM for PMSA, (as shown in Example 14 for the un-conjugated compound on LNCaP cells) showed a dose-dependent increase in cytotoxicity on PC3pip cells, consistent with targeted delivery of payload via PSMA binding and internalization. As shown in Table 19, C26-54FSY was 3.57× more potent at delivering payload than C26-54TYR, demonstrating that covalency enhances cellular potency of a single domain antibody drug conjugate. In a second example, C28-101TYR with a >500 nM affinity for PSMA had a low level of cytotoxic activity on PC3pip cells, whereas the crosslinking activity of C28-101FSY shifted the potency by over 400× confirming the impact of covalency on cellular potency. A comparison of the cytotoxicity of the test articles showing concentration versus cell viability curves for sdAbs conjugated to MMAE is shown in FIG. 21A (C26 constructs) and FIG. 21B (C28 constructs). After a 4-day incubation with sdAb-PEG8-VC-PABC-MMAE conjugates, the viability of PSMA+PC3pip and PSMA-PC3flu cells was evaluated by CellTiter-Glo assay. Dose-response analyses were performed in triplicate, symbols represent the mean and error bars represent STDEV.

TABLE 19
IC50 values for cellular cytotoxicity
of sdAb-PEG8-VC-PABC-MMAE conjugates

| | IC50 | IC50 |
|---|---|---|
| | PC3pip (M) | PC3flu (M) |
| C26-54TYR | 3.36e−9 | >1e−7 |
| C26-54FSY | 9.39e−10 | >1e−7 |
| C28-101TYR | 8.47e−8 | >1e−7 |
| C28-101FSY | 1.8e−10 | >1e−7 |

### Example 23 Delivery of a Cytotoxic Payload to a HER2 Target

A HER2-targeted sdAb (C17; SEQ ID NO: 31) was evaluated to identify individual positions for FSY insertion that provided crosslinking to a HER2 target. The sdAb was cloned expressed and purified generally according to the methods of Example 2, with the exception that specific single sites were selected for modification and testing, rather than use of pooled screening. Selected sites for FSY insertion are shown in Table 20.

TABLE 20
Focused FSY insertion sdAb library to target
HER2

| | Amino acid | Sites selected for FSY |
|---|---|---|
| | sequence | insertion |
| CDR1 | GYIFNSCGMG | 30, 31, 33 |
| CDR2 | RISGDGDTWHKESVKG | 50, 51, 52, 53, 54, 55, |
| | | 56, 57, 58, 59, 60 |
| CDR3 | CYNLETY | 99, 100, 101, 102, 103, |
| | | 104 |
| Non- | | 37, 44, 45, 47 |
| CDR | | |

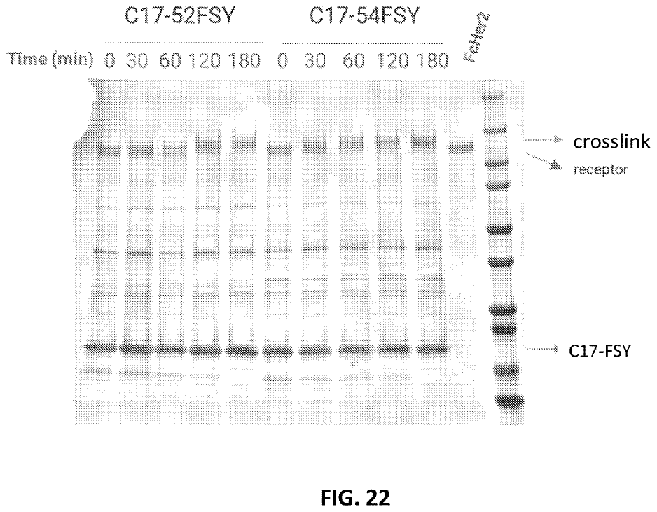

Positions 52 and 54 were identified as crosslinking to the HER2 target. To evaluate the FSY crosslinking kinetics, the 52FSY and 54FSY variants of C17 were incubated with Her2 receptor and examined for crosslinking efficiency. As shown in FIG. 22, the 54FSY site was found to have the highest crosslinking rate with crosslinking >90% within an hour, while 52FSY reached 90% crosslinking within 2 hr. These 2 constructs were selected, along with their non-FSY (TYR) counterparts for conjugation to a cytotoxic payload.

TABLE 21
Conjugates based on C17 variants
Name

C33-54FSY
C33-52FSY
C33-52TYR
C33-54TYR

To conjugate sdAb constructs to GGG-PEG8-vc-PABC-MMAE, C32-54TYR, C32-54 FSY, C32-52TYR and C32-52FSY compounds were purified as above. Compounds were incubated in PBS pH 7.4 were incubated with 1 mM CaCl₂, 10 equivalents of linker payload (stock solution 10 mM in 10% DMA), and sortase (2 mg sortase/10 mg sdAb), and incubated at 4° C. overnight. The reaction was batch bound to 2 mL Ni resin (PBS equilibrated) to remove sortase and unconjugated material, nutating at RT, 30 min. The beads were spun to facilitate separation of resin from unbound fraction (700 g×10 min, 20 C) and the flow through was collected by loading to a glass column, and washing beads with PBS to collect remaining protein. The flowthrough and wash were combined and concentrated (5K MWCO, 4 k rcf, 10 C, 1.5 h) and loaded on onto the HiLoad 26/600 Superdex 75 pg (Cytiva, #28-9893-34) to remove unconjugated dye. Fractions were collected and analyzed by SDS-PAGE and pooled and stored at −80° C.

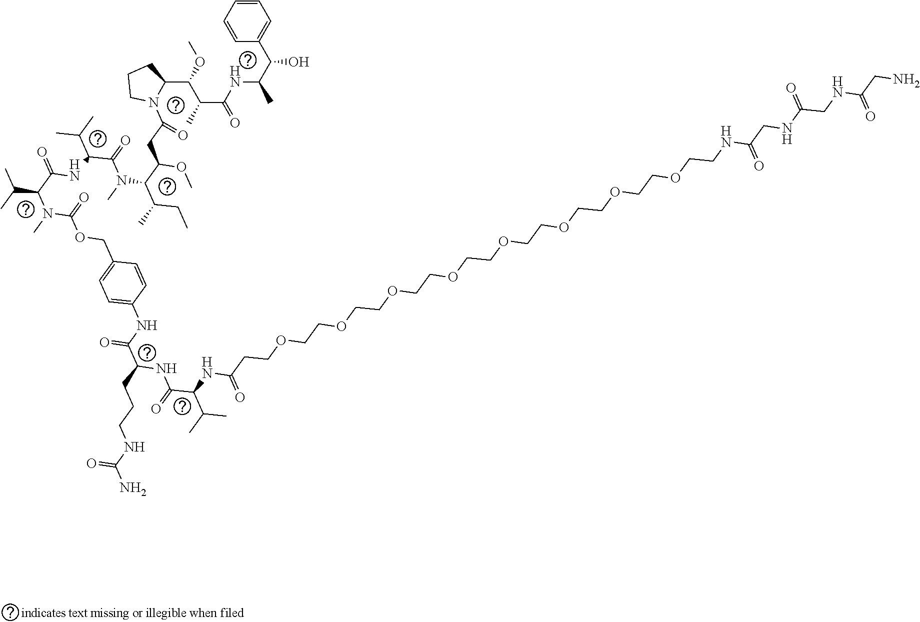 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCOCCOCCOCCOCCNC(=O)CNC(=O)CNC(=O)CN)C(C)C)cc1)C(C)C]

The C33 conjugates containing 52 and 54 position TYR or FSY (Table 21) were assessed for binding to the human breast cancer line BT-474 (HER2+). Proteins were diluted in FACS buffer (1×PBS+2% HI-FBS+5 mM EDTA) to 3 μM (1× final) and then serially diluted 3× to generate 8 concentration points for the assay, with the lowest concentration at 0.00384 μM (1× final).

The BT-474 cell line (ATCC HTB-20) was maintained in growth media consisting of RPMI-1640 (Thermo Scientific, 11875-903) supplemented with 10% heat inactivated fetal bovine serum (Thermo Fisher, FB-12) in a humidified environment with 5% CO2 at 37° C. Cells were harvested at exponential growth phase and resuspended in ice-cold pH 6.0 FACS buffer (PBS pH6.0/2% FBS/5 mM EDTA) at 1.0×10⁶/mL and aliquoted into a v-bottom 96-well plate (Corning 3897) at 100 μl/well. Cells were pelleted for 2 minutes at 2000 rpm then resuspended in titrations of test articles in pH6.0 FACS buffer, in duplicate at 100 μL/well. After incubation on ice for 2 hours, cells were pelleted at 2000 rpm for two minutes, then were washed with 200 μl FACS buffer twice. Cells were then resuspended in 100 μl of ice-cold FACS buffer pH 7.4 containing a 1:100 dilution of AF488 goat anti-alpaca VHH antibody (Jackson ImmunoResearch 128-547-232). After incubation on ice for 30 minutes, cells were pelleted at 2000 rpm for two minutes, washed with 200 μl FACS buffer twice, then were resuspended in 100 μL pH 7.4 FACS buffer and analyzed with NovoCyte flow cytometer (ACEA/Aglient).

Raw data (FCS files) were analyzed with FlowJo 10.7.2. FSC-A and SSC-A were used to identify total intact cells, then singlets were gated out with FSC-A against FSC-H. Geometric mean fluorescence intensity of AF488 of samples were used for further analysis and plotting. Geometric mean fluorescence intensity (GeoMean or GeoMFI) for the AF488 channel was used to calculate EC50 in GraphPad Prism (V9.3.1) using ‘log(agonist) vs. response—variable slope (four parameters). The test articles bound to BT-474 cells in a dose-dependent manner. Binding affinities for the test articles were measured from EC50 values after staining and flow cytometry of BT-474 cells as shown in Table 22.

TABLE 22
Binding affinity on BT-474 cells

| Test Article (Conjugate) | EC50 (nM) pH 6.0 |
|---|---|
| C33-54FSY | 4.6 |
| C33-54TYR | 2.5 |
| C33-52FSY | 49 |
| C33-52TYR | 160 |

The conjugates were then used in a cell based assay to measure the capacity of covalent sdAb conjugates to specifically deliver a cytotoxic payload to immortalized HER2-expressing tumor cell lines in vitro versus non-covalent sdAb conjugates. The BT-474 cell line was maintained as described above. Cells were harvested at exponential growth phase using TrypLE Express (Thermo Scientific, 14175-095), and seeded at 5000 cells/well in 100 μL growth media in black 96-well flat clear bottom plates (Costar, 3603). Cells were then incubated overnight in a humidified environment with 5% C02 at 37° C. to allow cells to adhere to the plate. The next day one plate was analyzed by CTG to determine background. Remaining plates were treated with C33-TYR and C33-FSY conjugates as follows: the conjugates listed in the Table 23 were diluted in growth media to 11× final concentration (1.1 μM), and then diluted in 5-fold increments to create a 10-point dilution series. A volume of 10 uL of each of the 11× test article dilution series was added to wells in triplicate to achieve a final concentration of 1×. One set of plates was returned to the incubator for 5 hours after which test articles were washed out by gently rinsing each well once with 100 μL growth media and then replacing the growth media (100 μL/well) and culturing the cells for 6 days in a humidified environment with 5% CO2 at 37° C. Another set of plates was incubated with test articles for the entire 6 days in a humidified environment with 5% C02 at 37° C. On day 6, to evaluate cell viability, growth media of all cultured plates was removed, CellTiter-Glo (Promega, G5737) was mixed with equal volume of PBS and added to all plates at 100 μl/well, then plates were shaken for one minute at 1000 rpm at room temp. Luminescence was then read on the Victor X5 plate reader (PerkinElmer) and reported as relative luminescence units (RLU). Viability was calculated in Excel (Microsoft® Excel® for Microsoft 365 MSO, Version 2202 Build 16.0.14931.20128, 64-bit) as % viability=100*(treated RLU-background RLU/untreated RLU-background RLU). Data were plotted as % viability versus concentration of test article in GraphPad Prism (V9.3.1) and IC50 was calculated using log [inhibitor] vs. response—variable slope (four parameters).

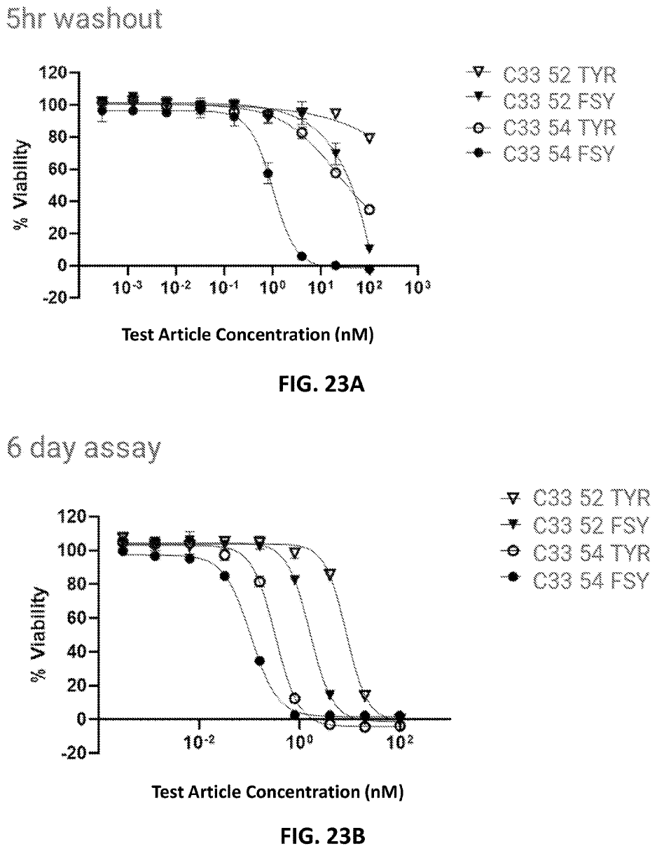

Results are shown in FIG. 23 and Table 23. FIG. 23A shows the 5 h washout and FIG. 23B shows the continuous 6 day exposure. All dose response analyses were performed in triplicate, symbols represent the mean and error bars represent STDEV. The FSY variants exhibited increased cytotoxicity relative to their TYR counterparts in the 5-hour washout study and in the 6-day continuous exposure experiment, showing the superior cellular potency of the covalent forms.

TABLE 23
IC50 values for cellular cytotoxicity
5 hour washout and 6 day exposure.

| Name | IC50 (nM) - 5 h | IC50 (nM) - 6 days |
|---|---|---|
| C33-52TYR | >100 | 8.417 |
| C33-52FSY | >100 | 1.599 |
| C33-54TYR | 21.56 | 0.3279 |
| C33-54 FSY | 1.00 | 0.106 |

### Example 24 Evaluation of Biparatopic Constructs—Binding

A series of monoparatopic sdAbs and biparatopic sdAbs with and without FSY were prepared as shown in Table 24 and compared for their ability to crosslink a PSMA target. Proteins were expressed following the methods generally of Example 2, with the modifications noted in Example 19.

TABLE 24
Mono- and Bi-paratopic constructs

| Compound ID | Construct |
|---|---|
| C34-TYR | C2-54TYR-C24-101TYR |
| C34-FSY | C2-54TYR-C24-101FSY |
| C35-TYR | C24-101TYR-C24-101TYR |
| C36-FSY | C24-101FSY-C24-101FSY |
| C2-54TYR | C2-54TYR |
| C24-101TYR | C24-101TYR |
| C24-101FSY | C24-101FSY |

Test articles were assessed for binding to PSMA+ LNCaP human prostate tumor cells using flow cytometry following the methods generally of Example 14, with the following modifications.

C34-TYR, C35-TYR and C2-54TYR were formulated with FACS buffer to 3 uM (lx final) and then serially diluted 5× to generate 12 concentration points for the assay, with the lowest concentration at 0.06 pM (1× final). In a separate experiment, C24-101TYR was formulated with FACS buffer and then serially diluted 5× to generate 8 concentration points for the assay, with the lowest concentration at 38.4 pM (1× final). LNCaP cells in the assay were at 120,000 cells/well for all except C24-101TYR which was at 160,000/well. After incubation with the test articles for two hours on ice, cells were washed twice with 200 μL/well FACS buffer per wash (500×g, 3 min) then incubated in the dark with 100 μL of ice-cold FACS buffer containing Alexa Fluor 488 AffiniPure Goat Anti-Alpaca IgG, VHH domain (Jackson ImmunoResearch, Code: 128-545-230) secondary antibody at 5 μg/ml on ice for 30 minutes. cells were then washed with FACS buffer twice (200 μL/wash) and resuspended in 100 μL FACS buffer for acquisition on a Novocyte 2060 (s/n 45-1-1511-2036-9) flow cytometer using NovoExpress software Version 1.5.0. Raw data (FCS 3.1 files) were analyzed to determine the geometric mean intensity of Alexa Fluor-488 (in the FITC channel) (FlowJo v.10.8.1, Windows). The mean and STDEV of the geometric mean intensity of Alexa Fluor-488 of samples was plotted versus log concentration of test article in GraphPad Prism (v 9.3.1). EC50s were calculated with the equation ‘log(agonist) vs. response—variable slope (four parameters). For visualizing data, binding curves were normalized in Prism for each test article, where the average of lowest value for each test article was set at 0% and the average highest value for each test article was set at 100%. Binding affinities for the test articles were measured from EC50 values after flow staining of LNCaP cells and are shown in Table 25. The biparatopic proteins bound to LNCaP cells in a dose dependent manner. The data suggest avidity effects in the binding of the biparatopic compounds, as affinities were observed to increase compared to the parental monomeric compounds.

TABLE 25
Binding affinity in LNCaP cells

| | C34-TYR | C35-TYR | C2-54TYR | C24-101TYR |
|---|---|---|---|---|
| EC50 | 2.30 nM | 12.26 nM | 6.75 nM | >500 nM |
| EC50 | 2.16 to | 11.48 to | 5.06 to | N/A |
| Range | 2.46 nM | 13.09 nM | 9.19 nM | |
| R squared | 0.99 | 0.99 | 0.99 | 0.99 |

### Example 25 Crosslinking of FSY-Modified Biparatopic Constructs to Target Cells

Crosslinking of C34-FSY and C36-FSY to cells expressing PSMA was compared with monovalent C24-101FSY. LNCaP cells in growth medium were seeded in 12-well plates coated with poly-L-lysine at the density of 250,000 cells/well. At 48 hours after seeding, the culture medium was removed and 0.4 mL culture medium containing the test compounds at the concentrations indicated in Table 26 were added for 1 or 6 hours. Cells were rinsed twice with 0.5 mL/well ice cold 1×PBS and 0.15 mL/well RIPA buffer with 1× protease inhibitor cocktail was added. After ten minutes incubation on ice, cell lysates were collected by scraping the plate with a trimmed 200 μL pipette tip. Lysates were transferred to 1.5 mL Eppendorf tubes and centrifugated at 12,000 rpm for 10 minutes at 4° C. After centrifugation, supernatant was transferred to new 1.5 mL Eppendorf tubes and 0.2 mL/well 0.25% EDTA-Trypsin (Gibco, Cat. No. 25200-056) was added.

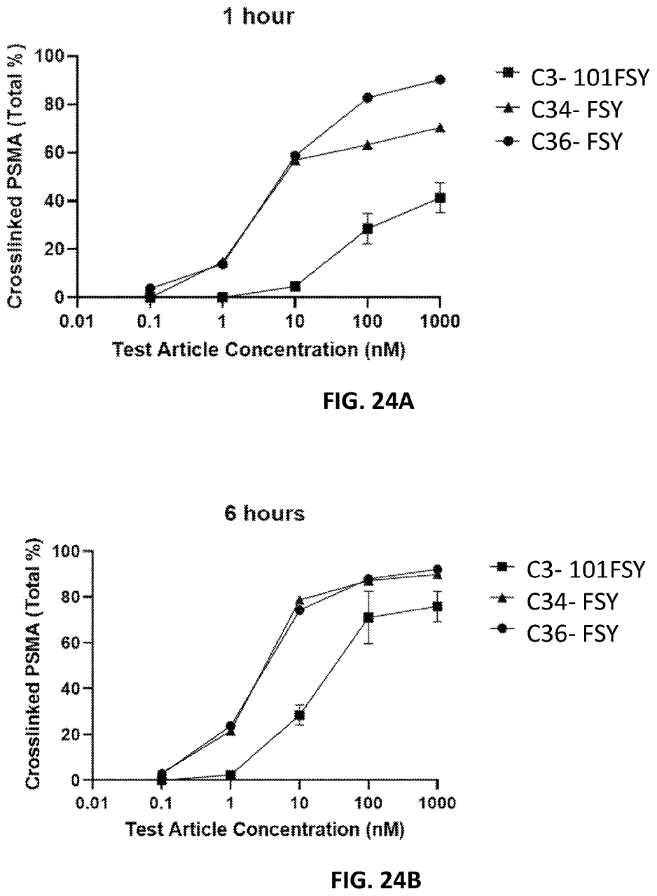

The denatured samples were analyzed by electrophoresis followed by western blot using the primary antibody anti-human PSMA (Invitrogen, Cat. No. 37-3900) or the internal standard anti-GAPDH (Cell Signaling Technology, Cat. No. CST-2118). Images were acquired by Azure Biosystem C600. GIMP 2.10.28 was used to process images. Density of bands in western blot was determined with ImageJ 1.51j8 following the instruction (https://imagej.nih.gov/ij/docs/guide/146-30.html #infobox:Densitometry, Section 30.13). Percentages of PSMA crosslinked versus total PSMA were calculated with following formula: Density of crosslinked PSMA/(density of crosslinked PSMA+density of un-crosslinked PSMA)×100%. Plots of density versus concentration of each test article (construct) are shown in FIGS. 24A and 24B.

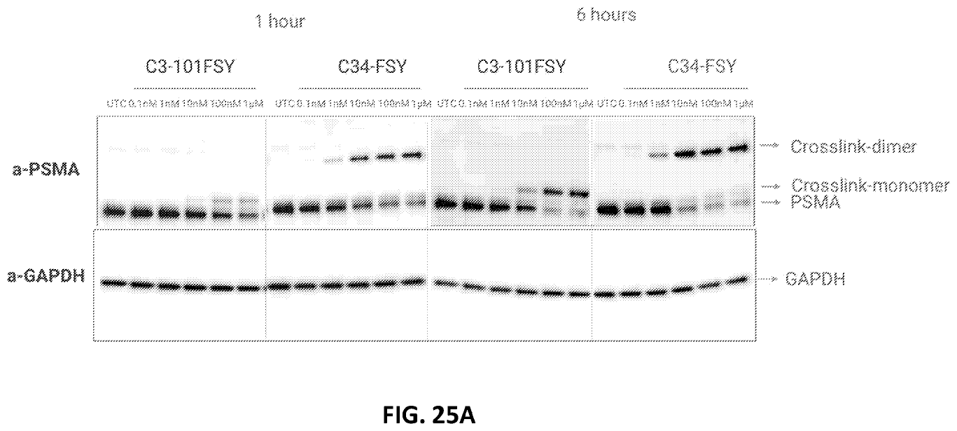

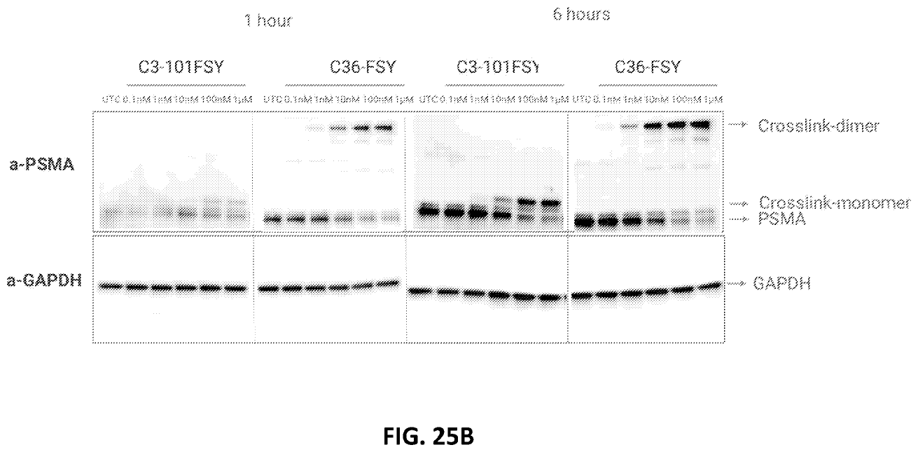

Western blot results are shown in FIGS. 25A and 25B. All 3 constructs tested were able to crosslink to PSMA. The biparatopic construct C34-FSY exhibited a crosslinked band that migrated with a higher apparent molecular weight than the monomeric, reflecting the increased mass of the complex, while the C36-FSY construct showed a crosslinked band with an even higher apparent molecular weight, indicative of crosslinking of two PSMA molecules. The intensity of the bands for all FSY compounds increased in a time and concentration dependent manner, indicating specific crosslinking. Quantitation of the crosslinking indicated that both biparatopic constructs exhibited more efficient and more potent crosslinking to PSMA than the monoparatopic construct. (Table 26 and FIGS. 25A and 25B).

TABLE 26
Crosslinking kinetics in LNCaP cells

| | Treatment | | | | | |
|---|---|---|---|---|---|---|
| | C24-101FSY | | C34-FSY | | C36-FSY | |
| | 1 hour | 6 hours | 1 hour | 6 hours | 1 hour | 6 hours |
| Concentration (nM) | Crosslinked PSMA (Total PSMA %) | | | | | |
| 0 | 0 | 0 | 0 | 0 | 0 | 0 |
| 0.1 | 0 | 0 | 0 | 3.2 | 3.8 | 2.8 |
| 1.0 | 0 | 2.4 | 14.8 | 21.5 | 13.8 | 23.7 |
| 10.0 | 4.6 | 28.45 | 56.9 | 78.7 | 58.7 | 74.2 |
| 100.0 | 28.5 | 71.0 | 63.3 | 87.1 | 82.7 | 87.9 |
| 1000.0 | 41.4 | 75.85 | 70.4 | 89.9 | 90.3 | 92 |

### Example 26 Biparatopic Conjugates with Cytotoxic Payloads

Test articles containing cytotoxic payloads conjugated to biparatopic sdAb compounds are made following the methods generally described in Examples 24 and 25 with the following modifications. To enable cysteine-maleimide conjugation of a PEG8-VC-PABC-MMAE linker payload to the biparatopic compounds, the plasmid sequence encoding compounds C34-FSY or C34-TYR is modified to contain a cys residue at or near the C-terminus, the compound is expressed to incorporate FSY or TYR at the 101 position, conjugated and purified.

To enable sortag-mediated conjugation of a PEG8-VC-PABC-MMAE linker payload to the biparatopic compound, the sequence encoding compounds C34-FSY or C34-TYR is modified to contain a C-terminal sortase recognition and His tag sequence, and the compound is expressed to incorporate FSY or TYR at the 101 position, conjugated and purified.

The PC3pip cell line engineered to express PSMA and PC3flu PSMA negative cell lines are maintained, seeded and prepared for the study as described in Example 22. The biparatopic sdAb-MMAE conjugates are diluted in growth media to 50× final concentration and diluted in 3-fold increments to create a 10-point dilution series. Each of the test article dilution series are added to wells in triplicate to achieve a final concentration of 1× (top concentrations) for each series.

One set of plates is returned to the incubator for 5 hours after which test articles are washed out by gently rinsing each well once with 100 μL growth media and then replacing the growth media (100 μL/well) and culturing the cells for 6 days in a humidified environment with 5% CO2 at 37° C. Another set of plates is incubated with test articles for the entire 6 days in a humidified environment with 5% CO2 at 37° C. On day 6, to evaluate cell viability, growth media of all cultured plates is removed, CellTiter-Glo (Promega, G5737) is mixed with equal volume of PBS and added to all plates at 100 μl/well, then plates are shaken for one minute at 1000 rpm at room temp. Luminescence is then read on the Victor X5 plate reader (PerkinElmer) or similar and reported as relative luminescence units (RLU). Viability is calculated in Excel or similar program as % viability=100*(treated RLU-background RLU/untreated RLU-background RLU). Data are plotted as % viability versus concentration of test article in GraphPad Prism (V9.3.1) or similar program and IC50 is calculated using log [inhibitor] vs. response—variable slope (four parameters).

### Example 27 Mouse Xenograft Model for Treatment of PSMA+ Tumors

To evaluate the effect of the tyrosine and FSY-containing monoparatopic and biparatopic conjugates in the control of tumor growth in a mouse xenograft tumor model, the cell lines LNCaP (PSMA+) and PC3 (PSMA−) are employed. Cell lines are maintained, expanded, and implanted into Balb/C nude mice as described in Examples 20 and 21. When tumor sizes reach ~200 mm³, the biparatopic C34-TYR or C34-FSY conjugates (Example 25), the monoparatopic C26-54TYR or C26-54FSY conjugates (Example 22) or vehicle, are injected through the tail vein at different doses and schedules, such as 5 mg/kg daily for 7 days. Tumor sizes are measured daily via caliper measurements to track tumor growth over time. Animals bearing tumors that reach 1000 mm³ are sacrificed, and survival time is recorded.

### Example 28—EGFR-Targeted Biparatopic Construct

An EGFR-targeted sdAb with FSY (C4-109FSY, SEQ ID NO: 17) and a biparatopic construct with two EGFR-targeted sdAbs joined by a linker (C4-109 TYR or FSY)-L1-C5, hereafter referred to as C37-TYR or C37-FSY, SEQ ID NO: 69) were constructed, cloned, and expressed as described in Example 2. The constructs were synthesized with a pelB leader sequence (SEQ ID NO: 15) that was cleaved off from the mature protein, and six C-terminal histidines were used for His-Tag purification. The biparatopic construct C37 contains a GGGGSGGGGS (SEQ ID NO: 14) linker between the first sdAb C4, and the second sdAb C5.

The FSY-modified sdAbs were assessed for kinetics of EGFR crosslinking. The proteins were incubated with EGFR at an 8:1 molar ratio (EGFR final concentration was 0.125 mg/mL, 1.25 μM). Samples were taken at time points from 0-180 minutes and the percentage of EGFR cross-linked assessed by SDS-PAGE. The crosslinking band percentage was calculated by quantifying the sdAb-EGFR crosslinking band and the EGFR band intensity was quantified using Image J.

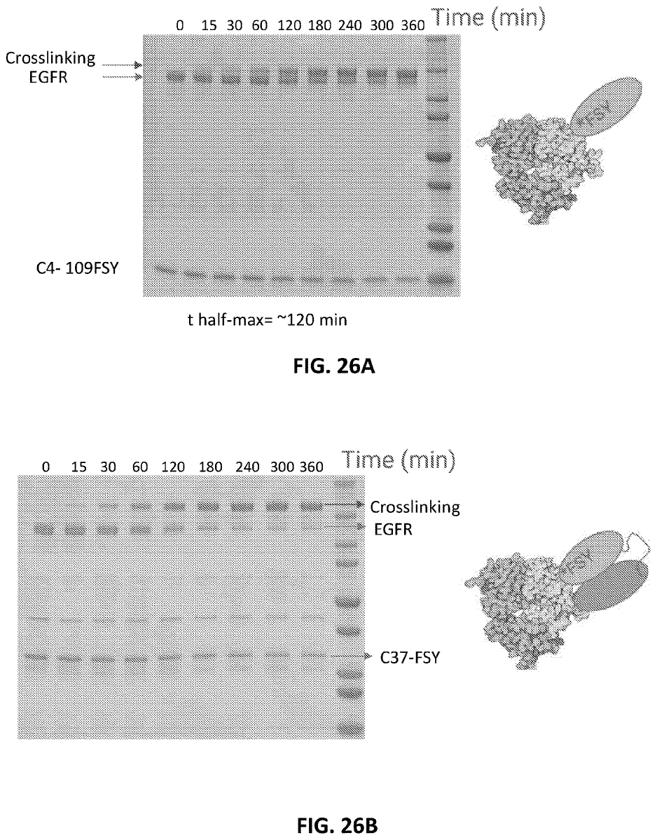

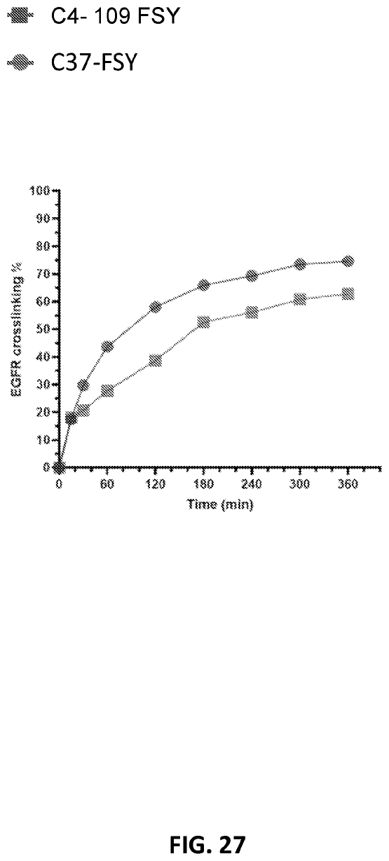

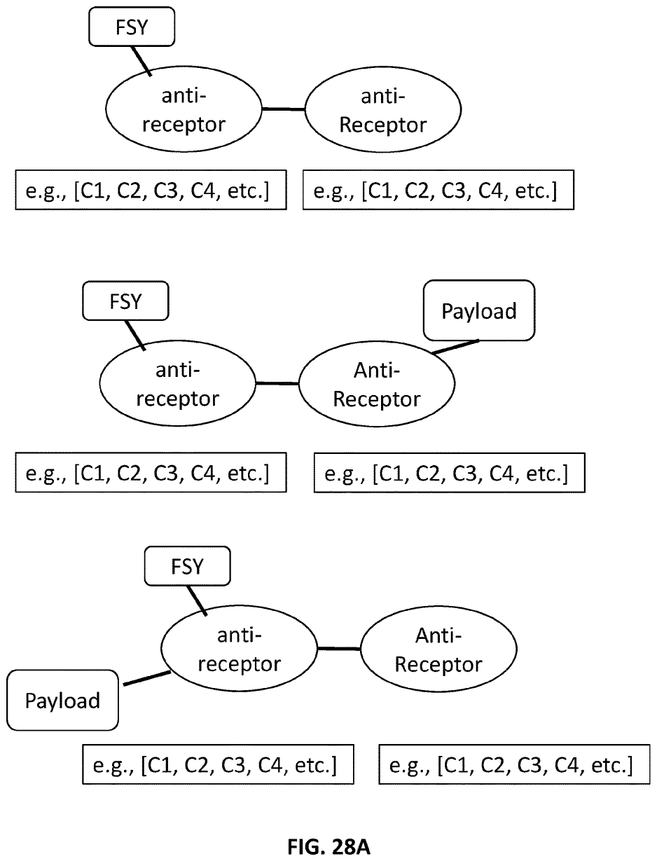

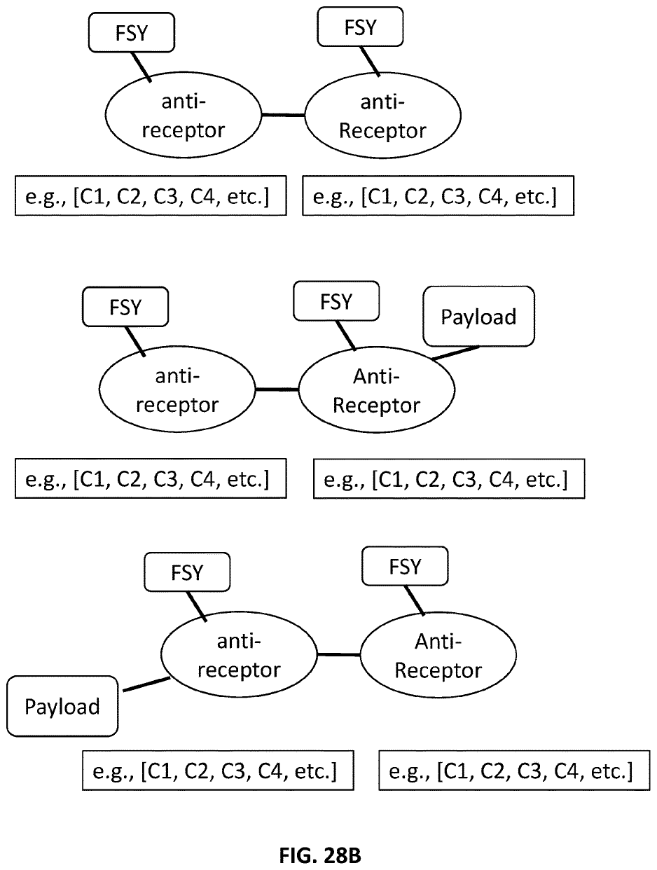

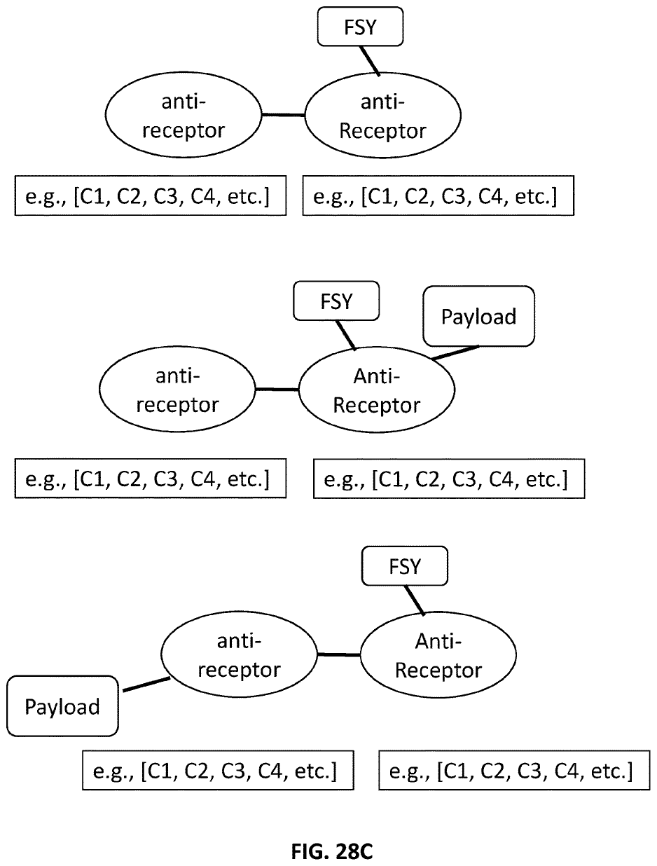

Samples were taken from time zero through 360 minutes. The kinetics of the coupling are shown in FIGS. 26-27. The biparatopic construct was compared to a mono-paratopic construct comprising only the sdAb C4 with the FSY modification (C4-109FSY). As shown in FIG. 27, the biparatopic construct C37-FSY coupled to EGFR more rapidly than the monoparatopic C4-109FSY sequence, as measured by a reduced time to half-maximal cross-linking compared to the mono-paratopic construct.

### Example 29: Construction of Hetero-Multispecific Binding Conjugate

Hetero-bispecific FSY-containing compounds that target multiple distinct antigens are constructed having a sdAb for a target (“target sdAb1”), such as a tumor cell antigen, joined by a linker to a sdAb targeted to a different tumor cell antigen (“target sdAb2”) that is expressed on the same cell. In each case, the target sdAb1, target sdAb2, or both, may contain FSY. The bispecific constructs are then cloned into pBAD vector and expressed as described in Example 2.

Hetero-bispecific FSY-containing compounds targeting the extracellular domains of EGFR and Her3 are created by linking the C4-109FSY (or variant thereof) from Example 2 with a sdAb targeting Her3 (without FSY). An alternative construct is generated that includes a sdAb targeting Her3 (with FSY) linked to C4 (without FSY). The constructs are designed by adding a linker containing repeats of the sequence GGGGS or similar between the two sdAb amino acid sequences. The bispecific constructs are then cloned into pBAD vector and expressed as described in Example 2. An example anticipated amino acid sequence for the bispecific construct is shown below.

MKYLLPTAAAGLLLLAAQPAMAMGQVQLVQSGGGLVQAGGSLRLSCAFSG
RTFSMYTMGWFRQAPGKEREFVAANRGRGLSPDIADSVNGRFTISRDNAK
NTLYLQMDSLKPEDTAVYYCAADLQYGSSWPQRSSAEYDYWGQGTTVTVS
SGGGGSGGGGSGGGGSQVKLEESGGGSVQTGGSLRLTCAASGRTSRSYGM
GWFRQAPGKEREFVSGISWRGDSTGYADSVKGRFTISRDNAKNTVDLQMN
SLKPEDTAIYYCAAAAGSAWYGTLXEYDYWGQGTQVTVSSHHHHHH

X indicates the position of the FSY incorporation site; bolded N terminal residues are the pelB leader sequence that are cleaved off from the mature protein. The C-terminal 6 histidines comprise a His-Tag used for affinity purification. Other bi-specific constructs for generation may include Her3-EGFR, EGFR-Her2, Her2-Her3, Her3-Her3 (same or different epitopes), EGFR-cMET, and EGFR-CEA.

Conjugates using bispecific FSY-modified sdAbs are prepared via engineering a unique conjugation site for chemical coupling of linker/payloads. The bispecific FSY-modified constructs described above are modified to contain a free Cys residue at the C-terminus of the construct or at alternative permissive sites within the construct that, when modified, do not disrupt binding to the respective targets. Alternative methods may include sortase-mediated or trans-glutaminase mediated ligation of linker/payloads, NHS-ester conjugation to lysine residues, or other methods common in the field. Conjugates to linker payloads are generated to connect the targeting domain (sdAb or other binding protein), a linker, and a payload, which may include toxic payloads, chelated radiometals, peptide or protein toxins or other functional cytotoxic compounds.

The bispecific FSY-modified sdAbs or conjugates thereof are assessed for in vitro biochemical crosslinking with EGFR and Her3 respectively. The proteins are incubated with EGFR or Her3 receptor domains at an excess stoichiometric ratio, such as 5:1 or 8:1. Samples are incubated and the percentage of EGFR or Her3 that is cross-linked to the sdAb-FSY compound is assessed via gel shift using SDS-PAGE and quantitated by densitometry.

The bispecific FSY-modified sdAbs or conjugates thereof are assessed for simultaneous binding to EGFR and Her3 using ELISA. The extracellular domain of Her3 or EGFR is adsorbed to the surface of a microtiter plate and washed extensively. The plate is blocked for non-specific interactions and the bispecific FSY-modified sdAbs are added and incubated to allow binding. The well is then washed to remove unbound material and then the second receptor subunit is added and incubated to allow binding. The plate is again washed to remove un-bound compounds and the second receptor is detected using anti-receptor antibody. Alternatively, the second receptor subunit is added as a fusion or conjugate between the receptor domain and a detection system, such as HRP or a fluorescent dye and detected by standard colorimetric or fluorescence detection. As controls for specificity, addition of the first receptor, bi-specific compound, or second receptor is omitted from the procedure. Binding of the anti-receptor antibody may be detected via anti-species HRP secondary antibody, biotinylated anti-receptor antibody detected with streptavidin HRP, fluorescent dye conjugated secondary antibody, or other similar methods standard for ELISA detection.

The bispecific FSY-modified sdAbs or conjugates thereof are assessed for simultaneous binding to EGFR and Her3 using Surface Plasmon Resonance (SPR). The extracellular domain of Her3 or EGFR receptor are immobilized to the surface of a SPR chip via NHS-ester chemistry or other standard methods. The bispecific FSY-modified sdAb compounds are then associated with the receptor 1 surface before washing to remove unbound material. To assemble and detect the ternary complex, the second receptor subunit is then injected and signal is measured. As controls for specificity, addition of the first receptor, bi-specific compound, or second receptor is omitted from the procedure as some affinity may be observed between receptor subunits.

The bispecific FSY-modified sdAbs or conjugates thereof are assessed for specific binding to EGFR and Her3 positive cells. A series of different concentrations of the test article are administered to the cell, and the dose-dependent binding of the test article may be detected by flow cytometry, or by cell-based ELISA or similar methods. To determine the contribution of each receptor specificity to the observed binding signal, competitive antibodies (cold competitors) are added in large stoichiometric excess to antagonize test article engagement of EGFR, Her3, or both receptors. After incubation and washing to remove un-bound antagonist antibody, the concentration series of the bi-specific test articles are added to the cells and allowed to bind. After incubation and washing to remove un-bound test compounds, the test articles are detected by antibodies specific for the compound, such as anti-epitope tag or anti-sdAb antibodies or other methods. Binding of the anti-test sample antibody may be detected via flow cytometry, or by ELISA via anti-species HRP secondary antibody, biotinylated anti-receptor antibody detected with streptavidin HRP, fluorescent dye conjugated secondary antibody, or other similar methods standard for ELISA detection.

The bispecific FSY-modified sdAbs or conjugates thereof are assessed for in vitro delivery of a toxic payload in vitro specifically to tumor cells that express the target antigen or antigens. Constructs containing an engineered C-terminal Cys residue are coupled to a cytotoxic payload such as MMAE or other class of highly-cytotoxic compound using the general methods described above. Dose-response curves are generated and samples are incubated with SKBR3 and Colo320DM cells in vitro. As controls for specificity, a cross-reacting antibody to the first receptor, second receptor, or both is added to certain samples. After incubation for various periods of time, cells are washed to remove free compound, and cell viability is measured using assays such as Promega CytoxGreen, or other assays that measure cell viability via reporter compound detection, detection of viable cells by Presto Blue, CCK-8 or similar reagents, and/or measuring apoptosis via Annexin V-FITC, propidium iodide staining, or similar reagents.

The bispecific FSY-modified sdAbs or conjugates thereof are assessed for in vivo delivery of payload to tumor tissues. To compare the tumor exposure of constructs versus non-covalent sdAb conjugates, a fluorophore label is used as a proxy payload to enable tracking/biodistribution measurements over time. An sdAb construct with a single residue substituted to either Tyr or FSY is conjugated via engineered cysteine residues via maleimide chemistry, or other method described above, to a chemical fluorophore (Alexa680 or similar) and purified as described above. The fluorophore-labeled sdADC conjugate molecules is administered via tail vein IV injection in male nude mice bearing tumor antigen + or − xenograft tumors, and the biodistribution of the test articles is observed over time via whole-animal imaging using an AmiX, IVIS spectrum imager or similar. The tumor-specific and peripheral exposures is quantitated via image densitometry and compared between FSY and Tyr versions of the sdAb. In addition to imaging, tumor samples are collected and processed for gel-based detection methods, such as western blot or fluorescence imaging, to detect the crosslinked sdAb-receptor complex and free sdAb in tumor tissues over time after administration.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A conjugate comprising a targeting domain and a payload, wherein the targeting domain comprises at least one unnatural amino acid (UAA) residue, wherein the targeting domain is configured to bind to a target, and wherein the UAA residue is in sufficient proximity to form a covalent bond with the target when the targeting domain is bound.

2. The conjugate of claim 1, wherein the payload is attached to an amino acid at position n+x from the UAA residue, wherein n is the position of the amino acid and x is at least 1.

3. The conjugate of claim 1, wherein the payload is attached to an amino acid at position n−x from the UAA residue, wherein n is the position of the amino acid and x is at least 1.

4. The conjugate of any one of claims 1 to 3, wherein when the targeting domain is bound to the target, the UAA residue is within 5-20 angstroms of the target.

5. The conjugate of claim 4, wherein the targeting domain binds to a cell surface molecule.

6. The conjugate of claim 5, wherein the cell surface molecule is a tumor associated antigen.

7. The conjugate of any one of claims 1-6, wherein the UAA residue is configured to form a covalent bond with a histidine, lysine, or tyrosine residue of the target.

8. The conjugate of any one of claims 1-7, wherein the UAA residue comprises a fluoro sulfate moiety.

9. The conjugate of any one of claims 1-7, wherein the UAA residue comprises an aryl-fluoro sulfate moiety.

10. The conjugate of claim 9, wherein the UAA residue comprises Formula I:

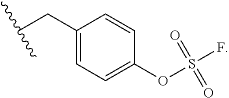 [SMILES: *Cc1ccc(OS(=O)(=O)F)cc1]

11. The conjugate of claim 9, wherein the UAA of the UAA residue has the structure:

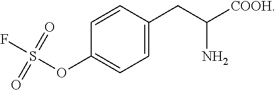 [SMILES: NC(Cc1ccc(OS(=O)(=O)F)cc1)C(=O)O]

12. The conjugate of claim 9, wherein the UAA of the UAA residue has the structure:

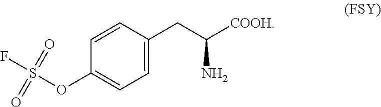 [SMILES: N[C@@H](Cc1ccc(OS(=O)(=O)F)cc1)C(=O)O]

13. The conjugate of claim 9, wherein the UAA residue comprises Formula II:

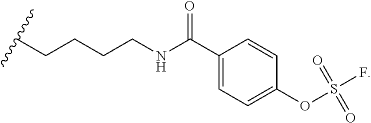 [SMILES: *CCCCNC(=O)c1ccc(OS(=O)(=O)F)cc1]

14. The conjugate of claim 9, wherein the UAA of the UAA residue has the structure:

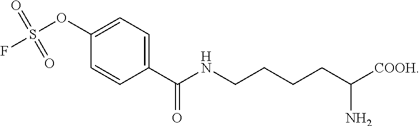 [SMILES: NC(CCCCNC(=O)c1ccc(OS(=O)(=O)F)cc1)C(=O)O]

15. The conjugate of claim 9, wherein the UAA of the UAA residue has the structure:

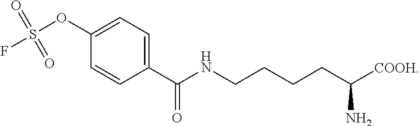 [SMILES: N[C@@H](CCCCNC(=O)c1ccc(OS(=O)(=O)F)cc1)C(=O)O]

16. The conjugate of claim 9, wherein the UAA residue comprises Formula III:

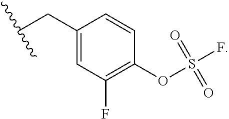 [SMILES: *Cc1ccc(OS(=O)(=O)F)c(F)c1]

17. The conjugate of claim 9, wherein the UAA of the UAA residue has the structure:

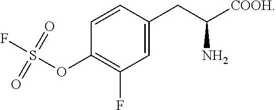 [SMILES: N[C@@H](Cc1ccc(OS(=O)(=O)F)c(F)c1)C(=O)O]

18. The conjugate of claim 9, wherein the UAA of the UAA residue has the structure:

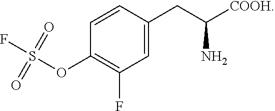 [SMILES: N[C@@H](Cc1ccc(OS(=O)(=O)F)c(F)c1)C(=O)O]

19. The conjugate of any one of claims 1-7, wherein the UAA of the UAA residue has a structure of Formula (IA): 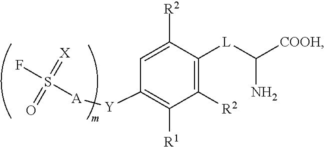
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, m is 1; when Y is —N═, m is 2.

20. The conjugate of claim 19, wherein the UAA of Formula (IA) has a structure of Formula (IA-a):

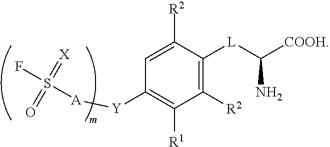

21. The conjugate of claim 19, wherein the UAA of Formula (IA) has a structure of Formula (IA-b):

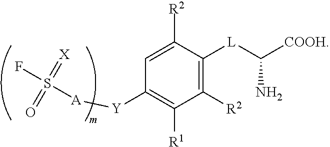

22. The conjugate of claim 19, wherein the UAA of Formula (IA) has a structure of Formula (IB):

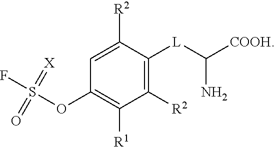

23. The conjugate of claim 19, wherein the UAA of Formula (IA) has a structure of Formula (IC):

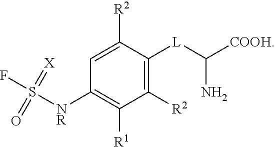

24. The conjugate of claim 19, wherein the UAA of Formula (IA) has a structure of Formula (ID): 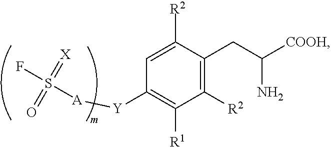
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

25. The conjugate of claim 19, wherein the UAA of Formula (IA) has a structure of Formula (IE): 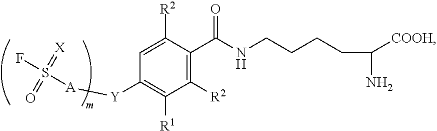
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

26. The conjugate of claim 19, wherein the UAA of Formula (IA) has a structure of Formula (IIA): 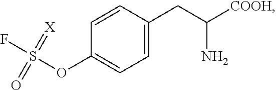
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

27. The conjugate of claim 19, wherein the UAA of Formula (IA) has a structure of Formula (IIB): 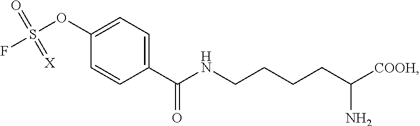
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

28. The conjugate of any one of claims 1-7, wherein the UAA of the UAA residue has a of 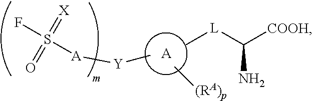
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NHR^{X}, N(R^{X})₂, or optionally substituted alkyl; each R^{X} is optionally substituted alkyl;
L is —(CH₂)ₚ— or —C(O)NH—(CH₂)ₚ—; p is an integer of 1 to 6; and
wherein when Y is a bond, —O— or —NR—, m is 1; when Y is —N═, m is 2.

29. The conjugate of any one of claims 1-28, further comprising at least one additional targeting domain.

30. The conjugate of any one of claims 1-29, wherein the payload comprises an imaging agent, a radioligand agent or a cytotoxic agent.

31. The conjugate of claim 30, wherein the cytotoxic moiety comprises a small molecule drug, or a chemotherapeutic drug.

32. The conjugate of claim 30, wherein the radioligand agent is selected from the group consisting of ³⁵S, ³H, ¹¹¹In, ¹¹²In, ¹⁴C, ¹⁸⁶Re, ¹⁸⁸Re, ³²P, ¹⁵³Sm, ¹⁷⁷Lu, ⁸⁶Y, ⁸⁸Y, ⁹⁰Y, ¹³¹I, ¹²³I, ¹²⁴I, ¹²⁵I, ¹⁴⁹Tb, ²¹¹At, ²¹²Pb/²¹²Bi, ²¹³Bi, ²²³Ra, ²²⁵Ac, ⁶⁴Cu, ⁶⁷Cu, and ²²⁷Th.

33. The conjugate of claim 31 or claim 32, wherein the radioligand agent further comprises a chelator.

34. The conjugate of claim 30 or claim 33, wherein the radioligand agent is selected from the group consisting of ^{99m}Tc, ¹³¹I, ²⁰¹Tl, ¹¹¹In, and ⁶⁷Ga.

35. The conjugate of any one of claims 1-34, wherein the payload is attached to the conjugate with a linker.

36. The conjugate of claim 35, wherein the linker comprises a polymer.

37. The conjugate of claim 35, wherein the linker is a cleavable or non-cleavable linker.

38. The conjugate of claim 35, wherein the linker is 0.01 kDa to 50 kDa.

39. The conjugate of claim 35, wherein the linker is 0.01 kDa to 10 kDa.

40. The conjugate of any one of claims 35-39, wherein the linker is linear, branched, multimeric, or dendrimeric.

41. The conjugate of claim 40, wherein the linker is a bifunctional or multifunctional linker or a bifunctional or multifunctional polymer.

42. The conjugate of any one of claims 35-41, wherein the linker comprises a water soluble polymer.

43. The conjugate of claim 42, wherein the water soluble polymer is polyethylene glycol (PEG).

44. The conjugate of claim 43, wherein the PEG has a molecular weight between 0.1 kDa and 2.5 kDa.

45. The conjugate of claim 43, wherein the PEG comprises 1-8 monomers.

46. The conjugate of any one of claims 1-45, wherein the targeting domain comprises an antibody, antibody fragment, or an antigen binding domain.

47. The conjugate of claim 46, wherein the targeting domain comprises an antigen binding domain, the conjugate comprises a CDR region, and the at least one UAA is within or in the proximity of the CDR region.

48. The conjugate of claim 47, wherein the UAA is comprised within the CDR region.

49. The conjugate of any one of claims 46-48, wherein the targeting domain comprises a single domain antibody (sdAb).

50. The conjugate of any one of claims 5-49, wherein the cell-surface molecule is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Muc1, ADAM-9, GPNMB, EDB fibronectin, tissue factor, GPNMB, FolRa, EphA2, ALPP, ALPPL2, MT1-MMP, CLDN18.2, CLDN6, CLDN9, pCadherin, CEACAM6, CD47, and EpCAM.

51. A conjugate comprising (i) a payload and (ii) an engineered single domain antibody (sdAb), comprising a sdAb having a CDR region and at least one unnatural amino acid (UAA) residue within or in the proximity of the CDR region, wherein the sdAb comprises any one of SEQ ID NOS: 1-4, or 16-64.

52. The conjugate of claim 51, wherein the UAA residue comprises a fluorosulfate moiety.

53. The conjugate of claim 52, wherein the UAA residue comprises an aryl-fluoro sulfate moiety.

54. The conjugate of claim 53, wherein the UAA residue comprises Formula I:

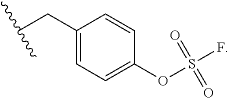 [SMILES: *Cc1ccc(OS(=O)(=O)F)cc1]

55. The conjugate of claim 53, wherein the UAA of the UAA residue has the structure:

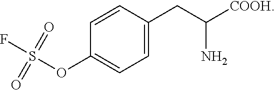 [SMILES: NC(Cc1ccc(OS(=O)(=O)F)cc1)C(=O)O]

56. The conjugate of claim 53, wherein the UAA of the UAA residue has the structure:

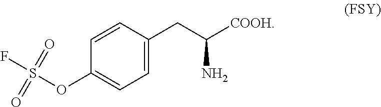 [SMILES: N[C@@H](Cc1ccc(OS(=O)(=O)F)cc1)C(=O)O]

57. The conjugate of claim 53, wherein the UAA residue comprises Formula II:

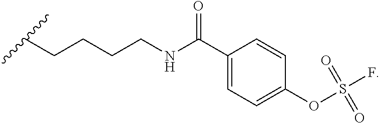 [SMILES: *CCCCNC(=O)c1ccc(OS(=O)(=O)F)cc1]

58. The conjugate of claim 53, wherein the UAA of the UAA residue has the structure:

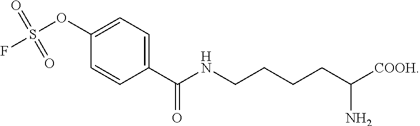 [SMILES: NC(CCCCNC(=O)c1ccc(OS(=O)(=O)F)cc1)C(=O)O]

59. The conjugate of claim 53, wherein the UAA of the UAA residue has the structure:

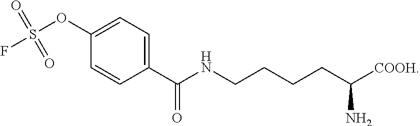 [SMILES: N[C@@H](CCCCNC(=O)c1ccc(OS(=O)(=O)F)cc1)C(=O)O]

60. The conjugate of claim 53, wherein the UAA residue comprises Formula III:

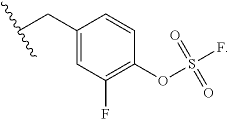 [SMILES: *Cc1ccc(OS(=O)(=O)F)c(F)c1]

61. The conjugate of claim 53, wherein the UAA of the UAA residue has the structure:

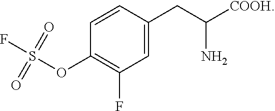 [SMILES: NC(Cc1ccc(OS(=O)(=O)F)c(F)c1)C(=O)O]

62. The conjugate of claim 53, wherein the UAA of the UAA residue has the structure:

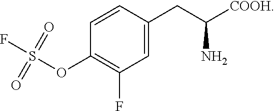 [SMILES: N[C@@H](Cc1ccc(OS(=O)(=O)F)c(F)c1)C(=O)O]

63. The conjugate of claim 53, wherein the UAA of the UAA residue has a structure of Formula (IA): 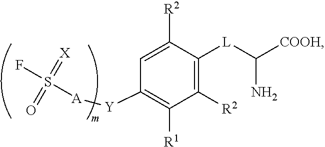
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, m is 1; when Y is —N═, m is 2.

64. The conjugate of claim 63, wherein the UAA of Formula (IA) has a structure of Formula (IA-a):

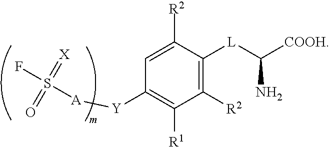

65. The conjugate of claim 63, wherein the UAA of Formula (IA) has a structure of Formula (IA-b):

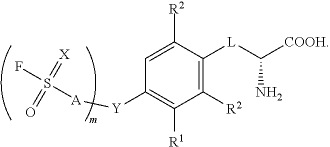

66. The conjugate of claim 63, wherein the UAA of Formula (IA) has a structure of Formula (IB):

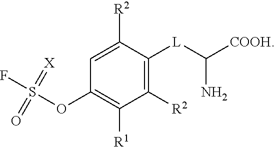

67. The conjugate of claim 63, wherein the UAA of Formula (IA) has a structure of Formula (IC):

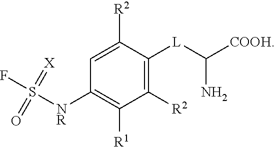

68. The conjugate of claim 63, wherein the UAA of Formula (IA) has a structure of Formula (ID): 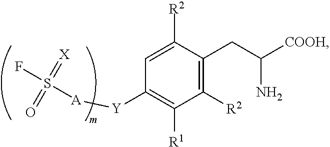
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

69. The conjugate of claim 62, wherein the UAA of Formula (IA) has a structure of Formula (IE): 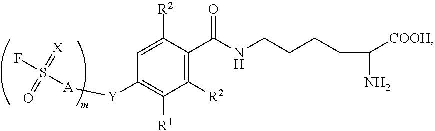
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

70. The conjugate of claim 63, wherein the UAA of Formula (IA) has a structure of Formula (IIA): 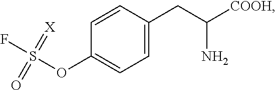
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

71. The conjugate of claim 63, wherein the UAA of Formula (IA) has a structure of Formula (IIB): 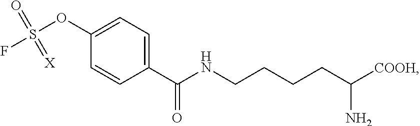
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

72. The conjugate of claim 51, wherein the UAA of the UAA residue has a structure of 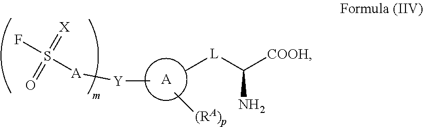
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NRX, N(R^{X})₂, or optionally substituted alkyl; each R^{X} is optionally substituted alkyl; and
wherein when Y is a bond, —O— or —NR—, m is 1; when Y is —N═, m is 2.

73. The conjugate of any one of claims 51-72, wherein the engineered sdAb comprises SEQ ID NO:1, and wherein the UAA is comprised in any one of SEQ ID NOS:5, 6, and 7.

74. The conjugate of claim 73, wherein the UAA is present at an amino acid position selected from the group consisting of 26, 28, 29, 30, 99, 102, 103, 105, 108, 110, 111, 112, 113, 114, and 115 relative to SEQ ID NO:1.

75. The conjugate of any one of claims 51-72, wherein the engineered sdAb comprises SEQ ID NO:2, and wherein the UAA is comprised in any one of SEQ ID NOS:8, 9, and 10.

76. The conjugate of claim 75, wherein the UAA is present at an amino acid position selected from the group consisting of 50, 52, 53, 54, 56, 58, and 100 relative to SEQ ID NO:2.

77. The conjugate of any one of claims 51-72, wherein the engineered sdAb comprises SEQ ID NO:3, and wherein the UAA is comprised in any one of SEQ ID NOS:11, 12, and 13.

78. The conjugate of claim 77, wherein the UAA is present at an amino acid position selected from the group consisting of 58, 62, 101, 103, and 107 relative to SEQ ID NO:3.

79. The conjugate of any one of claims 51-72, wherein the engineered sdAb comprises SEQ ID NO:16, and wherein the UAA is present at an amino acid position of 109 relative to SEQ ID NO: 16.

80. The conjugate of any one of claims 51-72, wherein the engineered sdAb comprises SEQ ID NO:25, and wherein the UAA is present at an amino acid position selected from the group consisting of 52, 53, 54, 55, 56, 58, 60, 62, and 64 relative to SEQ ID NO: 23.

81. The conjugate of any one of claims 51-72, wherein the engineered sdAb comprises SEQ ID NO: 26, and wherein the UAA is present at an amino acid position selected from the group consisting of 53, 55, 56, 57, 58, 60, 64, and 67 relative to SEQ ID NO: 24.

82. The conjugate of any of claim 51-81, wherein the payload is not linked via the UAA sidechain.

83. The conjugate of any of claim 51-82, wherein the payload comprises an imaging agent, a radioligand agent or a cytotoxic agent.

84. The conjugate of claim 83, wherein the cytotoxic moiety comprises a small molecule drug or a chemotherapeutic drug.

85. The conjugate of claim 83, wherein the radioligand agent is selected from the group consisting of ³⁵S, ³H, ¹¹¹In, ¹¹²In, ¹⁴C, ¹⁸⁶Re, ¹⁸⁸Re, ³²P ¹⁵³Sm, ¹⁷⁷Lu, ⁸⁶Y, ⁸⁸Y, ⁹⁰Y, ¹³¹I, ¹²³I, ¹²⁴I, ¹²⁵I, ¹⁴⁹Tb, ²¹¹At, ²¹²Pb/²¹²Bi, ²¹³Bi, ²²³Ra, ²²⁵Ac, ⁶⁴Cu, ⁶⁷Cu, and ²²⁷Th.

86. The conjugate of claim 83 or claim 85, wherein the radioligand agent further comprises a chelator.

87. The conjugate of claim 83, wherein the imaging agent comprises a fluorophore, or a radioligand agent selected from the group consisting of ^{99m}Tc, ¹³¹I, ²⁰¹Tl, ¹¹¹In, and ⁶⁷Ga.

88. A method comprising administering the conjugate of any one of claims 1-87, wherein the conjugate covalently binds a target on the surface of a cell.

89. The method of claim 88, wherein the cell comprises a tumor cell.

90. The method of claim 89, wherein the conjugate kills or inhibits the growth of the tumor cell.

91. The method of any one of claims 88-90, wherein the target is a tumor associated antigen.

92. The method of any one of claims 88-91, wherein the target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, EphA2, ALPP, ALPPL2, MT1-MMP, CLDN18.2, CLDN6, CLDN9, pCadherin, CEACAM6, CD47, and EpCAM.

93. A method of treating a disease or condition comprising administering the conjugate of any one of claims 1-92, to a subject in need thereof.

94. The method of claim 93, wherein the disease comprises one or more of PRAD (Prostate adenocarcinoma), mCRPC (Metastatic castration resistant prostate cancer), solid tumors (neovasculature), LUAD (Lung adenocarcinoma), LUSC (Lung squamous cell carcinoma), HNSC (Head and Neck squamous cell carcinoma), THCA (Thyroid carcinoma), ESCA (Esophageal Carcinoma) STAD (Stomach adenocarcinoma), GIST (Gastrointestinal Stromal Tumor), COAD (Colon Adenocarcinoma), READ (Rectum adenocarcinoma), SARC (Sarcoma), SCLC (small cell lung cancer), MESO (Mesothelioma), PAAD (Pancreatic adenocarcinoma), B-Cell malignancies, T-Cell malignancies, ALL (Acute Lymphoblastic Leukemia), NHL (Non-Hodgkin Lymphoma), HL (Hodgkin Lymphoma), CLL (Chronic Lymphocytic Leukemia), AML (Acute Myeloid Leukemia), MDS (Myelodysplastic syndromes), MSI-high tumors, SKCM (Skin cutaneous melanoma), UVM (Uveal Melanoma), DLBC (Diffuse Large B-cell Lymphoma), endometrial cancer, CESC (Cervical Carcinoma), bone cancer, BLCA (Bladder Urothelial Cancer), BRCA (Breast Invasive Carcinoma), TNBC (triple negative breast cancer), LIHC (Liver hepatocellular carcinoma), OV (Ovarian serous cystadenocarcinoma), UCEC (Uterine Corpus Endometrial Carcinoma), NE-PCa (Neuroendocrine prostate cancer), GBM (Glioblastoma multiforme), and KIRC (Kidney Renal Clear Cell carcinoma).

95. A method of manufacturing the conjugates of any one of claims 1-87, comprising:
(a) generating the targeting domain comprising at least one unnatural amino acid; and
(b) conjugating the targeting domain to a payload, optionally via a linker.

96. The method of claim 95, wherein the targeting domain comprising at least one unnatural amino acid is synthesized in-vivo.

97. The method of claim 96, wherein generating comprises use of an orthogonal tRNA synthetase/suppressor tRNA pair.

98. The method of claim 97, wherein generating comprises the orthogonal tRNA synthetase/suppressor tRNA pair is derived from pyrrolysine tRNA synthetase/tRNA^{Pyl}.

99. The method of claim 97, wherein the orthogonal tRNA synthetase comprises SEQ ID NOs: 84, 87, 92, or variant thereof.

100. A method of delivering a cytotoxic payload to a cell comprising administering the conjugate of any one of claims 30-33, 35-50 or 83-86, wherein the conjugate covalently binds a target on the surface of the cell, thereby delivering the cytotoxic payload.

101. The method of claim 100, wherein the cell is a tumor cell.

102. The method of claim 100, wherein the cell is comprised within a tumor microenvironment.

103. The method of claim 100, wherein the cell is comprised within a mammalian subject.

104. The method of claim 100, wherein the cell is comprised within a human subject.

105. The method of any one of claims 100-104, wherein the conjugate kills or inhibits the growth of the tumor cell.

106. The method of any one of claims 100-105, wherein the target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, ALPP, ALPPL2, MT1-MMP, CLDN18.2, CLDN6, CLDN9, pCadherin, CEACAM6, CD47, and EpCAM.

107. The method of any one of claims 100-106, wherein the human subject suffers from or is diagnosed with a disease or condition selected from the group consisting of PCa (prostate cancer), CRPCa (castration resistant prostate cancer), solid tumors (neovasculature), NSCLC (non-small cell lung cancer), HNSCC (head and neck squamous cell carcinoma), ESCC (esophageal cancer) GC (gastric cancer), CRC (colorectal cancer), SCLC (small cell lung cancer), MPM (mesothelioma), PDAC (Pancreatic ductal adenocarcinoma), ALL (Acute Lymphoblastic Leukemia), AML (Acute Myeloid Leukemia), MDS (Myelodysplastic syndromes), MSI-high tumors, melanoma, DLBCL (diffuse large B cell lymphoma), endometrial cancer, cervical cancer, bladder cancer, BrCa (breast cancer), TNBC (triple negative breast cancer), NE-PCa (Neuroendocrine prostate cancer), GBM (glioblastoma), and RCC (Renal cell carcinoma).

108. A conjugate comprising (i) a first targeting domain, (ii) a second targeting domain, and optionally (iii) a payload, wherein the first targeting domain comprises at least one first unnatural amino acid (UAA) whereby the first targeting domain is capable of covalently binding to a first target at the site of the UAA and the second targeting domain is configured to bind a second target.

109. The conjugate of claim 108, wherein the first target and the second target are on the same cell.

110. The conjugate of claim 108 or claim 109, wherein the first targeting domain comprises an antibody, antibody fragment, or an antigen binding domain.

111. The conjugate of claim 110, wherein the first targeting domain comprises a single domain antibody (sdAb).

112. The conjugate of any one of claims 108-111, wherein the first UAA is comprised within or within proximity of a region of the first targeting domain that interfaces with the first target.

113. The conjugate of any one of claims 108-112, wherein the second targeting domain comprises an antibody, antibody fragment, or an antigen binding domain.

114. The conjugate of claim 113, wherein the second targeting domain comprises a single domain antibody (sdAb).

115. The conjugate of any one of claims 108-114, wherein the first targeting domain and the second targeting domain are linked to form a fusion protein.

116. The conjugate of any one of claims 108-114, wherein the first targeting domain and the second targeting domain are linked by chemical conjugation.

117. The conjugate of claim 115 or claim 116, wherein the first targeting domain and the second domain are linked by a linker.

118. The conjugate of any one of claims 108-117, wherein the first targeting domain and the second targeting domain bind the same target.

119. The conjugate of claim 118, wherein the first targeting domain and the second targeting domain bind to different epitopes of the same target.

120. The conjugate of claim 118, wherein the first targeting domain and the second targeting domain bind to the same epitope of the same target.

121. The conjugate of any one of claims 118-120, wherein the same target is a monomer.

122. The conjugate of any one of claims 118-120, wherein the same target is a multimeric molecule.

123. The conjugate of any one of claims 108-117, wherein the first targeting domain and the second targeting domain bind different targets.

124. The conjugate of any one of claims 108-123, wherein the first target is a first cell surface molecule.

125. The conjugate of any one of claims 108-124, wherein the second target is a second cell surface molecule.

126. The conjugate of any one of claims 108-125, wherein the at least one first UAA comprises a fluoro sulfate moiety.

127. The conjugate of any one of claims 108-125, wherein the at least one UAA comprises an aryl-fluoro sulfate moiety.

128. The conjugate of claim 127, wherein the at least one first UAA comprises Formula I:

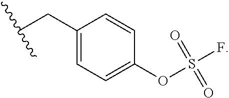 [SMILES: *Cc1ccc(OS(=O)(=O)F)cc1]

129. The conjugate of claim 127, wherein the at least one first UAA has the structure:

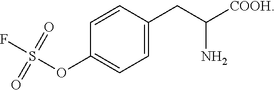 [SMILES: NC(Cc1ccc(OS(=O)(=O)F)cc1)C(=O)O]

130. The conjugate of claim 127, wherein the at least one first UAA has the structure:

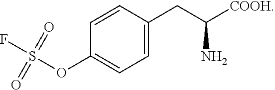 [SMILES: N[C@@H](Cc1ccc(OS(=O)(=O)F)cc1)C(=O)O]

131. The conjugate of claim 127, wherein the at least one first UAA comprises Formula II:

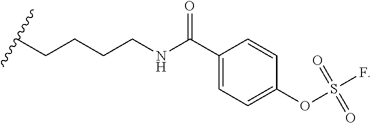 [SMILES: *CCCCNC(=O)c1ccc(OS(=O)(=O)F)cc1]

132. The conjugate of claim 127, wherein the at least one first UAA has the structure:

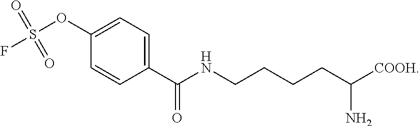 [SMILES: NC(CCCCNC(=O)c1ccc(OS(=O)(=O)F)cc1)C(=O)O]

133. The conjugate of claim 127, wherein the at least one first UAA has the structure:

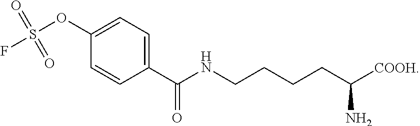 [SMILES: N[C@@H](CCCCNC(=O)c1ccc(OS(=O)(=O)F)cc1)C(=O)O]

134. The conjugate of claim 127, wherein the at least one first UAA comprises Formula III:

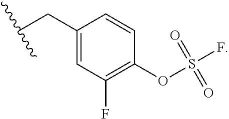 [SMILES: *Cc1ccc(OS(=O)(=O)F)c(F)c1]

135. The conjugate of claim 127, wherein the at least one first UAA has the structure:

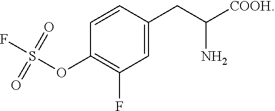 [SMILES: NC(Cc1ccc(OS(=O)(=O)F)c(F)c1)C(=O)O]

136. The conjugate of claim 127, wherein the at least one first UAA has the structure:

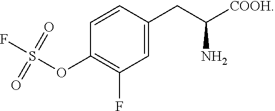 [SMILES: N[C@@H](Cc1ccc(OS(=O)(=O)F)c(F)c1)C(=O)O]

137. The conjugate of any one of claims 108-125, wherein the at least one first UAA has a structure of Formula (IA): 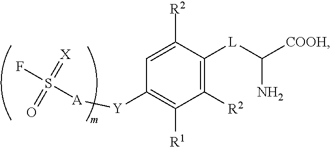
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, m is 1; when Y is —N═, m is 2.

138. The conjugate of claim 137, wherein the at least one first UAA has a structure of Formula (IAa):

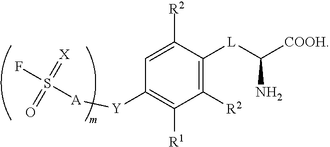

139. The conjugate of claim 137, wherein the at least one first UAA has a structure of Formula (IAb):

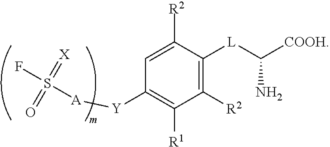

140. The conjugate of claim 137, wherein the at least one first UAA has a structure of Formula (IB):

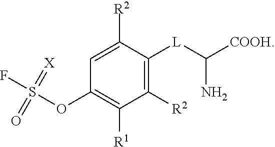

141. The conjugate of claim 137, wherein the at least one first UAA has a structure of Formula (IC):

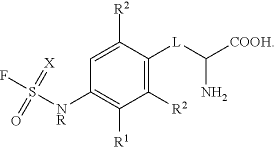

142. The conjugate of claim 137, wherein the at least one first UAA has a structure of Formula (ID): 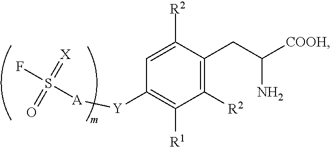
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

143. The conjugate of claim 137, wherein the at least one first UAA has a structure of Formula (IE): 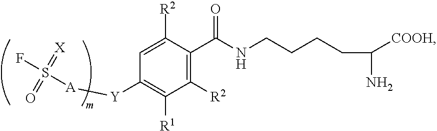
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

144. The conjugate of claim 137, wherein the at least one first UAA has a structure of Formula (IIA): 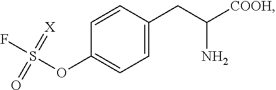
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

145. The conjugate of claim 137, wherein the at least one first UAA has a structure of Formula (IIB): 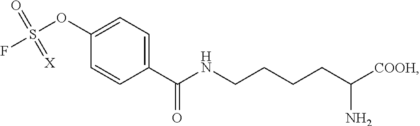
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

146. The conjugate of any one of claims 108-125, wherein the at least one first UAA has a structure of 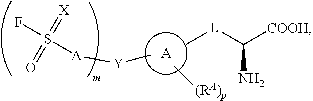
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NHR^{X}, N(R^{X})₂, or optionally substituted alkyl; p is 0, 1, 2, 3 or 4; each R^{X} is optionally substituted alkyl;
L is —(CH₂)ₚ— or —C(O)NH—(CH₂)ₚ—; p is an integer of 1 to 6; and
wherein when Y is a bond, —O— or —NR—, m is 1; when Y is —N═, m is 2.

147. The conjugate of any one of claims 124-146, wherein the first cell surface molecule is selected from the group consisting of PSMA, EGFR, HER2, HER3, PD-L1, EphA4, Fibronectin ED-B, EpCAM, CCR4, CD25, VEGF, VEGFR2, endo180, LIV-1, PTK7, CDH6, CSP-1, CD71, PRLR, SEZ6, DLL1, NOTCH3 rec, NaPi2b, DLK1, Muc16, cMET, CEA, LRP5, and LRP6.

148. The conjugate of any one of claims 125-147, wherein the second cell surface molecule is selected from the group consisting of PSMA, EGFR, HER2, HER3, PD-L1, EphA4, Fibronectin ED-B, EpCAM, CCR4, CD25, VEGF, VEGFR2, endo180, LIV-1, PTK7, CDH6, CSP-1, CD71, PRLR, SEZ6, DLL1, NOTCH3 rec, NaPi2b, DLK1, Muc16, cMET, CEA, LRP5, and LRP6.

149. The conjugate of any one of claims 108-148, wherein the second targeting domain comprises a second UAA, whereby the second targeting domain is capable of covalently binding to the second target at the site of the second UAA.

150. The conjugate of any claim 149, wherein the second UAA is different from the at least one UAA comprised in the first targeting domain.

151. The conjugate of claim 149, wherein the second UAA is the same as the at least one UAA comprised in the first targeting domain.

152. The conjugate of any one of claims 149-151, wherein the second UAA comprises a fluoro sulfate moiety.

153. The conjugate of any one of claims 149-151, wherein the second UAA comprises an aryl-fluoro sulfate moiety.

154. The conjugate of claim 153, wherein the second UAA comprises Formula I:

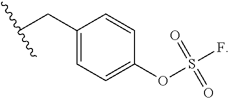 [SMILES: *Cc1ccc(OS(=O)(=O)F)cc1]

155. The conjugate of claim 153, wherein the second UAA has the structure:

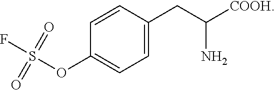 [SMILES: NC(Cc1ccc(OS(=O)(=O)F)cc1)C(=O)O]

156. The conjugate of claim 153, wherein the second UAA has the structure:

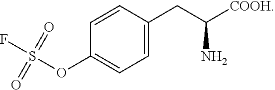 [SMILES: N[C@@H](Cc1ccc(OS(=O)(=O)F)cc1)C(=O)O]

157. The conjugate of claim 153, wherein the second UAA comprises Formula II:

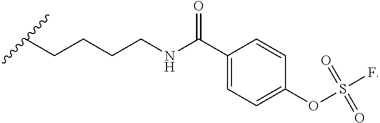 [SMILES: *CCCCNC(=O)c1ccc(OS(=O)(=O)F)cc1]

158. The conjugate of claim 153, wherein the second UAA has the structure:

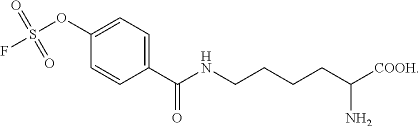 [SMILES: NC(CCCCNC(=O)c1ccc(OS(=O)(=O)F)cc1)C(=O)O]

159. The conjugate of claim 153, wherein the second UAA has the structure:

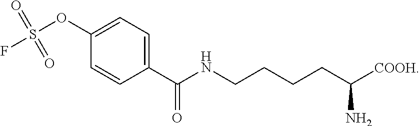 [SMILES: N[C@@H](CCCCNC(=O)c1ccc(OS(=O)(=O)F)cc1)C(=O)O]

160. The conjugate of claim 153, wherein the second UAA comprises Formula III:

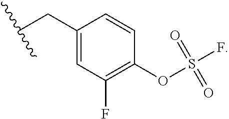 [SMILES: *Cc1ccc(OS(=O)(=O)F)c(F)c1]

161. The conjugate of claim 153, wherein the second UAA has the structure:

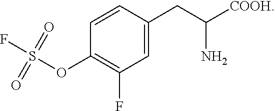 [SMILES: NC(Cc1ccc(OS(=O)(=O)F)c(F)c1)C(=O)O]

162. The conjugate of claim 153, wherein the second UAA has the structure:

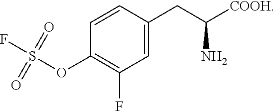 [SMILES: N[C@@H](Cc1ccc(OS(=O)(=O)F)c(F)c1)C(=O)O]

163. The conjugate of any one of claims 149-151, wherein the second UAA has a structure of Formula (IA): 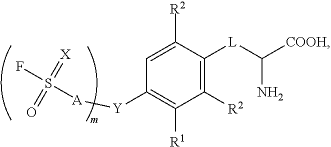
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, m is 1; when Y is —N═, m is 2.

164. The conjugate of claim 163, wherein the second UAA has a structure of Formula (IA-a):

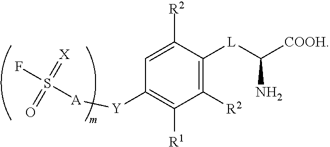

165. The conjugate of claim 163, wherein the second UAA has a structure of Formula (IA-b):

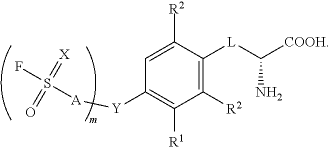

166. The conjugate of claim 163, wherein the second UAA has a structure of Formula (IB):

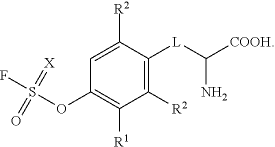

167. The conjugate of claim 163, wherein the second UAA has a structure of Formula (IC):

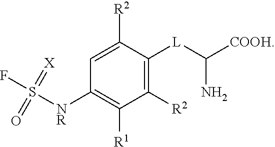

168. The conjugate of claim 163, wherein the second UAA has a structure of Formula (ID): 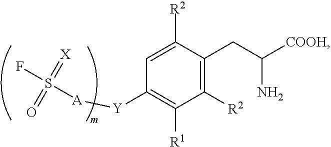
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

169. The conjugate of claim 163, wherein the second UAA has a structure of Formula (IE): 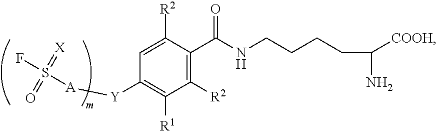
wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, m is 2.

170. The conjugate of claim 163, wherein the second UAA has a structure of Formula (IIA): 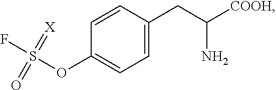
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

171. The conjugate of claim 163, wherein the second UAA has a structure of Formula (IIB): 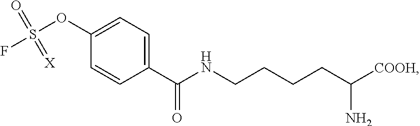
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

172. The conjugate of any one of claims 149-151, wherein the second UAA has a structure of 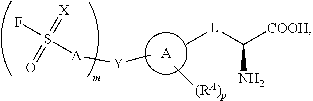
wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NRX, N(R^{X})₂, or optionally substituted alkyl; p is 0, 1, 2, 3 or 4; each R^{X} is optionally substituted alkyl;
L is —(CH₂)ₚ— or —C(O)NH—(CH₂)ₚ—; p is an integer of 1 to 6; and
wherein when Y is a bond, —O— or —NR—, m is 1; when Y is —N═, m is 2.

173. The conjugate of any one of claims 108-172, wherein the first targeting domain comprises any one of SEQ ID NOS: 1-4, or 16-64.

174. The conjugate of any one of claims 108-172, wherein the first targeting domain comprises a sequence having at least 70% sequence identity to any one of SEQ ID NOs: 1-4 or 16-64.

175. The conjugate of any one of claims 108-173, wherein the second targeting domain comprises any one of SEQ ID NOS: 1-4, 16-64.

176. The conjugate of any one of claims 108-173, wherein the second targeting domain comprises a sequence having at least 70% identity to any one of SEQ ID NOs: 1-4, 16-64.

177. The conjugate of any one of claims 1-87 or 108-172, wherein the conjugate comprises SEQ ID NO: 65-72.

178. The conjugate of any one of claims 1-87 or 108-172, wherein the conjugate comprises a sequence having at least 70% sequence identity to SEQ ID NO: 65-72.

179. The conjugate of any one of claims 1-87 or 108-172, wherein the conjugate comprises any one of SEQ ID NOS: 1-4, 16-64.

180. The conjugate of any one of claims 1-87 or 108-172, wherein the conjugate comprises a sequence having at least 70% sequence identity to any one of SEQ ID NOs: 1-4, 16-64.

181. The conjugate of any one of claims 1-87 or 108-172, wherein the conjugate comprises a sequence having at least 80% sequence identity to SEQ ID NOs: 65-72.

182. The conjugate of any one of claims 108-172, wherein at least one of the first targeting domain and the second targeting domain comprise SEQ ID NO: 1.

183. The conjugate of claim 182, wherein the UAA is present at an amino acid position selected from the group consisting of 26, 28, 29, 30, 99, 102, 103, 105, 108, 110, 111, 112, 113, 114, and 115 relative to SEQ ID NO:1.

184. The conjugate of any one of claims 108-172, wherein at least one of the first targeting domain and the second targeting domain comprise SEQ ID NO: 2.

185. The conjugate of claim 184, wherein the UAA is present at an amino acid position selected from the group consisting of 50, 52, 53, 54, 56, 58, and 100 relative to SEQ ID NO:2.

186. The conjugate of any one of claims 108-172, wherein at least one of the first targeting domain and the second targeting domain comprise SEQ ID NO: 3.

187. The conjugate of claim 186, wherein the UAA is present at an amino acid position selected from the group consisting of 58, 62, 101, 103, and 107 relative to SEQ ID NO:3.

188. The conjugate of any one of claims 108-172, wherein at least one of the first targeting domain and the second targeting domain comprise SEQ ID NO: 16.

189. The conjugate of claim 188, wherein the at least one of the first targeting domain and the second targeting domain comprising SEQ ID NO: 16 further comprises an unnatural amino acid at position 109 relative to SEQ ID NO: 16.

190. The conjugate of claim 108-172, wherein the at least one of the first targeting domain and the second targeting domain comprise SEQ ID NO: 18.

191. The conjugate of any one of claims 108-190, wherein the payload comprises an imaging agent, a radioligand agent or a cytotoxic agent.

192. The conjugate of claim 191, wherein the cytotoxic moiety comprises a small molecule drug, or a chemotherapeutic drug.

193. The conjugate of claim 191, wherein the radioligand agent is selected from the group consisting of ³⁵S, ³H, ¹¹¹In, ¹¹²In, ¹⁴C, ¹⁸⁶Re, ¹⁸⁸Re, ³²P, ¹⁵³Sm, ¹⁷⁷Lu, ⁸⁶Y, ⁸⁸Y, ⁹⁰Y, ¹³¹I, ¹²³I, ¹²⁴I, ¹²⁵I, ¹⁴⁹Tb, ²¹¹At, ²¹²Pb/²¹²Bi, ²¹³Bi, ²²³Ra, ²²⁵Ac, ⁶⁴Cu, ⁶⁷Cu, and ²²⁷Th.

194. The conjugate of claim 192 or claim 193, wherein the radioligand agent further comprises a chelator.

195. The conjugate of claim 192, wherein the radioligand agent is selected from the group consisting of ^{99m}Tc, ¹³¹I, ²⁰¹Tl, ¹¹¹In, and ⁶⁷Ga.

196. The conjugate of any one of claims 108-195, wherein the payload is attached to the conjugate with a linker.

197. A method comprising administering the conjugate of any one of claims 108-196, wherein the conjugate covalently binds the first target on the surface of a first cell.

198. The method of claim 197, wherein the conjugate binds the second target on the surface of the first cell.

199. The method of claim 197 or claim 198, wherein the first targeting domain and the second targeting domain bind to the same target.

200. The method of claim 199, wherein the first targeting domain and the second targeting domain bind to the same epitope of the same target.

201. The method of claim 199, wherein the first targeting domain and the second targeting domain bind to different epitopes of the same target.

202. The method of claim 197 or claim 198, wherein the first targeting domain and the second targeting domain bind to different targets on the surface of the first cell.

203. The method of any one of claims 197-202, wherein the second domain comprises a second UAA, and wherein the second UAA covalently binds to the second target.

204. The method of any one of claims 197-203, wherein the first cell is a tumor cell.

205. The method of claim 204, wherein the conjugate kills or inhibits the growth of the tumor cell when bound to the first target.

206. The method of claim 204, wherein the conjugate kills or inhibits the growth of the tumor cell when bound to the second target.

207. The method of claim 204, wherein the conjugate kills or inhibits the growth of the tumor cell when bound to the first target and the second target.

208. The method of any one of claims 197-207, wherein the first target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, EphA2, ALPP, ALPPL2, MT1-MMP, CLDN18.2, CLDN6, CLDN9, pCadherin, CEACAM6, CD47, and EpCAM.

209. The method of any one of claims 197-208, wherein the second target is selected from the group consisting of PSMA, EGFR, EGFRviii, MSLN, CEA, DLL3, FAP, CD33, HER3, PD-L1, EphA2, EphA4, HER2, SIRPa, DLK1, Muc16, LRP5, LRP6, endo180, LIV-1, SLAMF7, PTK7, GPR20, CDH6, CSP-1, CD71, PRLR, SEZ6, DLL1, NOTCH3 rec, NaPi2b, CD16, GCC, SSTR2, CAIX, CAXII, MC1R, CXCR4, B1R, GRPR, STEAP1, CD70, CD46, CD166, CLL-1, ADAM9, cKIT, CD36, CD73, ITGaVb3, ITGaVb6, GPC-1, CD38, CD51, FGFR3, Ly6E, CD44v6, ENPP3, CXCR3, CXCR5, FcRH5, VEGF, VEGFR2, CD45, CCR4, CD25, 5T4, ROR1, TROP-2, NECTIN4, cMET, CD19, CD22, CD30, CD33, CD123, BCMA, CD79b, AXL, RON, B7-H3, B7-H4, KAAG1, Mucd, ADAM-9, GPNMB, EDB fibronectin, tissue factor, GPNMB, FolRa, EphA2, ALPP, ALPPL2, MT1-MMP, CLDN18.2, CLDN6, CLDN9, pCadherin, CEACAM6, CD47, and EpCAM.

210. The method of any one of claims 197-209, wherein the payload is a radiolabel agent or a cytotoxic agent.

211. The method of any one of claims 198-209, wherein the payload is an imaging agent.

212. The method of claim 211, wherein the method images or identifies the first cell when the conjugate is bound to the first target and the second target on the first cell.

213. A method of manufacturing the conjugate of any one of claims 108-196, comprising:
(a) synthesizing the first targeting domain comprising at least one unnatural amino acid in vivo; and
(b) conjugating the payload to the first targeting domain or the second targeting domain, optionally via a linker.

214. The method of claim 213, further comprising synthesizing the second targeting domain in vivo as a fusion protein to the first targeting domain.

215. The method of claim 213 or claim 214, wherein synthesizing comprises use of an orthogonal tRNA synthetase/suppressor tRNA pair.

216. The method of claim 215, wherein synthesizing comprises the orthogonal tRNA synthetase/suppressor tRNA pair is derived from pyrrolysine tRNA synthetase/tRNA^{Pyl}.


SEQUENCE LISTING (129 sequences)

SEQ ID NO: 1 - protein, 140 aa, synthetic construct
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAACHFCDSLPKTQDEYILWGQGTQVTVSSAAAYPYDVPDYGS

SEQ ID NO: 2 - protein, 111 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 3 - protein, 137 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRDYSLSSSSDDFDYWGQGTQVTVSSAAAYPYDVPDYGS

SEQ ID NO: 4 - protein, 141 aa, synthetic construct
  SITE 102: note = Fluorosulfonyltyrosine
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAXFADSLPKTQDEYILWGQGTQVTVSSAAAYPYDVPDYGSC

SEQ ID NO: 5 - protein, 10 aa, unidentified
GYTDSNYYMS

SEQ ID NO: 6 - protein, 16 aa, unidentified
VNTGRGSTSYADSVKG

SEQ ID NO: 7 - protein, 18 aa, unidentified
AACHFCDSLPKTQDEYIL

SEQ ID NO: 8 - protein, 10 aa, unidentified
RFMISEYSMH

SEQ ID NO: 9 - protein, 16 aa, unidentified
TINPAGTTDYAESVKG

SEQ ID NO: 10 - protein, 5 aa, unidentified
DGYGY

SEQ ID NO: 11 - protein, 10 aa, unidentified
GWPYSTYSMN

SEQ ID NO: 12 - protein, 17 aa, synthetic construct
GISSTMSGIIFAESKAG

SEQ ID NO: 13 - protein, 15 aa, synthetic construct
RRDYSLSSSSDDFDY

SEQ ID NO: 14 - protein, 90 aa, synthetic construct
  VARIANT 1..90: note = This sequence may encompass 1-10 GGGGSGGGS repeating units
GGGGSGGGSGGGGSGGGSGGGGSGGGSGGGGSGGGSGGGGSGGGSGGGGSGGGSGGGGSGGGSGGGGSGGGSGGGGSGGGSGGGGSGGGS

SEQ ID NO: 15 - protein, 24 aa, synthetic construct
MKYLLPTAAAGLLLLAAQPAMAMG

SEQ ID NO: 16 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 17 - protein, 124 aa, synthetic construct
  SITE 109: note = Fluorosulfonyltyrosine
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSS

SEQ ID NO: 18 - protein, 132 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSLE

SEQ ID NO: 19 - protein, 141 aa, synthetic construct
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAHFADSLPKTQDEYILWGQGTQVTVSSAAAYPYDVPDYGSC

SEQ ID NO: 20 - protein, 111 aa, synthetic construct
  SITE 54: note = Fluorosulfonyltyrosine
EVQLVESGGGLVQPGGSLTLSCAASRFMISEYSMHWVRQAPGKGLEWVSTINPXGTTDYAESVKGRFTISRDNAKNTLYLQMNSLKPEDTAVYYCDGYGYRGQGTQVTVSS

SEQ ID NO: 21 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 22 - protein, 133 aa, synthetic construct
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSSSPSTPPTPSPSTPP

SEQ ID NO: 23 - protein, 130 aa, synthetic construct
QVQLQESGGGLVQTGGSLRLSCAASGSIFVGNAMGWYRQALGNQRELVAGITSDGITYYPDSVKGRFTISRDNDKNTIYLQMNSLKPEDTAVYYCNLWPPRIGFASWGQGTQVTVSSAAAYPYDVPDYGS

SEQ ID NO: 24 - protein, 127 aa, synthetic construct
QVQLVQSGGGLVQAGGSLRLSCAFSGRTFSMYTMGWFRQAPGKEREFVAANRGRGLSPDIADSVNGRFTISRDNAKNTLYLQMDSLKPEDTAVYYCAADLQYGSSWPQRSSAEYDYWGQGTTVTVSS

SEQ ID NO: 25 - protein, 117 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 26 - protein, 116 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 27 - protein, 125 aa, synthetic construct
EVQLLESGGGEVQPGGSLRLSCAASETFGVVFTLGWYRQAPGKGREFVARVTGTDTVDYAESVKGRFTISSDFARNTVYLQMNSLRAEDTAVYYCNTGAYWGQGTLVTVKPSPSTPPTPSPSTPP

SEQ ID NO: 28 - protein, 123 aa, synthetic construct [sequence omitted]

SEQ ID NO: 29 - protein, 167 aa, synthetic construct
MDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLAAALEHHHHHH

SEQ ID NO: 30 - protein, 128 aa, synthetic construct
RSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGS

SEQ ID NO: 31 - protein, 129 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSSPSTPPTPSPSTPP

SEQ ID NO: 32 - protein, 129 aa, synthetic construct
  SITE 52: note = Fluorosulfonyltyrosine
QVQLQESGGGSVQAGGSLKLTCAASGYIFNSCGMGWYRQSPGRERELVSRIXGDGDTWHKESVKGRFTISQDNVKKTLYLQMNSLKPEDTAVYFCAVCYNLETYWGQGTQVTVSSSPSTPPTPSPSTPP

SEQ ID NO: 33 - protein, 129 aa, synthetic construct
  SITE 54: note = Fluorosulfonyltyrosine
QVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSSSPSTPPTPSPSTPP

SEQ ID NO: 34 - protein, 138 aa, synthetic construct
EVQLVQSGGGLVQPGGSLRLSCAASGRTSSKYAMGWFRQAPGKGTEFVATISWSDGSTYYADSVEGRFTISRDNAKNTVYLQMNSLKPEDTAVYYCAAAVDVLAGTFEYEYDYWGQGTLVTVSSSPSTPPTPSPSTPP

SEQ ID NO: 35 - protein, 124 aa, synthetic construct
QVKLEESGGGSVQTGGSLRLTCAASGRTSRSYGMGWFRQAPGKSREFVSGISWRGDSTGYADSVKGRFTISRDNAKNTVDLQMNSLKPEDTAIYYCAAAAGSAWYGTRYEYDYWGQGTQVTVSS

SEQ ID NO: 36 - protein, 124 aa, synthetic construct
  SITE 109: note = Fluorosulfonyltyrosine
QVKLEESGGGSVQTGGSLRLTCAASGRTSRSYGMGWFRQAPGKSREFVSGISWRGDSTGYADSVKGRFTISRDNAKNTVDLQMNSLKPEDTAIYYCAAAAGSAWYGTRXEYDYWGQGTQVTVSS

SEQ ID NO: 37 - protein, 123 aa, synthetic construct
QVTLRESGGGLVQAGGSLRLSCKGSGRAINTYAMGWFRQAPGKEREFVAAISWNGGHTRYADSVQGRFAISRDNADNTMYLQMNSLKPEDTAVYHCAAYSDYHRIATMEADADSWGQGTQVTV

SEQ ID NO: 38 - protein, 140 aa, synthetic construct
EVQLVESGGGLVQPGGSLRLSCAASDFTFRNYEMSWVRQAPGKGLEWVSSISGSGGSTYYAHSLKGRFTISRDNSKNTLYLQMNSLRAEDTAIYYCARLRDGFNNGFDYWGQGTLVTVSSLPETGGSPSTPPTPSPSTPP

SEQ ID NO: 39 - protein, 146 aa, synthetic construct
MGQVKLEESGGGSVQTGGSLRLTCAASGRTSRSYGMGWFRQAPGKSREFVSGISWRGDSTGYADSVKGRFTISRDNAKNTVDLQMNSLKPEDTAIYYCAAAAGSAWYGTRYEYDYWGQGTQVTVSSLPETGGSPSTPPTPSPSTPP

SEQ ID NO: 40 - protein, 146 aa, synthetic construct
  SITE 111: note = Fluorosulfonyltyrosine
MGQVKLEESGGGSVQTGGSLRLTCAASGRTSRSYGMGWFRQAPGKSREFVSGISWRGDSTGYADSVKGRFTISRDNAKNTVDLQMNSLKPEDTAIYYCAAAAGSAWYGTRXEYDYWGQGTQVTVSSLPETGGSPSTPPTPSPSTPP

SEQ ID NO: 41 - protein, 133 aa, synthetic construct
  SITE 133: note = Alexa Fluor 680 modified residue
MGQVKLEESGGGSVQTGGSLRLTCAASGRTSRSYGMGWFRQAPGKSREFVSGISWRGDSTGYADSVKGRFTISRDNAKNTVDLQMNSLKPEDTAIYYCAAAAGSAWYGTRYEYDYWGQGTQVTVSSLPETGGG

SEQ ID NO: 42 - protein, 133 aa, synthetic construct
  SITE 111: note = Fluorosulfonyltyrosine
  SITE 133: note = Alexa Fluor 680 modified residue
MGQVKLEESGGGSVQTGGSLRLTCAASGRTSRSYGMGWFRQAPGKSREFVSGISWRGDSTGYADSVKGRFTISRDNAKNTVDLQMNSLKPEDTAIYYCAAAAGSAWYGTRXEYDYWGQGTQVTVSSLPETGGG

SEQ ID NO: 43 - protein, 137 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGS

SEQ ID NO: 44 - protein, 137 aa, synthetic construct
  SITE 101: note = Fluorosulfonyltyrosine
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGS

SEQ ID NO: 45 - protein, 126 aa, synthetic construct
EVQLVESGGGLVQPGGSLTLSCAASRFMISEYSMHWVRQAPGKGLEWVSTINPYGTTDYAESVKGRFTISRDNAKNTLYLQMNSLKPEDTAVYYCDGYGYRGQGTQVTVSSSPSTPPTPSPSTPPC

SEQ ID NO: 46 - protein, 126 aa, synthetic construct
  SITE 54: note = Fluorosulfonyltyrosine
EVQLVESGGGLVQPGGSLTLSCAASRFMISEYSMHWVRQAPGKGLEWVSTINPXGTTDYAESVKGRFTISRDNAKNTLYLQMNSLKPEDTAVYYCDGYGYRGQGTQVTVSSSPSTPPTPSPSTPPC

SEQ ID NO: 47 - protein, 132 aa, synthetic construct
  SITE 132: note = Polyethyleneglycol-valine-citrulline-p-aminobenzylc arbamyl-monomethyl auristatin E modified residue
EVQLVESGGGLVQPGGSLTLSCAASRFMISEYSMHWVRQAPGKGLEWVSTINPYGTTDYAESVKGRFTISRDNAKNTLYLQMNSLKPEDTAVYYCDGYGYRGQGTQVTVSSSPSTPPTPSPSTPPCHHHHHH

SEQ ID NO: 48 - protein, 132 aa, synthetic construct
  SITE 54: note = Fluorosulfonyltyrosine
  SITE 132: note = Polyethyleneglycol-valine-citrulline-p-aminobenzylc arbamyl-monomethyl auristatin E modified residue
EVQLVESGGGLVQPGGSLTLSCAASRFMISEYSMHWVRQAPGKGLEWVSTINPXGTTDYAESVKGRFTISRDNAKNTLYLQMNSLKPEDTAVYYCDGYGYRGQGTQVTVSSSPSTPPTPSPSTPPCHHHHHH

SEQ ID NO: 49 - protein, 152 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSSPSTPPTPSPSTPPC

SEQ ID NO: 50 - protein, 152 aa, synthetic construct
  SITE 101: note = Fluorosulfonyltyrosine
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPC

SEQ ID NO: 51 - protein, 158 aa, synthetic construct
  SITE 158: note = Polyethyleneglycol-valine-citrulline-p-aminobenzylc arbamyl-monomethyl auristatin E modified residue
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSSPSTPPTPSPSTPPCHHHHHH

SEQ ID NO: 52 - protein, 158 aa, synthetic construct
  SITE 101: note = Fluorosulfonyltyrosine
  SITE 158: note = Polyethyleneglycol-valine-citrulline-p-aminobenzylc arbamyl-monomethyl auristatin E modified residue
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPCHHHHHH

SEQ ID NO: 53 - protein, 157 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSLPETGGSPSTPPTPSPSTPP

SEQ ID NO: 54 - protein, 157 aa, synthetic construct
  SITE 101: note = Fluorosulfonyltyrosine
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSLPETGGSPSTPPTPSPSTPP

SEQ ID NO: 55 - protein, 144 aa, synthetic construct
  SITE 144: note = Alexa Fluor 680 modified residue
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSLPETGGG

SEQ ID NO: 56 - protein, 144 aa, synthetic construct
  SITE 101: note = Fluorosulfonyltyrosine
  SITE 144: note = Alexa Fluor 680 modified residue
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSLPETGGG

SEQ ID NO: 57 - protein, 148 aa, synthetic construct
QVQLQESGGGSVQAGGSLKLTCAASGYIFNSCGMGWYRQSPGRERELVSRIYGDGDTWHKESVKGRFTISQDNVKKTLYLQMNSLKPEDTAVYFCAVCYNLETYWGQGTQVTVSSAAAYPYDVPDYGSLPETGGSPSTPPTPSPSTPP

SEQ ID NO: 58 - protein, 148 aa, synthetic construct
  SITE 52: note = Fluorosulfonyltyrosine
QVQLQESGGGSVQAGGSLKLTCAASGYIFNSCGMGWYRQSPGRERELVSRIXGDGDTWHKESVKGRFTISQDNVKKTLYLQMNSLKPEDTAVYFCAVCYNLETYWGQGTQVTVSSAAAYPYDVPDYGSLPETGGSPSTPPTPSPSTPP

SEQ ID NO: 59 - protein, 148 aa, synthetic construct
QVQLQESGGGSVQAGGSLKLTCAASGYIFNSCGMGWYRQSPGRERELVSRISGYGDTWHKESVKGRFTISQDNVKKTLYLQMNSLKPEDTAVYFCAVCYNLETYWGQGTQVTVSSAAAYPYDVPDYGSLPETGGSPSTPPTPSPSTPP

SEQ ID NO: 60 - protein, 148 aa, synthetic construct
  SITE 54: note = Fluorosulfonyltyrosine
QVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSSAAAYPYDVPDYGSLPETGGSPSTPPTPSPSTPP

SEQ ID NO: 61 - protein, 135 aa, synthetic construct
  SITE 135: note = Polyethyleneglycol-valine-citrulline-p-aminobenzylc arbamyl-monomethyl auristatin E modified residue
QVQLQESGGGSVQAGGSLKLTCAASGYIFNSCGMGWYRQSPGRERELVSRIYGDGDTWHKESVKGRFTISQDNVKKTLYLQMNSLKPEDTAVYFCAVCYNLETYWGQGTQVTVSSAAAYPYDVPDYGSLPETGGG

SEQ ID NO: 62 - protein, 135 aa, synthetic construct
  SITE 52: note = Fluorosulfonyltyrosine
  SITE 135: note = Polyethyleneglycol-valine-citrulline-p-aminobenzylc arbamyl-monomethyl auristatin E modified residue
QVQLQESGGGSVQAGGSLKLTCAASGYIFNSCGMGWYRQSPGRERELVSRIXGDGDTWHKESVKGRFTISQDNVKKTLYLQMNSLKPEDTAVYFCAVCYNLETYWGQGTQVTVSSAAAYPYDVPDYGSLPETGGG

SEQ ID NO: 63 - protein, 135 aa, synthetic construct
  SITE 135: note = Polyethyleneglycol-valine-citrulline-p-aminobenzylc arbamyl-monomethyl auristatin E modified residue
QVQLQESGGGSVQAGGSLKLTCAASGYIFNSCGMGWYRQSPGRERELVSRISGYGDTWHKESVKGRFTISQDNVKKTLYLQMNSLKPEDTAVYFCAVCYNLETYWGQGTQVTVSSAAAYPYDVPDYGSLPETGGG

SEQ ID NO: 64 - protein, 135 aa, synthetic construct
  SITE 54: note = Fluorosulfonyltyrosine
  SITE 135: note = Polyethyleneglycol-valine-citrulline-p-aminobenzylc arbamyl-monomethyl auristatin E modified residue
QVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSSAAAYPYDVPDYGSLPETGGG

SEQ ID NO: 65 - protein, 283 aa, synthetic construct
EVQLVESGGGLVQPGGSLTLSCAASRFMISEYSMHWVRQAPGKGLEWVSTINPYGTTDYAESVKGRFTISRDNAKNTLYLQMNSLKPEDTAVYYCDGYGYRGQGTQVTVSSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSLPETGGSPSTPPTPSPSTPP

SEQ ID NO: 66 - protein, 283 aa, synthetic construct
  SITE 227: note = Fluorosulfonyltyrosine
EVQLVESGGGLVQPGGSLTLSCAASRFMISEYSMHWVRQAPGKGLEWVSTINPYGTTDYAESVKGRFTISRDNAKNTLYLQMNSLKPEDTAVYYCDGYGYRGQGTQVTVSSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSLPETGGSPSTPPTPSPSTPP

SEQ ID NO: 67 - protein, 296 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSLPETGGSPSTPPTPSPSTPP

SEQ ID NO: 68 - protein, 296 aa, synthetic construct
  SITE 101: note = Fluorosulfonyltyrosine
  SITE 240: note = Fluorosulfonyltyrosine
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSLPETGGSPSTPPTPSPSTPP

SEQ ID NO: 69 - protein, 266 aa, synthetic construct
  SITE 109: note = Fluorosulfonyltyrosine
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSLE

SEQ ID NO: 70 - protein, 289 aa, synthetic construct
  SITE 66: note = Fluorosulfonyltyrosine
MDLGKKLLEAARAGQDDEVRILMANGADVNAHDFYGITPLHLAANFGHLEIVEVLLKHGADVNAFXYDNTPLHLAADAGHLEIVEVLLKYGADVNASDRDGHTPLHLAAREGHLEIVEVLLKNGADVNAQDKFGKTAFDISIDNGNEDLAEILQKLGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGS

SEQ ID NO: 71 - protein, 140 aa, synthetic construct
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAHFADSLPKTQDEYILWGQGTQVTVSSAAAYPYDVPDYGS

SEQ ID NO: 72 - protein, 290 aa, synthetic construct
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAHFADSLPKTQDEYILWGQGTQVTVSSAAAYPYDVPDYGSGGGGSGGGGSQVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAHFADSLPKTQDEYILWGQGTQVTVSSAAAYPYDVPDYGS

SEQ ID NO: 73 - DNA, 558 bp, synthetic construct
  source 1..558: mol_type = other DNA
ctttaagaaggagatatacatatgaagtatttgttgcctacagcggctgccggattacttctgcttgccgcacagccagcgatggcccaagtccaactgcaagagtcgggtggcggctctgttcaagcgggaggctccttgcgcttgagctgtaccgctcctgggtacacagacagtaattattatatgagctggttccgccaagcgcccggtaaagaacgtgaatgggtagctggtgtgaacacaggacgcgggtcgacatcctacgcggacagtgttaaaggtcgcttcacaatctcgcaagataacgctaagaacacaatgttcttgcaaatgaactcgcttaagccagaagatactgctatttactactgcgctgtagccgcctgccacttctgtgatagtcttccaaagactcaagacgagtacattttatggggccagggcactcaagtcacagtttcgagcgccgccgcatacccatacgacgtcccagactatggctctcatcatcaccatcaccattaaaagcttggctgttttggcggatgagagaag

SEQ ID NO: 74 - DNA, 468 bp, synthetic construct
  source 1..468: mol_type = other DNA
ctttaagaaggagatatacatatgaagtatttattacctacagcggcagcagggttattgttattggccgcgcagcccgcaatggctgaagttcaacttgttgagagcgggggcggattggtacagccgggggggtcacttaccttatcatgtgcagcatcgcgcttcatgatctcagaatacagtatgcattgggttcgtcaagcgccggggaaaggtcttgagtgggttagtaccatcaacccagccggaaccactgattacgcggaaagcgtgaagggccgttttaccatctcccgcgacaatgctaaaaatactttatacttacagatgaacagtttgaaacccgaagatacggcggtgtactactgtgacgggtacggctatcgtggccaaggaacacaagttactgtatcctctcatcatcatcaccatcactaaaagcttggctgttttggcggatgagag

SEQ ID NO: 75 - DNA, 539 bp, synthetic construct
  source 1..539: mol_type = other DNA
ctttaagaaggagatatacatatgaagtaccttctgccgactgccgctgcaggtttgttgttattggccgctcaaccagcgatggcccaggtacagctgcaagaaagtggtggtggtagcgtagaggcgggtgggtctttacgtctgtcgtgcgctcgttcgggttggccctactcaacgtatagtatgaattggttccgtcaggcccccggtaaagaacgtgaggcagtcgcggggatttcgtcaacgatgtctgggattatctttgccgagtccaaagctggacagttcactattagccaagataacgccaagaacacagtgtatcttcagatgaataacctgaaaccggaagacaccgcaatttactactgcgcagcgcgccgtgactactccttgtcttcgagctcggacgatttcgattattggggtcaggggacccaagtcactgtgagttccgccgcagcatatccgtatgatgtacccgattacggctcgcaccaccatcatcaccattaaaagcttggctgttttggcgg

SEQ ID NO: 76 - DNA, 513 bp, synthetic construct
  source 1..513: mol_type = other DNA
ctttaagaaggagatatacatatgaaatacctgctgccgaccgctgctgctggtctgctgctcctcgctgcccagccggcgatggccatgggccaagtaaaactagaggaatcaggtggagggtccgtgcaaaccggtggcagtctccgtttgacctgtgctgcgagcggccgtaccagccgcagctacggcatgggttggtttcgtcaggcaccgggtaaggagcgcgagttcgtgtccggcatcagctggcgtggtgacagcacgggctatgcggatagcgttaaaggtcgcttcaccatttctcgtgataatgcaaagaacaccgttgatctgcaaatgaacagcctgaaaccggaagacaccgctatctactactgcgccgcggcggcgggttctgcctggtatggcactctgtatgaatacgactactggggtcagggtacgcaggttaccgtctcgtcccatcatcaccaccaccactaaaagcttggctgttttggcggatgagag

SEQ ID NO: 77 - DNA, 513 bp, synthetic construct
  source 1..513: mol_type = other DNA
ctttaagaaggagatatacatatgaaatacctgctgccgaccgctgctgctggtctgctgctcctcgctgcccagccggcgatggccatgggccaagtaaaactagaggaatcaggtggagggtccgtgcaaaccggtggcagtctccgtttgacctgtgctgcgagcggccgtaccagccgcagctacggcatgggttggtttcgtcaggcaccgggtaaggagcgcgagttcgtgtccggcatcagctggcgtggtgacagcacgggctatgcggatagcgttaaaggtcgcttcaccatttctcgtgataatgcaaagaacaccgttgatctgcaaatgaacagcctgaaaccggaagacaccgctatctactactgcgccgcggcggcgggttctgcctggtatggcactctgtaggaatacgactactggggtcagggtacgcaggttaccgtctcgtcccatcatcaccaccaccactaaaagcttggctgttttggcggatgagag

SEQ ID NO: 78 - DNA, 525 bp, synthetic construct
  source 1..525: mol_type = other DNA
ctttaagaaggagatatacatatgaaatacttacttccaacagcagccgcaggattattactgttggccgctcagcctgcaatggctcaagtgcagttacaagaaagcgggggcggacttgtgcaaacaggagggagtctgcgtctttcctgtgcggcgagtgggtcgatcttcgttggaaatgcgatgggctggtatcgccaagccttgggtaatcaacgtgaattagtcgcagggatcacgtccgatgggatcacatactacccggattcagttaagggccgctttacaatctctcgtgataacgataaaaacacgatttacttacaaatgaatagtttaaaaccagaggataccgctgtttactactgcaacctgtggccccctcgtatcgggttcgcctcctggggacaaggaactcaagtgacggtctcgagtgctgccgcttacccttacgacgtaccggattacgggtcgcatcatcatcatcaccactaaaagcttggctgttttggcggatgagag

SEQ ID NO: 79 - DNA, 522 bp, synthetic construct
  source 1..522: mol_type = other DNA
ctttaagaaggagatatacatatgaaatacttattacctaccgctgcagcggggctgttgttgttggccgcccagccagcaatggctatggcgcaagtccaattagttcaatcaggcggggggttggtccaggcggggggatccttacgtctgtcatgtgctttcagtggccgcacgttctcaatgtacacgatggggtggtttcgtcaagctccggggaaagaacgcgagtttgtggccgcaaatcgcggacgtgggttaagtcccgacattgctgacagtgttaatggacgcttcaccatttcacgcgacaatgctaagaatactctttaccttcagatggactctctgaaaccagaggacaccgccgtatattattgcgccgcagacttacagtacgggtcatcatggcctcaacgttcgtctgctgaatatgattactgggggcaaggaacgacagtcactgtatcctcgcatcaccaccatcaccattaaaagcttggctgttttggcggatgagag

SEQ ID NO: 80 - DNA, 45 bp, synthetic construct
  source 1..45: mol_type = other DNA
cttggctgttttggcggatgagagaagattttcagcctgatacag

SEQ ID NO: 81 - DNA, 60 bp, synthetic construct
  source 1..60: mol_type = other DNA
gtatatctccttcttaaagttaaacaaaattatttctagcccaaaaaaacgggtatggag

SEQ ID NO: 82 - DNA, 4457 bp, synthetic construct
  source 1..4457: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagaaataattttgtttaactttaagaaggagatatacatatgaaatacttacttccaacagcagccgcaggattattactgttggccgctcagcctgcaatggctcaagtgcagttacaagaaagcgggggcggacttgtgcaaacaggagggagtctgcgtctttcctgtgcggcgagtgggtcgatcttcgttggaaatgcgatgggctggtatcgccaagccttgggtaatcaacgtgaattagtcgcagggatcacgtccgatgggatcacatactacccggattcagttaagggccgctttacaatctctcgtgataacgataaaaacacgatttacttacaaatgaatagtttaaaaccagaggataccgctgtttactactgcaacctgtggccccctcgtatcgggttcgcctcctggggacaaggaactcaagtgacggtctcgagtgctgccgcttacccttacgacgtaccggattacgggtcgcatcatcatcatcaccactaaaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatcgtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 83 - DNA, 4453 bp, synthetic construct
  source 1..4453: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagaaataattttgtttaactttaagaaggagatatacatatgaaatacttattacctaccgctgcagcggggctgttgttgttggccgcccagccagcaatggctatggcgcaagtccaattagttcaatcaggcggggggttggtccaggcggggggatccttacgtctgtcatgtgctttcagtggccgcacgttctcaatgtacacgatggggtggtttcgtcaagctccggggaaagaacgcgagtttgtggccgcaaatcgcggacgtgggttaagtcccgacattgctgacagtgttaatggacgcttcaccatttcacgcgacaatgctaagaatactctttaccttcagatggactctctgaaaccagaggacaccgccgtatattattgcgccgcagacttacagtacgggtcatcatggcctcaacgttcgtctgctgaatatgattactgggggcaaggaacgacagtcactgtatcctcgcatcaccaccatcaccattaaaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 84 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLIPNLYNYLRKLDRALPDPIKIFEIGPCYRKESDGKEHLEEFTMLTFIQMGSGCTRENLESIITDFLNHLGIDFKIVGDSCMVLGDTLDVMHGDLELSSAVVGPIPLDREWGIDKPKIGAGFGLERLLKVKHDFKNIKRAARSESYYNGISTNL

SEQ ID NO: 85 - DNA, 1365 bp, synthetic construct
  source 1..1365: mol_type = other DNA
atggataaaaagcctttgaacactctgatttctgcgaccggtctgtggatgtcccgcaccggcaccatccacaaaatcaaacaccatgaagttagccgttccaaaatctacattgaaatggcttgcggcgatcacctggttgtcaacaactcccgttcttctcgtaccgctcgcgcactgcgccaccacaaatatcgcaaaacctgcaaacgttgccgtgttagcgatgaggacctgaacaaattcctgaccaaagctaacgaggatcagacctccgtaaaagtgaaggtagtaagcgctccgacccgtactaaaaaggctatgccaaaaagcgtggcccgtgccccgaaacctctggaaaacaccgaggcggctcaggctcaaccatccggttctaaattttctccggcgatcccagtgtccacccaagaatctgtttccgtaccagcaagcgtgtctaccagcattagcagcatttctaccggtgctaccgcttctgcgctggtaaaaggtaacactaacccgattactagcatgtctgcaccggtacaggcaagcgccccagctctgactaaatcccagacggaccgtctggaggtgctgctgaacccaaaggatgaaatctctctgaacagcggcaagcctttccgtgagctggaaagcgagctgctgtctcgtcgtaaaaaggatctgcaacagatctacgctgaggaacgcgagaactatctgggtaagctggagcgcgaaattactcgcttcttcgtggatcgcggtttcctggagatcaaatctccgattctgattccgctggaatacattgaacgtatgggcatcgataatgataccgaactgtctaaacagatcttccgtgtggataaaaacttctgtctgcgtccgatgctgattccgaacttgtacaactatttacgtaaactggaccgtgccctgccggacccgatcaaaatattcgagatcggtccttgctaccgtaaagagtccgacggtaaagagcacctggaagaattcaccatgctgacattcattcagatgggtagcggttgcacgcgtgaaaacctggaatccattatcaccgacttcctgaatcacctgggtatcgatttcaaaattgttggtgacagctgtatggtgttaggcgatacgctggatgttatgcacggcgatctggagctgtcttccgcagttgtgggcccaatcccgctggatcgtgagtggggtatcgacaaacctaaaatcggtgcgggttttggtctggagcgtctgctgaaagtaaaacacgacttcaagaacatcaaacgtgctgcacgttccgagtcctattacaatggtatttctactaacctgtaa

SEQ ID NO: 86 - DNA, 5652 bp, synthetic construct
  source 1..5652: mol_type = other DNA
actagtatggataaaaagcctttgaacactctgatttctgcgaccggtctgtggatgtcccgcaccggcaccatccacaaaatcaaacaccatgaagttagccgttccaaaatctacattgaaatggcttgcggcgatcacctggttgtcaacaactcccgttcttctcgtaccgctcgcgcactgcgccaccacaaatatcgcaaaacctgcaaacgttgccgtgttagcgatgaggacctgaacaaattcctgaccaaagctaacgaggatcagacctccgtaaaagtgaaggtagtaagcgctccgacccgtactaaaaaggctatgccaaaaagcgtggcccgtgccccgaaacctctggaaaacaccgaggcggctcaggctcaaccatccggttctaaattttctccggcgatcccagtgtccacccaagaatctgtttccgtaccagcaagcgtgtctaccagcattagcagcatttctaccggtgctaccgcttctgcgctggtaaaaggtaacactaacccgattactagcatgtctgcaccggtacaggcaagcgccccagctctgactaaatcccagacggaccgtctggaggtgctgctgaacccaaaggatgaaatctctctgaacagcggcaagcctttccgtgagctggaaagcgagctgctgtctcgtcgtaaaaaggatctgcaacagatctacgctgaggaacgcgagaactatctgggtaagctggagcgcgaaattactcgcttcttcgtggatcgcggtttcctggagatcaaatctccgattctgattccgctggaatacattgaacgtatgggcatcgataatgataccgaactgtctaaacagatcttccgtgtggataaaaacttctgtctgcgtccgatgctgattccgaacttgtacaactatttacgtaaactggaccgtgccctgccggacccgatcaaaatattcgagatcggtccttgctaccgtaaagagtccgacggtaaagagcacctggaagaattcaccatgctgacattcattcagatgggtagcggttgcacgcgtgaaaacctggaatccattatcaccgacttcctgaatcacctgggtatcgatttcaaaattgttggtgacagctgtatggtgttaggcgatacgctggatgttatgcacggcgatctggagctgtcttccgcagttgtgggcccaatcccgctggatcgtgagtggggtatcgacaaacctaaaatcggtgcgggttttggtctggagcgtctgctgaaagtaaaacacgacttcaagaacatcaaacgtgctgcacgttccgagtcctattacaatggtatttctactaacctgtaagtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttgtttgtgagctcccggtcatcaatcatccccataatccttgttagattatcaattttaaaaaactaacagttgtcagcctgtcccgctttaatatcatacgccgttatacgttgtttacgctttgaggaatcccatatgatacgcggccgctttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcaacatgtgagcaccggtttattgactaccggaagcagtgtgaccgtgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagatgcgccccgcattggaaacctgatcatgtagatcgaatggactctaaatccgttcagccgggttagattcccggggtttccgccaaattcgaaaagcctgctcaacgagcaggcttttttgcatgctcgagcagctcagggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttggtacccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcactcggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctaacaggaggaatt

SEQ ID NO: 87 - protein, 275 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLGSVARDLRDHTDGPVKIFEMGSCFRKESHSGMHLEEFTMLNLFDMGPRGDATEVLKNYISVVMKAAGLPDYDLVQEESDVYKETIDVEINGQEVCSAAVGPTPIDAAHDVHEPWSGAGFGLERLLTIREKYSTVKKGGASISYLNGAKIN

SEQ ID NO: 88 - DNA, 828 bp, synthetic construct
  source 1..828: mol_type = other DNA
atgaccgtgaagtacaccgacgcccagatccaacgcctgcgcgagtatggtaatggcacctatgaacagaaagtttttgaggacctggccagccgtgacgccgcgttcagcaaggaaatgagcgtggccagcaccgacaacgagaagaaaatcaagggtatgattgccaatccgagccgccacggcctgacccagctgatgaacgacatcgctgatgccctggtggcggagggtttcattgaagtgcgtaccccgatctttattagcaaggatgcgctggctcgcatgaccatcaccgaagacaagccgctgttcaaacaggtgttttggattgatgagaaacgtgccctgcgcccgatgctggcgccaaacctgggtagcgtggctcgtgacctgcgcgatcacaccgacggtccggtgaaaatcttcgaaatgggcagctgctttcgtaaagagagccacagcggtatgcacctggaggagttcaccatgctgaatctgtttgatatgggtccgcgcggcgacgctaccgaagtgctgaagaactacattagcgtggtgatgaaagctgccggcctgccggactacgatctggtgcaggaagagagcgacgtgtacaaggaaaccatcgacgtggaaattaacggtcaggaagtgtgcagcgcggctgtgggtccaactcctattgatgcggcccatgacgtgcatgagccgtggagcggtgctggttttggcctggaacgtctgctgaccatccgcgagaaatactccaccgtgaaaaagggcggcgcaagcatctcctacctgaatggtgccaaaatcaattaa

SEQ ID NO: 89 - DNA, 5114 bp, synthetic construct
  source 1..5114: mol_type = other DNA
actagtatgaccgtgaagtacaccgacgcccagatccaacgcctgcgcgagtatggtaatggcacctatgaacagaaagtttttgaggacctggccagccgtgacgccgcgttcagcaaggaaatgagcgtggccagcaccgacaacgagaagaaaatcaagggtatgattgccaatccgagccgccacggcctgacccagctgatgaacgacatcgctgatgccctggtggcggagggtttcattgaagtgcgtaccccgatctttattagcaaggatgcgctggctcgcatgaccatcaccgaagacaagccgctgttcaaacaggtgttttggattgatgagaaacgtgccctgcgcccgatgctggcgccaaacctgggtagcgtggctcgtgacctgcgcgatcacaccgacggtccggtgaaaatcttcgaaatgggcagctgctttcgtaaagagagccacagcggtatgcacctggaggagttcaccatgctgaatctgtttgatatgggtccgcgcggcgacgctaccgaagtgctgaagaactacattagcgtggtgatgaaagctgccggcctgccggactacgatctggtgcaggaagagagcgacgtgtacaaggaaaccatcgacgtggaaattaacggtcaggaagtgtgcagcgcggctgtgggtccaactcctattgatgcggcccatgacgtgcatgagccgtggagcggtgctggttttggcctggaacgtctgctgaccatccgcgagaaatactccaccgtgaaaaagggcggcgcaagcatctcctacctgaatggtgccaaaatcaattaagtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttgtttgtgagctcccggtcatcaatcatccccataatccttgttagattatcaattttaaaaaactaacagttgtcagcctgtcccgctttaatatcatacgccgttatacgttgtttacgctttgaggaatcccatatgatacgcggccgctttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcaacatgtgagcaccggtttattgactaccggaagcagtgtgaccgtgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagatgcgccccgcattgggggacggtccggcgaccagcgggtctctaaaacctagccagcggggttcgacgccccggtctctcgccaaattcgaaaagcctgctcaacgagcaggcttttttgcatgctcgagcagctcagggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttggtacccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcactcggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctaacaggaggaatt

SEQ ID NO: 90 - protein, 454 aa, Methanosarcina mazei
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 91 - DNA, 828 bp, synthetic construct
  source 1..828: mol_type = other DNA
atgaccgtgaagtacaccgacgcccagatccaacgcctgcgcgagtatggtaatggcacctatgaacagaaagtttttgaggacctggccagccgtgacgccgcgttcagcaaggaaatgagcattgccagcaccgacaacgagaagaaaatcaagggtatgattgccaatccgagccgccacggcctgacccagctgatgaacgacatcgctgatgccctggtggcggagggtttcattgaagtgcgtaccccgatctttattagcaaggatgcgctggctcgcatgaccatcaccgaagacaagccgctgttcaaacaggtgttttggattgatgagaaacgtgccctgcgcccgatgctggcgccaaacctgggtagcgtggctcgtgacctgcgcgatcacaccgacggtccggtgaaaatcttcgaaatgggcagctgctttcgtaaagagagccacagcggtatgcacctggaggagttcaccatgctgaatctgtttgatatgggtccgcgcggcgacgctaccgaagtgctggaaaactacattagcgtggtgatgaaagtggccggcctgccggactacgatctggtgcaggaagagagcgacgtgtacaaggaaaccatcgacgtggaaattaacggtcaggaagtgtgcagcgcggctgtgggtccaactcctattgatgcggcccatgacgtgcatgagccgtggagcggtgctggttttggcctggaacgtctgctgaccatccgcgagaaatactccaccgtgaaaaagggcggcgcaagcatctcctacctgaatggtgccaaaatcaattaa

SEQ ID NO: 92 - protein, 275 aa, synthetic construct
MTVKYTDAQIQRLREYGNGTYEQKVFEDLASRDAAFSKEMSIASTDNEKKIKGMIANPSRHGLTQLMNDIADALVAEGFIEVRTPIFISKDALARMTITEDKPLFKQVFWIDEKRALRPMLAPNLGSVARDLRDHTDGPVKIFEMGSCFRKESHSGMHLEEFTMLNLFDMGPRGDATEVLENYISVVMKVAGLPDYDLVQEESDVYKETIDVEINGQEVCSAAVGPTPIDAAHDVHEPWSGAGFGLERLLTIREKYSTVKKGGASISYLNGAKIN

SEQ ID NO: 93 - DNA, 5114 bp, synthetic construct
  source 1..5114: mol_type = other DNA
actagtatgaccgtgaagtacaccgacgcccagatccaacgcctgcgcgagtatggtaatggcacctatgaacagaaagtttttgaggacctggccagccgtgacgccgcgttcagcaaggaaatgagcattgccagcaccgacaacgagaagaaaatcaagggtatgattgccaatccgagccgccacggcctgacccagctgatgaacgacatcgctgatgccctggtggcggagggtttcattgaagtgcgtaccccgatctttattagcaaggatgcgctggctcgcatgaccatcaccgaagacaagccgctgttcaaacaggtgttttggattgatgagaaacgtgccctgcgcccgatgctggcgccaaacctgggtagcgtggctcgtgacctgcgcgatcacaccgacggtccggtgaaaatcttcgaaatgggcagctgctttcgtaaagagagccacagcggtatgcacctggaggagttcaccatgctgaatctgtttgatatgggtccgcgcggcgacgctaccgaagtgctggaaaactacattagcgtggtgatgaaagtggccggcctgccggactacgatctggtgcaggaagagagcgacgtgtacaaggaaaccatcgacgtggaaattaacggtcaggaagtgtgcagcgcggctgtgggtccaactcctattgatgcggcccatgacgtgcatgagccgtggagcggtgctggttttggcctggaacgtctgctgaccatccgcgagaaatactccaccgtgaaaaagggcggcgcaagcatctcctacctgaatggtgccaaaatcaattaagtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttgtttgtgagctcccggtcatcaatcatccccataatccttgttagattatcaattttaaaaaactaacagttgtcagcctgtcccgctttaatatcatacgccgttatacgttgtttacgctttgaggaatcccatatgatacgcggccgctttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcaacatgtgagcaccggtttattgactaccggaagcagtgtgaccgtgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagatgcgccccgcattgggggacggtccggcgaccagcgggtctctaaaacctagccagcggggttcgacgccccggtctctcgccaaattcgaaaagcctgctcaacgagcaggcttttttgcatgctcgagcagctcagggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttggtacccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcactcggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctaacaggaggaatt

SEQ ID NO: 94 - protein, 6 aa, synthetic construct
HHHHHH

SEQ ID NO: 95 - protein, 14 aa, synthetic construct
SPSTPPTPSPSTPP

SEQ ID NO: 96 - protein, 5 aa, synthetic construct
GGGGS

SEQ ID NO: 97 - protein, 4 aa, synthetic construct
GGGS

SEQ ID NO: 98 - protein, 296 aa, synthetic construct
  SITE 275: note = Fluorosulfonyltyrosine
MKYLLPTAAAGLLLLAAQPAMAMGQVQLVQSGGGLVQAGGSLRLSCAFSGRTFSMYTMGWFRQAPGKEREFVAANRGRGLSPDIADSVNGRFTISRDNAKNTLYLQMDSLKPEDTAVYYCAADLQYGSSWPQRSSAEYDYWGQGTTVTVSSGGGGSGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSHHHHHH

SEQ ID NO: 99 - protein, 10 aa, synthetic construct
GDTYGSYWMG

SEQ ID NO: 100 - protein, 17 aa, synthetic construct
AINRGGGYTVYADSVKG

SEQ ID NO: 101 - protein, 14 aa, synthetic construct
SGVLGGLHEDWFNY

SEQ ID NO: 102 - protein, 10 aa, synthetic construct
GSIFVGNAMG

SEQ ID NO: 103 - protein, 16 aa, synthetic construct
GITSDGITYYPDSVKG

SEQ ID NO: 104 - protein, 9 aa, synthetic construct
WPPRIGFAS

SEQ ID NO: 105 - protein, 10 aa, synthetic construct
ETFGVVFTLG

SEQ ID NO: 106 - protein, 16 aa, synthetic construct
RVTGTDTVDYAESVKG

SEQ ID NO: 107 - protein, 10 aa, synthetic construct
GIDLSLYRMR

SEQ ID NO: 108 - protein, 16 aa, synthetic construct
LITDDGTSYYEDSVKG

SEQ ID NO: 109 - protein, 9 aa, synthetic construct
ETPLSPVNY

SEQ ID NO: 110 - protein, 10 aa, synthetic construct
GSIFGIRTMD

SEQ ID NO: 111 - protein, 16 aa, synthetic construct
RITMDGRVFHADSVKG

SEQ ID NO: 112 - protein, 9 aa, synthetic construct
SGLTSREDY

SEQ ID NO: 113 - protein, 10 aa, synthetic construct
GRTSSKYAMG

SEQ ID NO: 114 - protein, 17 aa, synthetic construct
TISWSDGSTYYADSVEG

SEQ ID NO: 115 - protein, 15 aa, synthetic construct
AVDVLAGTFEYEYDY

SEQ ID NO: 116 - protein, 10 aa, synthetic construct
GRAINTYAMG

SEQ ID NO: 117 - protein, 17 aa, synthetic construct
AISWNGGHTRYADSVQG

SEQ ID NO: 118 - protein, 16 aa, synthetic construct
YSDYHRIATMEADADS

SEQ ID NO: 119 - protein, 10 aa, synthetic construct
DFTFRNYEMS

SEQ ID NO: 120 - protein, 10 aa, synthetic construct
DFTFRSYEMS

SEQ ID NO: 121 - protein, 17 aa, synthetic construct
SISGSGGSTYYAHSLKG

SEQ ID NO: 122 - protein, 11 aa, synthetic construct
LRDGFNNGFDY

SEQ ID NO: 123 - protein, 10 aa, synthetic construct
GYIFNSCGMG

SEQ ID NO: 124 - protein, 16 aa, synthetic construct
RISGDGDTWHKESVKG

SEQ ID NO: 125 - protein, 7 aa, synthetic construct
CYNLETY

SEQ ID NO: 126 - protein, 139 aa, synthetic construct
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAACHFCDSLPKTQDEYILWGQGTQVTVSSAAAYPYDVPDYG

SEQ ID NO: 127 - protein, 147 aa, synthetic construct
  SITE 102: note = fluorosulfonyltyrosine
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAXFADSLPKTQDEYILWGQGTQVTVSSAAAYPYDVPDYGSCHHHHHH

SEQ ID NO: 128 - protein, 10 aa, synthetic construct
GGGGSGGGGS

SEQ ID NO: 129 - protein, 161 aa, synthetic construct
MDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLAAALE